(19) United States
(12) Patent Application Publication
(10) Pub. No.: US 2022/0002436 A1
(43) Pub. Date: Jan. 6, 2022

(54) ANTI-HER2 POLYPEPTIDES AND METHODS OF USE THEREOF

(71) Applicant: Denali Therapeutics Inc., South San Francisco, CA (US)

(72) Inventors: Mark S. Dennis, South San Francisco, CA (US); Wanda Kwan, South San Francisco, CA (US); Joseph W. Lewcock, South San Francisco, CA (US); Jonathan Sockolosky, South San Francisco, CA (US); Joy Yu Zuchero, South San Francisco, CA (US)

(73) Assignee: Denali Therapeutics Inc., South San Francisco, CA (US)

(21) Appl. No.: 17/178,595

(22) Filed: Feb. 18, 2021

Related U.S. Application Data
(63) Continuation of application No. PCT/US2019/047728, filed on Aug. 22, 2019.
(60) Provisional application No. 62/721,505, filed on Aug. 22, 2018.

(51) Int. Cl. C07K 16/32; A61P 35/00

(52) U.S. Cl. CPC C07K 16/32; A61P 35/00; C07K 2319/00; C07K 2317/526; C07K 2317/71; C07K 2317/565

(57) ABSTRACT

The present disclosure relates to anti-HER2 constructs, such as Fc polypeptide dimer-antibody variable region fusion proteins, that cross the BBB and bind to HER2 in the brain parenchyma. In some embodiments, the anti-HER2 constructs (e.g., Fc polypeptide dimer-antibody variable region fusion proteins) retain effector function upon binding to HER2, but do not substantially deplete reticulocytes in vivo. The present disclosure also relates to methods for transcytosing an anti-HER2 antibody variable region across the BBB and treating HER2-positive cancers and metastatic lesions thereof.

The present application is a continuation of International Application No. PCT/US2019/047728, filed on Aug. 22, 2019, which claims priority to U.S. Provisional Patent Application No. 62/721,505, filed Aug. 22, 2018, the disclosure of which are incorporated herein by reference in their entirety for all purposes.

### SEQUENCE LISTING

This application contains a Sequence Listing which has been submitted electronically in ASCII format and is hereby incorporated by reference in its entirety for all purposes. Said ASCII copy, created on Feb. 9, 2021, is named 102342-003210US-1235824_SL.txt and is 834,580 bytes in size.

### BACKGROUND

Treatment of brain metastases of cancers such as breast cancer currently poses a daunting clinical challenge. Among breast cancer patients, the incidence of brain metastases is as high as 50%. Clinical data indicate that there is a proclivity for HER2-positive breast cancers to metastasize to the brain. Notably, anti-HER2 therapies have proven useful for the control of extracranial tumors but not intracranial lesions. The failure of these therapies to control metastatic lesions such as brain metastases of HER2-positive breast cancer is mostly attributed to an inability of the therapeutic agents to cross the blood brain barrier (BBB) and access the brain parenchyma. Thus, there is a need for new therapeutic agents that can cross the BBB and target HER2 in the brain parenchyma.

### SUMMARY

In some aspects, provided herein is an Fc polypeptide dimer-antibody variable region fusion protein comprising: (a) an antibody variable region that is capable of binding human epidermal growth factor receptor 2 (HER2), or an antigen-binding fragment thereof; and (b) a modified Fc polypeptide dimer comprising a first Fc polypeptide that contains amino acid modification(s) to create a TfR-binding site. The antibody variable region may include a heavy chain variable region and a light chain variable region.

In some embodiments, the protein includes (i) two copies of an antibody light chain, (ii) a first heavy chain variable region that is fused to the first Fc polypeptide, and (iii) a second heavy chain variable region that is fused to a second Fc polypeptide. Each light chain may be paired with one of the heavy chain variable regions, the first and second Fc polypeptides may together form the Fc dimer. The first heavy chain variable region may (from N-terminal to C-terminal) be fused to a CH1 domain, which, in turn, is fused to a hinge region, which in turn is fused to the first Fc polypeptide and/or the second heavy chain variable region may (from N-terminal to C-terminal) be fused to a CH1 domain, which, in turn, is fused to a hinge region, which in turn is fused to the second Fc polypeptide.

In some embodiments, the antibody variable region comprises one or more (e.g., all six) complementarity determining regions (CDRs) selected from the group consisting of: (a) a heavy chain CDR1 having at least 90% sequence identity to the amino acid sequence of SEQ ID NO:69 or having up to two amino acid substitutions relative to the amino acid sequence of SEQ ID NO:69; (b) a heavy chain CDR2 having at least 90% sequence identity to the amino acid sequence of SEQ ID NO:70 or having up to two amino acid substitutions relative to the amino acid sequence of SEQ ID NO:70; (c) a heavy chain CDR3 having at least 90% sequence identity to the amino acid sequence of SEQ ID NO:71 or having up to two amino acid substitutions relative to the amino acid sequence of SEQ ID NO:71; (d) a light chain CDR1 having at least 90% sequence identity to the amino acid sequence of SEQ ID NO:72 or having up to two amino acid substitutions relative to the amino acid sequence of SEQ ID NO:72; (e) a light chain CDR2 having up to two amino acid substitutions relative to the amino acid sequence of SEQ ID NO:73; and (f) a light chain CDR3 having up to two amino acid substitutions relative to the amino acid sequence of SEQ ID NO:74.

In some embodiments, the antibody variable region comprises one or more (e.g., all six) CDRs selected from the group consisting of: (a) a heavy chain CDR1 comprising the amino acid sequence of SEQ ID NO:69; (b) a heavy chain CDR2 comprising the amino acid sequence of SEQ ID NO:70; (c) a heavy chain CDR3 comprising the amino acid sequence of SEQ ID NO:71; (d) a light chain CDR1 comprising the amino acid sequence of SEQ ID NO:72; (e) a light chain CDR2 comprising the amino acid sequence of SEQ ID NO:73; and (f) a light chain CDR3 comprising the amino acid sequence of SEQ ID NO:74.

In some embodiments, the antibody variable region comprises two antibody heavy chain variable regions comprising the amino acid sequence of SEQ ID NO:59 and two light chain variable regions comprising the amino acid sequence of SEQ ID NO:60.

In some embodiments, the antibody variable region comprises one or more (e.g., all six) CDRs selected from the group consisting of: (a) a heavy chain CDR1 having at least 90% sequence identity to the amino acid sequence of SEQ ID NO:75 or having up to two amino acid substitutions relative to the amino acid sequence of SEQ ID NO:75; (b) a heavy chain CDR2 having at least 90% sequence identity to the amino acid sequence of SEQ ID NO:76 or having up to two amino acid substitutions relative to the amino acid sequence of SEQ ID NO:76; (c) a heavy chain CDR3 having at least 90% sequence identity to the amino acid sequence of SEQ ID NO:77 or having up to two amino acid substitutions relative to the amino acid sequence of SEQ ID NO:77; (d) a light chain CDR1 having at least 90% sequence identity to the amino acid sequence of SEQ ID NO:78 or having up to two amino acid substitutions relative to the amino acid sequence of SEQ ID NO:78; (e) a light chain CDR2 having up to two amino acid substitutions relative to the amino acid sequence of SEQ ID NO:79; and (f) a light chain CDR3 having up to two amino acid substitutions relative to the amino acid sequence of SEQ ID NO:80.

In some embodiments, the antibody variable region comprises one or more CDRs (e.g., all six) selected from the group consisting of: (a) a heavy chain CDR1 comprising the amino acid sequence of SEQ ID NO:75; (b) a heavy chain CDR2 comprising the amino acid sequence of SEQ ID NO:76; (c) a heavy chain CDR3 comprising the amino acid sequence of SEQ ID NO:77; (d) a light chain CDR1 comprising the amino acid sequence of SEQ ID NO:78; (e) a light chain CDR2 comprising the amino acid sequence of SEQ ID NO:79; and (f) a light chain CDR3 comprising the amino acid sequence of SEQ ID NO:80.

In some embodiments, the antibody variable region comprises two antibody heavy chain variable regions comprising the amino acid sequence of SEQ ID NO:61 and two light chain variable regions comprising the amino acid sequence of SEQ ID NO:62.

In some embodiments, the antibody variable region comprises one or more (e.g., all six) complementarity determining regions (CDRs) selected from the group consisting of: (a) a heavy chain CDR1 having at least 90% sequence identity to the amino acid sequence of SEQ ID NO:250 or having up to two amino acid substitutions relative to the amino acid sequence of SEQ ID NO:250; (b) a heavy chain CDR2 having at least 90% sequence identity to the amino acid sequence of SEQ ID NO:251 or having up to two amino acid substitutions relative to the amino acid sequence of SEQ ID NO:251; (c) a heavy chain CDR3 having at least 90% sequence identity to the amino acid sequence of SEQ ID NO:252 or having up to two amino acid substitutions relative to the amino acid sequence of SEQ ID NO:252; (d) a light chain CDR1 having at least 90% sequence identity to the amino acid sequence of SEQ ID NO:253 or having up to two amino acid substitutions relative to the amino acid sequence of SEQ ID NO:253; (e) a light chain CDR2 having up to two amino acid substitutions relative to the amino acid sequence of SEQ ID NO:254; and (f) a light chain CDR3 having up to two amino acid substitutions relative to the amino acid sequence of SEQ ID NO:255.

In some embodiments, the antibody variable region comprises one or more (e.g., all six) CDRs selected from the group consisting of: (a) a heavy chain CDR1 comprising the amino acid sequence of SEQ ID NO:250; (b) a heavy chain CDR2 comprising the amino acid sequence of SEQ ID NO:251; (c) a heavy chain CDR3 comprising the amino acid sequence of SEQ ID NO:252; (d) a light chain CDR1 comprising the amino acid sequence of SEQ ID NO:253; (e) a light chain CDR2 comprising the amino acid sequence of SEQ ID NO:254; and (f) a light chain CDR3 comprising the amino acid sequence of SEQ ID NO:255.

In some embodiments, the antibody variable region comprises two antibody heavy chain variable regions comprising the amino acid sequence of SEQ ID NO:256 and two light chain variable regions comprising the amino acid sequence of SEQ ID NO:257.

In some embodiments, the TfR-binding site is within a modified CH3 domain. In some embodiments, the modified CH3 domain is derived from a human IgG1, IgG2, IgG3, or IgG4 CH3 domain. In some embodiments, the modified CH3 domain comprises one, two, three, four, five, six, seven, eight, nine, ten, or eleven substitutions in a set of amino acid positions comprising 380, 384, 386, 387, 388, 389, 390, 413, 415, 416, and 421, according to EU numbering. In some embodiments, the modified CH3 domain comprises Glu, Leu, Ser, Val, Trp, Tyr, or Gln at position 380; Leu, Tyr, Phe, Trp, Met, Pro, or Val at position 384; Leu, Thr, His, Pro, Asn, Val, or Phe at position 386; Val, Pro, Ile, or an acidic amino acid at position 387; Trp at position 388; an aliphatic amino acid, Gly, Ser, Thr, or Asn at position 389; Gly, His, Gln, Leu, Lys, Val, Phe, Ser, Ala, Asp, Glu, Asn, Arg, or Thr at position 390; an acidic amino acid, Ala, Ser, Leu, Thr, Pro, Ile, or His at position 413; Glu, Ser, Asp, Gly, Thr, Pro, Gln, or Arg at position 415; Thr, Arg, Asn, or an acidic amino acid at position 416; and/or an aromatic amino acid, His, or Lys at position 421, according to EU numbering.

In some embodiments, the Fc polypeptide dimer-antibody variable region fusion protein binds to the apical domain of TfR. In some embodiments, one of the Fc polypeptides contains amino acid modifications that reduce FcγR binding when the Fc polypeptide dimer is bound to TfR (e.g., but has limited or no reduction of binding when not bound to TfR). These modifications may comprise Ala at position 234 and at position 235 on the first Fc polypeptide, according to EU numbering. In some embodiments, both Fc polypeptides contain the amino acid modifications that reduce FcγR binding (e.g., both polypeptides contain Ala at position 234 and at position 235).

In some embodiments, the Fc polypeptide dimer-antibody variable region fusion is fucose deficient or afucosylated (e.g., as described herein).

In some embodiments, the first Fc polypeptide and/or the second Fc polypeptide comprises amino acid modifications that increase serum half-life. In some embodiments, the amino acid modifications that increase serum half-life comprise (i) a Leu at position 428 and a Ser at position 434, or (ii) a Ser or Ala at position 434, according to EU numbering.

In some embodiments, the first Fc polypeptide further comprises a knob mutation T366W and the second Fc polypeptide comprises hole mutations T366S, L368A, and Y407V, according to EU numbering. In some embodiments, the first Fc polypeptide comprises the amino acid sequence of SEQ ID NO:63. In some embodiments, the second Fc polypeptide comprises the amino acid sequence of any one of SEQ ID NOS:67 and 68.

In other aspects, provided herein is an Fc polypeptide dimer-antibody variable region fusion protein, comprising (a) an antibody variable region that is capable of binding human HER2, or an antigen-binding fragment thereof; (b) a first Fc polypeptide that contains modifications that create a TfR-binding site and a knob mutation T366W, according to EU numbering, and (c) a second Fc polypeptide that comprises hole mutations T366S, L368A, and Y407V, according to EU numbering, and does not contain a TfR-binding site.

In some embodiments, the antibody variable region comprises one or more (e.g., all six) complementarity determining regions (CDRs) selected from the group consisting of: (a) a heavy chain CDR1 having at least 90% sequence identity to the amino acid sequence of SEQ ID NO:69 or having up to two amino acid substitutions relative to the amino acid sequence of SEQ ID NO:69; (b) a heavy chain CDR2 having at least 90% sequence identity to the amino acid sequence of SEQ ID NO:70 or having up to two amino acid substitutions relative to the amino acid sequence of SEQ ID NO:70; (c) a heavy chain CDR3 having at least 90% sequence identity to the amino acid sequence of SEQ ID NO:71 or having up to two amino acid substitutions relative to the amino acid sequence of SEQ ID NO:71; (d) a light chain CDR1 having at least 90% sequence identity to the amino acid sequence of SEQ ID NO:72 or having up to two amino acid substitutions relative to the amino acid sequence of SEQ ID NO:72; (e) a light chain CDR2 having up to two amino acid substitutions relative to the amino acid sequence of SEQ ID NO:73; and (f) a light chain CDR3 having up to two amino acid substitutions relative to the amino acid sequence of SEQ ID NO:74.

In some embodiments, the antibody variable region comprises one or more (e.g., all six) CDRs selected from the group consisting of: (a) a heavy chain CDR1 comprising the amino acid sequence of SEQ ID NO:69; (b) a heavy chain CDR2 comprising the amino acid sequence of SEQ ID NO:70; (c) a heavy chain CDR3 comprising the amino acid sequence of SEQ ID NO:71; (d) a light chain CDR1 comprising the amino acid sequence of SEQ ID NO:72; (e) a light chain CDR2 comprising the amino acid sequence of SEQ ID NO:73; and (f) a light chain CDR3 comprising the amino acid sequence of SEQ ID NO:74.

In some embodiments, the antibody variable region comprises two antibody heavy chain variable regions comprising the amino acid sequence of SEQ ID NO:59 and two light chain variable regions comprising the amino acid sequence of SEQ ID NO:60.

In some embodiments, the antibody variable region comprises one or more (e.g., all six) CDRs selected from the group consisting of: (a) a heavy chain CDR1 having at least 90% sequence identity to the amino acid sequence of SEQ ID NO:75 or having up to two amino acid substitutions relative to the amino acid sequence of SEQ ID NO:75; (b) a heavy chain CDR2 having at least 90% sequence identity to the amino acid sequence of SEQ ID NO:76 or having up to two amino acid substitutions relative to the amino acid sequence of SEQ ID NO:76; (c) a heavy chain CDR3 having at least 90% sequence identity to the amino acid sequence of SEQ ID NO:77 or having up to two amino acid substitutions relative to the amino acid sequence of SEQ ID NO:77; (d) a light chain CDR1 having at least 90% sequence identity to the amino acid sequence of SEQ ID NO:78 or having up to two amino acid substitutions relative to the amino acid sequence of SEQ ID NO:78; (e) a light chain CDR2 having up to two amino acid substitutions relative to the amino acid sequence of SEQ ID NO:79; and (f) a light chain CDR3 having up to two amino acid substitutions relative to the amino acid sequence of SEQ ID NO:80.

In some embodiments, the antibody variable region comprises one or more CDRs (e.g., all six) selected from the group consisting of: (a) a heavy chain CDR1 comprising the amino acid sequence of SEQ ID NO:75; (b) a heavy chain CDR2 comprising the amino acid sequence of SEQ ID NO:76; (c) a heavy chain CDR3 comprising the amino acid sequence of SEQ ID NO:77; (d) a light chain CDR1 comprising the amino acid sequence of SEQ ID NO:78; (e) a light chain CDR2 comprising the amino acid sequence of SEQ ID NO:79; and (f) a light chain CDR3 comprising the amino acid sequence of SEQ ID NO:80.

In some embodiments, the antibody variable region comprises two antibody heavy chain variable regions comprising the amino acid sequence of SEQ ID NO:61 and two light chain variable regions comprising the amino acid sequence of SEQ ID NO:62.

In some embodiments, the antibody variable region comprises one or more (e.g., all six) complementarity determining regions (CDRs) selected from the group consisting of: (a) a heavy chain CDR1 having at least 90% sequence identity to the amino acid sequence of SEQ ID NO:250 or having up to two amino acid substitutions relative to the amino acid sequence of SEQ ID NO:250; (b) a heavy chain CDR2 having at least 90% sequence identity to the amino acid sequence of SEQ ID NO:251 or having up to two amino acid substitutions relative to the amino acid sequence of SEQ ID NO:251; (c) a heavy chain CDR3 having at least 90% sequence identity to the amino acid sequence of SEQ ID NO:252 or having up to two amino acid substitutions relative to the amino acid sequence of SEQ ID NO:252; (d) a light chain CDR1 having at least 90% sequence identity to the amino acid sequence of SEQ ID NO:253 or having up to two amino acid substitutions relative to the amino acid sequence of SEQ ID NO:253; (e) a light chain CDR2 having up to two amino acid substitutions relative to the amino acid sequence of SEQ ID NO:254; and (f) a light chain CDR3 having up to two amino acid substitutions relative to the amino acid sequence of SEQ ID NO:255.

In some embodiments, the antibody variable region comprises one or more (e.g., all six) CDRs selected from the group consisting of: (a) a heavy chain CDR1 comprising the amino acid sequence of SEQ ID NO:250; (b) a heavy chain CDR2 comprising the amino acid sequence of SEQ ID NO:251; (c) a heavy chain CDR3 comprising the amino acid sequence of SEQ ID NO:252; (d) a light chain CDR1 comprising the amino acid sequence of SEQ ID NO:253; (e) a light chain CDR2 comprising the amino acid sequence of SEQ ID NO:254; and (f) a light chain CDR3 comprising the amino acid sequence of SEQ ID NO:255.

In some embodiments, the antibody variable region comprises two antibody heavy chain variable regions comprising the amino acid sequence of SEQ ID NO:256 and two light chain variable regions comprising the amino acid sequence of SEQ ID NO:257.

In some embodiments, the Fc polypeptide dimer-antibody variable region fusion protein comprises a first heavy chain comprising the amino acid sequence of any one of SEQ ID NOS:1, 9, 17, and 81. In some embodiments, the first Fc polypeptide further comprises amino acid modifications L234A and L235A. In some embodiments, the Fc polypeptide dimer-antibody variable region fusion protein comprises a first heavy chain comprising the amino acid sequence of any one of SEQ ID NOS:2, 10, 18, and 82. In some embodiments, the Fc polypeptide dimer-antibody variable region fusion protein comprises a second heavy chain comprising the amino acid sequence of SEQ ID NO:27. In some embodiments, the Fc polypeptide dimer-antibody variable region fusion protein comprises two light chains comprising the amino acid sequence of SEQ ID NO:57.

In some embodiments, the Fc polypeptide dimer-antibody variable region fusion protein comprises a first heavy chain comprising the amino acid sequence of any one of SEQ ID NOS:29, 37, 45, and 89. In some embodiments, the first Fc polypeptide further comprises amino acid modifications L234A and L235A. In some embodiments, the Fc polypeptide dimer-antibody variable region fusion protein comprises a first heavy chain comprising the amino acid sequence of any one of SEQ ID NOS:30, 38, 46, and 90. In some embodiments, the Fc polypeptide dimer-antibody variable region fusion protein comprises a second heavy chain comprising the amino acid sequence of SEQ ID NO:55. In some embodiments, the Fc polypeptide dimer-antibody variable region fusion protein comprises two light chains comprising the amino acid sequence of SEQ ID NO:58.

In some embodiments, the Fc polypeptide dimer-antibody variable region fusion protein comprises a first heavy chain comprising the amino acid sequence of any one of SEQ ID NOS:258, 266, 274, and 282. In some embodiments, the first Fc polypeptide further comprises amino acid modifications L234A and L235A. In some embodiments, the Fc polypeptide dimer-antibody variable region fusion protein comprises a first heavy chain comprising the amino acid sequence of any one of SEQ ID NOS:259, 267, 275, and 283. In some embodiments, the Fc polypeptide dimer-antibody variable region fusion protein comprises a second heavy chain comprising the amino acid sequence of SEQ ID NO:290. In some embodiments, the Fc polypeptide dimer-antibody variable region fusion protein comprises two light chains comprising the amino acid sequence of SEQ ID NO:293.

In other aspects, provided herein is an Fc polypeptide dimer-antibody variable region fusion protein, comprising: (a) an antibody variable region that is capable of binding human HER2, or an antigen-binding fragment thereof; (b) a first Fc polypeptide that contains modifications that create a TfR-binding site, a knob mutation T366W, and amino acid modification N434S with or without M428L, according to EU numbering, and (c) a second Fc polypeptide that comprises hole mutations T366S, L368A, and Y407V, according to EU numbering, and does not contain a TfR-binding site.

In some embodiments, the antibody variable region comprises one or more (e.g., all six) complementarity determining regions (CDRs) selected from the group consisting of: (a) a heavy chain CDR1 having at least 90% sequence identity to the amino acid sequence of SEQ ID NO:69 or having up to two amino acid substitutions relative to the amino acid sequence of SEQ ID NO:69; (b) a heavy chain CDR2 having at least 90% sequence identity to the amino acid sequence of SEQ ID NO:70 or having up to two amino acid substitutions relative to the amino acid sequence of SEQ ID NO:70; (c) a heavy chain CDR3 having at least 90% sequence identity to the amino acid sequence of SEQ ID NO:71 or having up to two amino acid substitutions relative to the amino acid sequence of SEQ ID NO:71; (d) a light chain CDR1 having at least 90% sequence identity to the amino acid sequence of SEQ ID NO:72 or having up to two amino acid substitutions relative to the amino acid sequence of SEQ ID NO:72; (e) a light chain CDR2 having up to two amino acid substitutions relative to the amino acid sequence of SEQ ID NO:73; and (f) a light chain CDR3 having up to two amino acid substitutions relative to the amino acid sequence of SEQ ID NO:74.

In some embodiments, the antibody variable region comprises one or more (e.g., all six) CDRs selected from the group consisting of: (a) a heavy chain CDR1 comprising the amino acid sequence of SEQ ID NO:69; (b) a heavy chain CDR2 comprising the amino acid sequence of SEQ ID NO:70; (c) a heavy chain CDR3 comprising the amino acid sequence of SEQ ID NO:71; (d) a light chain CDR1 comprising the amino acid sequence of SEQ ID NO:72; (e) a light chain CDR2 comprising the amino acid sequence of SEQ ID NO:73; and (f) a light chain CDR3 comprising the amino acid sequence of SEQ ID NO:74.

In some embodiments, the antibody variable region comprises two antibody heavy chain variable regions comprising the amino acid sequence of SEQ ID NO:59 and two light chain variable regions comprising the amino acid sequence of SEQ ID NO:60.

In some embodiments, the antibody variable region comprises one or more (e.g., all six) CDRs selected from the group consisting of: (a) a heavy chain CDR1 having at least 90% sequence identity to the amino acid sequence of SEQ ID NO:75 or having up to two amino acid substitutions relative to the amino acid sequence of SEQ ID NO:75; (b) a heavy chain CDR2 having at least 90% sequence identity to the amino acid sequence of SEQ ID NO:76 or having up to two amino acid substitutions relative to the amino acid sequence of SEQ ID NO:76; (c) a heavy chain CDR3 having at least 90% sequence identity to the amino acid sequence of SEQ ID NO:77 or having up to two amino acid substitutions relative to the amino acid sequence of SEQ ID NO:77; (d) a light chain CDR1 having at least 90% sequence identity to the amino acid sequence of SEQ ID NO:78 or having up to two amino acid substitutions relative to the amino acid sequence of SEQ ID NO:78; (e) a light chain CDR2 having up to two amino acid substitutions relative to the amino acid sequence of SEQ ID NO:79; and (f) a light chain CDR3 having up to two amino acid substitutions relative to the amino acid sequence of SEQ ID NO:80.

In some embodiments, the antibody variable region comprises one or more CDRs (e.g., all six) selected from the group consisting of: (a) a heavy chain CDR1 comprising the amino acid sequence of SEQ ID NO:75; (b) a heavy chain CDR2 comprising the amino acid sequence of SEQ ID NO:76; (c) a heavy chain CDR3 comprising the amino acid sequence of SEQ ID NO:77; (d) a light chain CDR1 comprising the amino acid sequence of SEQ ID NO:78; (e) a light chain CDR2 comprising the amino acid sequence of SEQ ID NO:79; and (f) a light chain CDR3 comprising the amino acid sequence of SEQ ID NO:80.

In some embodiments, the antibody variable region comprises two antibody heavy chain variable regions comprising the amino acid sequence of SEQ ID NO:61 and two light chain variable regions comprising the amino acid sequence of SEQ ID NO:62.

In some embodiments, the antibody variable region comprises one or more (e.g., all six) complementarity determining regions (CDRs) selected from the group consisting of: (a) a heavy chain CDR1 having at least 90% sequence identity to the amino acid sequence of SEQ ID NO:250 or having up to two amino acid substitutions relative to the amino acid sequence of SEQ ID NO:250; (b) a heavy chain CDR2 having at least 90% sequence identity to the amino acid sequence of SEQ ID NO:251 or having up to two amino acid substitutions relative to the amino acid sequence of SEQ ID NO:251; (c) a heavy chain CDR3 having at least 90% sequence identity to the amino acid sequence of SEQ ID NO:252 or having up to two amino acid substitutions relative to the amino acid sequence of SEQ ID NO:252; (d) a light chain CDR1 having at least 90% sequence identity to the amino acid sequence of SEQ ID NO:253 or having up to two amino acid substitutions relative to the amino acid sequence of SEQ ID NO:253; (e) a light chain CDR2 having up to two amino acid substitutions relative to the amino acid sequence of SEQ ID NO:254; and (f) a light chain CDR3 having up to two amino acid substitutions relative to the amino acid sequence of SEQ ID NO:255.

In some embodiments, the antibody variable region comprises one or more (e.g., all six) CDRs selected from the group consisting of: (a) a heavy chain CDR1 comprising the amino acid sequence of SEQ ID NO:250; (b) a heavy chain CDR2 comprising the amino acid sequence of SEQ ID NO:251; (c) a heavy chain CDR3 comprising the amino acid sequence of SEQ ID NO:252; (d) a light chain CDR1 comprising the amino acid sequence of SEQ ID NO:253; (e) a light chain CDR2 comprising the amino acid sequence of SEQ ID NO:254; and (f) a light chain CDR3 comprising the amino acid sequence of SEQ ID NO:255.

In some embodiments, the antibody variable region comprises two antibody heavy chain variable regions comprising the amino acid sequence of SEQ ID NO:256 and two light chain variable regions comprising the amino acid sequence of SEQ ID NO:257.

In some embodiments, the Fc polypeptide dimer-antibody variable region fusion protein comprises a first heavy chain comprising the amino acid sequence of any one of SEQ ID NOS:3, 11, 19, and 83. In some embodiments, the first Fc polypeptide further comprises amino acid modifications L234A and L235A. In some embodiments, the Fc polypeptide dimer-antibody variable region fusion protein comprises a first heavy chain comprising the amino acid sequence of any one of SEQ ID NOS:4, 12, 20, and 84. In some embodiments, the Fc polypeptide dimer-antibody variable region fusion protein comprises a second heavy chain comprising the amino acid sequence of SEQ ID NO:27. In some embodiments, the Fc polypeptide dimer-antibody variable region fusion protein comprises two light chains comprising the amino acid sequence of SEQ ID NO:57.

In some embodiments, the Fc polypeptide dimer-antibody variable region fusion protein comprises a first heavy chain comprising the amino acid sequence of any one of SEQ ID NOS:31, 39, 47, and 91. In some embodiments, the first Fc polypeptide further comprises amino acid modifications L234A and L235A. In some embodiments, the Fc polypeptide dimer-antibody variable region fusion protein comprises a first heavy chain comprising the amino acid sequence of any one of SEQ ID NOS:32, 40, 48, and 92. In some embodiments, the Fc polypeptide dimer-antibody variable region fusion protein comprises a second heavy chain comprising the amino acid sequence of SEQ ID NO:55. In some embodiments, the Fc polypeptide dimer-antibody variable region fusion protein comprises two light chains comprising the amino acid sequence of SEQ ID NO:58.

In some embodiments, the Fc polypeptide dimer-antibody variable region fusion protein comprises a first heavy chain comprising the amino acid sequence of any one of SEQ ID NOS:260, 268, 276, and 284. In some embodiments, the first Fc polypeptide further comprises amino acid modifications L234A and L235A. In some embodiments, the Fc polypeptide dimer-antibody variable region fusion protein comprises a first heavy chain comprising the amino acid sequence of any one of SEQ ID NOS:261, 269, 277, and 285. In some embodiments, the Fc polypeptide dimer-antibody variable region fusion protein comprises a second heavy chain comprising the amino acid sequence of SEQ ID NO:290. In some embodiments, the Fc polypeptide dimer-antibody variable region fusion protein comprises two light chains comprising the amino acid sequence of SEQ ID NO:293.

In other aspects, provided herein is an Fc polypeptide dimer-antibody variable region fusion protein, comprising: (a) an antibody variable region that is capable of binding human HER2, or an antigen-binding fragment thereof; (b) a first Fc polypeptide that contains modifications that create a TfR-binding site and a knob mutation T366W, according to EU numbering, and (c) a second Fc polypeptide that comprises hole mutations T366S, L368A, and Y407V and amino acid modification N434S with or without M428L, according to EU numbering, and does not contain a TfR-binding site.

In some embodiments, the antibody variable region comprises one or more (e.g., all six) complementarity determining regions (CDRs) selected from the group consisting of: (a) a heavy chain CDR1 having at least 90% sequence identity to the amino acid sequence of SEQ ID NO:69 or having up to two amino acid substitutions relative to the amino acid sequence of SEQ ID NO:69; (b) a heavy chain CDR2 having at least 90% sequence identity to the amino acid sequence of SEQ ID NO:70 or having up to two amino acid substitutions relative to the amino acid sequence of SEQ ID NO:70; (c) a heavy chain CDR3 having at least 90% sequence identity to the amino acid sequence of SEQ ID NO:71 or having up to two amino acid substitutions relative to the amino acid sequence of SEQ ID NO:71; (d) a light chain CDR1 having at least 90% sequence identity to the amino acid sequence of SEQ ID NO:72 or having up to two amino acid substitutions relative to the amino acid sequence of SEQ ID NO:72; (e) a light chain CDR2 having up to two amino acid substitutions relative to the amino acid sequence of SEQ ID NO:73; and (f) a light chain CDR3 having up to two amino acid substitutions relative to the amino acid sequence of SEQ ID NO:74.

In some embodiments, the antibody variable region comprises one or more (e.g., all six) CDRs selected from the group consisting of: (a) a heavy chain CDR1 comprising the amino acid sequence of SEQ ID NO:69; (b) a heavy chain CDR2 comprising the amino acid sequence of SEQ ID NO:70; (c) a heavy chain CDR3 comprising the amino acid sequence of SEQ ID NO:71; (d) a light chain CDR1 comprising the amino acid sequence of SEQ ID NO:72; (e) a light chain CDR2 comprising the amino acid sequence of SEQ ID NO:73; and (f) a light chain CDR3 comprising the amino acid sequence of SEQ ID NO:74.

In some embodiments, the antibody variable region comprises two antibody heavy chain variable regions comprising the amino acid sequence of SEQ ID NO:59 and two light chain variable regions comprising the amino acid sequence of SEQ ID NO:60.

In some embodiments, the antibody variable region comprises one or more (e.g., all six) CDRs selected from the group consisting of: (a) a heavy chain CDR1 having at least 90% sequence identity to the amino acid sequence of SEQ ID NO:75 or having up to two amino acid substitutions relative to the amino acid sequence of SEQ ID NO:75; (b) a heavy chain CDR2 having at least 90% sequence identity to the amino acid sequence of SEQ ID NO:76 or having up to two amino acid substitutions relative to the amino acid sequence of SEQ ID NO:76; (c) a heavy chain CDR3 having at least 90% sequence identity to the amino acid sequence of SEQ ID NO:77 or having up to two amino acid substitutions relative to the amino acid sequence of SEQ ID NO:77; (d) a light chain CDR1 having at least 90% sequence identity to the amino acid sequence of SEQ ID NO:78 or having up to two amino acid substitutions relative to the amino acid sequence of SEQ ID NO:78; (e) a light chain CDR2 having up to two amino acid substitutions relative to the amino acid sequence of SEQ ID NO:79; and (f) a light chain CDR3 having up to two amino acid substitutions relative to the amino acid sequence of SEQ ID NO:80.

In some embodiments, the antibody variable region comprises one or more CDRs (e.g., all six) selected from the group consisting of: (a) a heavy chain CDR1 comprising the amino acid sequence of SEQ ID NO:75; (b) a heavy chain CDR2 comprising the amino acid sequence of SEQ ID NO:76; (c) a heavy chain CDR3 comprising the amino acid sequence of SEQ ID NO:77; (d) a light chain CDR1 comprising the amino acid sequence of SEQ ID NO:78; (e) a light chain CDR2 comprising the amino acid sequence of SEQ ID NO:79; and (f) a light chain CDR3 comprising the amino acid sequence of SEQ ID NO:80.

In some embodiments, the antibody variable region comprises two antibody heavy chain variable regions comprising the amino acid sequence of SEQ ID NO:61 and two light chain variable regions comprising the amino acid sequence of SEQ ID NO:62.

In some embodiments, the antibody variable region comprises one or more (e.g., all six) complementarity determining regions (CDRs) selected from the group consisting of: (a) a heavy chain CDR1 having at least 90% sequence identity to the amino acid sequence of SEQ ID NO:250 or having up to two amino acid substitutions relative to the amino acid sequence of SEQ ID NO:250; (b) a heavy chain CDR2 having at least 90% sequence identity to the amino acid sequence of SEQ ID NO:251 or having up to two amino acid substitutions relative to the amino acid sequence of SEQ ID NO:251; (c) a heavy chain CDR3 having at least 90% sequence identity to the amino acid sequence of SEQ ID NO:252 or having up to two amino acid substitutions relative to the amino acid sequence of SEQ ID NO:252; (d) a light chain CDR1 having at least 90% sequence identity to the amino acid sequence of SEQ ID NO:253 or having up to two amino acid substitutions relative to the amino acid sequence of SEQ ID NO:253; (e) a light chain CDR2 having up to two amino acid substitutions relative to the amino acid sequence of SEQ ID NO:254; and (f) a light chain CDR3 having up to two amino acid substitutions relative to the amino acid sequence of SEQ ID NO:255.

In some embodiments, the antibody variable region comprises one or more (e.g., all six) CDRs selected from the group consisting of: (a) a heavy chain CDR1 comprising the amino acid sequence of SEQ ID NO:250; (b) a heavy chain CDR2 comprising the amino acid sequence of SEQ ID NO:251; (c) a heavy chain CDR3 comprising the amino acid sequence of SEQ ID NO:252; (d) a light chain CDR1 comprising the amino acid sequence of SEQ ID NO:253; (e) a light chain CDR2 comprising the amino acid sequence of SEQ ID NO:254; and (f) a light chain CDR3 comprising the amino acid sequence of SEQ ID NO:255.

In some embodiments, the antibody variable region comprises two antibody heavy chain variable regions comprising the amino acid sequence of SEQ ID NO:256 and two light chain variable regions comprising the amino acid sequence of SEQ ID NO:257.

In some embodiments, the Fc polypeptide dimer-antibody variable region fusion protein comprises a first heavy chain comprising the amino acid sequence of any one of SEQ ID NOS:1, 9, 17, and 81. In some embodiments, the first Fc polypeptide further comprises amino acid modifications L234A and L235A. In some embodiments, the Fc polypeptide dimer-antibody variable region fusion protein comprises a first heavy chain comprising the amino acid sequence of any one of SEQ ID NOS:2, 10, 18, and 82. In some embodiments, the Fc polypeptide dimer-antibody variable region fusion protein comprises a second heavy chain comprising the amino acid sequence of SEQ ID NO:28. In some embodiments, the Fc polypeptide dimer-antibody variable region fusion protein comprises two light chains comprising the amino acid sequence of SEQ ID NO:57.

In some embodiments, the Fc polypeptide dimer-antibody variable region fusion protein comprises a first heavy chain comprising the amino acid sequence of any one of SEQ ID NOS:29, 37, 45, and 89. In some embodiments, the first Fc polypeptide further comprises amino acid modifications L234A and L235A. In some embodiments, the Fc polypeptide dimer-antibody variable region fusion protein comprises a first heavy chain comprising the amino acid sequence of any one of SEQ ID NOS:30, 38, 46, and 90. In some embodiments, the Fc polypeptide dimer-antibody variable region fusion protein comprises a second heavy chain comprising the amino acid sequence of SEQ ID NO:56. In some embodiments, the Fc polypeptide dimer-antibody variable region fusion protein comprises two light chains comprising the amino acid sequence of SEQ ID NO:58.

In some embodiments, the Fc polypeptide dimer-antibody variable region fusion protein comprises a first heavy chain comprising the amino acid sequence of any one of SEQ ID NOS:258, 266, 274, and 282. In some embodiments, the first Fc polypeptide further comprises amino acid modifications L234A and L235A. In some embodiments, the Fc polypeptide dimer-antibody variable region fusion protein comprises a first heavy chain comprising the amino acid sequence of any one of SEQ ID NOS:259, 267, 275, and 283. In some embodiments, the Fc polypeptide dimer-antibody variable region fusion protein comprises a second heavy chain comprising the amino acid sequence of SEQ ID NO:291. In some embodiments, the Fc polypeptide dimer-antibody variable region fusion protein comprises two light chains comprising the amino acid sequence of SEQ ID NO:293.

In other aspects, provided herein is an Fc polypeptide dimer-antibody variable region fusion protein, comprising: (a) an antibody variable region that is capable of binding human HER2, or an antigen-binding fragment thereof; (b) a first Fc polypeptide that contains modifications that create a TfR-binding site, a knob mutation T366W, and amino acid modification N434S with or without M428L, according to EU numbering, and (c) a second Fc polypeptide that comprises hole mutations T366S, L368A, and Y407V and amino acid modification N434S with or without M428L, according to EU numbering, and does not contain a TfR-binding site.

In some embodiments, the antibody variable region comprises one or more (e.g., all six) complementarity determining regions (CDRs) selected from the group consisting of: (a) a heavy chain CDR1 having at least 90% sequence identity to the amino acid sequence of SEQ ID NO:69 or having up to two amino acid substitutions relative to the amino acid sequence of SEQ ID NO:69; (b) a heavy chain CDR2 having at least 90% sequence identity to the amino acid sequence of SEQ ID NO:70 or having up to two amino acid substitutions relative to the amino acid sequence of SEQ ID NO:70; (c) a heavy chain CDR3 having at least 90% sequence identity to the amino acid sequence of SEQ ID NO:71 or having up to two amino acid substitutions relative to the amino acid sequence of SEQ ID NO:71; (d) a light chain CDR1 having at least 90% sequence identity to the amino acid sequence of SEQ ID NO:72 or having up to two amino acid substitutions relative to the amino acid sequence of SEQ ID NO:72; (e) a light chain CDR2 having up to two amino acid substitutions relative to the amino acid sequence of SEQ ID NO:73; and (f) a light chain CDR3 having up to two amino acid substitutions relative to the amino acid sequence of SEQ ID NO:74.

In some embodiments, the antibody variable region comprises one or more (e.g., all six) CDRs selected from the group consisting of: (a) a heavy chain CDR1 comprising the amino acid sequence of SEQ ID NO:69; (b) a heavy chain CDR2 comprising the amino acid sequence of SEQ ID NO:70; (c) a heavy chain CDR3 comprising the amino acid sequence of SEQ ID NO:71; (d) a light chain CDR1 comprising the amino acid sequence of SEQ ID NO:72; (e) a light chain CDR2 comprising the amino acid sequence of SEQ ID NO:73; and (f) a light chain CDR3 comprising the amino acid sequence of SEQ ID NO:74.

In some embodiments, the antibody variable region comprises two antibody heavy chain variable regions comprising the amino acid sequence of SEQ ID NO:59 and two light chain variable regions comprising the amino acid sequence of SEQ ID NO:60.

In some embodiments, the antibody variable region comprises one or more (e.g., all six) CDRs selected from the group consisting of: (a) a heavy chain CDR1 having at least 90% sequence identity to the amino acid sequence of SEQ ID NO:75 or having up to two amino acid substitutions relative to the amino acid sequence of SEQ ID NO:75; (b) a heavy chain CDR2 having at least 90% sequence identity to the amino acid sequence of SEQ ID NO:76 or having up to two amino acid substitutions relative to the amino acid sequence of SEQ ID NO:76; (c) a heavy chain CDR3 having at least 90% sequence identity to the amino acid sequence of SEQ ID NO:77 or having up to two amino acid substitutions relative to the amino acid sequence of SEQ ID NO:77; (d) a light chain CDR1 having at least 90% sequence identity to the amino acid sequence of SEQ ID NO:78 or having up to two amino acid substitutions relative to the amino acid sequence of SEQ ID NO:78; (e) a light chain CDR2 having up to two amino acid substitutions relative to the amino acid sequence of SEQ ID NO:79; and (f) a light chain CDR3 having up to two amino acid substitutions relative to the amino acid sequence of SEQ ID NO:80.

In some embodiments, the antibody variable region comprises one or more CDRs (e.g., all six) selected from the group consisting of: (a) a heavy chain CDR1 comprising the amino acid sequence of SEQ ID NO:75; (b) a heavy chain CDR2 comprising the amino acid sequence of SEQ ID NO:76; (c) a heavy chain CDR3 comprising the amino acid sequence of SEQ ID NO:77; (d) a light chain CDR1 comprising the amino acid sequence of SEQ ID NO:78; (e) a light chain CDR2 comprising the amino acid sequence of SEQ ID NO:79; and (f) a light chain CDR3 comprising the amino acid sequence of SEQ ID NO:80.

In some embodiments, the antibody variable region comprises two antibody heavy chain variable regions comprising the amino acid sequence of SEQ ID NO:61 and two light chain variable regions comprising the amino acid sequence of SEQ ID NO:62.

In some embodiments, the antibody variable region comprises one or more (e.g., all six) complementarity determining regions (CDRs) selected from the group consisting of: (a) a heavy chain CDR1 having at least 90% sequence identity to the amino acid sequence of SEQ ID NO:250 or having up to two amino acid substitutions relative to the amino acid sequence of SEQ ID NO:250; (b) a heavy chain CDR2 having at least 90% sequence identity to the amino acid sequence of SEQ ID NO:251 or having up to two amino acid substitutions relative to the amino acid sequence of SEQ ID NO:251; (c) a heavy chain CDR3 having at least 90% sequence identity to the amino acid sequence of SEQ ID NO:252 or having up to two amino acid substitutions relative to the amino acid sequence of SEQ ID NO:252; (d) a light chain CDR1 having at least 90% sequence identity to the amino acid sequence of SEQ ID NO:253 or having up to two amino acid substitutions relative to the amino acid sequence of SEQ ID NO:253; (e) a light chain CDR2 having up to two amino acid substitutions relative to the amino acid sequence of SEQ ID NO:254; and (f) a light chain CDR3 having up to two amino acid substitutions relative to the amino acid sequence of SEQ ID NO:255.

In some embodiments, the antibody variable region comprises one or more (e.g., all six) CDRs selected from the group consisting of: (a) a heavy chain CDR1 comprising the amino acid sequence of SEQ ID NO:250; (b) a heavy chain CDR2 comprising the amino acid sequence of SEQ ID NO:251; (c) a heavy chain CDR3 comprising the amino acid sequence of SEQ ID NO:252; (d) a light chain CDR1 comprising the amino acid sequence of SEQ ID NO:253; (e) a light chain CDR2 comprising the amino acid sequence of SEQ ID NO:254; and (f) a light chain CDR3 comprising the amino acid sequence of SEQ ID NO:255.

In some embodiments, the antibody variable region comprises two antibody heavy chain variable regions comprising the amino acid sequence of SEQ ID NO:256 and two light chain variable regions comprising the amino acid sequence of SEQ ID NO:257.

In some embodiments, the Fc polypeptide dimer-antibody variable region fusion protein comprises a first heavy chain comprising the amino acid sequence of any one of SEQ ID NOS:3, 11, 19, and 83. In some embodiments, the first Fc polypeptide further comprises amino acid modifications L234A and L235A. In some embodiments, the Fc polypeptide dimer-antibody variable region fusion protein comprises a first heavy chain comprising the amino acid sequence of any one of SEQ ID NOS:4, 12, 20, and 84. In some embodiments, the Fc polypeptide dimer-antibody variable region fusion protein comprises a second heavy chain comprising the amino acid sequence of SEQ ID NO:28. In some embodiments, the Fc polypeptide dimer-antibody variable region fusion protein comprises two light chains comprising the amino acid sequence of SEQ ID NO:57.

In some embodiments, the Fc polypeptide dimer-antibody variable region fusion protein comprises a first heavy chain comprising the amino acid sequence of any one of SEQ ID NOS:31, 39, 47, and 91. In some embodiments, the first Fc polypeptide further comprises amino acid modifications L234A and L235A. In some embodiments, the Fc polypeptide dimer-antibody variable region fusion protein comprises a first heavy chain comprising the amino acid sequence of any one of SEQ ID NOS:32, 40, 48, and 92. In some embodiments, the Fc polypeptide dimer-antibody variable region fusion protein comprises a second heavy chain comprising the amino acid sequence of SEQ ID NO:56. In some embodiments, the Fc polypeptide dimer-antibody variable region fusion protein comprises two light chains comprising the amino acid sequence of SEQ ID NO:58.

In some embodiments, the Fc polypeptide dimer-antibody variable region fusion protein comprises a first heavy chain comprising the amino acid sequence of any one of SEQ ID NOS:260, 268, 276, and 284. In some embodiments, the first Fc polypeptide further comprises amino acid modifications L234A and L235A. In some embodiments, the Fc polypeptide dimer-antibody variable region fusion protein comprises a first heavy chain comprising the amino acid sequence of any one of SEQ ID NOS:261, 269, 277, and 285. In some embodiments, the Fc polypeptide dimer-antibody variable region fusion protein comprises a second heavy chain comprising the amino acid sequence of SEQ ID NO:291. In some embodiments, the Fc polypeptide dimer-antibody variable region fusion protein comprises two light chains comprising the amino acid sequence of SEQ ID NO:293.

In some embodiments, the modified Fc polypeptide dimer does not substantially deplete reticulocytes. In some embodiments, an amount of reticulocytes depleted after administering the Fc polypeptide dimer-antibody variable region fusion protein is less than an amount of reticulocytes depleted after administering a control. In some embodiments, the control is a corresponding TfR-binding polypeptide dimer-antibody variable region fusion protein with full effector function and/or contains no mutations that reduce FcγR binding.

In other aspects, provided herein is an antibody heavy chain comprising: (a) an anti-human HER2 antibody heavy chain variable region, or a fragment thereof, and (b) a modified Fc polypeptide that contains modifications that create a TfR-binding site. In some embodiments, the modified Fc polypeptide includes one or more amino acid modifications that reduce FcγR binding when bound to TfR.

In some embodiments, the antibody heavy chain variable region comprises one or more CDRs selected from the group consisting of: (a) a heavy chain CDR1 having at least 90% sequence identity to the amino acid sequence of SEQ ID NO:69 or having up to two amino acid substitutions relative to the amino acid sequence of SEQ ID NO:69; (b) a heavy chain CDR2 having at least 90% sequence identity to the amino acid sequence of SEQ ID NO:70 or having up to two amino acid substitutions relative to the amino acid sequence of SEQ ID NO:70; and (c) a heavy chain CDR3 having at least 90% sequence identity to the amino acid sequence of SEQ ID NO:71 or having up to two amino acid substitutions relative to the amino acid sequence of SEQ ID NO:71. In some embodiments, the antibody heavy chain variable region comprises one or more CDRs selected from the group consisting of: (a) a heavy chain CDR1 comprising the amino acid sequence of SEQ ID NO:69; (b) a heavy chain CDR2 comprising the amino acid sequence of SEQ ID NO:70; and (c) a heavy chain CDR3 comprising the amino acid sequence of SEQ ID NO:71. In some embodiments, the antibody heavy chain variable region comprises the amino acid sequence of SEQ ID NO:59.

In some embodiments, the antibody variable region comprises one or more CDRs selected from the group consisting of: (a) a heavy chain CDR1 having at least 90% sequence identity to the amino acid sequence of SEQ ID NO:75 or having up to two amino acid substitutions relative to the amino acid sequence of SEQ ID NO:75; (b) a heavy chain CDR2 having at least 90% sequence identity to the amino acid sequence of SEQ ID NO:76 or having up to two amino acid substitutions relative to the amino acid sequence of SEQ ID NO:76; and (c) a heavy chain CDR3 having at least 90% sequence identity to the amino acid sequence of SEQ ID NO:77 or having up to two amino acid substitutions relative to the amino acid sequence of SEQ ID NO:77. In some embodiments, the antibody variable region comprises one or more CDRs selected from the group consisting of: (a) a heavy chain CDR1 comprising the amino acid sequence of SEQ ID NO:75; (b) a heavy chain CDR2 comprising the amino acid sequence of SEQ ID NO:76; and (c) a heavy chain CDR3 comprising the amino acid sequence of SEQ ID NO:77. In some embodiments, the antibody heavy chain variable region comprises the amino acid sequence of SEQ ID NO:61.

In some embodiments, the antibody variable region comprises one or more CDRs selected from the group consisting of: (a) a heavy chain CDR1 having at least 90% sequence identity to the amino acid sequence of SEQ ID NO:250 or having up to two amino acid substitutions relative to the amino acid sequence of SEQ ID NO:250; (b) a heavy chain CDR2 having at least 90% sequence identity to the amino acid sequence of SEQ ID NO:251 or having up to two amino acid substitutions relative to the amino acid sequence of SEQ ID NO:251; and (c) a heavy chain CDR3 having at least 90% sequence identity to the amino acid sequence of SEQ ID NO:252 or having up to two amino acid substitutions relative to the amino acid sequence of SEQ ID NO:252. In some embodiments, the antibody variable region comprises one or more CDRs selected from the group consisting of: (a) a heavy chain CDR1 comprising the amino acid sequence of SEQ ID NO:250; (b) a heavy chain CDR2 comprising the amino acid sequence of SEQ ID NO:251; and (c) a heavy chain CDR3 comprising the amino acid sequence of SEQ ID NO:252. In some embodiments, the antibody heavy chain variable region comprises the amino acid sequence of SEQ ID NO:256.

In some embodiments, the TfR-binding site is within a modified CH3 domain. In some embodiments, the modified CH3 domain is derived from a human IgG1, IgG2, IgG3, or IgG4 CH3 domain. In some embodiments, the modified CH3 domain comprises one, two, three, four, five, six, seven, eight, nine, ten, or eleven substitutions in a set of amino acid positions comprising 380, 384, 386, 387, 388, 389, 390, 413, 415, 416, and 421, according to EU numbering. In some embodiments, the modified CH3 domain comprises Glu, Leu, Ser, Val, Trp, Tyr, or Gln at position 380; Leu, Tyr, Phe, Trp, Met, Pro, or Val at position 384; Leu, Thr, His, Pro, Asn, Val, or Phe at position 386; Val, Pro, Ile, or an acidic amino acid at position 387; Trp at position 388; an aliphatic amino acid, Gly, Ser, Thr, or Asn at position 389; Gly, His, Gln, Leu, Lys, Val, Phe, Ser, Ala, Asp, Glu, Asn, Arg, or Thr at position 390; an acidic amino acid, Ala, Ser, Leu, Thr, Pro, Ile, or His at position 413; Glu, Ser, Asp, Gly, Thr, Pro, Gln, or Arg at position 415; Thr, Arg, Asn, or an acidic amino acid at position 416; and/or an aromatic amino acid, His, or Lys at position 421, according to EU numbering.

In some embodiments, the amino acid modifications that reduce FcγR binding when bound to TfR comprise Ala at position 234 and at position 235, according to EU numbering. In some embodiments, the modified Fc polypeptide further comprises amino acid modifications that increase serum half-life. In some embodiments, the amino acid modifications that increase serum half-life comprise (i) a Leu at position 428 and a Ser at position 434, or (ii) a Ser or Ala at position 434, according to EU numbering. In some embodiments, the modified Fc polypeptide further comprises a knob mutation T366W, according to EU numbering. In some embodiments, the modified Fc polypeptide comprises the amino acid sequence of SEQ ID NO:63.

In other aspects, provided herein is an antibody heavy chain comprising: (a) an anti-human HER2 antibody heavy chain variable region, or a fragment thereof, and (b) a modified Fc polypeptide that contains modifications that create a TfR-binding site and a knob mutation T366W, according to EU numbering.

In some embodiments, the antibody heavy chain variable region comprises one or more CDRs selected from the group consisting of: (a) a heavy chain CDR1 having at least 90% sequence identity to the amino acid sequence of SEQ ID NO:69 or having up to two amino acid substitutions relative to the amino acid sequence of SEQ ID NO:69; (b) a heavy chain CDR2 having at least 90% sequence identity to the amino acid sequence of SEQ ID NO:70 or having up to two amino acid substitutions relative to the amino acid sequence of SEQ ID NO:70; and (c) a heavy chain CDR3 having at least 90% sequence identity to the amino acid sequence of SEQ ID NO:71 or having up to two amino acid substitutions relative to the amino acid sequence of SEQ ID NO:71. In some embodiments, the antibody heavy chain variable region comprises one or more CDRs selected from the group consisting of: (a) a heavy chain CDR1 comprising the amino acid sequence of SEQ ID NO:69; (b) a heavy chain CDR2 comprising the amino acid sequence of SEQ ID NO:70; and (c) a heavy chain CDR3 comprising the amino acid sequence of SEQ ID NO:71. In some embodiments, the antibody heavy chain variable region comprises the amino acid sequence of SEQ ID NO:59.

In some embodiments, the antibody variable region comprises one or more CDRs selected from the group consisting of: (a) a heavy chain CDR1 having at least 90% sequence identity to the amino acid sequence of SEQ ID NO:75 or having up to two amino acid substitutions relative to the amino acid sequence of SEQ ID NO:75; (b) a heavy chain CDR2 having at least 90% sequence identity to the amino acid sequence of SEQ ID NO:76 or having up to two amino acid substitutions relative to the amino acid sequence of SEQ ID NO:76; and (c) a heavy chain CDR3 having at least 90% sequence identity to the amino acid sequence of SEQ ID NO:77 or having up to two amino acid substitutions relative to the amino acid sequence of SEQ ID NO:77. In some embodiments, the antibody variable region comprises one or more CDRs selected from the group consisting of: (a) a heavy chain CDR1 comprising the amino acid sequence of SEQ ID NO:75; (b) a heavy chain CDR2 comprising the amino acid sequence of SEQ ID NO:76; and (c) a heavy chain CDR3 comprising the amino acid sequence of SEQ ID NO:77. In some embodiments, the antibody heavy chain variable region comprises the amino acid sequence of SEQ ID NO:61.

In some embodiments, the antibody variable region comprises one or more CDRs selected from the group consisting of: (a) a heavy chain CDR1 having at least 90% sequence identity to the amino acid sequence of SEQ ID NO:250 or having up to two amino acid substitutions relative to the amino acid sequence of SEQ ID NO:250; (b) a heavy chain CDR2 having at least 90% sequence identity to the amino acid sequence of SEQ ID NO:251 or having up to two amino acid substitutions relative to the amino acid sequence of SEQ ID NO:251; and (c) a heavy chain CDR3 having at least 90% sequence identity to the amino acid sequence of SEQ ID NO:252 or having up to two amino acid substitutions relative to the amino acid sequence of SEQ ID NO:252. In some embodiments, the antibody variable region comprises one or more CDRs selected from the group consisting of: (a) a heavy chain CDR1 comprising the amino acid sequence of SEQ ID NO:250; (b) a heavy chain CDR2 comprising the amino acid sequence of SEQ ID NO:251; and (c) a heavy chain CDR3 comprising the amino acid sequence of SEQ ID NO:252. In some embodiments, the antibody heavy chain variable region comprises the amino acid sequence of SEQ ID NO:256.

In some embodiments, the antibody heavy chain comprises the amino acid sequence of any one of SEQ ID NOS:1, 9, 17, and 81. In some embodiments, the antibody heavy chain comprises the amino acid sequence of any one of SEQ ID NOS:29, 37, 45, and 89. In some embodiments, the antibody heavy chain comprises the amino acid sequence of any one of SEQ ID NOS:258, 266, 274, and 282. In some embodiments, the modified Fc polypeptide further comprises amino acid modifications L234A and L235A. In some embodiments, the antibody heavy chain comprises the amino acid sequence of any one of SEQ ID NOS:2, 10, 18, and 82. In some embodiments, the antibody heavy chain comprises the amino acid sequence of any one of SEQ ID NOS:30, 38, 46, and 90. In some embodiments, the antibody heavy chain comprises the amino acid sequence of any one of SEQ ID NOS:259, 267, 275, and 283.

In other aspects, provided herein is an antibody heavy chain comprising: (a) an anti-human HER2 antibody heavy chain variable region, or a fragment thereof, and (b) a modified Fc polypeptide that contains modifications that create a TfR-binding site, a knob mutation T366W, and amino acid modification N434S with or without M428L, according to EU numbering.

In some embodiments, the antibody heavy chain variable region comprises one or more CDRs selected from the group consisting of: (a) a heavy chain CDR1 having at least 90% sequence identity to the amino acid sequence of SEQ ID NO:69 or having up to two amino acid substitutions relative to the amino acid sequence of SEQ ID NO:69; (b) a heavy chain CDR2 having at least 90% sequence identity to the amino acid sequence of SEQ ID NO:70 or having up to two amino acid substitutions relative to the amino acid sequence of SEQ ID NO:70; and (c) a heavy chain CDR3 having at least 90% sequence identity to the amino acid sequence of SEQ ID NO:71 or having up to two amino acid substitutions relative to the amino acid sequence of SEQ ID NO:71. In some embodiments, the antibody heavy chain variable region comprises one or more CDRs selected from the group consisting of: (a) a heavy chain CDR1 comprising the amino acid sequence of SEQ ID NO:69; (b) a heavy chain CDR2 comprising the amino acid sequence of SEQ ID NO:70; and (c) a heavy chain CDR3 comprising the amino acid sequence of SEQ ID NO:71. In some embodiments, the antibody heavy chain variable region comprises the amino acid sequence of SEQ ID NO:59.

In some embodiments, the antibody variable region comprises one or more CDRs selected from the group consisting of: (a) a heavy chain CDR1 having at least 90% sequence identity to the amino acid sequence of SEQ ID NO:75 or having up to two amino acid substitutions relative to the amino acid sequence of SEQ ID NO:75; (b) a heavy chain CDR2 having at least 90% sequence identity to the amino acid sequence of SEQ ID NO:76 or having up to two amino acid substitutions relative to the amino acid sequence of SEQ ID NO:76; and (c) a heavy chain CDR3 having at least 90% sequence identity to the amino acid sequence of SEQ ID NO:77 or having up to two amino acid substitutions relative to the amino acid sequence of SEQ ID NO:77. In some embodiments, the antibody variable region comprises one or more CDRs selected from the group consisting of: (a) a heavy chain CDR1 comprising the amino acid sequence of SEQ ID NO:75; (b) a heavy chain CDR2 comprising the amino acid sequence of SEQ ID NO:76; and (c) a heavy chain CDR3 comprising the amino acid sequence of SEQ ID NO:77. In some embodiments, the antibody heavy chain variable region comprises the amino acid sequence of SEQ ID NO:61.

In some embodiments, the antibody variable region comprises one or more CDRs selected from the group consisting of: (a) a heavy chain CDR1 having at least 90% sequence identity to the amino acid sequence of SEQ ID NO:250 or having up to two amino acid substitutions relative to the amino acid sequence of SEQ ID NO:250; (b) a heavy chain CDR2 having at least 90% sequence identity to the amino acid sequence of SEQ ID NO:251 or having up to two amino acid substitutions relative to the amino acid sequence of SEQ ID NO:251; and (c) a heavy chain CDR3 having at least 90% sequence identity to the amino acid sequence of SEQ ID NO:252 or having up to two amino acid substitutions relative to the amino acid sequence of SEQ ID NO:252. In some embodiments, the antibody variable region comprises one or more CDRs selected from the group consisting of: (a) a heavy chain CDR1 comprising the amino acid sequence of SEQ ID NO:250; (b) a heavy chain CDR2 comprising the amino acid sequence of SEQ ID NO:251; and (c) a heavy chain CDR3 comprising the amino acid sequence of SEQ ID NO:252. In some embodiments, the antibody heavy chain variable region comprises the amino acid sequence of SEQ ID NO:256.

In some embodiments, the antibody heavy chain comprises the amino acid sequence of any one of SEQ ID NOS:3, 11, 19, and 83. In some embodiments, the antibody heavy chain comprises the amino acid sequence of any one of SEQ ID NOS:31, 39, 47, and 91. In some embodiments, the antibody heavy chain comprises the amino acid sequence of any one of SEQ ID NOS:260, 268, 276, and 284. In some embodiments, the modified Fc polypeptide further comprises amino acid modifications L234A and L235A. In some embodiments, the antibody heavy chain comprises the amino acid sequence of any one of SEQ ID NOS:4, 12, 20, and 84. In some embodiments, the antibody heavy chain comprises the amino acid sequence of any one of SEQ ID NOS:32, 40, 48, and 92. In some embodiments, the antibody heavy chain comprises the amino acid sequence of any one of SEQ ID NOS:261, 269, 277, and 285.

In other aspects, provided herein is a pharmaceutical composition comprising an Fc polypeptide dimer-antibody variable region fusion protein described herein and a pharmaceutically acceptable carrier.

In other aspects, provided herein is a method of transcytosis of an antibody variable region that is capable of binding human HER2, or an antigen-binding fragment thereof, across an endothelium, the method comprising contacting the endothelium with a composition comprising an Fc polypeptide dimer-antibody variable region fusion protein described herein. In some embodiments, the endothelium is the BBB.

In other aspects, provided herein is a method for treating a cancer in a subject, the method comprising administering to the subject a therapeutically effective amount of a composition comprising an Fc polypeptide dimer-antibody variable region fusion protein described herein. In some embodiments, the cancer is a HER2-positive cancer. In some embodiments, the HER2-positive cancer is a HER2-positive breast cancer. In some embodiments, the HER2-positive cancer is a HER2-positive gastric adenocarcinoma and/or a HER2-positive gastroesophageal junction adnocarcinoma. the HER2-positive cancer is a metastatic cancer.

In other aspects, provided herein is a method for treating brain metastasis of a HER2-positive cancer in a subject, the method comprising administering to the subject a therapeutically effective amount of a composition comprising an Fc polypeptide dimer-antibody variable region fusion protein described herein. In some embodiments, the HER2-positive cancer is a HER2-positive breast cancer. In some embodiments, the HER2-positive cancer is a HER2-positive gastric adenocarcinoma and/or a HER2-positive gastroesophageal junction adnocarcinoma.

In some embodiments, a combination of different Fc polypeptide dimer-antibody variable region fusion proteins (e.g., a combination of Fc polypeptide dimer-antibody variable region fusion proteins that bind to subdomains IV and II of HER2) is administered. In some embodiments, a first Fc polypeptide dimer-antibody variable region fusion protein and a second Fc polypeptide dimer-antibody variable region fusion protein are administered to the subject, wherein the antibody variable region of the first Fc polypeptide dimer-antibody variable region fusion protein comprises two antibody heavy chain variable regions comprising the amino acid sequence of SEQ ID NO:59 and two light chain variable regions comprising the amino acid sequence of SEQ ID NO:60 (i.e., an anti-HER2 subdomain IV Fc polypeptide dimer-antibody variable region fusion protein), and wherein the antibody variable region of the second Fc polypeptide dimer-antibody variable region fusion protein comprises two antibody heavy chain variable regions comprising the amino acid sequence of SEQ ID NO:61 and two light chain variable regions comprising the amino acid sequence of SEQ ID NO:62 (i.e., an anti-HER2 subdomain II Fc polypeptide dimer-antibody variable region fusion protein). In some embodiments, an anti-HER2 subdomain IV Fc polypeptide dimer-antibody variable region fusion protein can be administered alone or in combination with an anti-HER2 subdomain II Fc polypeptide dimer-antibody variable region fusion protein. In some embodiments, an anti-HER2 subdomain II Fc polypeptide dimer-antibody variable region fusion protein can be administered alone or in combination with an anti-HER subdomain IV Fc polypeptide dimer-antibody variable region fusion protein. In certain embodiments, an anti-HER2 subdomain IV Fc polypeptide dimer-antibody variable region fusion protein can be administered alone. In certain embodiments, an anti-HER2 subdomain II Fc polypeptide dimer-antibody variable region fusion protein can be administered alone.

In some embodiments, a combination of different Fc polypeptide dimer-antibody variable region fusion proteins (e.g., a combination of Fc polypeptide dimer-antibody variable region fusion proteins that bind to subdomains II and I of HER2) is administered. In some embodiments, a first Fc polypeptide dimer-antibody variable region fusion protein and a second Fc polypeptide dimer-antibody variable region fusion protein are administered to the subject, wherein the antibody variable region of the first Fc polypeptide dimer-antibody variable region fusion protein comprises two antibody heavy chain variable regions comprising the amino acid sequence of SEQ ID NO:61 and two light chain variable regions comprising the amino acid sequence of SEQ ID NO:62 (i.e., an anti-HER2 subdomain II Fc polypeptide dimer-antibody variable region fusion protein), and wherein the antibody variable region of the second Fc polypeptide dimer-antibody variable region fusion protein comprises two antibody heavy chain variable regions comprising the amino acid sequence of SEQ ID NO:256 and two light chain variable regions comprising the amino acid sequence of SEQ ID NO:257 (i.e., an anti-HER2 subdomain I Fc polypeptide dimer-antibody variable region fusion protein). In some embodiments, an anti-HER2 subdomain II Fc polypeptide dimer-antibody variable region fusion protein can be administered alone or in combination with an anti-HER2 subdomain I Fc polypeptide dimer-antibody variable region fusion protein. In some embodiments, an anti-HER2 subdomain I Fc polypeptide dimer-antibody variable region fusion protein can be administered alone or in combination with an anti-HER subdomain II Fc polypeptide dimer-antibody variable region fusion protein. In certain embodiments, an anti-HER2 subdomain II Fc polypeptide dimer-antibody variable region fusion protein can be administered alone. In certain embodiments, an anti-HER2 subdomain I Fc polypeptide dimer-antibody variable region fusion protein can be administered alone.

In some embodiments, a combination of different Fc polypeptide dimer-antibody variable region fusion proteins (e.g., a combination of Fc polypeptide dimer-antibody variable region fusion proteins that bind to subdomains IV and I of HER2) is administered. In some embodiments, a first Fc polypeptide dimer-antibody variable region fusion protein and a second Fc polypeptide dimer-antibody variable region fusion protein are administered to the subject, wherein the antibody variable region of the first Fc polypeptide dimer-antibody variable region fusion protein comprises two antibody heavy chain variable regions comprising the amino acid sequence of SEQ ID NO:59 and two light chain variable regions comprising the amino acid sequence of SEQ ID NO:60 (i.e., an anti-HER2 subdomain IV Fc polypeptide dimer-antibody variable region fusion protein), and wherein the antibody variable region of the second Fc polypeptide dimer-antibody variable region fusion protein comprises two antibody heavy chain variable regions comprising the amino acid sequence of SEQ ID NO:256 and two light chain variable regions comprising the amino acid sequence of SEQ ID NO:257 (i.e., an anti-HER2 subdomain I Fc polypeptide dimer-antibody variable region fusion protein). In some embodiments, an anti-HER2 subdomain IV Fc polypeptide dimer-antibody variable region fusion protein can be administered alone or in combination with an anti-HER2 subdomain I Fc polypeptide dimer-antibody variable region fusion protein. In some embodiments, an anti-HER2 subdomain I Fc polypeptide dimer-antibody variable region fusion protein can be administered alone or in combination with an anti-HER subdomain IV Fc polypeptide dimer-antibody variable region fusion protein. In certain embodiments, an anti-HER2 subdomain IV Fc polypeptide dimer-antibody variable region fusion protein can be administered alone. In certain embodiments, an anti-HER2 subdomain I Fc polypeptide dimer-antibody variable region fusion protein can be administered alone.

In some embodiments, the composition comprising the Fc polypeptide dimer-antibody variable region fusion protein antagonizes HER2 activity. In some embodiments, the subject has not been previously treated with an anti-HER2 therapy and/or a chemotherapy for metastatic disease.

In yet another aspect, the disclosure features a method for treating a cancer or treating brain metastasis of a cancer in a subject, the method comprising administering to the subject a therapeutically effective amount of an anti-HER2 construct that binds to (a) subdomain I or II of human HER2 and (b) a transferrin receptor (TfR), wherein the anti-HER2 construct alone is therapeutically effective for treating the cancer.

In some embodiments of this aspect, the anti-HER2 construct comprises an antibody variable region that binds to subdomain I or II of human HER2. The anti-HER2 construct can comprise a modified Fc polypeptide dimer that comprises a first Fc polypeptide that contains modifications that create a TfR-binding site. For example, the anti-HER2 construct is an Fc polypeptide dimer-antibody variable region fusion protein.

In other embodiments of this aspect, the anti-HER2 construct comprises an antibody variable region that binds TfR. For example, the anti-HER2 construct can be a bispecific construct comprising an antibody variable region that binds to subdomain I or II of human HER2 and an antibody variable region that binds TfR.

In some embodiments, the anti-HER2 construct is administered to the subject as a monotherapy. In some embodiments, the anti-HER2 construct is adminstered in combination with a chemotherapy or radiation therapy.

In some embodiments, the anti-HER2 construct specifically binds to HER2 and TfR on the same cell.

In another aspect, the disclosure features a method for treating a cancer or treating brain metastasis of a cancer in a subject, the method comprising administering to the subject a therapeutically effective amount of:

(a) a first anti-HER2 construct that binds to subdomain II of human HER2; and
(b) a second anti-HER2 construct that binds to subdomain IV of human HER2,
or
(a) a first anti-HER2 construct that binds to subdomain I of human HER2; and
(b) a second anti-HER2 construct that binds to subdomain IV of human HER2,
or
(a) a first anti-HER2 construct that binds to subdomain I of human HER2; and
(b) a second anti-HER2 construct that binds to subdomain II of human HER2,
wherein the first and/or the second anti-HER2 construct also binds TfR.

In some embodiments of this aspect, the first anti-HER2 construct, but not the second anti-HER2 construct, binds TfR. The first anti-HER2 construct can specifically bind to TfR and HER2 on the same cell.

In some embodiments, the second anti-HER2 construct, but not the first anti-HER2 construct, binds TfR. The second anti-HER2 construct can specifically bind to TfR and HER2 on the same cell.

In some embodiments of this aspect, the first and/or the second anti-HER2 construct comprises an antibody variable region that binds to subdomain I, II, or IV of human HER2 and a modified Fc polypeptide dimer that comprises a first Fc polypeptide that contains modifications that create a TfR-binding site. In certain embodiments, the first anti-HER2 construct is an Fc polypeptide dimer-antibody variable region fusion protein. In certain embodiments, the second anti-HER2 construct is an Fc polypeptide dimer-antibody variable region fusion protein. In certain embodiments, the first and second anti-HER2 constructs are Fc polypeptide dimer-antibody variable region fusion proteins.

In some embodiments, the Fc polypeptide dimer-antibody variable region fusion protein comprises two heavy chain variable regions and two light chain variable regions that bind to subdomain II of HER2, wherein each of the two heavy chain variable regions comprises a heavy chain CDR1 (CDR-H1), a CDR H2, and a CDR H3, and each of the two light chain variable regions comprises a light chain CDR1 (CDR-L1), a CDR L2, and a CDR L3, and wherein:

(1) the CDR-H1 comprises a sequence having at least 90% sequence identity to or having up to two amino acid substitutions relative to the amino acid sequence of SEQ ID NO:75;
(2) the CDR-H2 comprises a sequence having at least 90% sequence identity to or having up to two amino acid substitutions relative to the amino acid sequence of SEQ ID NO:76;
(3) the CDR-H3 comprises a sequence having at least 90% sequence identity to or having up to two amino acid substitutions relative to the amino acid sequence of SEQ ID NO:77;
(4) the CDR-L1 comprises a sequence having at least 90% sequence identity to or having up to two amino acid substitutions relative to the amino acid sequence of SEQ ID NO:78;
(5) the CDR-L2 comprises a sequence having up to two amino acid substitutions relative to the amino acid sequence of SEQ ID NO:79; and
(6) the CDR-L3 comprises a sequence having up to two amino acid substitutions relative to the amino acid sequence of SEQ ID NO:80.

In some embodiments of the Fc polypeptide dimer-antibody variable region fusion protein that binds to subdomain II of HER2, each of the two heavy chain variable regions comprises a sequence having at least 90% sequence identity to the amino acid sequence of SEQ ID NO:61 and each of the two light chain variable regions comprises a sequence having at least 90% sequence identity to the amino acid sequence of SEQ ID NO:62.

In some embodiments, the Fc polypeptide dimer-antibody variable region fusion protein comprises two heavy chain variable regions and two light chain variable regions that bind to subdomain I of HER2, wherein each of the two heavy chain variable regions comprises a heavy chain CDR1 (CDR-H1), a CDR H2, and a CDR H3, and each of the two light chain variable regions comprises a light chain CDR1 (CDR-L1), a CDR L2, and a CDR L3, and wherein:

(1) the CDR-H1 comprises a sequence having at least 90% sequence identity to or having up to two amino acid substitutions relative to the amino acid sequence of SEQ ID NO:250;
(2) the CDR-H2 comprises a sequence having at least 90% sequence identity to or having up to two amino acid substitutions relative to the amino acid sequence of SEQ ID NO:251;
(3) the CDR-H3 comprises a sequence having at least 90% sequence identity to or having up to two amino acid substitutions relative to the amino acid sequence of SEQ ID NO:252;
(4) the CDR-L1 comprises a sequence having at least 90% sequence identity to or having up to two amino acid substitutions relative to the amino acid sequence of SEQ ID NO:253;
(5) the CDR-L2 comprises a sequence having up to two amino acid substitutions relative to the amino acid sequence of SEQ ID NO:254; and
(6) the CDR-L3 comprises a sequence having up to two amino acid substitutions relative to the amino acid sequence of SEQ ID NO:255.

In some embodiments of the Fc polypeptide dimer-antibody variable region fusion protein that binds to subdomain I of HER2, each of the two heavy chain variable regions comprises a sequence having at least 90% sequence identity to the amino acid sequence of SEQ ID NO:256 and each of the two light chain variable regions comprises a sequence having at least 90% sequence identity to the amino acid sequence of SEQ ID NO:257.

In some embodiments, the Fc polypeptide dimer-antibody variable region fusion protein comprises two heavy chain variable regions and two light chain variable regions that bind to subdomain IV of HER2, wherein each of the two heavy chain variable regions comprises a heavy chain CDR1 (CDR-H1), a CDR H2, and a CDR H3, and each of the two light chain variable regions comprises a light chain CDR1 (CDR-L1), a CDR L2, and a CDR L3, and wherein:

(1) the CDR-H1 comprises a sequence having at least 90% sequence identity to or having up to two amino acid substitutions relative to the amino acid sequence of SEQ ID NO:69;
(2) the CDR-H2 comprises a sequence having at least 90% sequence identity to or having up to two amino acid substitutions relative to the amino acid sequence of SEQ ID NO:70;
(3) the CDR-H3 comprises a sequence having at least 90% sequence identity to or having up to two amino acid substitutions relative to the amino acid sequence of SEQ ID NO:71;
(4) the CDR-L1 comprises a sequence having at least 90% sequence identity to or having up to two amino acid substitutions relative to the amino acid sequence of SEQ ID NO:72;
(5) the CDR-L2 comprises a sequence having up to two amino acid substitutions relative to the amino acid sequence of SEQ ID NO:73; and
(6) the CDR-L3 comprises a sequence having up to two amino acid substitutions relative to the amino acid sequence of SEQ ID NO:74.

In some embodiments of the Fc polypeptide dimer-antibody variable region fusion protein that binds to subdomain IV of HER2, each of the two heavy chain variable regions comprises a sequence having at least 90% sequence identity to the amino acid sequence of SEQ ID NO:59 and each of the two light chain variable regions comprises a sequence having at least 90% sequence identity to the amino acid sequence of SEQ ID NO:60.

In some embodiments of this aspect, the TfR-binding site in the Fc polypeptide of the Fc polypeptide dimer-antibody variable region fusion protein comprises a modified CH3 domain. The modified CH3 domain can be derived from a human IgG1, IgG2, IgG3, or IgG4 CH3 domain. The modified CH3 domain can comprise one, two, three, four, five, six, seven, eight, nine, ten, or eleven substitutions in a set of amino acid positions comprising 380, 384, 386, 387, 388, 389, 390, 413, 415, 416, and 421, according to EU numbering.

In some embodiments, the modified CH3 domain comprises Glu, Leu, Ser, Val, Trp, Tyr, or Gln at position 380; Leu, Tyr, Phe, Trp, Met, Pro, or Val at position 384; Leu, Thr, His, Pro, Asn, Val, or Phe at position 386; Val, Pro, Ile, or an acidic amino acid at position 387; Trp at position 388; an aliphatic amino acid, Gly, Ser, Thr, or Asn at position 389; Gly, His, Gln, Leu, Lys, Val, Phe, Ser, Ala, Asp, Glu, Asn, Arg, or Thr at position 390; an acidic amino acid, Ala, Ser, Leu, Thr, Pro, Ile, or His at position 413; Glu, Ser, Asp, Gly, Thr, Pro, Gln, or Arg at position 415; Thr, Arg, Asn, or an acidic amino acid at position 416; and/or an aromatic amino acid, His, or Lys at position 421, according to EU numbering.

In some embodiments of this aspect of the disclosure, the anti-HER2 construct binds to the apical domain of TfR. In some embodiments, the modified Fc polypeptide dimer comprises a first Fc polypeptide comprising amino acid modifications that reduce FcγR binding when bound to TfR. In certain embodiments, the amino acid modifications comprise Ala at position 234 and at position 235, according to EU numbering. In some embodiments, one or both Fc polypeptides that are present in the Fc polypeptide dimer comprise amino acid modifications that increase serum half-life. In certain embodiments, the amino acid modifications that increase serum half-life comprise (i) a Leu at position 428 and a Ser at position 434, or (ii) a Ser or Ala at position 434, according to EU numbering.

In some embodiments of this aspect of the disclosure, the first Fc polypeptide further comprises a knob mutation T366W and a second Fc polypeptide in the Fc polypeptide dimer comprises hole mutations T366S, L368A, and Y407V, according to EU numbering. For example, in some embodiments, the first Fc polypeptide comprises the amino acid sequence of SEQ ID NO:63. In some embodiments, the second Fc polypeptide comprises the amino acid sequence of any one of SEQ ID NOS:67 and 68.

In some embodiments, the Fc polypeptide dimer-antibody variable region fusion protein that binds to subdomain II of human HER2 comprises:

(a) a first heavy chain having the sequence of SEQ ID NO:38, a second heavy chain having the sequence of SEQ ID NO:55;
or
(b) a first heavy chain having the sequence of SEQ ID NO:46, a second heavy chain having the sequence of SEQ ID NO:55;
or
(c) a first heavy chain having the sequence of SEQ ID NO:30, a second heavy chain having the sequence of SEQ ID NO:55.

In some embodiments, the Fc polypeptide dimer-antibody variable region fusion protein that binds to subdomain I of human HER2 comprises:

(a) a first heavy chain having the sequence of SEQ ID NO:267, a second heavy chain having the sequence of SEQ ID NO:290;
or
(b) a first heavy chain having the sequence of SEQ ID NO:275, a second heavy chain having the sequence of SEQ ID NO:290;
or
(c) a first heavy chain having the sequence of SEQ ID NO:259, a second heavy chain having the sequence of SEQ ID NO:290.

In some embodiments, the Fc polypeptide dimer-antibody variable region fusion protein that binds to subdomain IV of human HER2 comprises:

(a) a first heavy chain having the sequence of SEQ ID NO:10, a second heavy chain having the sequence of SEQ ID NO:27;
or
(b) a first heavy chain having the sequence of SEQ ID NO:18, a second heavy chain having the sequence of SEQ ID NO:27;
or
(c) a first heavy chain having the sequence of SEQ ID NO:2, a second heavy chain having the sequence of SEQ ID NO:27.

In other embodiments of this aspect, the first and/or the second anti-HER2 construct comprises an antibody variable region that binds TfR. For example, the first anti-HER2 construct can be a bispecific construct comprising an antibody variable region that binds to human HER2 and an antibody variable region that binds TfR. The second anti-HER2 construct can be a bispecific construct comprising an antibody variable region that binds to human HER2 and an antibody variable region that binds TfR. Both the first and the second anti-HER2 constructs can be bispecific constructs comprising an antibody variable region that binds to human HER2 and an antibody variable region that binds TfR.

In some embodiments of this aspect of the disclosure, the cancer is a HER2-positive breast cancer. The HER2-positive cancer can be a HER2-positive gastric adenocarcinoma and/or a HER2-positive gastroesophageal junction adnocarcinoma.

In another aspect, the disclosure features a method of reducing TfR expression level on the surface of a cell by contacting the cell with an anti-HER2 construct that binds to (a) subdomain I, II, or IV of human HER2 and (b) a transferrin receptor (TfR), wherein the anti-HER2 construct alone is effective in reducing TfR expression level on the cell surface of the cell, and wherein the anti-HER2 construct binds to both TfR and HER2 on the same cell. In this aspect, the anti-HER2 construct can be any of the constructs described herein, such as an Fc polypeptide dimer-antibody variable region fusion protein (which includes an antibody variable region that is capable of binding to human HER2 and a modified Fc polypeptide dimer that comprises a first Fc polypeptide that contains modifications that create a TfR-binding site) or an anti-HER2 bispecific construct (which includes an antibody variable region that is capable of binding to human HER2 and an antibody variable region that binds TfR).

In yet another aspect, the disclosure features a method of binding a construct to TfR and human HER2 that are expressed on a cell (e.g., TfR and human HER2 expressed on the same cell), comprising contacting the cell with an anti-HER2 construct that binds to (a) subdomain I, II, or IV of human HER2 and (b) a transferrin receptor (TfR), wherein the anti-HER2 construct reduces TfR expression level on the cell surface of the cell when the cell is in contact with the anti-HER2 construct. In this aspect, the construct that binds to TfR and human HER2 can be any of the constructs described herein, such as an Fc polypeptide dimer-antibody variable region fusion protein (which includes an antibody variable region that is capable of binding to human HER2 and a modified Fc polypeptide dimer that comprises a first Fc polypeptide that contains modifications that create a TfR-binding site) or an anti-HER2 bispecific construct (which includes an antibody variable region that is capable of binding to human HER2 and an antibody variable region that binds TfR).

In some embodiments, the anti-HER2 construct reduces TfR expression level on the cell surface of the cell by at least 10% (e.g., at least 15%, 20%, 25%, 30%, 35%, 40%, 45%, or 50%) when the cell is in contact with the fusion protein.

In some embodiments, the anti-HER2 construct comprises an antibody variable region that binds to subdomain I, II, or IV of human HER2. In certain embodiments, the anti-HER2 construct can comprise an antibody variable region that binds to subdomain I, II, or IV of human HER2 and a modified Fc polypeptide dimer that comprises a first Fc polypeptide that contains modifications that create a TfR-binding site. For example, the anti-HER2 construct can be an Fc polypeptide dimer-antibody variable region fusion protein. In yet other embodiments, the anti-HER2 constructs can be a bispecific construct comprising an antibody variable region that binds to human HER2 and an antibody variable region that binds TfR.

In any of the aspects described herein, in some embodiments, the cell is a cancer cell (e.g., a metastatic cancer cell). In certain embodiments, the cancer cell expresses both HER2 and TfR. In some embodiments, the cell can be a breast cancer cell (e.g., a HER2 positive cancer cell). In some embodiments of any of the aspects described herein, the cell is a mammalian cell, such as a human cell (e.g., a human cancer cell).

In any of the aspects described herein, in some embodiments, the cell is in a mammal, such as a human. In some embodiments, the human has or has been diagnosed with a HER2-positive cancer. In certain embodiments, the HER2-positive cancer is a metastatic cancer. In particular embodiments, the HER2-positive, metastatic cancer has metastasized to the brain.

### BRIEF DESCRIPTION OF THE DRAWINGS

FIGS. 1A and 1B show Biacore™ data. FIG. 1A shows binding affinities to HER2 extracellular domain using anti-HER2_DIV, HER2_DIV-35.23.1.1^{cisLALA} anti-HER2_DII, and HER2_DII-35.23.1.1^{cisLALA}. FIG. 1B shows binding affinities to human apical hTfR using HER2_DIV-35.23.1.1^{cisLALA} and HTER2_DII-35.23.1.1^{cisLALA}

FIG. 2 shows a growth inhibition assay of BT474 cells using WST1 reagent showing the inhibition of cancer cell proliferation on Day 6 by anti-HER2_DIV, HER2_DIV-35.23.1.1^{cisLALA}, a combination of anti-HER2_DIV and anti-HER2 DII, and a combination of HER2_DIV-35.23.1.1^{cisLALA} and HER2_DII-35.23.1.1^{cisLALA}

FIG. 3 shows Western blot data demonstrating the decrease of phosphorylated AKT (p-AKT) in BT474 cells treated with anti-HER2_DIV and HER2_DIV-35.23.1.1^{cisLALA}

FIG. 4 shows a growth inhibition assay of BT474 cells using WST1 reagent showing the response of cancer cell proliferation on Day 6 by anti-HER2_DIV, HER2_DIV-35.23.1.1^{cisLALA}, a combination of anti-HER2_DIV and anti-HER2 DII, and a combination of HER2_DIV-35.23.1.1^{cisLALA} and HER2_DII-35.23.1.1^{cisLALA} in the presence of neuregulin-1.

FIG. 5 shows Western blot data of protein levels of phosphorylated AKT (p-AKT) in BT474 cells treated with anti-HER2_DIV and HER2_DIV-35.23.1.1^{cisLALA} a combination of anti-HER2_DIV and anti-HER2_DII, and a combination of HER2_DIV-35.23.1.1^{cisLALA} and HER2_DII-35.23.1.1^{cisLALA} in the presence of neuregulin-1.

FIG. 6 shows ADCC activity in SK-BR-3 cells using HER2_DIV-35.23.1.1^{cisLALA}

FIGS. 7A-7C show the in vivo anti-tumor efficacy of ATV:HER2-DIV and ATV:HER2-DII in BT474 xenograft tumor model in SCID mice. FIG. 7A shows a significantly higher tumor growth inhibition in the animals treated with ATV:HER2-DIV+ATV:HER2-DII compared to anti-HER2-DIV+anti-HER2-DII. FIG. 7B shows a dose-response relationship using doses 3, 10, and 20 mg/kg of each test article showing that ATV:HER2-DIV+ATV:HER2-DII is more potent than anti-HER2-DIV+anti-HER2-DII. FIG. 7C shows that treatment of both anti-HER2-DIV+anti-HER2-DII and ATV:HER2-DIV+ATV:HER2-DII significantly reduced pAKT levels, which is consistent with the mechanism in which targeting against HER2 could abrogate the PI3K/Akt signaling pathway that is activated in HER2⁺ tumors. “ATV” refers to a TfR-binding Fc polypeptide.

FIGS. 8A-8C show the plasma exposure, brain uptake, and brain to plasma ratio of TfR^{mu/hu} KI mice treated with ATV:HER2-DIV and ATV:HER2-DII.

FIGS. 9A and 9B show growth inhibition assays of BT474 cells and OE19 cells using ATV:HER2-DIV showing that ATV:HER2-DIV has increased anti-proliferative effect compared to anti-HER2_DIV in the anti-HER2_DIV-resistant HER2⁺ cancer cell lines.

FIGS. 10A and 10B show growth inhibition assays of BT474 cells and OE19 cells using ATV:HER2-DII showing that ATV:HER2-DII has superior growth inhibition than anti-HER2-DII while ATV:ctrl and anti-TfR with or without anti-HER2-DII groups have minimal effects in the HER2⁺ cancer cell lines.

FIG. 11 shows a growth inhibition assay of OE19 cells using ATV:HER2-DI showing that ATV:HER2-DI has superior growth inhibition than anti-HER2-DI in HER2⁺ cancer cell lines.

FIG. 12 shows growth inhibition assay of BT474 cells without NRG1 using ATV:HER2-DIV+ATV:HER2-DII showing that the combination of ATV:HER2-DIV+ATV:HER2-DII is more potent in growth inhibition than the combination of anti-HER2-DIV+anti-HER2-DII with or without NRG1.

FIGS. 13A-13C show the cell surface TfR expression in BT474 cells treated with ATV:HER2-DIV, ATV:HER2-DII, and the combination of ATV:HER2-DIV and ATV:HER2-DII. Treatment of ATV:HER2-DIV and ATV:HER2-DII enhanced down-regulation of cell surface TfR expression upon internalization conditions (37° C. for 30 min).

### DETAILED DESCRIPTION

### I. Introduction

We have developed anti-HER2 constructs that are capable of crossing the BBB. In general, these anti-HER2 constructs include an antibody variable region that is capable of binding to human HER2 (e.g., subdomain I, II, or IV of human HER2). In some embodiments, the anti-HER2 constructs include an antibody variable region that is capable of binding HER2 fused to a modified Fc polypeptide that has been engineered to include a non-native TfR-binding site, which is also referred to as “an Fc polypeptide dimer-antibody variable region fusion protein” herein. For example, an Fc polypeptide dimer-antibody variable region fusion protein can include an anti-HER2 Fab fused to a modified Fc polypeptide dimer that includes a TfR-binding site.

In other embodiments, the anti-HER2 constructs include an antibody variable region that is capable of binding HER2 (e.g., subdomain I, II, or IV of human HER2) and an antibody variable region that is capable of binding TfR.

Thus, the present disclosure relates, in part, to Fc polypeptide dimer-antibody variable region fusion proteins, and fragments thereof, that have been engineered to bind subdomain IV, subdomain II, or subdomain I of HER2, bind TfR, have reduced effector function (e.g., ADCC or CDC) when bound to TfR, but still retain and exhibit a level of effector function (e.g., ADCC or CDC) when the Fc polypeptide dimer-antibody variable region fusion protein is bound to HER2. The present disclosure also relates, in part, to methods for delivering anti-HER2 therapeutic constructs across the BBB and treating HER2-positive cancers, as well as metastases of HER2-positive cancers. Surprisingly, as described herein, it was found that using a combination of Fc polypeptide dimer-antibody variable region fusion proteins that target HER2 subdomains IV and II was more effective for inhibiting breast cancer cell growth than using a combination of anti-HER2 subdomain IV and anti-HER2 subdomain II antibodies. Moreover, in some embodiments, using a combination of Fc polypeptide dimer-antibody variable region fusion proteins that target HER2 subdomains IV and I is more effective for inhibiting breast cancer cell growth than using a combination of anti-HER2 subdomain IV and anti-HER2 subdomain I antibodies. Further, in some embodiments, using a combination of Fc polypeptide dimer-antibody variable region fusion proteins that target HER2 subdomains II and I is more effective for inhibiting breast cancer cell growth than using a combination of anti-HER2 subdomain II and anti-HER2 subdomain I antibodies.

Moreover, using an Fc polypeptide dimer-antibody variable region fusion protein that targets HER2 subdomain IV, subdomain II, or subdomain I alone is more effective for inhibiting breast cancer cell growth than using an anti-HER2 subdomain IV antibody, an anti-HER2 subdomain II antibody, or an anti-HER2 subdomain I antibody, respectively. Further, using an Fc polypeptide dimer-antibody variable region fusion protein that targets HER2 subdomain IV, subdomain II, or subdomain I alone is more effective for inhibiting breast cancer cell growth than using a combination of an anti-HER2 subdomain IV antibody and an anti-TfR antibody, a combination of an anti-HER2 subdomain II antibody and an anti-TfR antibody, or a combination of an anti-HER2 subdomain I antibody and an anti-TfR antibody, respectively.

In other embodiments, the present disclosure also relates, in part, to using an anti-HER2 construct that includes an antibody variable region that is capable of binding HER2 (e.g., subdomain I, II, or IV of human HER2) and an antibody variable region that is capable of binding TfR to inhibit breast cancer cell growth. Using such anti-HER2 constructs having bispecificity to HER2 (e.g., subdomain I, II, or IV of human HER2) and TfR is more effective for inhibiting breast cancer cell growth than using an anti-HER2 antibody (e.g., an anti-HER2 subdomain I antibody, an anti-HER2 subdomain II antibody, or an anti-HER2 subdomain IV antibody) alone.

### II. Definitions

As used herein, the singular forms “a,” “an,” and “the” include plural referents unless the content clearly dictates otherwise. Thus, for example, reference to “a polypeptide” may include two or more such molecules, and the like.

As used herein, the terms “about” and “approximately,” when used to modify an amount specified in a numeric value or range, indicate that the numeric value as well as reasonable deviations from the value known to the skilled person in the art, for example±20%, ±10%, or ±5%, are within the intended meaning of the recited value.

The terms “human epidermal growth factor receptor 2,” “HER2,” “HER2/neu,” and “ERBB2” (also known as CD340, receptor tyrosine-protein kinase erbB-2, proto-oncogene and Neu) refer to a tyrosine receptor kinase protein encoded by the ERBB2 gene in humans that is a member of the human epidermal growth factor receptor (HER/EGFR/ERBB) family. Amplification or overexpression of HER2 plays a significant role in the development and progression of certain aggressive types of cancer, including breast cancer. Non-limiting examples of human HER2 nucleotide sequences are set forth in GenBank reference numbers NP_001005862, NP_001289936, NP_001289937, NP_001289938, and NP_004448. Non-limiting examples of human HER2 peptide sequences are set forth in GenBank reference numbers NP_001005862, NP_001276865, NP_001276866, NP_001276867, and NP_004439.

The extracellular domain of HER2, which contains approximately 600 amino acids, includes four subdomains (subdomains I, II, III, and IV). Subdomains I and III form a ligand binding site. The cysteine-rich subdomains II and IV are involved in receptor homodimerization and heterodimerization. Anti-HER2 therapeutic constructs can bind to specific subdomains (e.g., subdomain II or subdomain IV).

When HER2 is amplified or overexpressed in a cell, the cell is referred to as being “HER2-positive” or “HER2+.” The level of HER2 amplification or overexpression in a HER2-positive cell is commonly expressed as a score ranging from 0 to 3 (i.e., HER2 0, HER2 1+, HER2 2+, or HER2 3+), with higher scores corresponding to greater degrees of expression.

HER2 testing methods include immunohistochemistry (IHC), fluorescence in situ hybridization (FISH), ELISA, and RNA quantification (e.g., of HER2 expression) methods such as RT-PCR and microarray analysis. HER2 testing can be performed on a subject (e.g., a patient) who is being considered for an anti-HER2 therapy.

As used herein, the term “anti-HER2 construct” refers to a molecule (e.g., a protein) construct that binds to (a) subdomain I, II, or IV of human HER2 and (b) a transferrin receptor (TfR). An anti-HER2 construct can include an antibody variable region that is capable of binding to human HER2. In some embodiments, an anti-HER2 construct is an Fc polypeptide dimer-antibody variable region fusion protein, which includes an antibody variable region that is capable of binding to human HER2 and a modified Fc polypeptide dimer that comprises a first Fc polypeptide that contains modifications that create a TfR-binding site. In other embodiments, an anti-HER2 construct is a bispecific construct, which includes an antibody variable region that is capable of binding to human HER2 and an antibody variable region that binds TfR. The anti-HER2 constructs as described herein can bind to subdomain I, II, or IV of human HER2.

As used herein, the term “anti-HER2-DI,” “anti-HER2-DII,” or “anti-HER2-DIV” refer to an antibody that binds to subdomain I, II, or IV, respectively, of human HER2.

As used herein, the term “Fc polypeptide” refers to the C-terminal region of a naturally occurring immunoglobulin heavy chain polypeptide that is characterized by an Ig fold as a structural domain. An Fc polypeptide contains constant region sequences including at least the CH2 domain and/or the CH3 domain and may contain at least part of the hinge region. In general, an Fc polypeptide does not contain a variable region.

A “modified Fc polypeptide” refers to an Fc polypeptide that has at least one mutation, e.g., a substitution, deletion or insertion, as compared to a wild-type immunoglobulin heavy chain Fc polypeptide sequence, but retains the overall Ig fold or structure of the native Fc polypeptide.

As used herein, the term “Fc polypeptide dimer” refers to a dimer of two Fc polypeptides. In some embodiments, an Fc polypeptide dimer is capable of binding an Fc receptor (e.g., FcγR). In an Fc polypeptide dimer, the two Fc polypeptides dimerize by the interaction between the two CH3 antibody constant domains. In some embodiments, the two Fc polypeptides may also dimerize via one or more disulfide bonds that form between the hinge domains of the two dimerizing Fc domain monomers. An Fc polypeptide dimer may be a wild-type Fc polypeptide dimer or a modified Fc polypeptide dimer. A wild-type Fc polypeptide dimer is formed by the dimerization of two wild-type Fc polypeptides. An Fc polypeptide dimer can be a heterodimer or a homodimer.

As used herein, the term “modified Fc polypeptide dimer” refers to an Fc polypeptide dimer that contains at least one modified Fc polypeptide. In some embodiments, a modified Fc polypeptide dimer contains two modified Fc polypeptides. A modified Fc polypeptide dimer may be a homodimer (i.e., contains two identical modified Fc polypeptides) or a heterodimer (i.e., contains two different Fc polypeptides in which at least one of the two Fc polypeptides is a modified Fc polypeptide).

A “transferrin receptor” or “TfR” as used herein refers to transferrin receptor protein 1. The human transferrin receptor 1 polypeptide sequence is set forth in SEQ ID NO:102. Transferrin receptor protein 1 sequences from other species are also known (e.g., chimpanzee, accession number XP_003310238.1; rhesus monkey, NP_001244232.1; dog, NP_001003111.1; cattle, NP_001193506.1; mouse, NP_035768.1; rat, NP_073203.1; and chicken, NP_990587.1). The term “transferrin receptor” also encompasses allelic variants of exemplary reference sequences, e.g., human sequences, that are encoded by a gene at a transferrin receptor protein 1 chromosomal locus. Full-length TfR protein includes a short N-terminal intracellular region, a transmembrane region, and a large extracellular domain. The extracellular domain is characterized by three domains: a protease-like domain, a helical domain, and an apical domain. The apical domain sequence of human transferrin receptor 1 is set forth in SEQ ID NO:103.

As used herein, the term “Fcγ receptor” or “FcγR” refers to one type of Fc receptors, which are classified based on the type of antibody that they recognize. FcγRs include several members, FcγRI (CD64), FcγRIIA (CD32), FcγRIIB (CD32), FcγRIIIA (CD16a), and FcγRIIIB (CD16b), which differ in their antibody affinities due to different molecular structures. FcγRs bind to the Fc portion of the IgG class of antibodies and are crucial for inducing phagocytosis of opsonized microbes. FcγRs are found on the cell surface of cells in the immune system. FcγRs are responsible for eliciting immune system effector functions and are activated upon binding of the Fc portion of an antibody to the receptor. FcγRs mediate immune functions, e.g., binding to antibodies that are attached to infected cells or invading pathogens, stimulating phagocytic or cytotoxic cells to destroy microbes or infected cells by antibody-mediated phagocytosis or ADCC.

As used herein, the term “reduce FcγR binding” refers to a modified Fc polypeptide or a modified Fc polypeptide dimer that contains mutations in the CH3 domain of the modified Fc polypeptide, in which the mutations decrease the affinity of the modified Fc polypeptide to the FcγR by 0.01% to 90% (e.g., 0.1%, 0.5%, 1%, 5%, 10%, 15%, 20%, 25%, 30%, 35%, 40%, 45%, 50%, 55%, 60%, 65%, 70%, 75%, 80%, 85%, or 90%) compared the affinity of an Fc polypeptide that does not contain mutations to reduce FcγR binding (e.g., a wild-type Fc polypeptide dimer). FcγR binding may be measured using, e.g., Surface Plasmon Resonance (SPR) methods (e.g., a Biacore™ system). Alternatively, FcγR binding can be measured using a functional assay, for example, an ADCC assay such as one described herein (e.g., an in vivo or in vitro assay of cell killing). The reduction of FcγR binding may be measured when the modified Fc polypeptide dimer is bound to TfR. In some embodiments, the modified Fc polypeptide or Fc polypeptide dimer may have reduced FcγR binding when bound to TfR, but limited (e.g., less than 25%, 20%, 15%, 10%, 8%, 5%, 3%, 2%, or 1% reduction) or no reduction when not bound to TfR).

As described further herein, a modified Fc polypeptide dimer may contain a first Fc polypeptide that has both a TfR-binding site and mutations that reduce FcγR binding when bound to TfR and a second Fc polypeptide that has neither a TfR-binding site nor mutations that reduce FcγR binding. Thus, upon TfR engagement, the resulting asymmetrical Fc polypeptide dimer having the first and second Fc polypeptides may have an overall reduced affinity for FcγR. By contrast, there may be limited (e.g., as described above) or no reduction in FcγR binding when not bound to TfR.

The term “FcRn” refers to the neonatal Fc receptor. Binding of Fc polypeptides to FcRn reduces clearance and increases serum half-life of the Fc polypeptide. The human FcRn protein is a heterodimer that is composed of a protein of about 50 kDa in size that is similar to a major histocompatibility (MHC) class I protein and a 02-microglobulin of about 15 kDa in size.

As used herein, an “FcRn binding site” refers to the region of an Fc polypeptide that binds to FcRn. In human IgG, the FcRn binding site, as numbered using the EU numbering scheme, includes L251, M252, 1253, S254, R255, T256, M428, H433, N434, H435, and Y436. These positions correspond to positions 21 to 26, 198, and 203 to 206 of SEQ ID NO:99.

As used herein, a “native FcRn binding site” refers to a region of an Fc polypeptide that binds to FcRn and that has the same amino acid sequence as the region of a naturally occurring Fc polypeptide that binds to FcRn.

As used herein, the term “does not substantially deplete reticulocytes” or “does not substantially deplete reticulocytes in vivo” means that the reduction in reticulotyes (e.g., the reduction in bone marrow recticulocytes or circulating reticulotyes) caused by an effector function-positive, TfR-binding Fc polypeptide dimer described herein, or an Fc polypeptide dimer-antibody variable region fusion protein described herein that contains an effector function-positive, TfR-binding Fc polypeptide dimer, is less than (e.g., less than 80%, 75%, 70%, 65%, 60%, 55%, 50%, 45%, 40%, 35%, 30%, 25%, 20%, 15%, 10%, 8%, 5%, 3%, 2%, or 1% of) the reduction in reticulocytes (e.g., the reduction in bone marrow recticulocytes or circulating reticulotyes) caused by a control, e.g., a corresponding TfR-binding Fc dimer or Fc polypeptide dimer-antibody variable region fusion protein with full effector function and/or contains no mutations that reduce FcγR binding, or an antibody containing a corresponding TfR-binding Fc dimer with full effector function and/or contains no mutations that reduce FcγR binding.

The term “does not substantially deplete reticulocytes” or “does not substantially deplete reticulocytes in vivo” can also mean that the amount or percentage of the remaining reticulotyes (e.g., the remaining reticulotyes in the bone marrow or in circulation) after dosing an effector function-positive, TfR-binding Fc polypeptide dimer described herein, or an Fc polypeptide dimer-antibody variable region fusion protein described herein that contains an effector function-positive, TfR-binding Fc polypeptide dimer, is more than (e.g., at least 1%, 5%, 10%, 15%, 20%, 25%, 30%, 35%, 40%, 45%, or 50% more than) the amount or percentage of the remaining reticulocytes (e.g., the remaining reticulotyes in the bone marrow or in circulation) after dosing a control (e.g., a corresponding TfR-binding Fc dimer or Fc polypeptide dimer-antibody variable region fusion protein with full effector function and/or contains no mutations that reduce FcγR binding, or an antibody containing a corresponding TfR-binding Fc dimer with full effector function and/or contains no mutations that reduce FcγR binding).

The amount or percentage of reticulocyte depletion (e.g., reticulocyte depletion in the bone marrow or in circulation), or the amount or percentage of remaining reticulocytes (e.g., remaining reticulocytes in the bone marrow or in circulation), may be measured in human TfR knock-in (TfR^{ms/hu} KI) mice (e.g., human TfR apical domain knock-in mice (“hTfR^{apical} knock-in mice”)), which are engineered to replace the mouse TfR with human apical domain/mouse chimeric TfR protein or in a non-human primate, such as a cynomolgus monkey. The measurement may be made by dosing the modified Fc dimer or control, e.g., 25 to 50 mg/kg intravenously (e.g., to the TfR^{ms/hu} KI mice) and circulating reticulocytes may be measured at 24 h post-dose by cytochemical reactions using the Advia 120 Hematology System, as described herein. Bone marrow reticulocytes can be measured using FACS sorting to determine the population of Ter 19⁺, hCD71^{hi}, and FSC^{low} population, as described herein.

The terms “CH3 domain” and “CH2 domain” as used herein refer to immunoglobulin constant region domain polypeptides. In the context of IgG antibodies, a CH3 domain polypeptide refers to the segment of amino acids from about position 341 to about position 447 as numbered according to the EU numbering scheme, and a CH2 domain polypeptide refers to the segment of amino acids from about position 231 to about position 340 as numbered according to the EU numbering scheme. CH2 and CH3 domain polypeptides may also be numbered by the IMGT (ImMunoGeneTics) numbering scheme in which the CH2 domain numbering is 1-110 and the CH3 domain numbering is 1-107, according to the IMGT Scientific chart numbering (IMGT website). CH2 and CH3 domains are part of the Fc region of an immunoglobulin. In the context of IgG antibodies, an Fc region refers to the segment of amino acids from about position 231 to about position 447 as numbered according to the EU numbering scheme. As used herein, the term “Fc region” may also include at least a part of a hinge region of an antibody. An illustrative hinge region sequence is set forth in SEQ ID NO:104.

The term “variable region” refers to a domain in an antibody heavy chain or light chain that derived from a germline Variable (V) gene, Diversity (D) gene, or Joining (J) gene (and not derived from a Constant (C and CS) gene segment), and that gives an antibody its specificity for binding to an antigen. Typically, an antibody variable region comprises four conserved “framework” regions interspersed with three hypervariable “complementarity determining regions.”

The terms “wild-type,” “native,” and “naturally occurring” with respect to a CH3 or CH2 domain are used herein to refer to a domain that has a sequence that occurs in nature.

As used herein, the term “mutant” with respect to a mutant polypeptide or mutant polynucleotide is used interchangeably with “variant.” A variant with respect to a given wild-type CH3 or CH2 domain reference sequence can include naturally occurring allelic variants. A “non-naturally” occurring CH3 or CH2 domain refers to a variant or mutant domain that is not present in a cell in nature and that is produced by genetic modification, e.g., using genetic engineering technology or mutagenesis techniques, of a native CH3 domain or CH2 domain polynucleotide or polypeptide. A “variant” includes any domain comprising at least one amino acid mutation with respect to wild-type. Mutations may include substitutions, insertions, and deletions.

The term “amino acid” refers to naturally occurring and synthetic amino acids, as well as amino acid analogs and amino acid mimetics that function in a manner similar to the naturally occurring amino acids.

Naturally occurring amino acids are those encoded by the genetic code, as well as those amino acids that are later modified, e.g., hydroxyproline, γ-carboxyglutamate and O-phosphoserine. “Amino acid analogs” refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that function in a manner similar to a naturally occurring amino acid.

Naturally occurring α-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof. Stereoisomers of a naturally occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof.

Amino acids may be referred to herein by either their commonly known three letter symbols or by the one-letter symbols recommended by the IUPAC-IUB Biochemical Nomenclature Commission.

The terms “polypeptide” and “peptide” are used interchangeably herein to refer to a polymer of amino acid residues in a single chain. The terms apply to amino acid polymers in which one or more amino acid residue is an artificial chemical mimetic of a corresponding naturally occurring amino acid, as well as to naturally occurring amino acid polymers and non-naturally occurring amino acid polymers. Amino acid polymers may comprise entirely L-amino acids, entirely D-amino acids, or a mixture of L and D amino acids.

The term “protein” as used herein refers to either a polypeptide or a dimer (i.e, two) or multimer (i.e., three or more) of single chain polypeptides. The single chain polypeptides of a protein may be joined by a covalent bond, e.g., a disulfide bond, or non-covalent interactions.

The term “conservative substitution,” “conservative mutation,” or “conservatively modified variant” refers to an alteration that results in the substitution of an amino acid with another amino acid that can be categorized as having a similar feature. Examples of categories of conservative amino acid groups defined in this manner can include: a “charged/polar group” including Glu (Glutamic acid or E), Asp (Aspartic acid or D), Asn (Asparagine or N), Gln (Glutamine or Q), Lys (Lysine or K), Arg (Arginine or R), and His (Histidine or H); an “aromatic group” including Phe (Phenylalanine or F), Tyr (Tyrosine or Y), Trp (Tryptophan or W), and (Histidine or H); and an “aliphatic group” including Gly (Glycine or G), Ala (Alanine or A), Val (Valine or V), Leu (Leucine or L), Ile (Isoleucine or I), Met (Methionine or M), Ser (Serine or S), Thr (Threonine or T), and Cys (Cysteine or C). Within each group, subgroups can also be identified. For example, the group of charged or polar amino acids can be sub-divided into sub-groups including: a “positively-charged sub-group” comprising Lys, Arg and His; a “negatively-charged sub-group” comprising Glu and Asp; and a “polar sub-group” comprising Asn and Gln. In another example, the aromatic or cyclic group can be sub-divided into sub-groups including: a “nitrogen ring sub-group” comprising Pro, His and Trp; and a “phenyl sub-group” comprising Phe and Tyr. In another further example, the aliphatic group can be sub-divided into sub-groups, e.g., an “aliphatic non-polar sub-group” comprising Val, Leu, Gly, and Ala; and an “aliphatic slightly-polar sub-group” comprising Met, Ser, Thr, and Cys. Examples of categories of conservative mutations include amino acid substitutions of amino acids within the sub-groups above, such as, but not limited to: Lys for Arg or vice versa, such that a positive charge can be maintained; Glu for Asp or vice versa, such that a negative charge can be maintained; Ser for Thr or vice versa, such that a free —OH can be maintained; and Gln for Asn or vice versa, such that a free —NH₂ can be maintained. In some embodiments, hydrophobic amino acids are substituted for naturally occurring hydrophobic amino acid, e.g., in the active site, to preserve hydrophobicity.

The terms “identical” or percent “identity,” in the context of two or more polypeptide sequences, refer to two or more sequences or subsequences that are the same or have a specified percentage of amino acid residues, e.g., at least 60% identity, at least 65%, at least 70%, at least 75%, at least 80%, at least 85%, at least 90%, or at least 95% or greater, that are identical over a specified region when compared and aligned for maximum correspondence over a comparison window, or designated region as measured using one a sequence comparison algorithm or by manual alignment and visual inspection.

For sequence comparison of polypeptides, typically one amino acid sequence acts as a reference sequence, to which a candidate sequence is compared. Alignment can be performed using various methods available to one of skill in the art, e.g., visual alignment or using publicly available software using known algorithms to achieve maximal alignment. Such programs include the BLAST programs, ALIGN, ALIGN-2 (Genentech, South San Francisco, Calif.) or Megalign (DNASTAR). The parameters employed for an alignment to achieve maximal alignment can be determined by one of skill in the art. For sequence comparison of polypeptide sequences for purposes of this application, the BLASTP algorithm standard protein BLAST for aligning two proteins sequence with the default parameters is used.

The terms “corresponding to,” “determined with reference to,” or “numbered with reference to” when used in the context of the identification of a given amino acid residue in a polypeptide sequence, refers to the position of the residue of a specified reference sequence when the given amino acid sequence is maximally aligned and compared to the reference sequence. Thus, for example, an amino acid residue in a polypeptide “corresponds to” an amino acid in the region of SEQ ID NO:99, when the residue aligns with the amino acid in SEQ ID NO:99 when optimally aligned to SEQ ID NO:99. The polypeptide that is aligned to the reference sequence need not be the same length as the reference sequence.

As used herein, the term “specifically binds” or “selectively binds” to a target, e.g., TfR or FcγR, when referring to a polypeptide comprising a modified CH3 domain as described herein, refers to a binding reaction whereby the polypeptide binds to the target with greater affinity, greater avidity, and/or greater duration than it binds to a structurally different target. In typical embodiments, the polypeptide has at least 5-fold, 6-fold, 7-fold, 8-fold, 9-fold, 10-fold, 20-fold, 25-fold, 50-fold, 100-fold, 1,000-fold, 10,000-fold, or greater affinity for a specific target, e.g., TfR or FcγR, compared to an unrelated target when assayed under the same affinity assay conditions. The term “specific binding,” “specifically binds to,” or “is specific for” a particular target (e.g., e.g., TfR or FcγR), as used herein, can be exhibited, for example, by a molecule having an equilibrium dissociation constant K_{D} for the target to which it binds of, e.g., 10⁻⁴ M or smaller, e.g., 10⁻⁵ M, 10⁻⁶ M, 10⁻⁷ M, 10⁻⁸ M, 10⁻⁹ M, 10⁻¹⁰ M, 10⁻¹¹ M, or 10⁻¹² M. In some embodiments, a modified CH3 domain polypeptide specifically binds to an epitope on a TfR that is conserved among species (e.g., structurally conserved among species), e.g., conserved between non-human primate and human species (e.g., structurally conserved between non-human primate and human species). In some embodiments, a polypeptide may bind exclusively to a human TfR.

The term “binding affinity” as used herein refers to the strength of the non-covalent interaction between two molecules, e.g., a single binding site on a polypeptide and a target, e.g., TfR, to which it binds. Thus, for example, the term may refer to 1:1 interactions between a polypeptide and its target, unless otherwise indicated or clear from context. Binding affinity may be quantified by measuring an equilibrium dissociation constant (K_{D}), which refers to the dissociation rate constant (k_{d}, time⁻¹) divided by the association rate constant (k_{d}, time⁻¹ M⁻¹). K_{D} can be determined by measurement of the kinetics of complex formation and dissociation, e.g., using Surface Plasmon Resonance (SPR) methods, e.g., a Biacore™ system; kinetic exclusion assays such as KinExA®; and BioLayer interferometry (e.g., using the ForteBio® Octet® platform). As used herein, “binding affinity” includes not only formal binding affinities, such as those reflecting 1:1 interactions between a polypeptide and its target, but also apparent affinities for which K_{D}'s are calculated that may reflect avid binding.

The terms “antigen-binding portion” and “antigen-binding fragment” are used interchangeably herein and refer to one or more fragments of an antibody variable region that retains the ability to specifically bind to an antigen (e.g., HER2). Examples of antigen-binding fragments include, but are not limited to, a Fab fragment (a monovalent fragment consisting of the VL, VH, CL, and CH1 domains), a F(ab′)2 fragment (a bivalent fragment comprising two Fab fragments linked by a disulfide bridge at the hinge region), a single chain Fv (scFv), a disulfide-linked Fv (dsFv), complementarity determining regions (CDRs), a VL (light chain variable region), a VH (heavy chain variable region), nanobodies, diabodies, each of which bind the antigen via a variable region, and other formats as described in Spiess et al., Mol. Immun. 67 (2015) 95-106, which is incorporated herein by reference.

The term “complementarity determining region” or “CDR” refers to the three hypervariable regions in each chain that interrupt the four framework regions established by the light and heavy chain variable regions. The CDRs are primarily responsible for antibody binding to an epitope of an antigen. The CDRs of each chain are typically referred to as CDR1, CDR2, and CDR3, numbered sequentially starting from the N-terminus, and are also typically identified by the chain in which the particular CDR is located. Thus, a VH CDR3 or CDR-H3 is located in the variable region of the heavy chain of the antibody in which it is found, whereas a VL CDR1 or CDR-L1 is the CDR1 from the variable region of the light chain of the antibody in which it is found.

The “framework regions” or “FRs” of different light or heavy chains are relatively conserved within a species. The framework region of an antibody, that is the combined framework regions of the constituent light and heavy chains, serves to position and align the CDRs in three-dimensional space. Framework sequences can be obtained from public DNA databases or published references that include germline antibody gene sequences. For example, germline DNA sequences for human heavy and light chain variable region genes can be found in the “VBASE2” germline variable gene sequence database for human and mouse sequences.

The amino acid sequences of the CDRs and framework regions can be determined using various well known definitions in the art, e.g., Kabat, Chothia, international ImMunoGeneTics database (IMGT), AbM, and observed antigen contacts (“Contact”). In some embodiments, CDRs are determined according to the Contact definition. See, MacCallum et al., J. Mol. Biol., 262:732-745 (1996). In some embodiments, CDRs are determined by a combination of Kabat, Chothia, and Contact CDR definitions.

The term “subject,” “individual,” and “patient,” as used interchangeably herein, refer to a mammal, including but not limited to humans, non-human primates, rodents (e.g., rats, mice, and guinea pigs), rabbits, cows, pigs, horses, and other mammalian species. In one embodiment, the patient is a human.

The terms “treatment,” “treating,” and the like are used herein to generally mean obtaining a desired pharmacologic and/or physiologic effect. “Treating” or “treatment” may refer to any indicia of success in the treatment or amelioration of a cancer (e.g., a HER2-positive and/or metastatic cancer), including any objective or subjective parameter such as abatement, remission, improvement in patient survival, increase in survival time or rate, diminishing of symptoms or making the disease more tolerable to the patient, slowing in the rate of degeneration or decline, or improving a patient's physical or mental well-being. The treatment or amelioration of symptoms can be based on objective or subjective parameters. The effect of treatment can be compared to an individual or pool of individuals not receiving the treatment, or to the same patient prior to treatment or at a different time during treatment.

The term “pharmaceutically acceptable excipient” refers to a non-active pharmaceutical ingredient that is biologically or pharmacologically compatible for use in humans or animals, such as but not limited to a buffer, carrier, or preservative.

As used herein, a “therapeutic amount” or “therapeutically effective amount” of a construct (e.g., an antibody as described herein) is an amount of the construct that treats, alleviates, abates, or reduces the severity of symptoms of a disease in a subject. A “therapeutic amount” or “therapeutically effective amount” of a construct (e.g., an Fc polypeptide dimer-antibody variable region fusion protein or antibody heavy chain) may improve patient survival, increase survival time or rate, diminish symptoms, make an injury, disease, or condition (e.g., a cancer such as a HER2-positive and/or metastatic cancer) more tolerable, slow the rate of degeneration or decline, or improve a patient's physical or mental well-being.

The term “administer” refers to a method of delivering constructs, compounds, or compositions to the desired site of biological action. These methods include, but are not limited to, topical delivery, parenteral delivery, intravenous delivery, intradermal delivery, intramuscular delivery, intrathecal delivery, colonic delivery, rectal delivery, or intraperitoneal delivery. In one embodiment, an antibody as described herein is administered intravenously.

### III. Fc Polypeptide Dimer-Antibody Variable Region Fusion Proteins

In some aspects, the present disclosure provides Fc polypeptide dimer-antibody variable region fusion proteins that are capable of binding to human epidermal growth factor 2 (HER2) and are modified to bind to transferrin receptor (TfR), thus enabling the Fc polypeptide dimer-antibody variable region fusion proteins to cross the blood brain barrier (BBB)). In some embodiments The Fc polypeptide dimer-antibody variable region fusion proteins provided herein retain effector function upon binding to HER2, but have reduced effector function upon TfR binding. In this manner, the Fc polypeptide dimer-antibody variable region fusion proteins are able to transport an anti-HER2 antibody variable region (e.g., that form part of Fab domain) across the BBB without substantial depletion of reticulocytes (which also contain TfR on the cell surface), and still exhibit effector function that can target cancer cells (e.g., HER2-positive cancer cells or metastases thereof).

In some embodiments, provided herein are effector function-positive, Fc polypeptide dimer-antibody variable region fusion proteins that have a cis configuration, which means that only one (not both) of the Fc polypeptides in the Fc polypeptide dimer is modified to have a TfR-binding site and modifications that reduce FcγR binding when bound to TfR. In these embodiments, the other Fc polypeptide in the Fc polypeptide dimer does not contain either a TfR-binding site or modifications that substantially reduce FcγR binding. A trans configuration of the modified Fc polypeptide dimers refers to an Fc polypeptide dimer in which one of the two Fc polypeptides contains a TfR-binding site, while the other Fc polypeptide contains modifications, e.g., that reduce effector function, for example, when bound to TfR. Modified Fc polypeptide dimers having the cis configuration, but not the trans configuration, are able to reduce reticulocyte depletion in the blood and bone marrow.

In some embodiments, the Fc polypeptide dimer-antibody variable region fusion proteins provided herein do not substantially deplete reticulocytes (e.g, in bone marrow and/or in circulation). In some embodiments, the Fc polypeptide dimer-antibody variable region fusion proteins do not substantially deplete reticulocytes in vivo. In some embodiments, the amount of reticulocytes depleted after administering the Fc polypeptide dimer-antibody variable region fusion protein is less than an amount of reticulocytes depleted after administering a control. In some embodiments, the control is a corresponding TfR-binding polypeptide dimer-antibody variable region fusion protein with full effector function and/or contains no mutations that reduce FcγR binding. In some instances, the control is an Fc polypeptide dimer-antibody variable region fusion protein in which the first Fc polypeptide comprises the amino acid sequence set forth in SEQ ID NO:1, 3, 5, 7, 9, 11, 13, 15, 17, 19, 21, 23, 29, 31, 33, 35, 37, 39, 41, 43, 45, 47, 49, 51, 81, 83, 85, 87, 89, 91, 93, 95, 258, 260, 262, 264, 266, 268, 270, 272, 274, 276, 278, or 280 (i.e., a first Fc polypeptide that specifically binds TfR comprising a TfR-binding site but contains no LALA substitutions or other modifications that reduce FcγR binding) and the second Fc polypeptide does not contain a TfR-binding site or any modifications that reduce FcγR binding.

In some embodiments, the Fc polypeptide dimer-antibody variable region fusion protein comprises: (a) an antibody variable region that is capable of binding HER2 (e.g., subdomain II or IV of HER2), or an antigen-binding fragment thereof, and (b) a modified Fc polypeptide dimer comprising a first Fc polypeptide that contains modifications that create a TfR-binding site. In some embodiments, the modified Fc polypeptide dimer comprises a second Fc polypeptide that does not contain a TfR-binding site. In some embodiments, the first Fc polypeptide includes amino acid modifications that reduce FcγR binding when bound to TfR. In some embodiments, the second Fc polypeptide includes amino acid modifications that reduce FcγR binding when bound to TfR. In some embodiments, the first and second Fc polypeptides include amino acid modifications that reduce FcγR binding when bound to TfR. In some embodiments, the first and/or second Fc polypeptides include amino acid modifications that reduce FcγR binding when bound to TfR. In some embodiments, the amino acid modifications that reduce FcγR binding when bound to TfR comprise Ala at position 234 and at position 235, according to EU numbering.

In some embodiments, the first and/or second Fc polypeptides comprise amino acid modifications that increase serum half-life. In some embodiments, the first Fc polypeptide comprises amino acid modifications that increase serum half-life. In some embodiments, the second Fc polypeptide comprises amino acid modifications that increase serum half-life. In some embodiments, the first and second Fc polypeptides comprise amino acid modifications that increase serum half-life. In some embodiments, the amino acid modifications that increase serum half-life comprise (i) a Leu at position 428 and a Ser at position 434, or (ii) a Ser or Ala at position 434, according to EU numbering.

In some embodiments, the antibody variable region forms part of a Fab domain.

Anti-HER2 Variable Regions

Anti-HER2 DIV

In some embodiments, the antibody variable region comprises one or more complementarity determining regions (CDRs) selected from the group consisting of: (a) a heavy chain CDR1 having at least 90% sequence identity to the amino acid sequence of SEQ ID NO:69 or having up to two amino acid substitutions relative to the amino acid sequence of SEQ ID NO:69; (b) a heavy chain CDR2 having at least 90% sequence identity to the amino acid sequence of SEQ ID NO:70 or having up to two amino acid substitutions relative to the amino acid sequence of SEQ ID NO:70; (c) a heavy chain CDR3 having at least 90% sequence identity to the amino acid sequence of SEQ ID NO:71 or having up to two amino acid substitutions relative to the amino acid sequence of SEQ ID NO:71; (d) a light chain CDR1 having at least 90% sequence identity to the amino acid sequence of SEQ ID NO:72 or having up to two amino acid substitutions relative to the amino acid sequence of SEQ ID NO:72; (e) a light chain CDR2 having up to two amino acid substitutions relative to the amino acid sequence of SEQ ID NO:73; and (f) a light chain CDR3 having up to two amino acid substitutions relative to the amino acid sequence of SEQ ID NO:74.

In some embodiments, the antibody variable region comprises two, three, four, five, or all six of (a)-(f). In some embodiments, the antibody variable region comprises the heavy chain CDR1 of (a), the heavy chain CDR2 of (b), and the heavy chain CDR3 of (c). In some embodiments, the antibody variable region comprises the light chain CDR1 of (d), the light chain CDR2 of (e), and the light chain CDR3 of (f). In some embodiments, a CDR having up to two amino acid substitutions has one amino acid substitution relative to the reference sequence. In some embodiments, a CDR having up to two amino acid substitutions has two amino acid substitutions relative to the reference sequence. In some embodiments, the up to two amino acid substitutions are conservative substitutions.

In some embodiments, the antibody variable region comprises one or more CDRs selected from the group consisting of: (a) a heavy chain CDR1 comprising the amino acid sequence of SEQ ID NO:69; (b) a heavy chain CDR2 comprising the amino acid sequence of SEQ ID NO:70; (c) a heavy chain CDR3 comprising the amino acid sequence of SEQ ID NO:71; (d) a light chain CDR1 comprising the amino acid sequence of SEQ ID NO:72; (e) a light chain CDR2 comprising the amino acid sequence of SEQ ID NO:73; and (f) a light chain CDR3 comprising the amino acid sequence of SEQ ID NO:74.

In some embodiments, the antibody variable region comprises two, three, four, five, or all six of (a)-(f). In some embodiments, the antibody variable region comprises the heavy chain CDR1 of (a), the heavy chain CDR2 of (b), and the heavy chain CDR3 of (c). In some embodiments, the antibody variable region comprises the light chain CDR1 of (d), the light chain CDR2 of (e), and the light chain CDR3 of (f).

In some embodiments, the antibody variable region comprises: (a) a heavy chain variable region comprising (i) at least 75% sequence identity (e.g., at least 80%, at least 85%, at least 90%, at least 91%, at least 92%, at least 93%, at least 94%, at least 95%, at least 96%, at least 97%, at least 98%, or at least 99% sequence identity) to SEQ ID NO:59 and (ii) a CDR-H1, CDR-H2, and CDR-H3 that is identical to SEQ ID NOs:69, 70, and 71, respectively; and/or (b) a light chain variable region comprising (i) at least 75% sequence identity (e.g., at least 80%, at least 85%, at least 90%, at least 91%, at least 92%, at least 93%, at least 94%, at least 95%, at least 96%, at least 97%, at least 98%, or at least 99% sequence identity) to SEQ ID NO:60 and (ii) a CDR-L1, CDR-L2, and CDR-L3 that is identical to SEQ ID NOs:72, 73, and 74, respectively.

In some embodiments, the antibody variable region comprises a heavy chain variable region comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:59. In some embodiments, the antibody variable region comprises a heavy chain variable region comprising the amino acid sequence of SEQ ID NO:59.

In some embodiments, the antibody variable region comprises a light chain variable region comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:60. In some embodiments, the antibody variable region comprises a light chain variable region comprising the amino acid sequence of SEQ ID NO:60.

In some embodiments, the antibody variable region comprises two antibody heavy chain variable regions comprising the amino acid sequence of SEQ ID NO:59 and two light chain variable regions comprising the amino acid sequence of SEQ ID NO:60.

Anti-HER2_DII

In some embodiments, the antibody variable region comprises one or more complementarity determining regions (CDRs) selected from the group consisting of: (a) a heavy chain CDR1 having at least 90% sequence identity to the amino acid sequence of SEQ ID NO:75 or having up to two amino acid substitutions relative to the amino acid sequence of SEQ ID NO:75; (b) a heavy chain CDR2 having at least 90% sequence identity to the amino acid sequence of SEQ ID NO:76 or having up to two amino acid substitutions relative to the amino acid sequence of SEQ ID NO:76; (c) a heavy chain CDR3 having at least 90% sequence identity to the amino acid sequence of SEQ ID NO:77 or having up to two amino acid substitutions relative to the amino acid sequence of SEQ ID NO:77; (d) a light chain CDR1 having at least 90% sequence identity to the amino acid sequence of SEQ ID NO:78 or having up to two amino acid substitutions relative to the amino acid sequence of SEQ ID NO:78; (e) a light chain CDR2 having up to two amino acid substitutions relative to the amino acid sequence of SEQ ID NO:79; and (f) a light chain CDR3 having up to two amino acid substitutions relative to the amino acid sequence of SEQ ID NO:80.

In some embodiments, the antibody variable region comprises two, three, four, five, or all six of (a)-(f). In some embodiments, the antibody variable region comprises the heavy chain CDR1 of (a), the heavy chain CDR2 of (b), and the heavy chain CDR3 of (c). In some embodiments, the antibody variable region comprises the light chain CDR1 of (d), the light chain CDR2 of (e), and the light chain CDR3 of (f). In some embodiments, a CDR having up to two amino acid substitutions has one amino acid substitution relative to the reference sequence. In some embodiments, a CDR having up to two amino acid substitutions has two amino acid substitutions relative to the reference sequence. In some embodiments, the up to two amino acid substitutions are conservative substitutions.

In some embodiments, the antibody variable region comprises one or more CDRs selected from the group consisting of: (a) a heavy chain CDR1 comprising the amino acid sequence of SEQ ID NO:75; (b) a heavy chain CDR2 comprising the amino acid sequence of SEQ ID NO:76; (c) a heavy chain CDR3 comprising the amino acid sequence of SEQ ID NO:77; (d) a light chain CDR1 comprising the amino acid sequence of SEQ ID NO:78; (e) a light chain CDR2 comprising the amino acid sequence of SEQ ID NO:79; and (f) a light chain CDR3 comprising the amino acid sequence of SEQ ID NO: 80.

In some embodiments, the antibody variable region comprises two, three, four, five, or all six of (a)-(f). In some embodiments, the antibody variable region comprises the heavy chain CDR1 of (a), the heavy chain CDR2 of (b), and the heavy chain CDR3 of (c). In some embodiments, the antibody variable region comprises the light chain CDR1 of (d), the light chain CDR2 of (e), and the light chain CDR3 of (f).

In some embodiments, the antibody variable region comprises: (a) a heavy chain variable region comprising (i) at least 75% sequence identity (e.g., at least 80%, at least 85%, at least 90%, at least 91%, at least 92%, at least 93%, at least 94%, at least 95%, at least 96%, at least 97%, at least 98%, or at least 99% sequence identity) to SEQ ID NO:61 and (ii) a CDR-H1, CDR-H2, and CDR-H3 that is identical to SEQ ID NOs:75, 76, and 77, respectively; and/or (b) a light chain variable region comprising (i) at least 75% sequence identity (e.g., at least 80%, at least 85%, at least 90%, at least 91%, at least 92%, at least 93%, at least 94%, at least 95%, at least 96%, at least 97%, at least 98%, or at least 99% sequence identity) to SEQ ID NO:62 and (ii) a CDR-L1, CDR-L2, and CDR-L3 that is identical to SEQ ID NOs:78, 79, and 80, respectively.

In some embodiments, the antibody variable region comprises a heavy chain variable region comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:61. In some embodiments, the antibody variable region comprises a heavy chain variable region comprising the amino acid sequence of SEQ ID NO:61.

In some embodiments, the antibody variable region comprises a light chain variable region comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:62. In some embodiments, the antibody variable region comprises a light chain variable region comprising the amino acid sequence of SEQ ID NO:62.

In some embodiments, the antibody variable region comprises two antibody heavy chain variable regions comprising the amino acid sequence of SEQ ID NO:61 and two light chain variable regions comprising the amino acid sequence of SEQ ID NO:62.

Anti-HER2_DI

In some embodiments, the antibody variable region comprises one or more complementarity determining regions (CDRs) selected from the group consisting of: (a) a heavy chain CDR1 having at least 90% sequence identity to the amino acid sequence of SEQ ID NO:250 or having up to two amino acid substitutions relative to the amino acid sequence of SEQ ID NO:250; (b) a heavy chain CDR2 having at least 90% sequence identity to the amino acid sequence of SEQ ID NO:251 or having up to two amino acid substitutions relative to the amino acid sequence of SEQ ID NO:251; (c) a heavy chain CDR3 having at least 90% sequence identity to the amino acid sequence of SEQ ID NO:252 or having up to two amino acid substitutions relative to the amino acid sequence of SEQ ID NO:252; (d) a light chain CDR1 having at least 90% sequence identity to the amino acid sequence of SEQ ID NO:253 or having up to two amino acid substitutions relative to the amino acid sequence of SEQ ID NO:253; (e) a light chain CDR2 having up to two amino acid substitutions relative to the amino acid sequence of SEQ ID NO:254; and (f) a light chain CDR3 having up to two amino acid substitutions relative to the amino acid sequence of SEQ ID NO:255.

In some embodiments, the antibody variable region comprises two, three, four, five, or all six of (a)-(f). In some embodiments, the antibody variable region comprises the heavy chain CDR1 of (a), the heavy chain CDR2 of (b), and the heavy chain CDR3 of (c). In some embodiments, the antibody variable region comprises the light chain CDR1 of (d), the light chain CDR2 of (e), and the light chain CDR3 of (f). In some embodiments, a CDR having up to two amino acid substitutions has one amino acid substitution relative to the reference sequence. In some embodiments, a CDR having up to two amino acid substitutions has two amino acid substitutions relative to the reference sequence. In some embodiments, the up to two amino acid substitutions are conservative substitutions.

In some embodiments, the antibody variable region comprises one or more CDRs selected from the group consisting of: (a) a heavy chain CDR1 comprising the amino acid sequence of SEQ ID NO:250; (b) a heavy chain CDR2 comprising the amino acid sequence of SEQ ID NO:251; (c) a heavy chain CDR3 comprising the amino acid sequence of SEQ ID NO:252; (d) a light chain CDR1 comprising the amino acid sequence of SEQ ID NO:253; (e) a light chain CDR2 comprising the amino acid sequence of SEQ ID NO:254; and (f) a light chain CDR3 comprising the amino acid sequence of SEQ ID NO:255.

In some embodiments, the antibody variable region comprises two, three, four, five, or all six of (a)-(f). In some embodiments, the antibody variable region comprises the heavy chain CDR1 of (a), the heavy chain CDR2 of (b), and the heavy chain CDR3 of (c). In some embodiments, the antibody variable region comprises the light chain CDR1 of (d), the light chain CDR2 of (e), and the light chain CDR3 of (f).

In some embodiments, the antibody variable region comprises: (a) a heavy chain variable region comprising (i) at least 75% sequence identity (e.g., at least 80%, at least 85%, at least 90%, at least 91%, at least 92%, at least 93%, at least 94%, at least 95%, at least 96%, at least 97%, at least 98%, or at least 99% sequence identity) to SEQ ID NO:256 and (ii) a CDR-H1, CDR-H2, and CDR-H3 that is identical to SEQ ID NOs:250, 251, and 252, respectively; and/or (b) a light chain variable region comprising (i) at least 75% sequence identity (e.g., at least 80%, at least 85%, at least 90%, at least 91%, at least 92%, at least 93%, at least 94%, at least 95%, at least 96%, at least 97%, at least 98%, or at least 99% sequence identity) to SEQ ID NO:257 and (ii) a CDR-L1, CDR-L2, and CDR-L3 that is identical to SEQ ID NOs:253, 254, and 255, respectively.

In some embodiments, the antibody variable region comprises a heavy chain variable region comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:256. In some embodiments, the antibody variable region comprises a heavy chain variable region comprising the amino acid sequence of SEQ ID NO:256.

In some embodiments, the antibody variable region comprises a light chain variable region comprising an amino acid sequence that has at least 90% sequence identity (e.g., at least 91%, at least 92%, at least 93%, at least 94%, at least 95%, at least 96%, at least 97%, at least 98%, or at least 99% sequence identity) to SEQ ID NO:257. In some embodiments, the antibody variable region comprises a light chain variable region comprising the amino acid sequence of SEQ ID NO:257.

In some embodiments, the antibody variable region comprises two antibody heavy chain variable regions comprising the amino acid sequence of SEQ ID NO:256 and two light chain variable regions comprising the amino acid sequence of SEQ ID NO:257.

Illustrative Fc Polypeptide Dimer-Antibody Variable Region Fusion Proteins

Fc polypeptide dimer-antibody variable region fusion proteins described herein can comprise any combination of the anti-HER2 variable regions described above. In some embodiments, the first Fc polypeptide comprises a TfR-binding site that comprises a modified CH3 domain. Non-limiting examples of modified CH3 domains that can be used in compositions and methods are described herein in the section titled “TfR-Binding Fc Polypeptides.” In some embodiments, the first Fc polypeptide further comprises a knob mutation T366W and the second Fc polypeptide comprises hole mutations T366S, L368A, and Y407V, according to EU numbering. In some embodiments, the first Fc polypeptide comprises an amino acid sequence having at least about 80%, 85%, 90%, 95,%, 96%, 97%, 98%, or 99% identity to SEQ ID NO:63. In some instances, the first Fc polypeptide comprises the amino acid sequence of SEQ ID NO:63. In some embodiments, the second Fc polypeptide comprises an amino acid sequence having at least about 80%, 85%, 90%, 95,%, 96%, 97%, 98%, or 99% identity to SEQ ID NO:67 or 68. In some instances, the second Fc polypeptide comprises the amino acid sequence of any one of SEQ ID NOS:67 and 68.

In some embodiments, the first Fc polypeptide further comprises hole mutations T366S, L368A, and Y407V and the second Fc polypeptide comprises a knob mutation T366W, according to EU numbering. In some embodiments, the first Fc polypeptide comprises an amino acid sequence having at least about 80%, 85%, 90%, 95,%, 96%, 97%, 98%, or 99% identity to SEQ ID NO:64. In some instances, the first Fc polypeptide comprises the amino acid sequence of SEQ ID NO:64. In some embodiments, the second Fc polypeptide comprises an amino acid sequence having at least about 80%, 85%, 90%, 95,%, 96%, 97%, 98%, or 99% identity to SEQ ID NO:65 or 66. In some instances, the second FC polypeptide comprises the amino acid sequence of any one of SEQ ID NOS:65 and 66.

In some embodiments, an Fc polypeptide dimer-antibody variable region fusion protein comprises: (a) an antibody variable region that is capable of binding HER2 (e.g., human HER2), or an antigen-binding fragment thereof, (b) a first Fc polypeptide that contains modifications that create a TfR-binding site, amino acid modifications L234A and L235A, and a knob mutation T366W, according to EU numbering, and (c) a second Fc polypeptide that comprises hole mutations T366S, L368A, and Y407V, according to EU numbering, and does not contain a TfR-binding site or any modifications that reduce FcγR binding.

In some embodiments, the Fc polypeptide dimer-antibody variable region fusion protein comprises a first heavy chain comprising the amino acid sequence of any one of SEQ ID NOS:2, 10, 18, and 82. In some embodiments, the Fc polypeptide dimer-antibody variable region fusion protein comprises a second heavy chain comprising the amino acid sequence of SEQ ID NO:27. In some embodiments, the Fc polypeptide dimer-antibody variable region fusion protein comprises two light chains comprising the amino acid sequence of SEQ ID NO:57.

In some embodiments, the Fc polypeptide dimer-antibody variable region fusion protein comprises a first heavy chain comprising the amino acid sequence of any one of SEQ ID NOS:30, 38, 46, and 90. In some embodiments, the Fc polypeptide dimer-antibody variable region fusion protein comprises a second heavy chain comprising the amino acid sequence of SEQ ID NO:55. In some embodiments, the Fc polypeptide dimer-antibody variable region fusion protein comprises two light chains comprising the amino acid sequence of SEQ ID NO:58.

In some embodiments, the Fc polypeptide dimer-antibody variable region fusion protein comprises a first heavy chain comprising the amino acid sequence of any one of SEQ ID NOS:259, 267, 275, and 283. In some embodiments, the Fc polypeptide dimer-antibody variable region fusion protein comprises a second heavy chain comprising the amino acid sequence of SEQ ID NO:290. In some embodiments, the Fc polypeptide dimer-antibody variable region fusion protein comprises two light chains comprising the amino acid sequence of SEQ ID NO:293.

In some embodiments, an Fc polypeptide dimer-antibody variable region fusion protein comprises: (a) an antibody variable region that is capable of binding HER2 (e.g., human HER2), or an antigen-binding fragment thereof, (b) a first Fc polypeptide that contains modifications that create a TfR-binding site, amino acid modifications L234A and L235A, a knob mutation T366W, and amino acid modification N434S with or without M428L, according to EU numbering, and (c) a second Fc polypeptide that comprises hole mutations T366S, L368A, and Y407V, according to EU numbering, and does not contain a TfR-binding site.

In some embodiments, the Fc polypeptide dimer-antibody variable region fusion protein comprises a first heavy chain comprising the amino acid sequence of any one of SEQ ID NOS:4, 12, 20, and 84. In some embodiments, the Fc polypeptide dimer-antibody variable region fusion protein comprises a second heavy chain comprising the amino acid sequence of SEQ ID NO:27. In some embodiments, the Fc polypeptide dimer-antibody variable region fusion protein comprises two light chains comprising the amino acid sequence of SEQ ID NO:57.

In some embodiments, the Fc polypeptide dimer-antibody variable region fusion protein comprises a first heavy chain comprising the amino acid sequence of any one of SEQ ID NOS:32, 40, 48, and 92. In some embodiments, the Fc polypeptide dimer-antibody variable region fusion protein comprises a second heavy chain comprising the amino acid sequence of SEQ ID NO:55. In some embodiments, the Fc polypeptide dimer-antibody variable region fusion protein comprises two light chains comprising the amino acid sequence of SEQ ID NO:58.

In some embodiments, the Fc polypeptide dimer-antibody variable region fusion protein comprises a first heavy chain comprising the amino acid sequence of any one of SEQ ID NOS:261, 269, 277, and 285. In some embodiments, the Fc polypeptide dimer-antibody variable region fusion protein comprises a second heavy chain comprising the amino acid sequence of SEQ ID NO:290. In some embodiments, the Fc polypeptide dimer-antibody variable region fusion protein comprises two light chains comprising the amino acid sequence of SEQ ID NO:293.

In some embodiments, an Fc polypeptide dimer-antibody variable region fusion protein comprises: (a) an antibody variable region that is capable of binding HER2 (e.g., human HER2), or an antigen-binding fragment thereof, (b) a first Fc polypeptide that contains modifications that create a TfR-binding site, amino acid modifications L234A and L235A, and a knob mutation T366W, according to EU numbering, and (c) a second Fc polypeptide that comprises hole mutations T366S, L368A, and Y407V and amino acid modification N434S with or without M428L, according to EU numbering, and does not contain a TfR-binding site.

In some embodiments, the Fc polypeptide dimer-antibody variable region fusion protein comprises a first heavy chain comprising the amino acid sequence of any one of SEQ ID NOS:2, 10, 18, and 82. In some embodiments, the Fc polypeptide dimer-antibody variable region fusion protein comprises a second heavy chain comprising the amino acid sequence of SEQ ID NO:28. In some embodiments, the Fc polypeptide dimer-antibody variable region fusion protein comprises two light chains comprising the amino acid sequence of SEQ ID NO:57.

In some embodiments, the Fc polypeptide dimer-antibody variable region fusion protein comprises a first heavy chain comprising the amino acid sequence of any one of SEQ ID NOS:30, 38, 46, and 90. In some embodiments, the Fc polypeptide dimer-antibody variable region fusion protein comprises a second heavy chain comprising the amino acid sequence of SEQ ID NO:56. In some embodiments, the Fc polypeptide dimer-antibody variable region fusion protein comprises two light chains comprising the amino acid sequence of SEQ ID NO:58.

In some embodiments, the Fc polypeptide dimer-antibody variable region fusion protein comprises a first heavy chain comprising the amino acid sequence of any one of SEQ ID NOS:259, 267, 275, and 283. In some embodiments, the Fc polypeptide dimer-antibody variable region fusion protein comprises a second heavy chain comprising the amino acid sequence of SEQ ID NO:291. In some embodiments, the Fc polypeptide dimer-antibody variable region fusion protein comprises two light chains comprising the amino acid sequence of SEQ ID NO:293.

In some embodiments, an Fc polypeptide dimer-antibody variable region fusion protein comprises: (a) an antibody variable region that is capable of binding HER2 (e.g., human HER2), or an antigen-binding fragment thereof, (b) a first Fc polypeptide that contains modifications that create a TfR-binding site, amino acid modifications L234A and L235A, a knob mutation T366W, and amino acid modification N434S with or without M428L, according to EU numbering, and (c) a second Fc polypeptide that comprises hole mutations T366S, L368A, and Y407V and amino acid modification N434S with or without M428L, according to EU numbering, and does not contain a TfR-binding site.

In some embodiments, the Fc polypeptide dimer-antibody variable region fusion protein comprises a first heavy chain comprising the amino acid sequence of any one of SEQ ID NOS:4, 12, 20, and 84. In some embodiments, the Fc polypeptide dimer-antibody variable region fusion protein comprises a second heavy chain comprising the amino acid sequence of SEQ ID NO:28. In some embodiments, the Fc polypeptide dimer-antibody variable region fusion protein comprises two light chains comprising the amino acid sequence of SEQ ID NO:57.

In some embodiments, the Fc polypeptide dimer-antibody variable region fusion protein comprises a first heavy chain comprising the amino acid sequence of any one of SEQ ID NOS:32, 40, 48, and 92. In some embodiments, the Fc polypeptide dimer-antibody variable region fusion protein comprises a second heavy chain comprising the amino acid sequence of SEQ ID NO:56. In some embodiments, the Fc polypeptide dimer-antibody variable region fusion protein comprises two light chains comprising the amino acid sequence of SEQ ID NO:58.

In some embodiments, the Fc polypeptide dimer-antibody variable region fusion protein comprises a first heavy chain comprising the amino acid sequence of any one of SEQ ID NOS:261, 269, 277, and 285. In some embodiments, the Fc polypeptide dimer-antibody variable region fusion protein comprises a second heavy chain comprising the amino acid sequence of SEQ ID NO:291. In some embodiments, the Fc polypeptide dimer-antibody variable region fusion protein comprises two light chains comprising the amino acid sequence of SEQ ID NO:293.

In some embodiments, an Fc polypeptide dimer-antibody variable region fusion protein comprises: (a) an antibody variable region that is capable of binding HER2 (e.g., human HER2), or an antigen-binding fragment thereof, (b) a first Fc polypeptide that contains modifications that create a TfR-binding site, amino acid modifications L234A and L235A, and hole mutations T366S, L368A, and Y407V, according to EU numbering, and (c) a second Fc polypeptide that comprises a knob mutation T366W, according to EU numbering, and does not contain a TfR-binding site.

In some embodiments, the Fc polypeptide dimer-antibody variable region fusion protein comprises a first heavy chain comprising the amino acid sequence of any one of SEQ ID NOS:6, 14, 22, and 86. In some embodiments, he Fc polypeptide dimer-antibody variable region fusion protein comprises a second heavy chain comprising the amino acid sequence of SEQ ID NO:25. In some embodiments, the Fc polypeptide dimer-antibody variable region fusion protein comprises two light chains comprising the amino acid sequence of SEQ ID NO:57.

In some embodiments, the Fc polypeptide dimer-antibody variable region fusion protein comprises a first heavy chain comprising the amino acid sequence of any one of SEQ ID NOS:34, 42, 50, and 94. In some embodiments, the Fc polypeptide dimer-antibody variable region fusion protein comprises a second heavy chain comprising the amino acid sequence of SEQ ID NO:53. In some embodiments, the Fc polypeptide dimer-antibody variable region fusion protein comprises two light chains comprising the amino acid sequence of SEQ ID NO:58.

In some embodiments, the Fc polypeptide dimer-antibody variable region fusion protein comprises a first heavy chain comprising the amino acid sequence of any one of SEQ ID NOS:263, 271, 279, and 287. In some embodiments, he Fc polypeptide dimer-antibody variable region fusion protein comprises a second heavy chain comprising the amino acid sequence of SEQ ID NO:294. In some embodiments, the Fc polypeptide dimer-antibody variable region fusion protein comprises two light chains comprising the amino acid sequence of SEQ ID NO:293.

In some embodiments, an Fc polypeptide dimer-antibody variable region fusion protein comprises: (a) an antibody variable region that is capable of binding HER2 (e.g., human HER2), or an antigen-binding fragment thereof, (b) a first Fc polypeptide that contains modifications that create a TfR-binding site, amino acid modifications L234A and L235A, hole mutations T366S, L368A, and Y407V, and amino acid modification N434S with or without M428L, according to EU numbering, and (c) a second Fc polypeptide that comprises a knob mutation T366W, according to EU numbering, and does not contain a TfR-binding site.

In some embodiments, the Fc polypeptide dimer-antibody variable region fusion protein comprises a first heavy chain comprising the amino acid sequence of any one of SEQ ID NOS:8, 16, 24, and 88. In some embodiments, the Fc polypeptide dimer-antibody variable region fusion protein comprises a second heavy chain comprising the amino acid sequence of SEQ ID NO:25. In some embodiments, the Fc polypeptide dimer-antibody variable region fusion protein comprises two light chains comprising the amino acid sequence of SEQ ID NO:57.

In some embodiments, the Fc polypeptide dimer-antibody variable region fusion protein comprises a first heavy chain comprising the amino acid sequence of any one of SEQ ID NOS:36, 44, 52, and 96. In some embodiments, the Fc polypeptide dimer-antibody variable region fusion protein comprises a second heavy chain comprising the amino acid sequence of SEQ ID NO:53. In some embodiments, the Fc polypeptide dimer-antibody variable region fusion protein comprises two light chains comprising the amino acid sequence of SEQ ID NO:58.

In some embodiments, the Fc polypeptide dimer-antibody variable region fusion protein comprises a first heavy chain comprising the amino acid sequence of any one of SEQ ID NOS:265, 273, 281, and 289. In some embodiments, the Fc polypeptide dimer-antibody variable region fusion protein comprises a second heavy chain comprising the amino acid sequence of SEQ ID NO:294. In some embodiments, the Fc polypeptide dimer-antibody variable region fusion protein comprises two light chains comprising the amino acid sequence of SEQ ID NO:293.

In some embodiments, an Fc polypeptide dimer-antibody variable region fusion protein comprises: (a) an antibody variable region that is capable of binding HER2 (e.g., human HER2), or an antigen-binding fragment thereof, (b) a first Fc polypeptide contains modifications that create a TfR-binding site, amino acid modifications L234A and L235A, and hole mutations T366S, L368A, and Y407V, according to EU numbering, and (c) a second Fc polypeptide that comprises a knob mutation T366W and amino acid modification N434S with or without M428L, according to EU numbering, and does not contain a TfR-binding site.

In some embodiments, the Fc polypeptide dimer-antibody variable region fusion protein comprises a first heavy chain comprising the amino acid sequence of any one of SEQ ID NOS:6, 14, 22, and 86. In some embodiments, the Fc polypeptide dimer-antibody variable region fusion protein comprises a second heavy chain comprising the amino acid sequence of SEQ ID NO:26. In some embodiments, the Fc polypeptide dimer-antibody variable region fusion protein comprises two light chains comprising the amino acid sequence of SEQ ID NO:57.

In some embodiments, the Fc polypeptide dimer-antibody variable region fusion protein comprises a first heavy chain comprising the amino acid sequence of any one of SEQ ID NOS:34, 42, 50, and 94. In some embodiments, the Fc polypeptide dimer-antibody variable region fusion protein comprises a second heavy chain comprising the amino acid sequence of SEQ ID NO:54. In some embodiments, the Fc polypeptide dimer-antibody variable region fusion protein comprises two light chains comprising the amino acid sequence of SEQ ID NO:58.

In some embodiments, the Fc polypeptide dimer-antibody variable region fusion protein comprises a first heavy chain comprising the amino acid sequence of any one of SEQ ID NOS:263, 271, 279, and 287. In some embodiments, the Fc polypeptide dimer-antibody variable region fusion protein comprises a second heavy chain comprising the amino acid sequence of SEQ ID NO:295. In some embodiments, the Fc polypeptide dimer-antibody variable region fusion protein comprises two light chains comprising the amino acid sequence of SEQ ID NO:293.

In some embodiments, an Fc polypeptide dimer-antibody variable region fusion protein comprises: (a) an antibody variable region that is capable of binding HER2 (e.g., human HER2), or an antigen-binding fragment thereof, (b) a first Fc polypeptide that contains modifications that create a TfR-binding site, amino acid modifications L234A and L235A, hole mutations T366S, L368A, and Y407V, and amino acid modification N434S with or without M428L, according to EU numbering, and (c) a second Fc polypeptide that comprises a knob mutation T366W and amino acid modification N434S with or without M428L, according to EU numbering, and does not contain a TfR-binding site.

In some embodiments, the Fc polypeptide dimer-antibody variable region fusion protein comprises a first heavy chain comprising the amino acid sequence of any one of SEQ ID NOS:8, 16, 24, and 88. In some embodiments, the Fc polypeptide dimer-antibody variable region fusion protein comprises a second heavy chain comprising the amino acid sequence of SEQ ID NO:26. In some embodiments, the Fc polypeptide dimer-antibody variable region fusion protein comprises two light chains comprising the amino acid sequence of SEQ ID NO:57.

In some embodiments, the Fc polypeptide dimer-antibody variable region fusion protein comprises a first heavy chain comprising the amino acid sequence of any one of SEQ ID NOS:36, 44, 52, and 96. In some embodiments, the Fc polypeptide dimer-antibody variable region fusion protein comprises a second heavy chain comprising the amino acid sequence of SEQ ID NO:54. In some embodiments, the Fc polypeptide dimer-antibody variable region fusion protein comprises two light chains comprising the amino acid sequence of SEQ ID NO:58.

In some embodiments, the Fc polypeptide dimer-antibody variable region fusion protein comprises a first heavy chain comprising the amino acid sequence of any one of SEQ ID NOS:265, 273, 281, and 289. In some embodiments, the Fc polypeptide dimer-antibody variable region fusion protein comprises a second heavy chain comprising the amino acid sequence of SEQ ID NO:295. In some embodiments, the Fc polypeptide dimer-antibody variable region fusion protein comprises two light chains comprising the amino acid sequence of SEQ ID NO:293.

In some embodiments, an Fc polypeptide dimer-antibody variable region fusion protein comprises: (a) an antibody variable region that is capable of binding HER2 (e.g., human HER2), or an antigen-binding fragment thereof, (b) a first Fc polypeptide that contains modifications that create a TfR-binding site and a knob mutation T366W, according to EU numbering, and (c) a second Fc polypeptide that comprises hole mutations T366S, L368A, and Y407V, according to EU numbering, and does not contain a TfR-binding site.

In some embodiments, the Fc polypeptide dimer-antibody variable region fusion protein comprises a first heavy chain comprising the amino acid sequence of any one of SEQ ID NOS:1, 9, 17, and 81. In some embodiments, the Fc polypeptide dimer-antibody variable region fusion protein comprises a second heavy chain comprising the amino acid sequence of SEQ ID NO:27. In some embodiments, the Fc polypeptide dimer-antibody variable region fusion protein comprises two light chains comprising the amino acid sequence of SEQ ID NO:57.

In some embodiments, the Fc polypeptide dimer-antibody variable region fusion protein comprises a first heavy chain comprising the amino acid sequence of any one of SEQ ID NOS:29, 37, 45, and 89. In some embodiments, the Fc polypeptide dimer-antibody variable region fusion protein comprises a second heavy chain comprising the amino acid sequence of SEQ ID NO:55. In some embodiments, the Fc polypeptide dimer-antibody variable region fusion protein comprises two light chains comprising the amino acid sequence of SEQ ID NO:58.

In some embodiments, the Fc polypeptide dimer-antibody variable region fusion protein comprises a first heavy chain comprising the amino acid sequence of any one of SEQ ID NOS:258, 266, 274, and 282. In some embodiments, the Fc polypeptide dimer-antibody variable region fusion protein comprises a second heavy chain comprising the amino acid sequence of SEQ ID NO:290. In some embodiments, the Fc polypeptide dimer-antibody variable region fusion protein comprises two light chains comprising the amino acid sequence of SEQ ID NO:293.

In some embodiments, an Fc polypeptide dimer-antibody variable region fusion protein comprises: (a) an antibody variable region that is capable of binding HER2 (e.g., human HER2), or an antigen-binding fragment thereof, (b) a first Fc polypeptide that contains modifications that create a TfR-binding site, a knob mutation T366W, and amino acid modification N434S with or without M428L, according to EU numbering, and (c) a second Fc polypeptide that comprises hole mutations T366S, L368A, and Y407V, according to EU numbering, and does not contain a TfR-binding site.

In some embodiments, the Fc polypeptide dimer-antibody variable region fusion protein comprises a first heavy chain comprising the amino acid sequence of any one of SEQ ID NOS:3, 11, 19, and 83. In some embodiments, the Fc polypeptide dimer-antibody variable region fusion protein comprises a second heavy chain comprising the amino acid sequence of SEQ ID NO:27. In some embodiments, the Fc polypeptide dimer-antibody variable region fusion protein comprises two light chains comprising the amino acid sequence of SEQ ID NO:57.

In some embodiments, the Fc polypeptide dimer-antibody variable region fusion protein comprises a first heavy chain comprising the amino acid sequence of any one of SEQ ID NOS:31, 39, 47, and 91. In some embodiments, the Fc polypeptide dimer-antibody variable region fusion protein comprises a second heavy chain comprising the amino acid sequence of SEQ ID NO:55. In some embodiments, the Fc polypeptide dimer-antibody variable region fusion protein comprises two light chains comprising the amino acid sequence of SEQ ID NO:58.

In some embodiments, the Fc polypeptide dimer-antibody variable region fusion protein comprises a first heavy chain comprising the amino acid sequence of any one of SEQ ID NOS:260, 268, 276, and 284. In some embodiments, the Fc polypeptide dimer-antibody variable region fusion protein comprises a second heavy chain comprising the amino acid sequence of SEQ ID NO:290. In some embodiments, the Fc polypeptide dimer-antibody variable region fusion protein comprises two light chains comprising the amino acid sequence of SEQ ID NO:293.

In some embodiments, an Fc polypeptide dimer-antibody variable region fusion protein comprises: (a) an antibody variable region that is capable of binding HER2 (e.g., human HER2), or an antigen-binding fragment thereof, (b) a first Fc polypeptide that contains modifications that create a TfR-binding site and a knob mutation T366W, according to EU numbering, and (c) a second Fc polypeptide that comprises hole mutations T366S, L368A, and Y407V and amino acid modification N434S with or without M428L, according to EU numbering, and does not contain a TfR-binding site.

In some embodiments, the Fc polypeptide dimer-antibody variable region fusion protein comprises a first heavy chain comprising the amino acid sequence of any one of SEQ ID NOS:1, 9, 17, and 81. In some embodiments, the Fc polypeptide dimer-antibody variable region fusion protein comprises a second heavy chain comprising the amino acid sequence of SEQ ID NO:28. In some embodiments, the Fc polypeptide dimer-antibody variable region fusion protein comprises two light chains comprising the amino acid sequence of SEQ ID NO:57.

In some embodiments, the Fc polypeptide dimer-antibody variable region fusion protein comprises a first heavy chain comprising the amino acid sequence of any one of SEQ ID NOS:29, 37, 45, and 89. In some embodiments, the Fc polypeptide dimer-antibody variable region fusion protein comprises a second heavy chain comprising the amino acid sequence of SEQ ID NO:56. In some embodiments, the Fc polypeptide dimer-antibody variable region fusion protein comprises two light chains comprising the amino acid sequence of SEQ ID NO:58.

In some embodiments, the Fc polypeptide dimer-antibody variable region fusion protein comprises a first heavy chain comprising the amino acid sequence of any one of SEQ ID NOS:258, 266, 274, and 282. In some embodiments, the Fc polypeptide dimer-antibody variable region fusion protein comprises a second heavy chain comprising the amino acid sequence of SEQ ID NO:291. In some embodiments, the Fc polypeptide dimer-antibody variable region fusion protein comprises two light chains comprising the amino acid sequence of SEQ ID NO:293.

In some embodiments, an Fc polypeptide dimer-antibody variable region fusion protein comprises: (a) an antibody variable region that is capable of binding HER2 (e.g., human HER2), or an antigen-binding fragment thereof, (b) a first Fc polypeptide that contains modifications that create a TfR-binding site, a knob mutation T366W, and amino acid modification N434S with or without M428L, according to EU numbering, and (c) a second Fc polypeptide that comprises hole mutations T366S, L368A, and Y407V and amino acid modification N434S with or without M428L, according to EU numbering, and does not contain a TfR-binding site.

In some embodiments, the Fc polypeptide dimer-antibody variable region fusion protein comprises a first heavy chain comprising the amino acid sequence of any one of SEQ ID NOS:3, 11, 19, and 83. In some embodiments, the Fc polypeptide dimer-antibody variable region fusion protein comprises a second heavy chain comprising the amino acid sequence of SEQ ID NO:28. In some embodiments, the Fc polypeptide dimer-antibody variable region fusion protein comprises two light chains comprising the amino acid sequence of SEQ ID NO:57.

In some embodiments, the Fc polypeptide dimer-antibody variable region fusion protein comprises a first heavy chain comprising the amino acid sequence of any one of SEQ ID NOS:31, 39, 47, and 91. In some embodiments, the Fc polypeptide dimer-antibody variable region fusion protein comprises a second heavy chain comprising the amino acid sequence of SEQ ID NO:56. In some embodiments, the Fc polypeptide dimer-antibody variable region fusion protein comprises two light chains comprising the amino acid sequence of SEQ ID NO:58.

In some embodiments, the Fc polypeptide dimer-antibody variable region fusion protein comprises a first heavy chain comprising the amino acid sequence of any one of SEQ ID NOS:260, 268, 276, and 284. In some embodiments, the Fc polypeptide dimer-antibody variable region fusion protein comprises a second heavy chain comprising the amino acid sequence of SEQ ID NO:291. In some embodiments, the Fc polypeptide dimer-antibody variable region fusion protein comprises two light chains comprising the amino acid sequence of SEQ ID NO:293.

In some embodiments, an Fc polypeptide dimer-antibody variable region fusion protein comprises: (a) an antibody variable region that is capable of binding HER2 (e.g., human HER2), or an antigen-binding fragment thereof, (b) a first Fc polypeptide that contains modifications that create a TfR-binding site and hole mutations T366S, L368A, and Y407V, according to EU numbering, and (c) a second Fc polypeptide that comprises a knob mutation T366W, according to EU numbering, and does not contain a TfR-binding site.

In some embodiments, the Fc polypeptide dimer-antibody variable region fusion protein comprises a first heavy chain comprising the amino acid sequence of any one of SEQ ID NOS:5, 13, 21, and 85. In some embodiments, he Fc polypeptide dimer-antibody variable region fusion protein comprises a second heavy chain comprising the amino acid sequence of SEQ ID NO:25. In some embodiments, the Fc polypeptide dimer-antibody variable region fusion protein comprises two light chains comprising the amino acid sequence of SEQ ID NO:57.

In some embodiments, the Fc polypeptide dimer-antibody variable region fusion protein comprises a first heavy chain comprising the amino acid sequence of any one of SEQ ID NOS:33, 41, 49, and 93. In some embodiments, the Fc polypeptide dimer-antibody variable region fusion protein comprises a second heavy chain comprising the amino acid sequence of SEQ ID NO:53. In some embodiments, the Fc polypeptide dimer-antibody variable region fusion protein comprises two light chains comprising the amino acid sequence of SEQ ID NO:58.

In some embodiments, the Fc polypeptide dimer-antibody variable region fusion protein comprises a first heavy chain comprising the amino acid sequence of any one of SEQ ID NOS:262, 270, 278, and 286. In some embodiments, he Fc polypeptide dimer-antibody variable region fusion protein comprises a second heavy chain comprising the amino acid sequence of SEQ ID NO:294. In some embodiments, the Fc polypeptide dimer-antibody variable region fusion protein comprises two light chains comprising the amino acid sequence of SEQ ID NO:293.

In some embodiments, an Fc polypeptide dimer-antibody variable region fusion protein comprises: (a) an antibody variable region that is capable of binding HER2 (e.g., human HER2), or an antigen-binding fragment thereof, (b) a first Fc polypeptide that contains modifications that create a TfR-binding site, hole mutations T366S, L368A, and Y407V, and amino acid modification N434S with or without M428L, according to EU numbering, and (c) a second Fc polypeptide that comprises a knob mutation T366W, according to EU numbering, and does not contain a TfR-binding site.

In some embodiments, the Fc polypeptide dimer-antibody variable region fusion protein comprises a first heavy chain comprising the amino acid sequence of any one of SEQ ID NOS:7, 15, 23, and 87. In some embodiments, the Fc polypeptide dimer-antibody variable region fusion protein comprises a second heavy chain comprising the amino acid sequence of SEQ ID NO:25. In some embodiments, the Fc polypeptide dimer-antibody variable region fusion protein comprises two light chains comprising the amino acid sequence of SEQ ID NO:57.

In some embodiments, the Fc polypeptide dimer-antibody variable region fusion protein comprises a first heavy chain comprising the amino acid sequence of any one of SEQ ID NOS:35, 43, 51, and 95. In some embodiments, the Fc polypeptide dimer-antibody variable region fusion protein comprises a second heavy chain comprising the amino acid sequence of SEQ ID NO:53. In some embodiments, the Fc polypeptide dimer-antibody variable region fusion protein comprises two light chains comprising the amino acid sequence of SEQ ID NO:58.

In some embodiments, the Fc polypeptide dimer-antibody variable region fusion protein comprises a first heavy chain comprising the amino acid sequence of any one of SEQ ID NOS:264, 272, 280, and 288. In some embodiments, the Fc polypeptide dimer-antibody variable region fusion protein comprises a second heavy chain comprising the amino acid sequence of SEQ ID NO:294. In some embodiments, the Fc polypeptide dimer-antibody variable region fusion protein comprises two light chains comprising the amino acid sequence of SEQ ID NO:293.

In some embodiments, an Fc polypeptide dimer-antibody variable region fusion protein comprises: (a) an antibody variable region that is capable of binding HER2 (e.g., human HER2), or an antigen-binding fragment thereof, (b) a first Fc polypeptide that contains modifications that create a TfR-binding site and hole mutations T366S, L368A, and Y407V, according to EU numbering, and (c) a second Fc polypeptide that comprises a knob mutation T366W and amino acid modification N434S with or without M428L, according to EU numbering, and does not contain a TfR-binding site.

In some embodiments, the Fc polypeptide dimer-antibody variable region fusion protein comprises a first heavy chain comprising the amino acid sequence of any one of SEQ ID NOS:5, 13, 21, and 85. In some embodiments, the Fc polypeptide dimer-antibody variable region fusion protein comprises a second heavy chain comprising the amino acid sequence of SEQ ID NO:26. In some embodiments, the Fc polypeptide dimer-antibody variable region fusion protein comprises two light chains comprising the amino acid sequence of SEQ ID NO:57.

In some embodiments, the Fc polypeptide dimer-antibody variable region fusion protein comprises a first heavy chain comprising the amino acid sequence of any one of SEQ ID NOS:33, 41, 49, and 93. In some embodiments, the Fc polypeptide dimer-antibody variable region fusion protein comprises a second heavy chain comprising the amino acid sequence of SEQ ID NO:54. In some embodiments, the Fc polypeptide dimer-antibody variable region fusion protein comprises two light chains comprising the amino acid sequence of SEQ ID NO:58.

In some embodiments, the Fc polypeptide dimer-antibody variable region fusion protein comprises a first heavy chain comprising the amino acid sequence of any one of SEQ ID NOS:262, 270, 278, and 286. In some embodiments, the Fc polypeptide dimer-antibody variable region fusion protein comprises a second heavy chain comprising the amino acid sequence of SEQ ID NO:295. In some embodiments, the Fc polypeptide dimer-antibody variable region fusion protein comprises two light chains comprising the amino acid sequence of SEQ ID NO:293.

In some embodiments, an Fc polypeptide dimer-antibody variable region fusion protein comprises: (a) an antibody variable region that is capable of binding HER2 (e.g., human HER2), or an antigen-binding fragment thereof, (b) a first Fc polypeptide that contains modifications that create a TfR-binding site, hole mutations T366S, L368A, and Y407V, and amino acid modification N434S with or without M428L, according to EU numbering, and (c) a second Fc polypeptide that comprises a knob mutation T366W and amino acid modification N434S with or without M428L, according to EU numbering, and does not contain a TfR-binding site.

In some embodiments, the Fc polypeptide dimer-antibody variable region fusion protein comprises a first heavy chain comprising the amino acid sequence of any one of SEQ ID NOS:7, 15, 23, and 87. In some embodiments, the Fc polypeptide dimer-antibody variable region fusion protein comprises a second heavy chain comprising the amino acid sequence of SEQ ID NO:26. In some embodiments, the Fc polypeptide dimer-antibody variable region fusion protein comprises two light chains comprising the amino acid sequence of SEQ ID NO:57.

In some embodiments, the Fc polypeptide dimer-antibody variable region fusion protein comprises a first heavy chain comprising the amino acid sequence of any one of SEQ ID NOS:35, 43, 51, and 95. In some embodiments, the Fc polypeptide dimer-antibody variable region fusion protein comprises a second heavy chain comprising the amino acid sequence of SEQ ID NO:54. In some embodiments, the Fc polypeptide dimer-antibody variable region fusion protein comprises two light chains comprising the amino acid sequence of SEQ ID NO:58.

In some embodiments, the Fc polypeptide dimer-antibody variable region fusion protein comprises a first heavy chain comprising the amino acid sequence of any one of SEQ ID NOS:264, 272, 280, and 288. In some embodiments, the Fc polypeptide dimer-antibody variable region fusion protein comprises a second heavy chain comprising the amino acid sequence of SEQ ID NO:295. In some embodiments, the Fc polypeptide dimer-antibody variable region fusion protein comprises two light chains comprising the amino acid sequence of SEQ ID NO:293.

Antibody Heavy Chains

In other aspects, provided herein are antibody heavy chains. In some embodiments, the antibody heavy chains comprise: (a) an anti-HER2 (e.g., human HER2) antibody heavy chain variable region, or a fragment thereof; and (b) a modified Fc polypeptide that contains modifications that create a TfR-binding site. The antibody heavy chains can comprise any of the anti-HER2 variable heavy chain CDR and/or heavy chain variable region sequences described above in the section titled “Anti-HER2 Variable Regions.” The modified Fc polypeptide can comprise any of the TfR-binding sites (e.g., modified CH3 domains) described herein and/or any of the modifications that increase serum half-life or reduce FcγR binding (e.g., when bound to TfR) described herein.

In some embodiments, the modified Fc polypeptide further comprises a knob mutation T366W, according to EU numbering. In some embodiments, the modified polypeptide comprises an amino acid sequence having at least about 80%, 85%, 90%, 95,%, 96%, 97%, 98%, or 99% identity to SEQ ID NO:63. In some instances, the modified Fc polypeptide comprises the amino acid sequence of SEQ ID NO:63. In some embodiments, the modified Fc polypeptide further comprises hole mutations T366S, L368A, and Y407V, according to EU numbering. In some embodiments, the modified polypeptide comprises an amino acid sequence having at least about 80%, 85%, 90%, 95,%, 96%, 97%, 98%, or 99% identity to SEQ ID NO:64. In some instances, the modified Fc polypeptide comprises the amino acid sequence of SEQ ID NO:64.

In some embodiments, the antibody heavy chain comprises: (a) an anti-HER2 (e.g., human HER2) antibody heavy chain variable region, or a fragment thereof, and (b) a modified Fc polypeptide that contains modifications that create a TfR-binding site, amino acid modifications L234A and L235A, and a knob mutation T366W, according to EU numbering. In some embodiments, the antibody heavy chain comprises the amino acid sequence of any one of SEQ ID NOS:2, 10, 18, and 82. In some embodiments, the antibody heavy chain comprises the amino acid sequence of any one of SEQ ID NOS:30, 38, 46, and 90. In some embodiments, the antibody heavy chain comprises the amino acid sequence of any one of SEQ ID NOS:259, 267, 275, and 283.

In some embodiments, the antibody heavy chain comprises: (a) an anti-HER2 (e.g., human HER2) antibody heavy chain variable region, or a fragment thereof, and (b) a modified Fc polypeptide that contains modifications that create a TfR-binding site, amino acid modifications L234A and L235A, a knob mutation T366W, and amino acid modification N434S with or without M428L, according to EU numbering. In some embodiments, the antibody heavy chain comprises the amino acid sequence of any one of SEQ ID NOS:4, 12, 20, and 84. In some embodiments, the antibody heavy chain comprises the amino acid sequence of any one of SEQ ID NOS:32, 40, 48, and 92. In some embodiments, the antibody heavy chain comprises the amino acid sequence of any one of SEQ ID NOS:261, 269, 277, and 285.

In some embodiments, the antibody heavy chain comprises: (a) an anti-HER2 (e.g., human HER2) antibody heavy chain variable region, or a fragment thereof, and (b) a modified Fc polypeptide that contains modifications that create a TfR-binding site, amino acid modifications L234A and L235A, and hole mutations T366S, L368A, and Y407V, according to EU numbering. In some embodiments, the antibody heavy chain comprises the amino acid sequence of any one of SEQ ID NOS: NOS:6, 14, 22, and 86. In some embodiments, the antibody heavy chain comprises the amino acid sequence of any one of SEQ ID NOS:34, 42, 50, and 94. In some embodiments, the antibody heavy chain comprises the amino acid sequence of any one of SEQ ID NOS:263, 271, 279, and 287.

In some embodiments, the antibody heavy chain comprises: (a) an anti-HER2 (e.g., human HER2) antibody heavy chain variable region, or a fragment thereof, and (b) a modified Fc polypeptide that contains modifications that create a TfR-binding site, amino acid modifications L234A and L235A, hole mutations T366S, L368A, and Y407V, and amino acid modification N434S with or without M428L, according to EU numbering. In some embodiments, the antibody heavy chain comprises the amino acid sequence of any one of SEQ ID NOS:8, 16, 24, and 88. In some embodiments, the antibody heavy chain comprises the amino acid sequence of any one of SEQ ID NOS:36, 44, 52, and 96. In some embodiments, the antibody heavy chain comprises the amino acid sequence of any one of SEQ ID NOS:265, 273, 281, and 289.

In some embodiments, the antibody heavy chain comprises: (a) an anti-HER2 (e.g., human HER2) antibody heavy chain variable region, or a fragment thereof, and (b) a modified Fc polypeptide that contains modifications that create a TfR-binding site and a knob mutation T366W, according to EU numbering. In some embodiments, the antibody heavy chain comprises the amino acid sequence of any one of SEQ ID NOS:1, 9, 17, and 81. In some embodiments, the antibody heavy chain comprises the amino acid sequence of any one of SEQ ID NOS:29, 37, 45, and 89. In some embodiments, the antibody heavy chain comprises the amino acid sequence of any one of SEQ ID NOS:258, 266, 274, and 282.

In some embodiments, the antibody heavy chain comprises: (a) an anti-HER2 (e.g., human HER2) antibody heavy chain variable region, or a fragment thereof, and (b) a modified Fc polypeptide that contains modifications that create a TfR-binding site, a knob mutation T366W, and amino acid modification N434S with or without M428L, according to EU numbering. In some embodiments, the antibody heavy chain comprises the amino acid sequence of any one of SEQ ID NOS:3, 11, 19, and 83. In some embodiments, the antibody heavy chain comprises the amino acid sequence of any one of SEQ ID NOS:31, 39, 47, and 91. In some embodiments, the antibody heavy chain comprises the amino acid sequence of any one of SEQ ID NOS:260, 268, 276, and 284.

In some embodiments, the antibody heavy chain comprises: (a) an anti-HER2 (e.g., human HER2) antibody heavy chain variable region, or a fragment thereof, and (b) a modified Fc polypeptide that contains modifications that create a TfR-binding site and hole mutations T366S, L368A, and Y407V, according to EU numbering. In some embodiments, the antibody heavy chain comprises the amino acid sequence of any one of SEQ ID NOS: NOS:5, 13, 21, and 85. In some embodiments, the antibody heavy chain comprises the amino acid sequence of any one of SEQ ID NOS: 33, 41, 49, and 93. In some embodiments, the antibody heavy chain comprises the amino acid sequence of any one of SEQ ID NOS: NOS:262, 270, 278, and 286.

In some embodiments, the antibody heavy chain comprises: (a) an anti-HER2 (e.g., human HER2) antibody heavy chain variable region, or a fragment thereof, and (b) a modified Fc polypeptide that contains modifications that create a TfR-binding site, hole mutations T366S, L368A, and Y407V, and amino acid modification N434S with or without M428L, according to EU numbering. In some embodiments, the antibody heavy chain comprises the amino acid sequence of any one of SEQ ID NOS:7, 15, 23, and 87. In some embodiments, the antibody heavy chain comprises the amino acid sequence of any one of SEQ ID NOS:35, 43, 51, and 95. In some embodiments, the antibody heavy chain comprises the amino acid sequence of any one of SEQ ID NOS:264, 272, 280, and 288.

### IV. TfR-Binding Fc Polypeptides

This section describes modified Fc polypeptides that bind to TfR and are capable of being transported across the blood-brain barrier (BBB).

CH3 TfR-Binding Polypeptides

In some embodiments, the modified Fc polypeptide contains a modified human Ig CH3 domain, such as an IgG CH3 domain. The CH3 domain can be of any IgG subtype, i.e., from IgG1, IgG2, IgG3, or IgG4. In the context of IgG antibodies, a CH3 domain refers to the segment of amino acids from about position 341 to about position 447 as numbered according to the EU numbering scheme. The positions in the CH3 domain for purposes of identifying the corresponding set of amino acid positions for TfR binding are determined with reference to EU numbering scheme, SEQ ID NO:101, or amino acids 111-217 of SEQ ID NO:99 unless otherwise specified. Substitutions are also determined with reference to EU numbering scheme or SEQ ID NO:99, i.e., an amino acid is considered to be a substitution relative to the amino acid at the corresponding position in EU numbering scheme or SEQ ID NO:99.

As indicated above, sets of residues of a CH3 domain that can be modified are numbered herein with reference to EU numbering scheme or SEQ ID NO:99. Any CH3 domain, e.g., an IgG1, IgG2, IgG3, or IgG4 CH3 domain, may have modifications, e.g., amino acid substitutions, in one or more sets of residues that correspond to residues at the noted positions in EU numbering scheme or SEQ ID NO:99. The positions of each of the IgG1, IgG2, IgG3, and IgG4 sequences that correspond to any given position of EU numbering scheme or SEQ ID NO:99 can be readily determined.

One of skill understands that CH3 domains of other immunoglobulin isotypes, e.g., IgM, IgA, IgE, IgD, etc. may be similarly modified by identifying the amino acids in those domains that correspond to the amino acid positions described herein. Modifications may also be made to corresponding domains from immunoglobulins from other species, e.g., non-human primates, monkey, mouse, rat, rabbit, dog, pig, chicken, and the like.

In one embodiment, a modified CH3 domain polypeptide that specifically binds TfR binds to the apical domain of the TfR at an epitope that comprises position 208 of the full length human TfR sequence (SEQ ID NO:102), which corresponds to position 11 of the human TfR apical domain sequence set forth in SEQ ID NO:103. SEQ ID NO:103 corresponds to amino acids 198-378 of the human TfR-1 uniprotein sequence P02786 (SEQ ID NO:102). In some embodiments, the modified CH3 domain polypeptide binds to the apical domain of the TfR at an epitope that comprises positions 158, 188, 199, 207, 208, 209, 210, 211, 212, 213, 214, 215, and/or 294 of the full length human TfR sequence (SEQ ID NO:102). The modified CH3 domain polypeptide may bind to the TfR without blocking or otherwise inhibiting binding of transferrin to the receptor. In some embodiments, binding of transferrin to TfR is not substantially inhibited. In some embodiments, binding of transferrin to TfR is inhibited by less than about 50% (e.g., less than about 45%, 40%, 35%, 30%, 25%, 20%, 15%, 10%, or 5%). In some embodiments, binding of transferrin to TfR is inhibited by less than about 20% (e.g., less than about 19%, 18%, 17%, 16%, 15%, 14%, 13%, 12%, 11%, 10%, 9%, 8%, 7%, 6%, 5%, 4%, 3%, 2%, or 1%). Illustrative CH3 domain polypeptides that exhibit this binding specificity include polypeptides having amino acid substitutions at positions 380, 384, 386, 387, 388, 389, 390, 413, 415, 416, and 421, according to the EU numbering scheme.

CH3 TfR Binding Set: 380, 384, 386, 387, 388, 389, 390, 413, 415, 416, and 421

In some embodiments, a modified CH3 domain polypeptide comprises one, two, three, four, five, six, seven, eight, nine, ten, or eleven substitutions in a set of amino acid positions comprising 380, 384, 386, 387, 388, 389, 390, 413, 415, 416, and 421, according to the EU numbering scheme (set CH3C). Illustrative substitutions that may be introduced at these positions are shown in Table 5. Additional substitutions are shown in Table 6. In some embodiments, the amino acid at position 388 and/or 421 is an aromatic amino acid, e.g., Trp, Phe, or Tyr. In some embodiments, the amino acid at position 388 is Trp. In some embodiments, the amino acid at position 388 is Gly. In some embodiments, the aromatic amino acid at position 421 is Trp or Phe.

In certain embodiments, the modified CH3 domain polypeptide comprises one, two, three, four, five, six, seven, eight, nine, ten, or eleven positions selected from the following: Glu, Leu, Ser, Val, Trp, Tyr, or Gln at position 380; Leu, Tyr, Phe, Trp, Met, Pro, or Val at position 384; Leu, Thr, His, Pro, Asn, Val, or Phe at position 386; Val, Pro, Ile, or an acidic amino acid at position 387; Trp at position 388; an aliphatic amino acid, Gly, Ser, Thr, or Asn at position 389; Gly, His, Gln, Leu, Lys, Val, Phe, Ser, Ala, Asp, Glu, Asn, Arg, or Thr at position 390; an acidic amino acid, Ala, Ser, Leu, Thr, Pro, Ile, or His at position 413; Glu, Ser, Asp, Gly, Thr, Pro, Gln, or Arg at position 415; Thr, Arg, Asn, or an acidic amino acid at position 416; and/or an aromatic amino acid, His, or Lys at position 421

In some embodiments, a modified CH3 domain polypeptide that specifically binds TfR has at least 70% identity, at least 75% identity, at least 80% identity, at least 85% identity, at least 90% identity, or at least 95% identity to amino acids 111-217 of any one of SEQ ID NOS:177-180. In some embodiments, such a modified CH3 domain polypeptide comprises amino acids 154-160 and/or 183-191 of any one of SEQ ID NOS:177-180. In some embodiments, such a modified CH3 domain polypeptide comprises amino acids 150-160 and/or 183-191 of any one of SEQ ID NOS:177-180. In some embodiments, a modified CH3 domain polypeptide comprises amino acids 150-160 and/or 183-196 of any one of SEQ ID NOS:177-180.

In some embodiments, a modified CH3 domain polypeptide has at least 70% identity, at least 75% identity, at least 80% identity, at least 85% identity, at least 90% identity, or at least 95% identity to amino acids 111-217 of SEQ ID NO:99, with the proviso that the percent identity does not include the set of positions 154, 156, 157, 158, 159, 160, 183, 186, and 191 of SEQ ID NO:99 (positions 384, 386, 387, 388, 389, 390, 413, 416, and 421, according to EU numbering scheme). In some embodiments, the modified CH3 domain polypeptide comprises amino acids 154-160 and/or amino acids 183-191 as set forth in any one of SEQ ID NOS:177-180.

In some embodiments, a modified CH3 domain polypeptide has at least 70% identity, at least 75% identity, at least 80% identity, at least 85% identity, at least 90% identity, or at least 95% identity to any one of SEQ ID NOS:177-180, with the proviso that at least five, six, seven, eight, nine, ten, eleven, twelve, thirteen, fourteen, fifteen or sixteen of the positions that correspond to positions 150, 154, 156, 157, 158, 159, 160, 161, 162, 183, 184, 185, 186, 191, 194, and 196 of any one of SEQ ID NOS:177-180 (positions 380, 384, 386, 384, 388, 389, 390, 391, 392, 413, 414, 415, 416, 421, 424, and 426, according to EU numbering scheme) are not deleted or substituted.

In some embodiments, the modified CH3 domain polypeptide has at least 75% identity, at least 80% identity, at least 85% identity, at least 90% identity, or at least 95% identity to any one of SEQ ID NOS:177-180 and also comprises at at least five, six, seven, eight, nine, ten, eleven, twelve, thirteen, fourteen, fifteen or sixteen of the positions as follows: Trp, Tyr, Leu, Gln, or Glu at position 380; Leu, Tyr, Met, or Val at position 384; Leu, Thr, His, or Pro at position 386; Val, Pro, or an acidic amino acid at position 387; an aromatic amino acid, e.g., Trp, at position 388; Val, Ser, or Ala at position 389; Ser or Asn at position 390; Ser, Thr, Gln, or Phe at position 391; Gln, Phe, or His at position 392; an acidic amino acid, Ala, Ser, Leu, Thr, or Pro at position 413; Lys, Arg, Gly or Pro at position 414; Glu or Ser at position 415; Thr or an acidic amino acid at position 416; Trp, Tyr, His or Phe at position 421; Ser, Thr, Glu or Lys at position 424; and Ser, Trp, or Gly at position 426.

In additional embodiments, a TfR-binding polypeptide comprises amino acids 157-194, amino acids 153-194, or amino acids 153-199, of any one of SEQ ID NOS:177-180. In further embodiments, the polypeptide comprises an amino acid sequence having at least 75% identity, at least 80% identity, at least 85% identity, at least 90% identity, or at least 95% identity to amino acids 157-194 of any one of SEQ ID NOS:177-180, or to amino acids 153-194, or to amino acids 153-199, of any one of SEQ ID NOS:177-180.

In some embodiments, the polypeptide comprises any one of SEQ ID NOS:177-180. In further embodiments, the polypeptide may have at least 75% identity, at least 80% identity, at least 85% identity, at least 90% identity, or at least 95% identity to any one of SEQ ID NOS:177-180.

FcRn Binding Sites

A polypeptide described herein that can be transported across the BBB additionally may comprise an FcRn binding site. In some embodiments, the FcRn binding site is within the modified Fc polypeptide or a fragment thereof.

In some embodiments, the FcRn binding site comprises a native FcRn binding site. In some embodiments, the FcRn binding site does not comprise amino acid changes relative to the amino acid sequence of a native FcRn binding site. In some embodiments, the native FcRn binding site is an IgG binding site, e.g., a human IgG binding site. In some embodiments, the FcRn binding site comprises a modification that alters FcRn binding.

In some embodiments, an FcRn binding site has one or more amino acid residues that are mutated, e.g., substituted, wherein the mutation(s) increase serum half-life or do not substantially reduce serum half-life (i.e., reduce serum half-life by no more than 25% compared to a counterpart protein having the wild-type residues at the mutated positions when assayed under the same conditions). In some embodiments, an FcRn binding site has one or more amino acid residues that are substituted at positions 21 to 26, 198, and 203 to 206, wherein the positions are determined with reference to SEQ ID NO:99.

In some embodiments, the FcRn binding site comprises one or more mutations, relative to a native human IgG sequence, that extend serum half-life of the modified polypeptide. In some embodiments, a mutation, e.g., a substitution, is introduced at one or more of positions 14-27, 49-54, 77-87, 153-160, and 198-205 as determined with reference to SEQ ID NO:99 (which positions correspond to positions 244-257, 279-284, 307-317, 383-390, and 428-435 using EU numbering). In some embodiments, one or more mutations are introduced at positions 21, 22, 24, 25, 26, 77, 78, 79, 81, 82, 84, 155, 156, 157, 159, 198, 203, 204, or 206 as determined with reference to SEQ ID NO:99 (which positions correspond to positions 251, 252, 254, 255, 256, 307, 308, 309, 311, 312, 314, 385, 386, 387, 389, 428, 433, 434, or 436 using EU numbering). In some embodiments, mutations are introduced into one, two, or three of positions 22, 24, and 25 as determined with reference to SEQ ID NO:99 (which correspond to positions 252, 254, and 256 using EU numbering). In some embodiments, the mutations are M22Y, S24T, and T26E as numbered with reference to SEQ ID NO:99. In some embodiments, a modified Fc polypeptide described herein further comprises mutations M22Y, S24T, and T26E. In some embodiments, mutations are introduced into one or two of positions 198 and 204 as determined with reference to SEQ ID NO:99 (which correspond to positions 428 and 434 using EU numbering). In some embodiments, the mutations are M198L and N204S as numbered with reference to SEQ ID NO:99. In some embodiments, a modified Fc polypeptide described herein further comprises mutation N204S with or without M198L. In some embodiments, a modified Fc polypeptide comprises a substitution at one, two or all three of positions T307, E380, and N434 according to EU numbering (which correspond to T77, E150, and N204 as numbered with reference to SEQ ID NO:99). In some embodiments, the mutations are T307Q and N434A (SEQ ID NO:99, T77Q and N204A). In some embodiments, a modified Fc polypeptide comprises mutations T307A, E380A, and N434A (SEQ ID NO:99, T77A, E150A, and N204A). In some embodiments, a modified Fc polypeptide comprises substitutions at positions T250 and M428 (which correspond to T20 and M198 as numbered with reference to SEQ ID NO:99). In some embodiments, the Fc polypeptide comprises mutations T250Q and/or M428L (SEQ ID NO:99, T20Q and M198L). In some embodiments, a modified Fc polypeptide comprises substitutions at positions M428 and N434 (which correspond to M198 and N204 as numbered with reference to SEQ ID NO:99). In some embodiments, a modified Fc polypeptide comprises substitutions M428L and N434S (which correspond to M198L and N204S as numbered with reference to SEQ ID NO:99). In some embodiments, a modified Fc polypeptide comprises an N434S or N434A substitution (which corresponds to N204S or N204A as numbered with reference to SEQ ID NO:99).

### V. Mutations that Reduce Effector Function or FcγR Binding

An Fc polypeptide as provided herein (e.g., that is modified to bind TfR and initiate transport across the BBB) may also comprise additional mutations to reduce effector function. As described herein, by introducing both the TfR-binding site and mutations that reduce TfR-mediated FcγR binding to the same Fc polypeptide of the Fc polypeptide dimer, it was possible to reduce effector function upon TfR binding, leading to TfR binding without substantial depletion of reticulocytes, but still maintain and exhibit a level of effector function (e.g., ADCC or CDC) when the Fc polypeptide dimer is fused to a therapeutic Fab and bound to the Fab's target antigen.

In some embodiments, an Fc polypeptide comprising a modified CH3 domain has an effector function, i.e., the ability to induce certain biological functions upon binding to an Fc receptor expressed on an effector cell that mediates the effector function. Effector cells include, but are not limited to, monocytes, macrophages, neutrophils, dendritic cells, eosinophils, mast cells, platelets, B cells, large granular lymphocytes, Langerhans' cells, natural killer (NK) cells, and cytotoxic T cells.

Examples of effector functions include, but are not limited to, C1q binding and CDC, Fc receptor binding, ADCC, antibody-dependent cell-mediated phagocytosis (ADCP), down-regulation of cell surface receptors (e.g., B cell receptor), and B-cell activation. Effector functions may vary with the antibody class. For example, native human IgG1 and IgG3 antibodies can elicit ADCC and CDC activities upon binding to an appropriate Fc receptor present on an immune system cell; and native human IgG1, IgG2, IgG3, and IgG4 can elicit ADCP functions upon binding to the appropriate Fc receptor present on an immune cell.

In some embodiments, an Fc polypeptide having an TfR-binding site as described herein may include additional modifications that reduce effector function, i.e., reduce effector function upon TfR binding. Having reduced effector function upon TfR binding of the Fc polypeptide dimer is desirable because it leads to reduced reticulocyte depletion since reticulocytes also have TfR on the cell surface. As described in detail herein, Fc polypeptide dimers having the cis configuration, i.e., Fc polypeptide dimers having both the TfR-binding site and mutations that reduce effector function on the same Fc polypeptide of the Fc polypeptide dimer, exhibit TfR binding without substantial depletion of reticulocytes, but still maintain a level of effector function (e.g., ADCC) when the Fc polypeptide dimer is fused to a therapeutic Fab and bound to the Fab's target antigen. Having effector function when the Fc polypeptide dimer is fused to a therapeutic Fab that is bound to the Fab's target antigen is desirable in, e.g., cancer therapeutics (e.g., brain cancer therapeutics).

Illustrative Fc polypeptide mutations that modulate an effector function include, but are not limited to, substitutions in a CH2 domain, e.g., at positions corresponding to positions 4 and 5 of SEQ ID NO:99 (positions 234 and 235 according to EU numbering scheme). In some embodiments, the substitutions in a modified CH2 domain comprise Ala at positions 4 and 5 of SEQ ID NO:99. In some embodiments, the substitutions in a modified CH2 domain comprise Ala at positions 4 and 5 and Gly at position 99 of SEQ ID NO:99.

Additional Fc polypeptide mutations that modulate an effector function include, but are not limited to, one or more substitutions at positions 238, 265, 269, 270, 297, 327 and 329 (EU numbering scheme, which correspond to positions 8, 35, 39, 40, 67, 97, and 99 as numbered with reference to SEQ ID NO:99). Illustrative substitutions (as numbered with EU numbering scheme), include the following: position 329 may have a mutation in which proline is substituted with a glycine or arginine or an amino acid residue large enough to destroy the Fc/Fcγ receptor interface that is formed between proline 329 of the Fc and tryptophan residues Trp 87 and Trp 110 of FcγRIII. Additional illustrative substitutions include S228P, E233P, L235E, N297A, N297D, and P331S. Multiple substitutions may also be present, e.g., L234A and L235A of a human IgG1 Fc region; L234A, L235A, and P329G of a human IgG1 Fc region; S228P and L235E of a human IgG4 Fc region; L234A and G237A of a human IgG1 Fc region; L234A, L235A, and G237A of a human IgG1 Fc region; V234A and G237A of a human IgG2 Fc region; L235A, G237A, and E318A of a human IgG4 Fc region; and S228P and L236E of a human IgG4 Fc region. In some embodiments, an Fc polypeptide may have one or more amino acid substitutions that modulate ADCC, e.g., substitutions at positions 298, 333, and/or 334 of the Fc region, according to the EU numbering scheme.

In some embodiments, a polypeptide as described herein may have one or more amino acid substitutions that increase or decrease ADCC or may have mutations that alter C1q binding and/or CDC.

In particular embodiments, an Fc polypeptide having a TfR-binding site may be modified to reduce effector function, i.e., reduce FcγR binding. In some embodiments, an Fc polypeptide having a TfR-binding site may include mutations L234A and L235A (EU numbering scheme, which correspond to positions 4 and 5 as numbered with reference to SEQ ID NO:99). In other embodiments, an Fc polypeptide having a TfR-binding site may include mutations L234A, L235A, and P329G (EU numbering scheme, which correspond to positions 4, 5, and 99 as numbered with reference to SEQ ID NO:99).

### VI. Measuring Effector Function or FcγR Binding

Methods for analyzing binding affinity, binding kinetics, and cross-reactivity between an Fc polypeptide or an Fc polypeptide dimer and FcγR are known in the art. These methods include, but are not limited to, solid-phase binding assays (e.g., ELISA assay), immunoprecipitation, surface plasmon resonance (e.g., Biacore™ (GE Healthcare, Piscataway, N.J.)), kinetic exclusion assays (e.g., KinExA®), flow cytometry, fluorescence-activated cell sorting (FACS), BioLayer interferometry (e.g., Octet® (ForteBio, Inc., Menlo Park, Calif.)), and Western blot analysis. In some embodiments, ELISA is used to determine binding affinity and/or cross-reactivity. Methods for performing ELISA assays are known in the art. In some embodiments, surface plasmon resonance (SPR) is used to determine binding affinity, binding kinetics, and/or cross-reactivity. In some embodiments, kinetic exclusion assays are used to determine binding affinity, binding kinetics, and/or cross-reactivity. In some embodiments, BioLayer interferometry assays are used to determine binding affinity, binding kinetics, and/or cross-reactivity.

ADCC is a type of immune response in which antibodies bind to antigens on the surface of pathogenic or tumorigenic target cells and identifies them for destruction by effector cells, e.g., peripheral blood mononuclear cells (e.g., natural killer (NK) cells, T cells, and B cells). Effector cells bearing FcγR recognize and bind the Fc region of the antibodies bound to the target cell. The antibodies thus confer specificity to the target cell killing. CDC is initiated when C1q, the initiating component of the classical complement pathway, is bound to the Fc region of target-bound antibodies. ADCC and CDC activities may be determined in a standard in vivo or in vitro assay of cell killing. Methods for determining ADCC and CDC activities are available in the art. In some embodiments, the methods may involve labeling target cells with a radioactive material, such as ⁵¹Cr, or a fluorescent dye, such as Calcein-AM. The labeled cells may be incubated with the antibody and effector cells and killing of the target cells by ADCC or CDC may be detected by the release of radioactivity or fluorescence.

Other assays to measure ADCC and CDC activities include, e.g., a lactate dehydrogenase (LDH) release assay. When the cell membranes are compromised or damaged in any way, LDH, a soluble yet stable enzyme in the cytoplasm, is released into the surrounding extracellular space. The presence of this enzyme in the culture medium can be used as a cell death marker. The relative amounts of live and dead cells within the medium can then be quantitated by measuring the amount of released LDH using a colorimetric or fluorometric LDH cytotoxicity assay.

### VII. Additional Mutations in an Fc Region that Comprises A Modified CH3 Domain Polypeptide

An Fc polypeptide as provided herein (e.g., that is modified to bind TfR and initiate transport across the BBB) may also comprise additional mutations, e.g., to increase serum stability or serum half-life, to modulate effector function, to influence glyscosylation, to reduce immunogenicity in humans, and/or to provide for knob and hole heterodimerization of Fc polypeptides.

In some embodiments, a modified Fc polypeptide described herein has an amino acid sequence identity of at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% to a corresponding wild-type Fc polypeptide (e.g., a human IgG1, IgG2, IgG3, or IgG4 Fc polypeptide).

A modified Fc polypeptide described herein may also have other mutations introduced outside of the specified sets of amino acids, e.g., to influence glyscosylation, to increase serum half-life or, for CH3 domains, to provide for knob and hole heterodimerization of polypeptides that comprise the modified CH3 domain. Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g., tyrosine or tryptophan). Compensatory cavities of identical or similar size to the protuberances are created in the interface of the second polypeptide by replacing large amino acid side chains with smaller ones (e.g., alanine or threonine). Such additional mutations are at a position in the polypeptide that does not have a negative effect on binding of the modified CH3 domain to the TfR.

In one illustrative embodiment of a knob and hole approach for dimerization, a position corresponding to position 136 of SEQ ID NO:99 of a first Fc polypeptide subunit to be dimerized has a tryptophan in place of a native threonine and a second Fc polypeptide subunit of the dimer has a valine at a position corresponding to position 177 of SEQ ID NO:99 in place of the native tyrosine. The second subunit of the Fc polypeptide may further comprise a substitution in which the native threonine at the position corresponding to position 136 of SEQ ID NO:99 is substituted with a serine and a native leucine at the position corresponding to position 138 of SEQ ID NO:99 is substituted with an alanine.

A modified Fc polypeptide as described herein may also be engineered to contain other modifications for heterodimerization, e.g., electrostatic engineering of contact residues within a CH3-CH3 interface that are naturally charged or hydrophobic patch modifications.

In some embodiments, modifications to enhance serum half-life may be introduced. For example, in some embodiments, a modified Fc polypeptide as described herein comprises a CH2 domain comprising a Tyr at a position corresponding to position 22 of SEQ ID NO:99, Thr at a position corresponding to 24 of SEQ ID NO:99, and Glu at a position corresponding to position 26 of SEQ ID NO:99. Alternatively, a modified Fc polypeptide as described herein may comprise M198L and N204S substitutions as numbered with reference to SEQ ID NO:99. Alternatively, a modified Fc polypeptide as described herein may comprise an N204S or N204A substitution as numbered with reference to SEQ ID NO:99.

Illustrative Fc Polypeptides Comprising Additional Mutations

A modified Fc polypeptide as described herein (e.g., any one of clones CH3C.35.23.3, CH3C.35.23.4, CH3C.35.23, and CH3C.35.23.1.1) may comprise additional mutations including a knob mutation (e.g., T136W as numbered with reference to SEQ ID NO:99), hole mutations (e.g., T136S, L138A, and Y177V as numbered with reference to SEQ ID NO:99), mutations that modulate effector function (e.g., L4A, L5A, and/or P99G (e.g., L4A and L5A) as numbered with reference to SEQ ID NO:99), and/or mutations that increase serum stability or serum half-life (e.g., (i) M22Y, S24T, and T26E as numbered with reference to SEQ ID NO:99, or (ii) N204S with or without M198L as numbered with reference to SEQ ID NO:99).

In some embodiments, a modified Fc polypeptide as described herein (e.g., any one of clones CH3C.35.23.3, CH3C.35.23.4, CH3C.35.23, and CH3C.35.23.1.1) may have a knob mutation (e.g., T136W as numbered with reference to SEQ ID NO:99) and at least 85% identity, at least 90% identity, or at least 95% identity to the sequence of any one of SEQ ID NOS:177-180. In some embodiments, a modified Fc polypeptide having the sequence of any one of SEQ ID NOS:177-180 may be modified to have a knob mutation.

In some embodiments, a modified Fc polypeptide as described herein (e.g., any one of clones CH3C.35.23.3, CH3C.35.23.4, CH3C.35.23, and CH3C.35.23.1.1) may have a knob mutation (e.g., T136W as numbered with reference to SEQ ID NO:99), mutations that modulate effector function (e.g., L4A, L5A, and/or P99G (e.g., L4A and L5A) as numbered with reference to SEQ ID NO:99), and at least 85% identity, at least 90% identity, or at least 95% identity to the sequence of any one of SEQ ID NOS:177-180. In some embodiments, a modified Fc polypeptide having the sequence of any one of SEQ ID NOS:177-180 may be modified to have a knob mutation and mutations that modulate effector function.

In some embodiments, a modified Fc polypeptide as described herein (e.g., any one of clones CH3C.35.23.3, CH3C.35.23.4, CH3C.35.23, and CH3C.35.23.1.1) may have a knob mutation (e.g., T136W as numbered with reference to SEQ ID NO:99), mutations that increase serum stability or serum half-life (e.g., (i) M22Y, S24T, and T26E as numbered with reference to SEQ ID NO:99, or (ii) N204S with or without M198L as numbered with reference to SEQ ID NO:99), and at least 85% identity, at least 90% identity, or at least 95% identity to the sequence of any one of SEQ ID NOS:177-180. In some embodiments, a modified Fc polypeptide having the sequence of any one of SEQ ID NOS:177-180 may be modified to have a knob mutation and mutations that increase serum stability or serum half-life.

In some embodiments, a modified Fc polypeptide as described herein (e.g., any one of clones CH3C.35.23.3, CH3C.35.23.4, CH3C.35.23, and CH3C.35.23.1.1) may have a knob mutation (e.g., T136W as numbered with reference to SEQ ID NO:99), mutations that modulate effector function (e.g., L4A, L5A, and/or P99G (e.g., L4A and L5A) as numbered with reference to SEQ ID NO:99), mutations that increase serum stability or serum half-life (e.g., (i) M22Y, S24T, and T26E as numbered with reference to SEQ ID NO:99, or (ii) N204S with or without M198L as numbered with reference to SEQ ID NO:99), and at least 85% identity, at least 90% identity, or at least 95% identity to the sequence of any one of SEQ ID NOS:177-180. In some embodiments, a modified Fc polypeptide having the sequence of any one of SEQ ID NOS:177-180 may be modified to have a knob mutation, mutations that modulate effector function, and mutations that increase serum stability or serum half-life.

In some embodiments, a modified Fc polypeptide as described herein (e.g., any one of clones CH3C.35.23.3, CH3C.35.23.4, CH3C.35.23, and CH3C.35.23.1.1) may have hole mutations (e.g., T136S, L138A, and Y177V as numbered with reference to SEQ ID NO:99) and at least 85% identity, at least 90% identity, or at least 95% identity to the sequence of any one of SEQ ID NOS:177-180. In some embodiments, a modified Fc polypeptide having the sequence of any one of SEQ ID NOS:177-180 may be modified to have hole mutations.

In some embodiments, a modified Fc polypeptide as described herein (e.g., any one of clones CH3C.35.23.3, CH3C.35.23.4, CH3C.35.23, and CH3C.35.23.1.1) may have hole mutations (e.g., T136S, L138A, and Y177V as numbered with reference to SEQ ID NO:99), mutations that modulate effector function (e.g., L4A, L5A, and/or P99G (e.g., L4A and L5A) as numbered with reference to SEQ ID NO:99), and at least 85% identity, at least 90% identity, or at least 95% identity to the sequence of any one of SEQ ID NOS:177-180. In some embodiments, a modified Fc polypeptide having the sequence of any one of SEQ ID NOS:177-180 may be modified to have hole mutations and mutations that modulate effector function.

In some embodiments, a modified Fc polypeptide as described herein (e.g., any one of clones CH3C.35.23.3, CH3C.35.23.4, CH3C.35.23, and CH3C.35.23.1.1) may have hole mutations (e.g., T136S, L138A, and Y177V as numbered with reference to SEQ ID NO:99), mutations that increase serum stability or serum half-life (e.g., (i) M22Y, S24T, and T26E as numbered with reference to SEQ ID NO:99, or (ii) N204S with or without M198L as numbered with reference to SEQ ID NO:99), and at least 85% identity, at least 90% identity, or at least 95% identity to the sequence of any one of SEQ ID NOS:177-180. In some embodiments, a modified Fc polypeptide having the sequence of any one of SEQ ID NOS:177-180 may be modified to have hole mutations and mutations that increase serum stability or serum half-life.

In some embodiments, a modified Fc polypeptide as described herein (e.g., any one of clones CH3C.35.23.3, CH3C.35.23.4, CH3C.35.23, and CH3C.35.23.1.1) may have hole mutations (e.g., T136S, L138A, and Y177V as numbered with reference to SEQ ID NO:99), mutations that modulate effector function (e.g., L4A, L5A, and/or P99G (e.g., L4A and L5A) as numbered with reference to SEQ ID NO:99), mutations that increase serum stability or serum half-life (e.g., (i) M22Y, S24T, and T26E as numbered with reference to SEQ ID NO:99, or (ii) N204S with or without M198L as numbered with reference to SEQ ID NO:99), and at least 85% identity, at least 90% identity, or at least 95% identity to the sequence of any one of SEQ ID NOS:177-180. In some embodiments, a modified Fc polypeptide having the sequence of any one of SEQ ID NOS: 177-180 may be modified to have hole mutations, mutations that modulate effector function, and mutations that increase serum stability or serum half-life.

Clone CH3C.35.23.3

In some embodiments, clone CH3C.35.23.3 may have a knob mutation (e.g., T136W as numbered with reference to SEQ ID NO:99) and at least 85% identity, at least 90% identity, or at least 95% identity to the sequence of SEQ ID NO:105. In some embodiments, clone CH3C.35.23.3 with the knob mutation has the sequence of SEQ ID NO:105.

In some embodiments, clone CH3C.35.23.3 may have a knob mutation (e.g., T136W as numbered with reference to SEQ ID NO:99), mutations that modulate effector function (e.g., L4A, L5A, and/or P99G (e.g., L4A and L5A) as numbered with reference to SEQ ID NO:99), and at least 85% identity, at least 90% identity, or at least 95% identity to the sequence of SEQ ID NO:106 or 107. In some embodiments, clone CH3C.35.23.3 with the knob mutation and the mutations that modulate effector function has the sequence of SEQ ID NO:106 or 107.

In some embodiments, clone CH3C.35.23.3 may have a knob mutation (e.g., T136W as numbered with reference to SEQ ID NO:99), mutations that increase serum stability or serum half-life (e.g., M22Y, S24T, and T26E as numbered with reference to SEQ ID NO:99), and at least 85% identity, at least 90% identity, or at least 95% identity to the sequence of SEQ ID NO:108. In some embodiments, clone CH3C.35.23.3 with the knob mutation and the mutations that increase serum stability or serum half-life has the sequence of SEQ ID NO:108.

In some embodiments, clone CH3C.35.23.3 may have a knob mutation (e.g., T136W as numbered with reference to SEQ ID NO:99), mutations that increase serum stability or serum half-life (e.g., N204S with or without M198L as numbered with reference to SEQ ID NO:99), and at least 85% identity, at least 90% identity, or at least 95% identity to the sequence of SEQ ID NO:109. In some embodiments, clone CH3C.35.23.3 with the knob mutation and the mutations that increase serum stability or serum half-life has the sequence of SEQ ID NO:109.

In some embodiments, clone CH3C.35.23.3 may have a knob mutation (e.g., T136W as numbered with reference to SEQ ID NO:99), mutations that modulate effector function (e.g., L4A, L5A, and/or P99G (e.g., L4A and L5A) as numbered with reference to SEQ ID NO:99), mutations that increase serum stability or serum half-life (e.g., M22Y, S24T, and T26E as numbered with reference to SEQ ID NO:99), and at least 85% identity, at least 90% identity, or at least 95% identity to the sequence of SEQ ID NO:110 or 111. In some embodiments, clone CH3C.35.23.3 with the knob mutation, the mutations that modulate effector function, and the mutations that increase serum stability or serum half-life has the sequence of SEQ ID NO:110 or 111.

In some embodiments, clone CH3C.35.23.3 may have a knob mutation (e.g., T136W as numbered with reference to SEQ ID NO:99), mutations that modulate effector function (e.g., L4A, L5A, and/or P99G (e.g., L4A and L5A) as numbered with reference to SEQ ID NO:99), mutations that increase serum stability or serum half-life (e.g., N204S with or without M198L as numbered with reference to SEQ ID NO:99), and at least 85% identity, at least 90% identity, or at least 95% identity to the sequence of SEQ ID NO:112 or 113. In some embodiments, clone CH3C.35.23.3 with the knob mutation, the mutations that modulate effector function, and the mutations that increase serum stability or serum half-life has the sequence of SEQ ID NO:112 or 113.

In some embodiments, clone CH3C.35.23.3 may have hole mutations (e.g., T136S, L138A, and Y177V as numbered with reference to SEQ ID NO:99) and at least 85% identity, at least 90% identity, or at least 95% identity to the sequence of SEQ ID NO:114. In some embodiments, clone CH3C.35.23.3 with the hole mutations and the sequence of SEQ ID NO:114.

In some embodiments, clone CH3C.35.23.3 may have hole mutations (e.g., T136S, L138A, and Y177V as numbered with reference to SEQ ID NO:99), mutations that modulate effector function (e.g., L4A, L5A, and/or P99G (e.g., L4A and L5A) as numbered with reference to SEQ ID NO:99), and at least 85% identity, at least 90% identity, or at least 95% identity to the sequence of SEQ ID NO:115 or 116. In some embodiments, clone CH3C.35.23.3 with the hole mutations and the mutations that modulate effector function has the sequence of SEQ ID NO:115 or 116.

In some embodiments, clone CH3C.35.23.3 may have hole mutations (e.g., T136S, L138A, and Y177V as numbered with reference to SEQ ID NO:99), mutations that increase serum stability or serum half-life (e.g., M22Y, S24T, and T26E as numbered with reference to SEQ ID NO:99), and at least 85% identity, at least 90% identity, or at least 95% identity to the sequence of SEQ ID NO:117. In some embodiments, clone CH3C.35.23.3 with the hole mutations and the mutations that increase serum stability or serum half-life has the sequence of SEQ ID NO:117.

In some embodiments, clone CH3C.35.23.3 may have hole mutations (e.g., T136S, L138A, and Y177V as numbered with reference to SEQ ID NO:99), mutations that increase serum stability or serum half-life (e.g., N204S with or without M198L as numbered with reference to SEQ ID NO:99), and at least 85% identity, at least 90% identity, or at least 95% identity to the sequence of SEQ ID NO:118. In some embodiments, clone CH3C.35.23.3 with the hole mutations and the mutations that increase serum stability or serum half-life has the sequence of SEQ ID NO:118.

In some embodiments, clone CH3C.35.23.3 may have hole mutations (e.g., T136S, L138A, and Y177V as numbered with reference to SEQ ID NO:99), mutations that modulate effector function (e.g., L4A, L5A, and/or P99G (e.g., L4A and L5A) as numbered with reference to SEQ ID NO:99), mutations that increase serum stability or serum half-life (e.g., M22Y, S24T, and T26E as numbered with reference to SEQ ID NO:99), and at least 85% identity, at least 90% identity, or at least 95% identity to the sequence of SEQ ID NO:119 or 120. In some embodiments, clone CH3C.35.23.3 with the hole mutations, the mutations that modulate effector function, and the mutations that increase serum stability or serum half-life has the sequence of SEQ ID NO:119 or 120.

In some embodiments, clone CH3C.35.23.3 may have hole mutations (e.g., T136S, L138A, and Y177V as numbered with reference to SEQ ID NO:99), mutations that modulate effector function (e.g., L4A, L5A, and/or P99G (e.g., L4A and L5A) as numbered with reference to SEQ ID NO:99), mutations that increase serum stability or serum half-life (e.g., N204S with or without M198L as numbered with reference to SEQ ID NO:99), and at least 85% identity, at least 90% identity, or at least 95% identity to the sequence of SEQ ID NO:121 or 122. In some embodiments, clone CH3C.35.23.3 with the hole mutations, the mutations that modulate effector function, and the mutations that increase serum stability or serum half-life has the sequence of SEQ ID NO:121 or 122.

Clone CH3C.35.23.4

In some embodiments, clone CH3C.35.23.4 may have a knob mutation (e.g., T136W as numbered with reference to SEQ ID NO:99) and at least 85% identity, at least 90% identity, or at least 95% identity to the sequence of SEQ ID NO:123. In some embodiments, clone CH3C.35.23.4 with the knob mutation has the sequence of SEQ ID NO:123.

In some embodiments, clone CH3C.35.23.4 may have a knob mutation (e.g., T136W as numbered with reference to SEQ ID NO:99), mutations that modulate effector function (e.g., L4A, L5A, and/or P99G (e.g., L4A and L5A) as numbered with reference to SEQ ID NO:99), and at least 85% identity, at least 90% identity, or at least 95% identity to the sequence of SEQ ID NO:124 or 125. In some embodiments, clone CH3C.35.23.4 with the knob mutation and the mutations that modulate effector function has the sequence of SEQ ID NO:124 or 125.

In some embodiments, clone CH3C.35.23.4 may have a knob mutation (e.g., T136W as numbered with reference to SEQ ID NO:99), mutations that increase serum stability or serum half-life (e.g., M22Y, S24T, and T26E as numbered with reference to SEQ ID NO:99), and at least 85% identity, at least 90% identity, or at least 95% identity to the sequence of SEQ ID NO:126. In some embodiments, clone CH3C.35.23.4 with the knob mutation and the mutations that increase serum stability or serum half-life has the sequence of SEQ ID NO:126.

In some embodiments, clone CH3C.35.23.4 may have a knob mutation (e.g., T136W as numbered with reference to SEQ ID NO:99), mutations that increase serum stability or serum half-life (e.g., N204S with or without M198L as numbered with reference to SEQ ID NO:99), and at least 85% identity, at least 90% identity, or at least 95% identity to the sequence of SEQ ID NO:127. In some embodiments, clone CH3C.35.23.4 with the knob mutation and the mutations that increase serum stability or serum half-life has the sequence of SEQ ID NO:127.

In some embodiments, clone CH3C.35.23.4 may have a knob mutation (e.g., T136W as numbered with reference to SEQ ID NO:99), mutations that modulate effector function (e.g., L4A, L5A, and/or P99G (e.g., L4A and L5A) as numbered with reference to SEQ ID NO:99), mutations that increase serum stability or serum half-life (e.g., M22Y, S24T, and T26E as numbered with reference to SEQ ID NO:99), and at least 85% identity, at least 90% identity, or at least 95% identity to the sequence of SEQ ID NO:128 or 129. In some embodiments, clone CH3C.35.23.4 with the knob mutation, the mutations that modulate effector function, and the mutations that increase serum stability or serum half-life has the sequence of SEQ ID NO:128 or 129.

In some embodiments, clone CH3C.35.23.4 may have a knob mutation (e.g., T136W as numbered with reference to SEQ ID NO:99), mutations that modulate effector function (e.g., L4A, L5A, and/or P99G (e.g., L4A and L5A) as numbered with reference to SEQ ID NO:99), mutations that increase serum stability or serum half-life (e.g., N204S with or without M198L as numbered with reference to SEQ ID NO:99), and at least 85% identity, at least 90% identity, or at least 95% identity to the sequence of SEQ ID NO:130 or 131. In some embodiments, clone CH3C.35.23.4 with the knob mutation, the mutations that modulate effector function, and the mutations that increase serum stability or serum half-life has the sequence of SEQ ID NO:130 or 131.

In some embodiments, clone CH3C.35.23.4 may have hole mutations (e.g., T136S, L138A, and Y177V as numbered with reference to SEQ ID NO:99) and at least 85% identity, at least 90% identity, or at least 95% identity to the sequence of SEQ ID NO:132. In some embodiments, clone CH3C.35.23.4 with the hole mutations has the sequence of SEQ ID NO:132.

In some embodiments, clone CH3C.35.23.4 may have hole mutations (e.g., T136S, L138A, and Y177V as numbered with reference to SEQ ID NO:99), mutations that modulate effector function (e.g., L4A, L5A, and/or P99G (e.g., L4A and L5A) as numbered with reference to SEQ ID NO:99), and at least 85% identity, at least 90% identity, or at least 95% identity to the sequence of SEQ ID NO:133 or 134. In some embodiments, clone CH3C.35.23.4 with the hole mutations and the mutations that modulate effector function has the sequence of SEQ ID NO:133 or 134.

In some embodiments, clone CH3C.35.23.4 may have hole mutations (e.g., T136S, L138A, and Y177V as numbered with reference to SEQ ID NO:99), mutations that increase serum stability or serum half-life (e.g., M22Y, S24T, and T26E as numbered with reference to SEQ ID NO:99), and at least 85% identity, at least 90% identity, or at least 95% identity to the sequence of SEQ ID NO:135. In some embodiments, clone CH3C.35.23.4 with the hole mutations and the mutations that increase serum stability or serum half-life has the sequence of SEQ ID NO:135.

In some embodiments, clone CH3C.35.23.4 may have hole mutations (e.g., T136S, L138A, and Y177V as numbered with reference to SEQ ID NO:99), mutations that increase serum stability or serum half-life (e.g., N204S with or without M198L as numbered with reference to SEQ ID NO:99), and at least 85% identity, at least 90% identity, or at least 95% identity to the sequence of SEQ ID NO:136. In some embodiments, clone CH3C.35.23.4 with the hole mutations and the mutations that increase serum stability or serum half-life has the sequence of SEQ ID NO:136.

In some embodiments, clone CH3C.35.23.4 may have hole mutations (e.g., T136S, L138A, and Y177V as numbered with reference to SEQ ID NO:99), mutations that modulate effector function (e.g., L4A, L5A, and/or P99G (e.g., L4A and L5A) as numbered with reference to SEQ ID NO:99), mutations that increase serum stability or serum half-life (e.g., M22Y, S24T, and T26E as numbered with reference to SEQ ID NO:99), and at least 85% identity, at least 90% identity, or at least 95% identity to the sequence of SEQ ID NO:137 or 138. In some embodiments, clone CH3C.35.23.4 with the hole mutations, the mutations that modulate effector function, and the mutations that increase serum stability or serum half-life has the sequence of SEQ ID NO:137 or 138.

In some embodiments, clone CH3C.35.23.4 may have hole mutations (e.g., T136S, L138A, and Y177V as numbered with reference to SEQ ID NO:99), mutations that modulate effector function (e.g., L4A, L5A, and/or P99G (e.g., L4A and L5A) as numbered with reference to SEQ ID NO:99), mutations that increase serum stability or serum half-life (e.g., N204S with or without M198L as numbered with reference to SEQ ID NO:99), and at least 85% identity, at least 90% identity, or at least 95% identity to the sequence of SEQ ID NO:139 or 140. In some embodiments, clone CH3C.35.23.4 with the hole mutations, the mutations that modulate effector function, and the mutations that increase serum stability or serum half-life has the sequence of SEQ ID NO:139 or 140.

Clone CH3C.35.23

In some embodiments, clone CH3C.35.23 may have a knob mutation (e.g., T136W as numbered with reference to SEQ ID NO:99) and at least 85% identity, at least 90% identity, or at least 95% identity to the sequence of SEQ ID NO:141. In some embodiments, clone CH3C.35.23 with the knob mutation has the sequence of SEQ ID NO:141.

In some embodiments, clone CH3C.35.23 may have a knob mutation (e.g., T136W as numbered with reference to SEQ ID NO:99), mutations that modulate effector function (e.g., L4A, L5A, and/or P99G (e.g., L4A and L5A) as numbered with reference to SEQ ID NO:99), and at least 85% identity, at least 90% identity, or at least 95% identity to the sequence of SEQ ID NO:142 or 143. In some embodiments, clone CH3C.35.23 with the knob mutation and the mutations that modulate effector function has the sequence of SEQ ID NO:142 or 143.

In some embodiments, clone CH3C.35.23 may have a knob mutation (e.g., T136W as numbered with reference to SEQ ID NO:99), mutations that increase serum stability or serum half-life (e.g., M22Y, S24T, and T26E as numbered with reference to SEQ ID NO:99), and at least 85% identity, at least 90% identity, or at least 95% identity to the sequence of SEQ ID NO:144. In some embodiments, clone CH3C.35.23 with the knob mutation and the mutations that increase serum stability or serum half-life has the sequence of SEQ ID NO:144.

In some embodiments, clone CH3C.35.23 may have a knob mutation (e.g., T136W as numbered with reference to SEQ ID NO:99), mutations that increase serum stability or serum half-life (e.g., N204S with or without M198L as numbered with reference to SEQ ID NO:99), and at least 85% identity, at least 90% identity, or at least 95% identity to the sequence of SEQ ID NO:145. In some embodiments, clone CH3C.35.23 with the knob mutation and the mutations that increase serum stability or serum half-life has the sequence of SEQ ID NO:145.

In some embodiments, clone CH3C.35.23 may have a knob mutation (e.g., T136W as numbered with reference to SEQ ID NO:99), mutations that modulate effector function (e.g., L4A, L5A, and/or P99G (e.g., L4A and L5A) as numbered with reference to SEQ ID NO:99), mutations that increase serum stability or serum half-life (e.g., M22Y, S24T, and T26E as numbered with reference to SEQ ID NO:99), and at least 85% identity, at least 90% identity, or at least 95% identity to the sequence of SEQ ID NO:146 or 147. In some embodiments, clone CH3C.35.23 with the knob mutation, the mutations that modulate effector function, and the mutations that increase serum stability or serum half-life has the sequence of SEQ ID NO:146 or 147.

In some embodiments, clone CH3C.35.23 may have a knob mutation (e.g., T136W as numbered with reference to SEQ ID NO:99), mutations that modulate effector function (e.g., L4A, L5A, and/or P99G (e.g., L4A and L5A) as numbered with reference to SEQ ID NO:99), mutations that increase serum stability or serum half-life (e.g., N204S with or without M198L as numbered with reference to SEQ ID NO:99), and at least 85% identity, at least 90% identity, or at least 95% identity to the sequence of SEQ ID NO:148 or 149. In some embodiments, clone CH3C.35.23 with the knob mutation, the mutations that modulate effector function, and the mutations that increase serum stability or serum half-life has the sequence of SEQ ID NO:148 or 149.

In some embodiments, clone CH3C.35.23 may have hole mutations (e.g., T136S, L138A, and Y177V as numbered with reference to SEQ ID NO:99) and at least 85% identity, at least 90% identity, or at least 95% identity to the sequence of SEQ ID NO:150. In some embodiments, clone CH3C.35.23 with the hole mutations has the sequence of SEQ ID NO:150.

In some embodiments, clone CH3C.35.23 may have hole mutations (e.g., T136S, L138A, and Y177V as numbered with reference to SEQ ID NO:99), mutations that modulate effector function (e.g., L4A, L5A, and/or P99G (e.g., L4A and L5A) as numbered with reference to SEQ ID NO:99), and at least 85% identity, at least 90% identity, or at least 95% identity to the sequence of SEQ ID NO:151 or 152. In some embodiments, clone CH3C.35.23 with the hole mutations and the mutations that modulate effector function has the sequence of SEQ ID NO:151 or 152.

In some embodiments, clone CH3C.35.23 may have hole mutations (e.g., T136S, L138A, and Y177V as numbered with reference to SEQ ID NO:99), mutations that increase serum stability or serum half-life (e.g., M22Y, S24T, and T26E as numbered with reference to SEQ ID NO:99), and at least 85% identity, at least 90% identity, or at least 95% identity to the sequence of SEQ ID NO:153. In some embodiments, clone CH3C.35.23 with the hole mutations and the mutations that increase serum stability or serum half-life has the sequence of SEQ ID NO:153.

In some embodiments, clone CH3C.35.23 may have hole mutations (e.g., T136S, L138A, and Y177V as numbered with reference to SEQ ID NO:99), mutations that increase serum stability or serum half-life (e.g., N204S with or without M198L as numbered with reference to SEQ ID NO:99), and at least 85% identity, at least 90% identity, or at least 95% identity to the sequence of SEQ ID NO:154. In some embodiments, clone CH3C.35.23 with the hole mutations and the mutations that increase serum stability or serum half-life has the sequence of SEQ ID NO:154.

In some embodiments, clone CH3C.35.23 may have hole mutations (e.g., T136S, L138A, and Y177V as numbered with reference to SEQ ID NO:99), mutations that modulate effector function (e.g., L4A, L5A, and/or P99G (e.g., L4A and L5A) as numbered with reference to SEQ ID NO:99), mutations that increase serum stability or serum half-life (e.g., M22Y, S24T, and T26E as numbered with reference to SEQ ID NO:99), and at least 85% identity, at least 90% identity, or at least 95% identity to the sequence of SEQ ID NO:155 or 156. In some embodiments, clone CH3C.35.23 with the hole mutations, the mutations that modulate effector function, and the mutations that increase serum stability or serum half-life has the sequence of SEQ ID NO:155 or 156.

In some embodiments, clone CH3C.35.23 may have hole mutations (e.g., T136S, L138A, and Y177V as numbered with reference to SEQ ID NO:99), mutations that modulate effector function (e.g., L4A, L5A, and/or P99G (e.g., L4A and L5A) as numbered with reference to SEQ ID NO:99), mutations that increase serum stability or serum half-life (e.g., N204S with or without M198L as numbered with reference to SEQ ID NO:99), and at least 85% identity, at least 90% identity, or at least 95% identity to the sequence of SEQ ID NO:157 or 158. In some embodiments, clone CH3C.35.23 with the hole mutations, the mutations that modulate effector function, and the mutations that increase serum stability or serum half-life has the sequence of SEQ ID NO:157 or 158.

Clone CH3C.35.23.1.1

In some embodiments, clone CH3C.35.23.1.1 may have a knob mutation (e.g., T136W as numbered with reference to SEQ ID NO:1) and at least 85% identity, at least 90% identity, or at least 95% identity to the sequence of SEQ ID NO:159. In some embodiments, clone CH3C.35.23.1.1 with the knob mutation has the sequence of SEQ ID NO:159.

In some embodiments, clone CH3C.35.23.1.1 may have a knob mutation (e.g., T136W as numbered with reference to SEQ ID NO:1), mutations that modulate effector function (e.g., L4A, L5A, and/or P99G (e.g., L4A and L5A) as numbered with reference to SEQ ID NO:1), and at least 85% identity, at least 90% identity, or at least 95% identity to the sequence of SEQ ID NO:160 or 161. In some embodiments, clone CH3C.35.23.1.1 with the knob mutation and the mutations that modulate effector function has the sequence of SEQ ID NO:160 or 161.

In some embodiments, clone CH3C.35.23.1.1 may have a knob mutation (e.g., T136W as numbered with reference to SEQ ID NO:1), mutations that increase serum stability or serum half-life (e.g., M22Y, S24T, and T26E as numbered with reference to SEQ ID NO:1), and at least 85% identity, at least 90% identity, or at least 95% identity to the sequence of SEQ ID NO:162. In some embodiments, clone CH3C.35.23.1.1 with the knob mutation and the mutations that increase serum stability or serum half-life has the sequence of SEQ ID NO:162.

In some embodiments, clone CH3C.35.23.1.1 may have a knob mutation (e.g., T136W as numbered with reference to SEQ ID NO:1), mutations that increase serum stability or serum half-life (e.g., N204S with or without M198L as numbered with reference to SEQ ID NO:1), and at least 85% identity, at least 90% identity, or at least 95% identity to the sequence of SEQ ID NO:163. In some embodiments, clone CH3C.35.23.1.1 with the knob mutation and the mutations that increase serum stability or serum half-life has the sequence of SEQ ID NO:163.

In some embodiments, clone CH3C.35.23.1.1 may have a knob mutation (e.g., T136W as numbered with reference to SEQ ID NO:1), mutations that modulate effector function (e.g., L4A, L5A, and/or P99G (e.g., L4A and L5A) as numbered with reference to SEQ ID NO:1), mutations that increase serum stability or serum half-life (e.g., M22Y, S24T, and T26E as numbered with reference to SEQ ID NO:1), and at least 85% identity, at least 90% identity, or at least 95% identity to the sequence of SEQ ID NO:164 or 165. In some embodiments, clone CH3C.35.23.1.1 with the knob mutation, the mutations that modulate effector function, and the mutations that increase serum stability or serum half-life has the sequence of SEQ ID NO:164 or 165.

In some embodiments, clone CH3C.35.23.1.1 may have a knob mutation (e.g., T136W as numbered with reference to SEQ ID NO:1), mutations that modulate effector function (e.g., L4A, L5A, and/or P99G (e.g., L4A and L5A) as numbered with reference to SEQ ID NO:1), mutations that increase serum stability or serum half-life (e.g., N204S with or without M198L as numbered with reference to SEQ ID NO:1), and at least 85% identity, at least 90% identity, or at least 95% identity to the sequence of SEQ ID NO:166 or 167. In some embodiments, clone CH3C.35.23.1.1 with the knob mutation, the mutations that modulate effector function, and the mutations that increase serum stability or serum half-life has the sequence of SEQ ID NO:166 or 167.

In some embodiments, clone CH3C.35.23.1.1 may have hole mutations (e.g., T136S, L138A, and Y177V as numbered with reference to SEQ ID NO:1) and at least 85% identity, at least 90% identity, or at least 95% identity to the sequence of SEQ ID NO:168. In some embodiments, clone CH3C.35.23.1.1 with the hole mutations has the sequence of SEQ ID NO:168.

In some embodiments, clone CH3C.35.23.1.1 may have hole mutations (e.g., T136S, L138A, and Y177V as numbered with reference to SEQ ID NO:1), mutations that modulate effector function (e.g., L4A, L5A, and/or P99G (e.g., L4A and L5A) as numbered with reference to SEQ ID NO:1), and at least 85% identity, at least 90% identity, or at least 95% identity to the sequence of SEQ ID NO:169 or 170. In some embodiments, clone CH3C.35.23.1.1 with the hole mutations and the mutations that modulate effector function has the sequence of SEQ ID NO:169 or 170.

In some embodiments, clone CH3C.35.23.1.1 may have hole mutations (e.g., T136S, L138A, and Y177V as numbered with reference to SEQ ID NO:1), mutations that increase serum stability or serum half-life (e.g., M22Y, S24T, and T26E as numbered with reference to SEQ ID NO:1), and at least 85% identity, at least 90% identity, or at least 95% identity to the sequence of SEQ ID NO:171. In some embodiments, clone CH3C.35.23.1.1 with the hole mutations and the mutations that increase serum stability or serum half-life has the sequence of SEQ ID NO:171.

In some embodiments, clone CH3C.35.23.1.1 may have hole mutations (e.g., T136S, L138A, and Y177V as numbered with reference to SEQ ID NO:1), mutations that increase serum stability or serum half-life (e.g., N204S with or without M198L as numbered with reference to SEQ ID NO:1), and at least 85% identity, at least 90% identity, or at least 95% identity to the sequence of SEQ ID NO:172. In some embodiments, clone CH3C.35.23.1.1 with the hole mutations and the mutations that increase serum stability or serum half-life has the sequence of SEQ ID NO:172.

In some embodiments, clone CH3C.35.23.1.1 may have hole mutations (e.g., T136S, L138A, and Y177V as numbered with reference to SEQ ID NO:1), mutations that modulate effector function (e.g., L4A, L5A, and/or P99G (e.g., L4A and L5A) as numbered with reference to SEQ ID NO:1), mutations that increase serum stability or serum half-life (e.g., M22Y, S24T, and T26E as numbered with reference to SEQ ID NO:1), and at least 85% identity, at least 90% identity, or at least 95% identity to the sequence of SEQ ID NO:173 or 174. In some embodiments, clone CH3C.35.23.1.1 with the hole mutations, the mutations that modulate effector function, and the mutations that increase serum stability or serum half-life has the sequence of SEQ ID NO:173 or 174.

In some embodiments, clone CH3C.35.23.1.1 may have hole mutations (e.g., T136S, L138A, and Y177V as numbered with reference to SEQ ID NO:1), mutations that modulate effector function (e.g., L4A, L5A, and/or P99G (e.g., L4A and L5A) as numbered with reference to SEQ ID NO:1), mutations that increase serum stability or serum half-life (e.g., N204S with or without M198L as numbered with reference to SEQ ID NO:1), and at least 85% identity, at least 90% identity, or at least 95% identity to the sequence of SEQ ID NO:175 or 176. In some embodiments, clone CH3C.35.23.1.1 with the hole mutations, the mutations that modulate effector function, and the mutations that increase serum stability or serum half-life has the sequence of SEQ ID NO:175 or 176.

### VIII. Formats for TfR-Binding Proteins

In some embodiments, a modified TfR-binding polypeptide as described herein is a subunit of a protein dimer. In some embodiments, the dimer is a heterodimer. In some embodiments, the dimer is a homodimer. In some embodiments, the dimer comprises a single Fc polypeptide that binds to the TfR receptor, i.e., is monovalent for TfR receptor binding. In some embodiments, the dimer comprises a second polypeptide that binds to the TfR receptor. The second polypeptide may comprise the same modified Fc polypeptide to provide a bivalent homodimer protein, or a second modified Fc polypeptide described herein may provide a second TfR receptor-binding site.

TfR-binding polypeptides described herein and dimeric or multimeric proteins comprising polypeptides may have a broad range of binding affinities, e.g., based on the format of the polypeptide. For example, in some embodiments, a polypeptide comprising a modified Fc polypeptide as described herein has an affinity for the TfR ranging anywhere from 1 pM to 10 μM. In some embodiments, affinity may be measured in a monovalent format. In other embodiments, affinity may be measured in a bivalent format, e.g., as a protein dimer comprising a modified Fc polypeptide.

Methods for analyzing binding affinity, binding kinetics, and cross-reactivity to analyze binding to TfR are known in the art. These methods include, but are not limited to, solid-phase binding assays (e.g., ELISA assay), immunoprecipitation, surface plasmon resonance (e.g., Biacore™ (GE Healthcare, Piscataway, N.J.)), kinetic exclusion assays (e.g., KinExA®), flow cytometry, fluorescence-activated cell sorting (FACS), BioLayer interferometry (e.g., Octet® (ForteBio, Inc., Menlo Park, Calif.)), and Western blot analysis. In some embodiments, ELISA is used to determine binding affinity and/or cross-reactivity. Methods for performing ELISA assays are known in the art and are also described in the Example section below. In some embodiments, surface plasmon resonance (SPR) is used to determine binding affinity, binding kinetics, and/or cross-reactivity. In some embodiments, kinetic exclusion assays are used to determine binding affinity, binding kinetics, and/or cross-reactivity. In some embodiments, BioLayer interferometry assays are used to determine binding affinity, binding kinetics, and/or cross-reactivity. FcRn binding of TfR-binding polypeptide may also be evaluated using these types of assays. FcRn binding is typically assayed under acidic conditions, e.g., at a pH of about 5 to about 6.

### IX. TfR-Binding Protein Conjugates

In some embodiments, an anti-HER2 construct that binds TfR and initiates transport across the BBB, e.g., an Fc polypeptide dimer-antibody variable region fusion protein, or an antibody heavy chain comprising a modified Fc polypeptide as described herein, can further comprises a partial or full hinge region. The hinge region can be from any immunoglobulin subclass or isotype. An illustrative immunoglobulin hinge is an IgG hinge region, such as an IgG1 hinge region, e.g., human IgG1 hinge amino acid sequence EPKSCDKTHTCPPCP (SEQ ID NO:104).

In still other embodiments, an anti-HER2 construct described herein (e.g., an Fc polypeptide dimer-antibody variable region fusion protein) may be fused to a peptide or protein useful in protein purification, e.g., polyhistidine, epitope tags, e.g., FLAG, c-Myc, hemagglutinin tags and the like, glutathione S transferase (GST), thioredoxin, protein A, protein G, or maltose binding protein (MBP). In some embodiments, purification tags can be fused to an antibody heavy chain. In some cases, the peptide or protein to which the anti-HER2 construct is fused may comprise a protease cleavage site, such as a cleavage site for Factor Xa or Thrombin. In certain embodiments, the linkage is cleavable by an enzyme present in the central nervous system.

Non-polypeptide agents may also be attached to an anti-HER2 construct (e.g., an Fc polypeptide dimer-antibody variable region fusion protein). Such agents include cytotoxic agents, imaging agents, a DNA or RNA molecule, or a chemical compound. In some embodiments, the agent may be a therapeutic or imaging chemical compound. In some embodiments, the agent is a small molecule, e.g., less than 1000 Da, less than 750 Da, or less than 500 Da.

An agent, either a polypeptide or non-polypeptide, may be attached to the N-terminal or C-terminal region of the Fc polypeptide dimer-antibody variable region fusion protein or antibody heavy chain, or attached to any region of the Fc polypeptide dimer-antibody variable region fusion protein or antibody heavy chain, so long as the agent does not interfere with binding of the TfR-binding polypeptide to TfR.

In various embodiments, the conjugates can be generated using well-known chemical cross-linking reagents and protocols. For example, there are a large number of chemical cross-linking agents that are known to those skilled in the art and useful for cross-linking the anti-HER2 construct with an agent of interest. For example, the cross-linking agents are heterobifunctional cross-linkers, which can be used to link molecules in a stepwise manner.

Heterobifunctional cross-linkers provide the ability to design more specific coupling methods for conjugating proteins, thereby reducing the occurrences of unwanted side reactions such as homo-protein polymers.

The agent of interest may be a therapeutic agent, including cytotoxic agents and the like, or a chemical moiety. In some embodiments, the agent may be a peptide or small molecule therapeutic or imaging agent.

### X. Co-Targeting of TfR and HER2

In addition to the use of TfR-binding as a means to enable delivery across the BBB, TfR is also highly expressed in various cancers, including certain HER2⁺ breast cancers. The mechanism by which cancer cells acquire increased TfR expression likely relates to tumor cell proliferation and increased metabolic demand, such as iron uptake. Because the brain penetrating TfR-binding anti-HER2 constructs (e.g., Fc polypeptide dimer-anti-HER2 antibody variable region fusion proteins) described herein contain a TfR-binding portion (e.g., TfR-binding Fc polypeptides) to enable transport across the BBB, there may be additional anti-tumor benefits upon binding to HER2⁺ tumor cells which also express TfR. Specifically, since these anti-HER2 constructs can bind both TfR and HER2 (e.g., bind to both the TfR and HER2 expressed on the same cell) at the same time, this can enhance their potency and/or efficacy.

As demonstrated in the Examples, we have developed TfR-binding Fc polypeptide dimer-anti-HER2 antibody variable region fusion proteins and have shown that these fusion proteins are capable of crossing the BBB, as well as transporting therapeutics across the BBB. Furthermore, we have demonstrated that co-targeting TfR and HER2 (e.g., subdomain I, II, and/or IV of HER2) by these fusion proteins enhanced cell growth inhibition and cell killing. In particular, as shown in Examples 11-14, the fusion proteins were effective in enhancing cell growth inhibition when, in contrast, there was no effect when targeting HER2 alone or when separate anti-TfR and anti-HER2 molecules were used in a combination, suggesting that binding to both TfR and HER2 with the same molecule is beneficial to achieve cell killing in this context. Thus, the experimental results support that the simultaneous binding, and crosslinking, of TfR and HER2 by a single molecule can potentiate the growth inhibition of HER2⁺ cancer cell lines.

Accordingly, disclosed herein is a method for treating a cancer or treating brain metastasis of a cancer in a subject that comprises administering to the subject a therapeutically effective amount of an anti-HER2 construct that binds to (a) subdomain I, II, or IV of human HER2 (e.g., subdomain I or II of human HER2) and (b) a transferrin receptor (TfR), wherein the anti-HER2 construct alone is therapeutically effective for treating the cancer. The anti-HER2 construct can be administered to the subject as a monotherapy. In some embodiments, the anti-HER2 construct is adminstered in combination with a chemotherapy or radiation therapy. The anti-HER2 construct can bind to HER2 and TfR on the same cell.

Also disclosed herein is a method for treating a cancer or treating brain metastasis of a cancer in a subject that comprises administering to the subject a therapeutically effective amount of:

(a) a first anti-HER2 construct that binds to subdomain II of human HER2; and
(b) a second anti-HER2 construct that binds to subdomain IV of human HER2,
or
(a) a first anti-HER2 construct that binds to subdomain I of human HER2; and
(b) a second anti-HER2 construct that binds to subdomain IV of human HER2,
or
(a) a first anti-HER2 construct that binds to subdomain I of human HER2; and
(b) a second anti-HER2 construct that binds to subdomain II of human HER2,
wherein the first and/or the second anti-HER2 construct also binds TfR. In some embodiments of this method, only one of the first and second anti-HER2 constructs binds to both HER2 and TfR.

In some embodiments, an anti-HER2 construct that binds to both HER2 and TfR can be an Fc polypeptide dimer-antibody variable region fusion protein that comprises an antibody variable region that binds to subdomain I, II, or IV of human HER2 and a modified Fc polypeptide dimer that comprises a first Fc polypeptide that contains modifications that create a TfR-binding site.

For example, in some embodiments, the Fc polypeptide dimer-antibody variable region fusion protein that binds to subdomain II of human HER2 comprises:

(a) a first heavy chain having the sequence of SEQ ID NO:38, a second heavy chain having the sequence of SEQ ID NO:55;
or
(b) a first heavy chain having the sequence of SEQ ID NO:46, a second heavy chain having the sequence of SEQ ID NO:55;
or
(c) a first heavy chain having the sequence of SEQ ID NO:30, a second heavy chain having the sequence of SEQ ID NO:55.

For example, in some embodiments, the Fc polypeptide dimer-antibody variable region fusion protein that binds to subdomain I of human HER2 comprises:

(a) a first heavy chain having the sequence of SEQ ID NO:267, a second heavy chain having the sequence of SEQ ID NO:290;
or
(b) a first heavy chain having the sequence of SEQ ID NO:275, a second heavy chain having the sequence of SEQ ID NO:290;
or
(c) a first heavy chain having the sequence of SEQ ID NO:259, a second heavy chain having the sequence of SEQ ID NO:290.

For example, in some embodiments, the Fc polypeptide dimer-antibody variable region fusion protein that binds to subdomain IV of human HER2 comprises:

(a) a first heavy chain having the sequence of SEQ ID NO:10, a second heavy chain having the sequence of SEQ ID NO:27;
or
(b) a first heavy chain having the sequence of SEQ ID NO:18, a second heavy chain having the sequence of SEQ ID NO:27;
or
(c) a first heavy chain having the sequence of SEQ ID NO:2, a second heavy chain having the sequence of SEQ ID NO:27.

In other embodiments, an anti-HER2 construct that binds to both HER2 and TfR can be a bispecific construct that comprises an antibody variable region that binds to human HER2 (e.g., subdomain I, II, or IV of human HER2) and an antibody variable region that binds TfR.

### XI. Methods to Increase Effector Function

For some applications, it is desirable to introduce modifications into anti-HER2 constructs (e.g., Fc polypeptide dimer-antibody variable region fusion proteins) and antibody heavy chains described herein that increase effector function (e.g., ADCC). One method for increasing effector function involves producing anti-HER2 constructs and/or antibody heavy chains that are afucosylated or fucose-deficient.

One approach for generating fucose-deficient anti-HER2 constructs (e.g., Fc polypeptide dimer-antibody variable region fusion proteins) and antibody heavy chains is to use a fucose analog such as 2-fluorofucose (2-FF). Fucose analogs can deplete or decrease the availability of GDP-fucose, which is a substrate required by fucosyltransferases to incorporate fucose into proteins.

An alternative approach for generating fucose-deficient anti-HER2 constructs (e.g., Fc polypeptide dimer-antibody variable region fusion proteins) and antibody heavy chains, commonly used for commercial production, is to employ an alpha-1,6 fucosyltransferase (FUT8) knockout cell line for expression of the anti-HER2 constructs (e.g., Fc polypeptide dimer-antibody variable region fusion proteins) or antibody heavy chains. A non-limiting example of a suitable FUT8 knockout cell line is the Chinese hamster ovary (CHO) FUT8 knockout cell line available from Lonza Biologics. Furthermore, as described in Mori et al. (Biotechnol. Bioeng. (2004) 88:901-908; hereby incorporated by reference in its entirety), FUT8 small interfering RNA (siRNA) can be used to convert CHO cell lines (e.g., by constitutive expression of the FUT8 siRNA) for the production of fucose-deficient proteins.

### XII. Nucleic Acids, Vectors, and Host Cells

The anti-HER2 constructs (e.g., Fc polypeptide dimer-antibody variable region fusion proteins) and antibody heavy chains as described herein are typically prepared using recombinant methods. Accordingly, isolated nucleic acids comprising a nucleic acid sequence encoding any of the anti-HER2 constructs or antibody heavy chains as described herein, and host cells into which the nucleic acids are introduced that are used to replicate the polypeptide-encoding nucleic acids and/or to express the anti-HER2 constructs or antibody heavy chains are provided. In some embodiments, the host cell is eukaryotic, e.g., a human cell.

In another aspect, polynucleotides are provided that comprise a nucleotide sequence that encodes the anti-HER2 constructs (e.g., Fc polypeptide dimer-antibody variable region fusion proteins) or antibody heavy chains described herein. The polynucleotides may be single-stranded or double-stranded. In some embodiments, the polynucleotide is DNA. In particular embodiments, the polynucleotide is cDNA. In some embodiments, the polynucleotide is RNA.

In some embodiments, the polynucleotide is included within a nucleic acid construct. In some embodiments, the construct is a replicable vector. In some embodiments, the vector is selected from a plasmid, a viral vector, a phagemid, a yeast chromosomal vector, and a non-episomal mammalian vector.

In some embodiments, the polynucleotide is operably linked to one or more regulatory nucleotide sequences in an expression construct. In one series of embodiments, the nucleic acid expression constructs are adapted for use as a surface expression library. In some embodiments, the library is adapted for surface expression in yeast. In some embodiments, the library is adapted for surface expression in phage. In another series of embodiments, the nucleic acid expression constructs are adapted for expression of the anti-HER2 construct (e.g., Fc polypeptide dimer-antibody variable region fusion protein) or antibody heavy chain in a system that permits isolation of the polypeptide in milligram or gram quantities. In some embodiments, the system is a mammalian cell expression system. In some embodiments, the system is a yeast cell expression system.

Expression vehicles for production of a recombinant anti-HER2 construct (e.g., Fc polypeptide dimer-antibody variable region fusion protein) or antibody heavy chain include plasmids and other vectors. For instance, suitable vectors include plasmids of the following types: pBR322-derived plasmids, pEMBL-derived plasmids, pEX-derived plasmids, pBTac-derived plasmids, and pUC-derived plasmids for expression in prokaryotic cells, such as E. coli. The pcDNAI/amp, pcDNAI/neo, pRc/CMV, pSV2gpt, pSV2neo, pSV2-dhfr, pTk2, pRSVneo, pMSG, pSVT7, pko-neo, and pHyg-derived vectors are examples of mammalian expression vectors suitable for transfection of eukaryotic cells. Alternatively, derivatives of viruses such as the bovine papilloma virus (BPV-1), or Epstein-Barr virus (pHEBo, pREP-derived, and p205) can be used for transient expression of polypeptides in eukaryotic cells. In some embodiments, it may be desirable to express the recombinant anti-HER2 construct or antibody heavy chain by the use of a baculovirus expression system. Examples of such baculovirus expression systems include pVL-derived vectors (such as pVL1392, pVL1393, and pVL941), pAcUW-derived vectors (such as pAcUW1), and pBlueBac-derived vectors. Additional expression systems include adenoviral, adeno-associated virus, and other viral expression systems.

Vectors may be transformed into any suitable host cell. In some embodiments, the host cells, e.g., bacteria or yeast cells, may be adapted for use as a surface expression library. In some cells, the vectors are expressed in host cells to express relatively large quantities of the anti-HER2 construct (e.g., Fc polypeptide dimer-antibody variable region fusion protein) or antibody heavy chain. Such host cells include mammalian cells, yeast cells, insect cells, and prokaryotic cells. In some embodiments, the cells are mammalian cells, such as Chinese Hamster Ovary (CHO) cell, baby hamster kidney (BHK) cell, NSO cell, YO cell, HEK293 cell, COS cell, Vero cell, or HeLa cell.

A host cell transfected with an expression vector encoding an anti-HER2 construct (e.g., an Fc polypeptide dimer-antibody variable region fusion protein) or antibody heavy chain can be cultured under appropriate conditions to allow expression of the anti-HER2 construct or antibody heavy chain to occur. The anti-HER2 constructs (e.g., Fc polypeptide dimer-antibody variable region fusion proteins) or antibody heavy chains may be secreted and isolated from a mixture of cells and medium containing the anti-HER2 constructs or antibody heavy chains. Alternatively, the anti-HER2 construct (e.g., Fc polypeptide dimer-antibody variable region fusion protein) or antibody heavy chain may be retained in the cytoplasm or in a membrane fraction and the cells harvested, lysed, and the anti-HER2 construct or antibody heavy chain isolated using a desired method.

### XIII. Therapeutic Methods

In some aspects, provided herein are methods transcytosis of an antibody variable region that is capable of binding HER2 (e.g., human HER2), or an antigen-binding fragment thereof, across an endothelium. In some embodiments, the methods comprise contacting the endothelium with a composition comprising an anti-HER2 construct (e.g., an Fc polypeptide dimer-antibody variable region fusion protein) described herein. In some embodiments, the endothelium is the blood brain barrier (BBB).

In other aspects, provided herein are methods for treating cancer (e.g., a HER2-positive cancer) in a subject. In some embodiments, the methods comprise administering to the subject a therapeutically effective amount of an anti-HER2 construct (e.g., an Fc polypeptide dimer-antibody variable region fusion protein) described herein. Any number of HER2-positive cancers can be treated according to the methods provided herein. Non-limiting examples include HER2-positive breast, ovarian, bladder, salivary gland, endometrial, pancreatic, and non-small-cell lung cancer (NSCLC), as well as HER2-positive gastric adenocarcinoma and/or a HER2-positive gastroesophageal junction adnocarcinoma. In some embodiments, the HER2-positive cancer is a HER2-positive breast cancer. In some embodiments, the HER2-positive cancer is a HER2-positive gastric adenocarcinoma and/or a HER2-positive gastroesophageal junction adnocarcinoma. In some embodiments, the HER2-positive cancer is a metastatic cancer.

In still other aspects, provided herein are methods for treating metastasis of a cancer (e.g., a HER2-positive cancer). In some embodiments, the methods comprise administering to the subject a therapeutically effective amount of an anti-HER2 construct (e.g., an Fc polypeptide dimer-antibody variable region fusion protein) described herein. In some embodiments, the metastasis is a brain metastasis of a HER2-positive cancer described above. In some embodiments, the metastasis is a brain metastasis of a HER2-positive breast cancer. In some embodiments, the metastasis is a brain metastasis of a HER2-positive gastric adenocarcinoma and/or a HER2-positive gastroesophageal junction adnocarcinoma.

In some embodiments, the anti-HER2 construct (e.g., Fc polypeptide dimer-antibody variable region fusion protein) comprises an anti-HER2 subdomain IV antibody variable region. In some embodiments, the anti-HER2 construct (e.g., Fc polypeptide dimer-antibody variable region fusion protein) comprises an anti-HER2 subdomain II antibody variable region. In some embodiments, the anti-HER2 construct (e.g., Fc polypeptide dimer-antibody variable region fusion protein) comprises an anti-HER2 subdomain I antibody variable region. In some embodiments, both an anti-HER2 construct (e.g., Fc polypeptide dimer-antibody variable region fusion protein) that comprises an anti-HER2 subdomain IV antibody variable region and an anti-HER2 construct (e.g., Fc polypeptide dimer-antibody variable region fusion protein) that comprises an anti-HER2 subdomain II antibody variable region are administered to the subject. In some embodiments, both an anti-HER2 construct (e.g., Fc polypeptide dimer-antibody variable region fusion protein) that comprises an anti-HER2 subdomain IV antibody variable region and an anti-HER2 construct (e.g., Fc polypeptide dimer-antibody variable region fusion protein) that comprises an anti-HER2 subdomain I antibody variable region are administered to the subject. In some embodiments, both an anti-HER2 construct (e.g., Fc polypeptide dimer-antibody variable region fusion protein) that comprises an anti-HER2 subdomain I antibody variable region and an anti-HER2 construct (e.g., Fc polypeptide dimer-antibody variable region fusion protein) that comprises an anti-HER2 subdomain II antibody variable region are administered to the subject.

In some embodiments, a first Fc polypeptide dimer-antibody variable region fusion protein and a second Fc polypeptide dimer-antibody variable region fusion protein are administered to the subject. In some instances, the antibody variable region of the first Fc polypeptide dimer-antibody variable region fusion protein comprises two antibody heavy chain variable regions comprising the amino acid sequence of SEQ ID NO:59 and two light chain variable regions comprising the amino acid sequence of SEQ ID NO:60. In some instances, the antibody variable region of the second Fc polypeptide dimer-antibody variable region fusion protein comprises two antibody heavy chain variable regions comprising the amino acid sequence of SEQ ID NO:61 and two light chain variable regions comprising the amino acid sequence of SEQ ID NO:62. In some instances, the antibody variable region of the first Fc polypeptide dimer-antibody variable region fusion protein comprises two antibody heavy chain variable regions comprising the amino acid sequence of SEQ ID NO:256 and two light chain variable regions comprising the amino acid sequence of SEQ ID NO:257.

In some embodiments, administering a combination of Fc polypeptide dimer-antibody variable region fusion proteins that target both subdomain IV and subdomain II of HER2 produces a greater therapeutic benefit than when only an Fc polypeptide dimer-antibody variable region fusion protein that targets subdomain IV or subdomain II is administered. In some embodiments, administering a combination of Fc polypeptide dimer-antibody variable region fusion proteins that target both subdomain IV and subdomain I of HER2 produces a greater therapeutic benefit than when only an Fc polypeptide dimer-antibody variable region fusion protein that targets subdomain IV or subdomain I is administered. In some embodiments, administering a combination of Fc polypeptide dimer-antibody variable region fusion proteins that target both subdomain II and subdomain I of HER2 produces a greater therapeutic benefit than when only an Fc polypeptide dimer-antibody variable region fusion protein that targets subdomain II or subdomain I is administered.

In some embodiments, administering an Fc polypeptide dimer-antibody variable region fusion protein that targets HER2 subdomain IV alone is more effective for inhibiting breast cancer cell growth than using an anti-HER2 subdomain IV antibody alone. In some embodiments, administering an Fc polypeptide dimer-antibody variable region fusion protein that targets HER2 subdomain II alone is more effective for inhibiting breast cancer cell growth than using an anti-HER2 subdomain II antibody alone. In some embodiments, administering an Fc polypeptide dimer-antibody variable region fusion protein that targets HER2 subdomain I alone is more effective for inhibiting breast cancer cell growth than using an anti-HER2 subdomain I antibody alone.

Further, in some embodiments, administering an Fc polypeptide dimer-antibody variable region fusion protein that targets HER2 subdomain IV alone is more effective for inhibiting breast cancer cell growth than using a combination of an anti-HER2 subdomain IV antibody and an anti-TfR antibody. In some embodiments, administering an Fc polypeptide dimer-antibody variable region fusion protein that targets HER2 subdomain II alone was more effective for inhibiting breast cancer cell growth than using a combination of an anti-HER2 subdomain II antibody and an anti-TfR antibody. In some embodiments, administering an Fc polypeptide dimer-antibody variable region fusion protein that targets HER2 subdomain I alone was more effective for inhibiting breast cancer cell growth than using a combination of an anti-HER2 subdomain I antibody and an anti-TfR antibody.

In other embodiments, using an anti-HER2 construct that includes an antibody variable region that is capable of binding HER2 (e.g., subdomain I, II, or IV of human HER2) and an antibody variable region that is capable of binding TfR is more effective for inhibiting breast cancer cell growth than using an anti-HER2 antibody alone (e.g., an anti-HER2 subdomain I antibody, an anti-HER2 subdomain II antibody, or an anti-HER2 subdomain IV antibody) or using separate anti-TfR and anti-HER2 (e.g., an anti-HER2 subdomain I, an anti-HER2 subdomain II, or an anti-HER2 subdomain IV) antibodies in a combination.

In some embodiments, the therapeutic benefit can comprise a decrease in or slowing of tumor growth, a decrease in tumor size (e.g., volume), a decrease in tumor cell viability, a decrease in the number of metastatic lesions, amelioration in one or more signs or symptoms of a cancer (e.g., HER2-positive cancer), and/or an increase in patient survival. In some embodiments, tumor cell survival, tumor growth, tumor size, and/or the number of metastatic lesions is decreased by at least about 10%, 20%, 30%, 40%, 50%, 60%, 70%, 80%, 90%, or more.

In some embodiments, the anti-HER2 construct (e.g., Fc polypeptide dimer-antibody variable region fusion protein) antagonizes HER2 activity. In some embodiments, HER2 activity is inhibited (e.g., by at least about 10%, 20%, 30%, 40%, 50%, 60%, 70%, 80%, 90%, or more).

In some embodiments, the subject has not been previously treated with an anti-HER2 therapy. In some embodiments, the subject has not been previously treated with a chemotherapy for metastatic disease. In some embodiments, the subject has not been previously treated with an anti-HER2 therapy and/or a chemotherapy for metastatic disease.

In some embodiments, the methods further comprise administering to the subject one or more other therapeutic agents or treatments. The additional therapeutic agents or treatments can comprise, for example, chemotherapy, immunotherapy, radiotherapy, hormone therapy, a differentiating agent, a small-molecule drug, or a combination thereof.

In some embodiments, an anti-HER2 construct (e.g., an Fc polypeptide dimer-antibody variable region fusion protein) is administered to a subject at a therapeutically effective amount or dose. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied according to several factors, including the chosen route of administration, the formulation of the composition, patient response, the severity of the condition, the subject's weight, and the judgment of the prescribing physician. The dosage can be increased or decreased over time, as required by an individual patient. In certain instances, a patient initially is given a low dose, which is then increased to an efficacious dosage tolerable to the patient. Determination of an effective amount is well within the capability of those skilled in the art.

The route of administration of an anti-HER2 construct (e.g., an Fc polypeptide dimer-antibody variable region fusion protein) as described herein can be oral, intraperitoneal, transdermal, subcutaneous, intravenous, intramuscular, intrathecal, inhalational, topical, intralesional, rectal, intrabronchial, nasal, transmucosal, intestinal, ocular or otic delivery, or any other methods known in the art. In some embodiments, the anti-HER2 construct (e.g., Fc polypeptide dimer-antibody variable region fusion protein) is administered orally, intravenously, or intraperitoneally.

Co-administration of multiple anti-HER2 constructs (e.g., multiple Fc polypeptide dimer-antibody variable region fusion proteins) or an anti-HER2 construct (e.g., an Fc polypeptide dimer-antibody variable region fusion protein) and an additional therapeutic agent can be performed together or separately, simultaneously or at different times. When administered, the therapeutic agents independently can be administered once, twice, three, four times daily or more or less often, as needed. In some embodiments, the administered therapeutic agents are administered once daily. In some embodiments, the administered therapeutic agents are administered at the same time or times, for instance as an admixture. In some embodiments, one or more of the therapeutic agents is administered in a sustained-release formulation.

In some embodiments, the combination of multiple anti-HER2 constructs (e.g., multiple Fc polypeptide dimer-antibody variable region fusion proteins) or the combination of an anti-HER2 construct (e.g., an Fc polypeptide dimer-antibody variable region fusion protein) and another therapeutic agent is administered concurrently. In some embodiments, the agents are administered sequentially. For example, a first agent (e.g., a first Fc polypeptide dimer-antibody variable region fusion protein) can be administered for about 1, 2, 3, 4, 5, 6, 7, 8, 9, 10, 15, 20, 25, 30, 40, 50, 60, 70, 80, 90, 100 days or more prior to administering a second Fc polypeptide dimer-antibody variable region fusion protein or another agent, or vice versa.

In some embodiments, the anti-HER2 constructs (e.g., Fc polypeptide dimer-antibody variable region fusion proteins) (and optionally another therapeutic agent) are administered to the subject over an extended period of time, e.g., for at least 30, 40, 50, 60, 70, 80, 90, 100, 150, 200, 250, 300, 350 days or longer.

### XIV. Pharmaceutical Compositions and Kits

In another aspect, pharmaceutical compositions and kits comprising an anti-HER2 construct (e.g., an Fc polypeptide dimer-antibody variable region fusion protein) and/or an antibody heavy chain described herein are provided. In some embodiments, the pharmaceutical compositions and kits are for use in transcytosing an antibody variable region that is capable of binding HER2 across an endothelium (e.g., the BBB) in a subject. In some embodiments, the pharmaceutical compositions and kits are for use in treating a HER2-positive cancer or a metastatic lesion thereof (e.g., in a subject).

Pharmaceutical Compositions

In some embodiments, pharmaceutical compositions comprising an anti-HER2 construct (e.g., an Fc polypeptide dimer-antibody variable region fusion protein) or antibody heavy chain are provided. In some embodiments, the pharmaceutical compositions comprise an Fc polypeptide dimer-antibody variable region fusion protein or antibody heavy chain that comprises an anti-HER2 subdomain IV antibody variable region. In some embodiments, the pharmaceutical compositions comprise an Fc polypeptide dimer-antibody variable region fusion protein or antibody heavy chain that comprises an anti-HER2 subdomain II antibody variable region. In some embodiments, the pharmaceutical compositions comprise an Fc polypeptide dimer-antibody variable region fusion protein or antibody heavy chain that comprises an anti-HER2 subdomain I antibody variable region. In some embodiments, the pharmaceutical compositions comprise an Fc polypeptide dimer-antibody variable region fusion protein or antibody heavy chain that comprises an anti-HER2 subdomain IV antibody variable region and an Fc polypeptide dimer-antibody variable region fusion protein or antibody heavy chain that comprises an anti-HER2 subdomain II antibody variable region. In some embodiments, the pharmaceutical compositions comprise an Fc polypeptide dimer-antibody variable region fusion protein or antibody heavy chain that comprises an anti-HER2 subdomain IV antibody variable region and an Fc polypeptide dimer-antibody variable region fusion protein or antibody heavy chain that comprises an anti-HER2 subdomain I antibody variable region. In some embodiments, the pharmaceutical compositions comprise an Fc polypeptide dimer-antibody variable region fusion protein or antibody heavy chain that comprises an anti-HER2 subdomain I antibody variable region and an Fc polypeptide dimer-antibody variable region fusion protein or antibody heavy chain that comprises an anti-HER2 subdomain II antibody variable region.

In some embodiments, a pharmaceutical composition comprises an anti-HER2 construct (e.g., an Fc polypeptide dimer-antibody variable region fusion protein) or antibody heavy chain as described herein and further comprises one or more pharmaceutically acceptable carriers and/or excipients. A pharmaceutically acceptable carrier includes any solvents, dispersion media, or coatings that are physiologically compatible and that preferably does not interfere with or otherwise inhibit the activity of the active agent. Various pharmaceutically acceptable excipients are well-known in the art.

In some embodiments, the carrier is suitable for intravenous, intramuscular, oral, intraperitoneal, intrathecal, transdermal, topical, or subcutaneous administration. Pharmaceutically acceptable carriers can contain one or more physiologically acceptable compound(s) that act, for example, to stabilize the composition or to increase or decrease the absorption of the active agent(s). Physiologically acceptable compounds can include, for example, carbohydrates, such as glucose, sucrose, or dextrans, antioxidants, such as ascorbic acid or glutathione, chelating agents, low molecular weight proteins, compositions that reduce the clearance or hydrolysis of the active agents, or excipients or other stabilizers and/or buffers. Other pharmaceutically acceptable carriers and their formulations are well-known in the art.

The pharmaceutical compositions described herein can be manufactured in a manner that is known to those of skill in the art, e.g., by means of conventional mixing, dissolving, granulating, dragee-making, emulsifying, encapsulating, entrapping, or lyophilizing processes. The following methods and excipients are merely exemplary and are in no way limiting.

For oral administration, an anti-HER2 construct (e.g., an Fc polypeptide dimer-antibody variable region fusion protein) or antibody heavy chain can be formulated by combining it with pharmaceutically acceptable carriers that are well known in the art. Such carriers enable the compounds to be formulated as tablets, pills, dragees, capsules, emulsions, lipophilic and hydrophilic suspensions, liquids, gels, syrups, slurries, suspensions and the like, for oral ingestion by a patient to be treated. Pharmaceutical preparations for oral use can be obtained by mixing the compounds with a solid excipient, optionally grinding a resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents can be added, such as a cross-linked polyvinyl pyrrolidone, agar, or alginic acid or a salt thereof such as sodium alginate.

An anti-HER2 construct (e.g., An Fc polypeptide dimer-antibody variable region fusion protein) or antibody heavy chain can be formulated for parenteral administration by injection, e.g., by bolus injection or continuous infusion. For injection, the compound or compounds can be formulated into preparations by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives. In some embodiments, compounds can be formulated in aqueous solutions, preferably in physiologically compatible buffers such as Hanks's solution, Ringer's solution, or physiological saline buffer. Formulations for injection can be presented in unit dosage form, e.g., in ampules or in multi-dose containers, with an added preservative. The compositions can take such forms as suspensions, solutions or emulsions in oily or aqueous vehicles, and can contain formulatory agents such as suspending, stabilizing and/or dispersing agents.

Typically, a pharmaceutical composition for use in in vivo administration is sterile. Sterilization can be accomplished according to methods known in the art, e.g., heat sterilization, steam sterilization, sterile filtration, or irradiation.

Dosages and desired drug concentration of pharmaceutical compositions of the disclosure may vary depending on the particular use envisioned. The determination of the appropriate dosage or route of administration is well within the skill of one in the art. Suitable dosages are also described herein.

Kits

In some embodiments, kits comprising an anti-HER2 construct (e.g., an Fc polypeptide dimer-antibody variable region fusion protein) or antibody heavy chain as described herein (e.g., as described above), or a pharmaceutical composition described herein, are provided. In some embodiments, the kits comprise an Fc polypeptide dimer-antibody variable region fusion protein or antibody heavy chain that comprises an anti-HER2 subdomain IV antibody variable region. In some embodiments, the kits comprise an Fc polypeptide dimer-antibody variable region fusion protein or antibody heavy chain that comprises an anti-HER2 subdomain II antibody variable region. In some embodiments, the kits comprise an Fc polypeptide dimer-antibody variable region fusion protein or antibody heavy chain that comprises an anti-HER2 subdomain I antibody variable region. In some embodiments, the kits comprise an Fc polypeptide dimer-antibody variable region fusion protein or antibody heavy chain that comprises an anti-HER2 subdomain IV antibody variable region and an Fc polypeptide dimer-antibody variable region fusion protein or antibody heavy chain that comprises an anti-HER2 subdomain II antibody variable region. In some embodiments, the kits comprise an Fc polypeptide dimer-antibody variable region fusion protein or antibody heavy chain that comprises an anti-HER2 subdomain IV antibody variable region and an Fc polypeptide dimer-antibody variable region fusion protein or antibody heavy chain that comprises an anti-HER2 subdomain I antibody variable region. In some embodiments, the kits comprise an Fc polypeptide dimer-antibody variable region fusion protein or antibody heavy chain that comprises an anti-HER2 subdomain I antibody variable region and an Fc polypeptide dimer-antibody variable region fusion protein or antibody heavy chain that comprises an anti-HER2 subdomain II antibody variable region. In some embodiments, the kits are for use in transcytosing an antibody variable region that is capable of binding HER2 across an endothelium (e.g., the BBB) in a subject. In some embodiments, the kits are for use in treating a HER2-positive cancer or a metastatic lesion thereof (e.g., in a subject).

In some embodiments, the kit further comprises instructional materials containing directions (i.e., protocols) for the practice of the methods described herein (e.g., instructions for using the kit contents for treating a cancer or metastatic lesion thereof such as a HER2-positive cancer). While the instructional materials typically comprise written or printed materials they are not limited to such. Any medium capable of storing such instructions and communicating them to an end user is contemplated. Such media include, but are not limited to electronic storage media (e.g., magnetic discs, tapes, cartridges, chips), optical media (e.g., CD-ROM), and the like. Such media may include addresses to internet sites that provide such instructional materials.

### XV. Examples

The following examples are included to demonstrate specific embodiments of the disclosure. It should be appreciated by those of skill in the art that the techniques disclosed in the examples which follow represent techniques to function well in the practice of the disclosure, and thus can be considered to constitute specific modes for its practice. However, those of skill in the art should, in light of the present disclosure, appreciate that many changes can be made in the specific embodiments which are disclosed and still obtain a like or similar result without departing from the spirit and scope of the disclosure.

### Example 1. Generation of Fc Polypeptide Dimer-Fab Fusion Proteins Having a Cis LALA Configuration with HER2 Binding Sites Against Subdomains II and IV

We have engineered a TfR-binding polypeptide in which the Fc polypeptide dimers contains the LALA mutation in one Fc polypeptide in the cis configuration. This TfR-binding polypeptide was able to attenuate blood and bone marrow reticulocyte depletion in mice, which was previously seen in the TfR-binding polypeptide with WT IgG. Importantly, upon binding to the Fab target, the TfR-binding polypeptide having the cis LALA configuration was able to elicit in vitro Fab target-mediated ADCC and CDC, as well as elicit an in vivo effector function immune response towards the target of interest. Based on these results, we rationalize that this molecule could be paired with specific Fab arms and become a brain-penetrant therapeutic that could both retain reticulocyte safety and elicit effector function towards the therapeutic target of interest.

In this example, as well as Examples 2-5 below, Fc polypeptide dimer-antibody variable region fusion proteins were created in which HER2 binding sites were engineered into the Fab arms of a TfR-binding polypeptide. Treatment of HER2-positive breast cancer has been very successful with therapies using anti-HER2_DIV (comprising heavy and light chains having the amino acid sequences set forth in SEQ ID NOS:97 and 57, respectively) and anti-HER2_DII (comprising heavy and light chains having the amino acid sequences set forth in SEQ ID NOS:98 and 58, respectively).

The Fc polypeptide dimer-antibody variable region fusion proteins used in this example, and Examples 2-5 below, were generated with HER2 Fab-binding sites fused to a modified Fc polypeptide dimer having a first Fc polypeptide that was a TfR-binding polypeptide (CH3C.35.23.1.1, having a LALA mutation) and a second Fc polypeptide that did not have a TfR-binding site or any modifications that reduce FcγR binding—these anti-HER2 constructs being referred to as HER2-35.23.1^{cisLALA}. Specifically, the anti-HER2 subdomain IV construct, HER2_DIV-35.23.1.1^{cisLALA} was an Fc polypeptide dimer-antibody variable region fusion protein having a first heavy chain comprising SEQ ID NO:2, a second heavy chain comprising SEQ ID NO:27, and two light chains comprising SEQ ID NO:57, and the anti-HER2 subdomain II construct, HER2_DII-35.23.1.1^{cisLALA} was an Fc polypeptide dimer-antibody variable region fusion protein having a first heavy chain comprising SEQ ID NO:30, a second heavy chain comprising SEQ ID NO:55, and two light chains comprising SEQ ID NO:58.

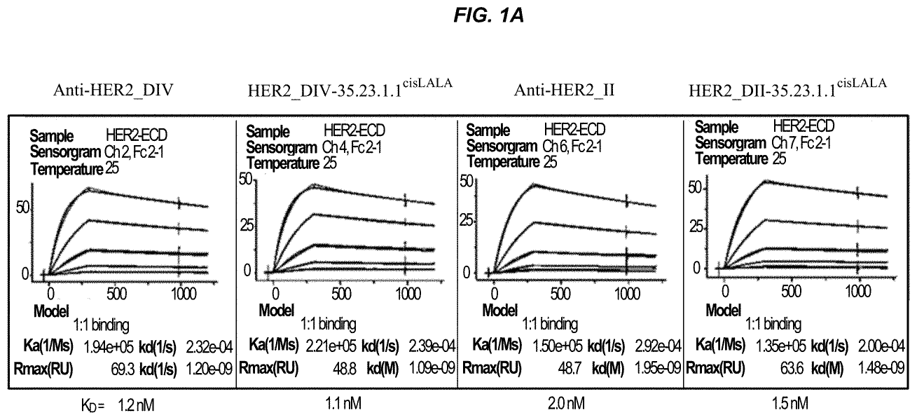

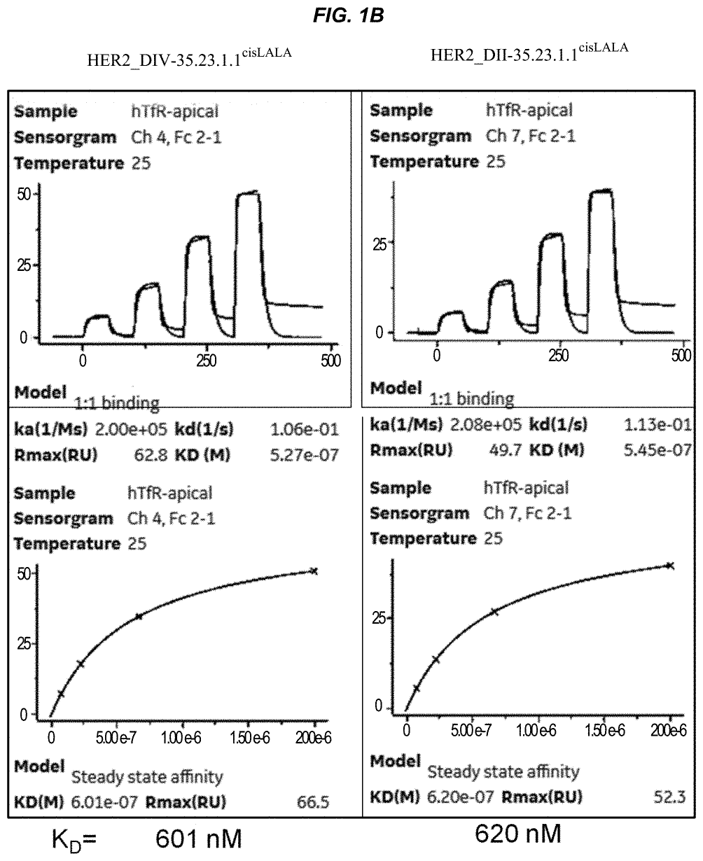

To confirm that the presence of a TfR-binding site in the Fc did not alter the binding affinity for HER2, we determined the binding coefficient (K_{D}) to HER2 extracellular domain protein using Biacore™. As expected, the binding affinity for HER2 using an extracellular domain protein was not altered in the T Fc polypeptide dimer-antibody variable region fusion protein that had either HER2 binding sequences against HER2 subdomain IV or II in the Fab arm (FIG. 1A ant Table 1). These results were compared to the binding affinities of anti-HER2 DIV and anti-HER2_DII.

TABLE 1
Binding affinities for HER2 extracellular domain.

| Molecule | Isotype | K_{D} (nM) |
|---|---|---|
| anti- HER2_DIV | huIgG1 | 1.2 |
| 35.23.1.1:HER2_DIV | huIgG1 | 0.8 |
| 35.23.1.1:HER2_DIV | huIgG1.LALA.knob | 1.1 |
| 35.23.1.1:HER2_DIV | huIgG1.LALA | 0.8 |
| anti-HER2_DII | huIgG1 | 2.0 |
| 35.23.1.1:HER2_DII | huIgG1.LALA.knob | 1.5 |

To confirm that the addition of HER2 binding sites did not alter the binding affinity for TfR at the TfR-binding domain in the Fc polypeptide, we also compared the TfR-binding affinities of various Fc polypeptide dimer-antibody variable region fusion proteins. The affinities for TfR did not differ significantly between HER2_DIV-35.23.1.1^{cisLALA} and HER2_DII-35.23.1.1^{cisLALA} (601 nM and 620 nM, respectively) (FIG. 1B and Table 2).

TABLE 2
Binding affinities for apical hTfR.

| Molecule | Isotype | K_{D} (nM) |
|---|---|---|
| ATV35.23.1.1:Her2_DIV | huIgG1 | 626 |
| ATV35.23.1.1:Her2_DIV | huIgG1.LALA.knob | 601 |
| ATV35.23.1.1:Her2_DIV | huIgG1.LALA | 607 |
| ATV35.23.1.1:Her2_DII | huIgG1.LALA.knob | 620 |

### Example 2. BT474 Inhibition by Fc Polypeptide Dimer-Fab Fusion Proteins Having HER2 Fab-Binding Sites

The ability of HER2_DIV-35.23.1.1^{cisLALA} to inhibit cancer cell proliferation was evaluated in a HER2-positive breast cancer cell line. BT474 cells were plated overnight at 10,000 cells/well in a 96-well plate, treated with 60 μL of 1:3 serial dilution of molecules of interest beginning at 25,000 ng/mL. Media and drugs were replenished on Day 3. On Day 6, cell growth was determined using 5 μL of WST-1 reagent in 50 μL of growth media. The plate was incubated for 4 hours in the presence of WST-1 reagent, and absorbance was determined at 440 nm. The percent of growth inhibition/proliferation was normalized to the untreated control.

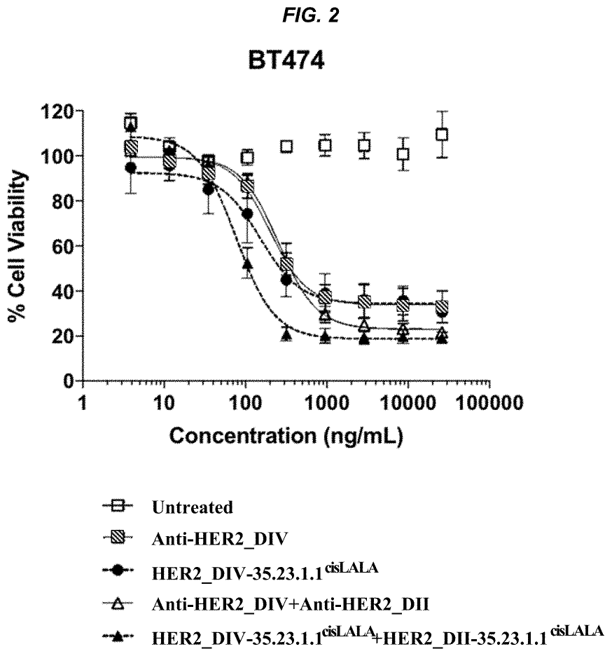

HER2_DIV-35.23.1.1^{cisLALA} had the ability to inhibit growth of BT474 cells in a manner similar to anti-HER2_DIV. The addition of a TfR-binding site at the Fc did not interfere with the degree of growth inhibition (FIG. 2 and Table 3).

TABLE 3
IC₅₀ values for BT474 growth inhibition assay.

| Molecule | IC₅₀ (nM) |
|---|---|
| anti-HER2_DIV | 1.30 ± 0.22 |
| 35.23.1.1^{cisLALA}:HER2_DIV | 0.986 ± 0.30 |
| anti-HER2_DIV + anti-Her2_DII | 1.66 ± 0.18 |
| 35.23.1.1^{cisLALA}:Her2_DIV + | 0.500 ± 0.056 |
| 35.23.1.1^{cisLALA}:Her2_DII | |

### Example 3. Decreased pAKT Protein Levels in HER2-Positive Breast Cancer Cell Line BT474

HER2 amplification is known to dysregulate the downstream PI3K/Akt signaling pathway. Small molecule inhibitors or antibodies that target HER2 decrease phosphorylated AKT (pAKT) and prevent EGFR activation and downstream signaling mechanisms in HER2-positive breast cancer cells. The reduction of pAKT protein level serves as a useful readout to determine target engagement. Importantly, anti-HER2_DIV robustly decreases pAkt protein level in various breast cancer cell lines, including BT474. Conversely, in tumors that have increased pAkt level, there is also increased resistance to anti-HER2_DIV treatment that is observed.

Immunoblotting was used to assess the protein expression level of pAKT in BT474 upon treatment with HER2_DIV-35.23.11^{cisLALA} BT474 cells were plated overnight at 100,000-200,000 cells/well in a 24-well plate. Cells were treated at 50 μg/mL for 2 hours, washed twice with PBS, and lysate was harvested using RIPA buffer with complete protease inhibitor. 8 μl of protein lysate was added to each well and proteins were blotted and analyzed using anti-pAKT, anti-AKT, and anti-b-actin at 1:1,000. Bands were visualized and analyzed using the Li-CORE imaging system.

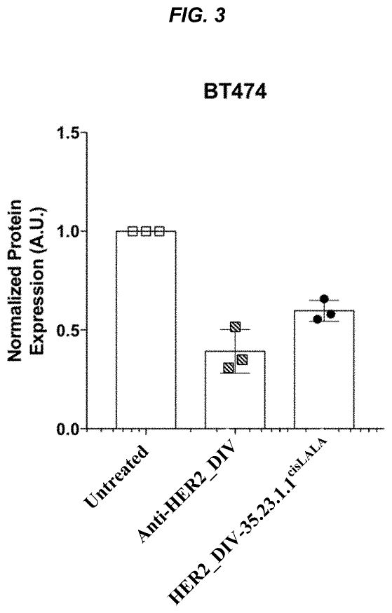

Consistent with growth inhibition properties in BT474 cells, HER2_DIV-35.23.1.1^{cisLALA} significantly decreased pAKT protein levels compared to untreated control (FIG. 3). This data demonstrates that HER2_DIV-35.23.1.1^{cisLALA} engaged HER2-positive breast cancer cells in a similar manner as anti-HER2_DIV, by inhibiting downstream PI3K/Akt signaling pathways.

### Example 4. Combination Treatment for Inhibition of BT474 Cells

The combination of anti-HER2_DIV and anti-HER2_DII has demonstrated superior clinical activity in metastatic cancer and is currently the standard of care for HER2-positive breast cancer patients. Anti-HER2_DII binds to subdomain II of HER2 and prevents heterodimerization with HER3 and EGFR. Interestingly, anti-HER2_DII alone is not as effective as anti-HER2_DIV alone. When it is combined with anti-HER2_DIV, it has stronger antitumor activity in HER2-positive breast cancer better than anti-HER2_DIV monotherapy. Importantly, the combination therapy has also demonstrated more effectiveness in tumors that are resistant to anti-HER2_DIV.

As mentioned above, in addition to HER2_DIV-35.23.1.1^{cisLALA}, an Fc polypeptide dimer-antibody variable region fusion protein having binding sites against HER2 subdomain II (HER2_DII-35.23.1.1^{cisLALA}) was also generated. BT474 cells were treated with the combination of both Fc polypeptide dimer-antibody variable region fusion proteins; growth inhibition was evaluated by WST1 viability assay as described above. Remarkably, the combination of HER2_DIV-35.23.1.1^{cisLALA} and HER2_DII-35.23.1.1^{cisLALA} led to more potent growth inhibition than the combination of anti-HER2_DIV and anti-HER2_DII (FIG. 2).

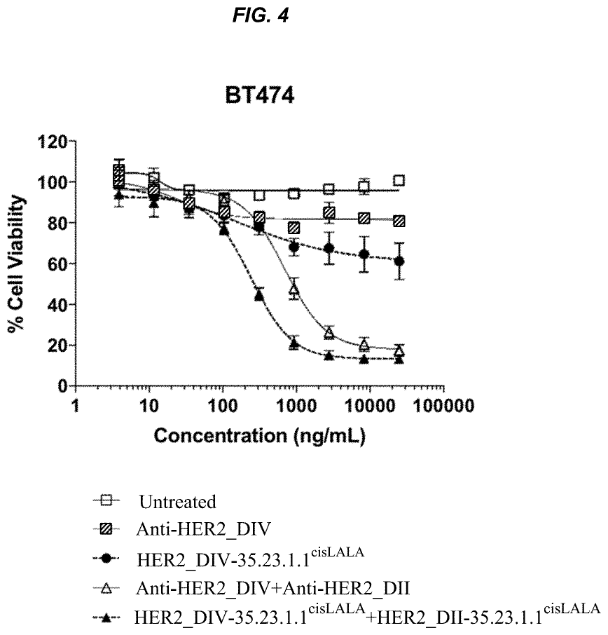

Because combination therapy of anti-HER2_DIV and anti-HER2_DII is also known to overcome tumor resistance, we next tested the ability of the combination of HER2_DIV-35.23.1.1^{cisLALA} and HER2_DII-35.23.1.1^{cisLALA} to overcome tumor resistance in a neuregulin 1-induced resistance model. Neuregulin 1 (NRG-1) is a ligand for HER3 and is enriched in the brain microenvironment and has been shown to be a potent inducer of resistance to PI3K inhibitor in HER2-amplified breast cancer cell lines. Specifically, NRG-1 activates HER3 signaling pathways and initiates HER2-HER3 dimerization. This in turn leads to activation of the PI3K/Akt pathway and render the tumor cells more resistant to anti-HER2_DIV treatment. In the presence of NRG1 (50 ng/mL), BT474 breast cancer cells were indeed resistant to anti-HER2_DIV or HER2_DIV-35.23.1.1^{cisLALA} (FIG. 4). Nonetheless, binding HER2 at subdomain IV and II through the combination treatment of anti-HER2_DIV and anti-HER2_DII overcame this resistance, consistent with published data (FIG. 4). Furthermore, in the presence of NRG1, the combination of HER2_DIV-35.23.1.1^{cisLALA} and HER2_DII-35.23.1.1^{cisLALA} was more potent than the combination of anti-HER2_DIV and anti-HER2_DII, as determined by WST1 growth inhibition assay in BT474 cells (FIG. 4 and Table 4). These results demonstrate that the combination of HER2_DIV-35.23.1.1^{cisLALA} and HER2_DII-35.23.1.1^{cisLALA} not only decreased breast cancer cell proliferation in vitro, but surprisingly, was also more potent than the combination of anti-HER2_DIV and anti-HER2_DII.

Because increasing preclinical and clinical data suggest that HER2-positive breast cancer brain metastasis (BCBM) is more resistant to anti-HER2_DIV than peripheral tumor, and that targeting HER2-HER3 dimerization is a strategy to overcome this resistance, the combination of HER2_DIV-35.23.1.1^{cisLALA} and HER2_DII-35.23.1.1^{cisLALA} can enable 1) delivery of the therapeutic across the blood brain barrier to access HER2-positive BCBM, and 2) inhibit cancer cell proliferation that have become resistant to anti-HER2_DIV as a result of the brain microenvironment.

TABLE 4
IC₅₀ values for BT474 growth inhibition assay.

| Molecule | IC₅₀ (nM) |
|---|---|
| anti-HER2_DIV | — |
| 35.23.1.1^{cisLALA}:Her2_DIV | — |
| anti-HER2_DIV + anti-Her2_DII | 4.63 ± 0.55 |
| 35.23.1.1^{cisLALA}:Her2_DIV + | 1.61 ± 0.18 |
| 35.23.1.1^{cisLALA}:Her2_DII | |

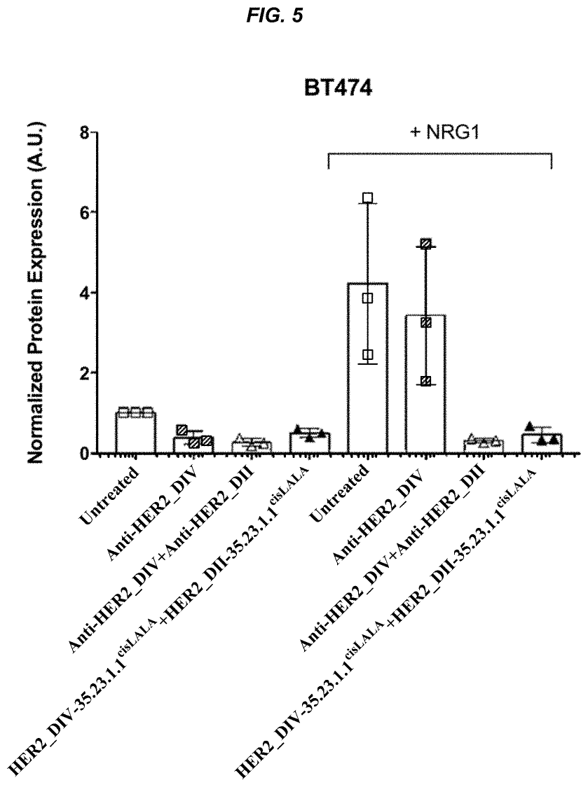

Similar to anti-HER2_DIV, a combination of anti-HER2_DIV and anti-HER2_DII robustly decreased pAKT protein levels. The combination of HER2_DIV-35.23.1.1^{cisLALA} and HER2_DII-35.23.1.1^{cisLALA} also robustly decreased pAKT protein level (FIG. 5). In the presence of NRG1 (50 ng/mL), which leads to increased tumor cell resistance as demonstrated by increased pAKT protein levels, the combination of HER2_DIV-35.23.1.1^{cisLALA} and HER2_DII-35.23.1.1^{cisLALA}] (at 50 μg/mL each, for 2 hours) also robustly decreased pAKT protein levels (FIG. 5). This result was consistent with the growth inhibition that was observed upon NRG1-induced resistance in BT474 cells (FIG. 4).

### Example 5. Fc Polypeptide Dimer-Fab Fusion Proteins Elicit Fab-Mediated ADCC in HER2-Positive Breast Cancer Cells

Increasing literature indicates that effector function such as ADCC is an important component of the antitumor mechanism of anti-HER2_DIV. In addition, studies with FcγR knock-out mice have shown that the anti-tumor effect of anti-HER2_DIV is drastically blunted. One challenge associated with polypeptide therapies that target TfR for BBB access is reticulocyte safety. For this reason, LALA mutations are introduced in order to disengage FcγR-binding in TfR-expressing polypeptides. On the other hand, for applications in which effector function is required, the LALA mutations impose therapeutic limitations in therapies that require blood brain barrier access and effector function response.

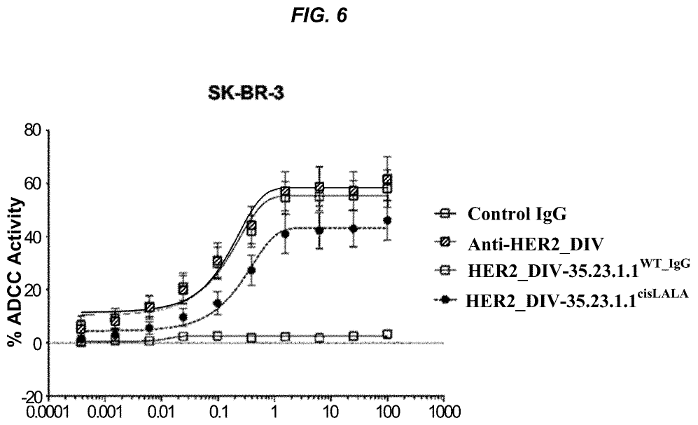

The ability of Fc polypeptide dimer-antibody variable region fusion proteins to elicit Fab-mediated ADCC was evaluated in a HER2-positive breast cancer cell line. Target cells that express high HER2 levels (SK-BR-3) were used to evaluate HER2-mediated ADCC to ensure that effector function was retained. Target cells were plated target cells were plated at 10,000 cells/well, opsonized, incubated with NK cells at 25:1 effector:target cells ratio, and evaluated for cytotoxicity by LDH expression. Target cells were opsonized with (1) control IgG, (2) anti-HER2_DIV, (3) hIgG1 with a TfR-binding site and a HER2 Fab-binding site (HER2_DIV-35.23.1.1^{WT IgG}) and (4) HER2_DIV-35.23.1.1^{cisLALA}. Consistent with Fab-mediated ADCC that was previously observed with a TfR-binding polypeptide with cis configuration, ADCC was observed in HER2-positive breast cancer cell line SK-BR-3 with HER2_DIV-35.23.1.1^{cisLALA}, demonstrating that effector function was retained (FIG. 6).

### Example 6. Fc Polypeptide Dimer-Fab Fusion Proteins have Superior Anti-Tumor Potency in a HER2⁺ Xenograft Model with BT474 Cell Line

We next utilized a BT474 xenograft tumor model in SCID mice to examine in vivo anti-tumor efficacy of the combination of HER2_DIV-35.23.4^{cisLALA} and HER2_DII-35.23.4^{cisLALA} compared to the combination of anti-HER2-DIV and anti-HER2-DII. Specifically, HER2_DIV-35.23.4^{cisLALA} has a first heavy chain comprising SEQ ID NO:18, a second heavy chain comprising SEQ ID NO:27, and two light chains comprising SEQ ID NO:57. HER2_DII-35.23.4^{cisLALA} has a first heavy chain comprising SEQ ID NO:46, a second heavy chain comprising SEQ ID NO:55, and two light chains comprising SEQ ID NO:58. Anti-HER2_DIV has two heavy chains comprising SEQ ID NO:97 and two light chains comprising SEQ ID NO:57. Anti-HER2_DII has two heavy chains comprising SEQ ID NO:98 and two light chains comprising SEQ ID NO:58.

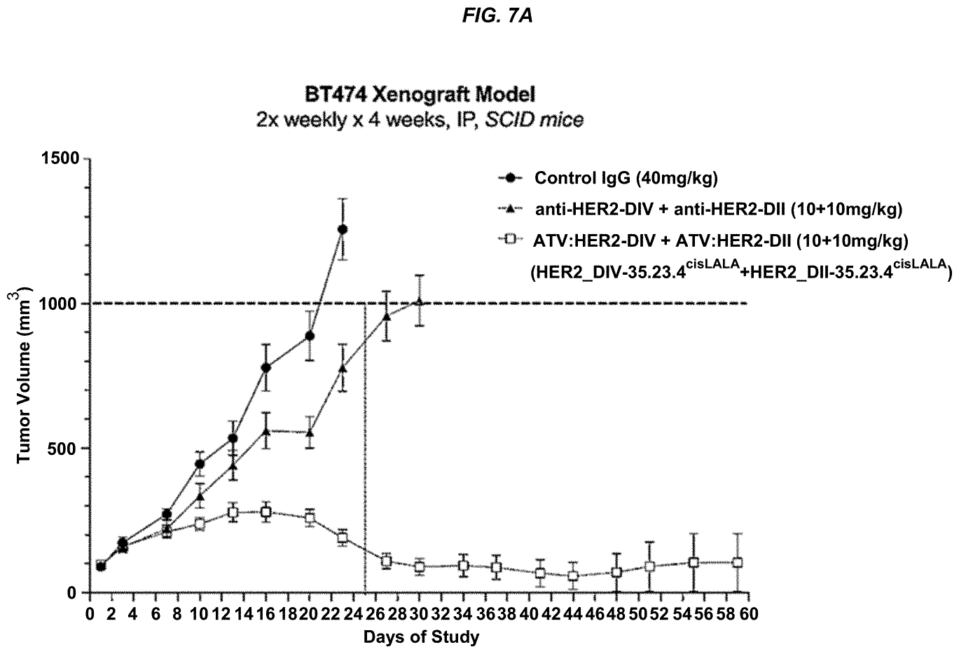

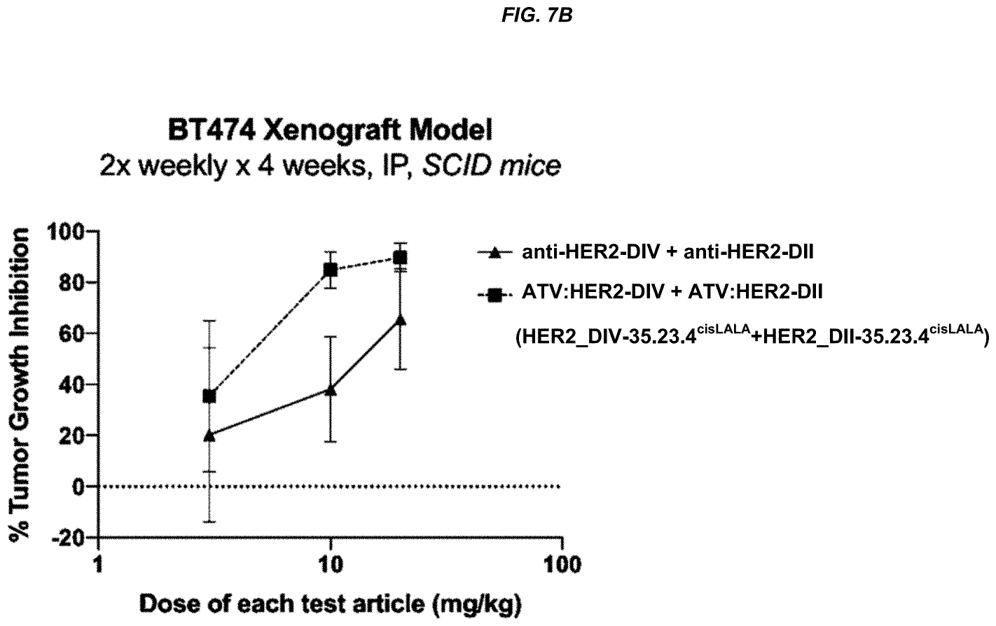

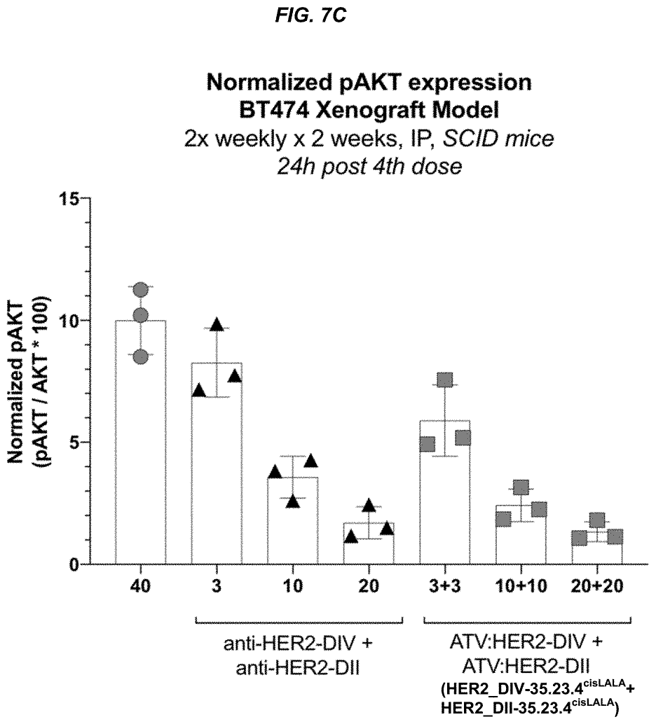

Tumor fragments derived from BT474 cells were implanted subcutaneously at the flank of SCID mice. When the tumor size has reached an average of 100 mm³, animals were treated twice a week for 4 weeks at 3, 10, or 20 mg/kg of each test article, and 40 mg/kg of control IgG as the control. Tumor volume was measured twice a week; animals were sacrificed when they have reached an experimental endpoint of 1000 mm³ or until 60 days of the study. As shown in FIG. 7A, at the medium dose 10 mg/kg, there is significantly higher tumor growth inhibition in the animals treated with ATV:HER2-DIV+ATV:HER2-DII (HER2_DIV-35.23.4^{cisLALA} and HER2_DII-35.23.4^{cisLALA}) compared to anti-HER2-DIV+anti-HER2-DII. A dose-response relationship using doses 3, 10, and 20 mg/kg of each test article shows that ATV:HER2-DIV+ATV:HER2-DII is more potent than anti-HER2-DIV+anti-HER2-DII (FIG. 7B). A cohort of animals were sacrificed 24h post 4^{th} dose. Tumors were harvested and lysates were used to determine pAKT and total AKT protein expression levels using a MSD phospho AKT (Ser473)/Total AKT Assay Whole Cell Lysate Kit as per manufacturer's protocol. Briefly, assay plate was blocked for 1h with manufacturer's blocking solution. Lysate supernatant samples and assay controls were incubated onto the plate for 1h. Detection antibody was subsequently added and the plate was read using an MSD plate reader. As expected, treatment of both anti-HER2-DIV+anti-HER2-DII and ATV:HER2-DIV+ATV:HER2-DII significantly reduced pAKT levels, which is consistent with the mechanism in which targeting against HER2 could abrogate the PI3K/Akt signaling pathway that is activated in HER2⁺ tumors (FIG. 7C).

### Example 7. Fc Polypeptide Dimer-Fab Fusion Proteins have Increased Brain Concentrations and Brain:Plasma Ratio Compared to Standard hIgG

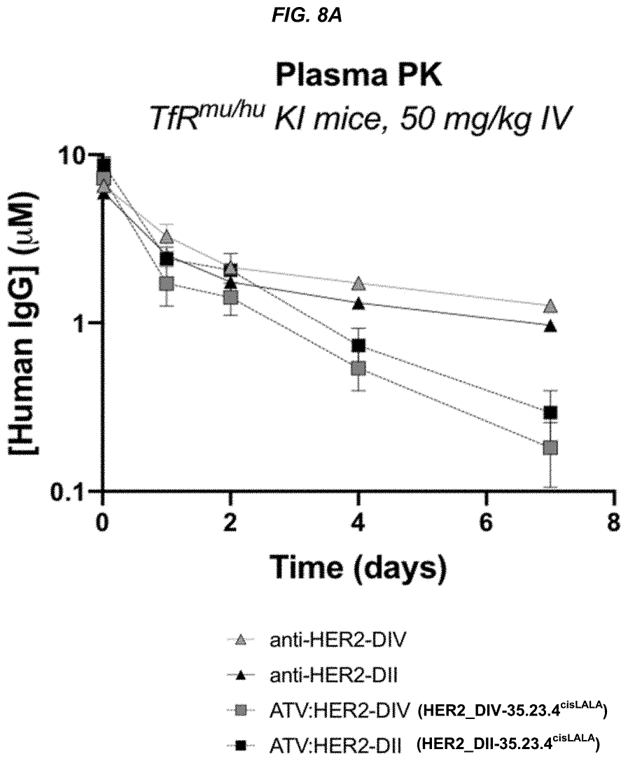

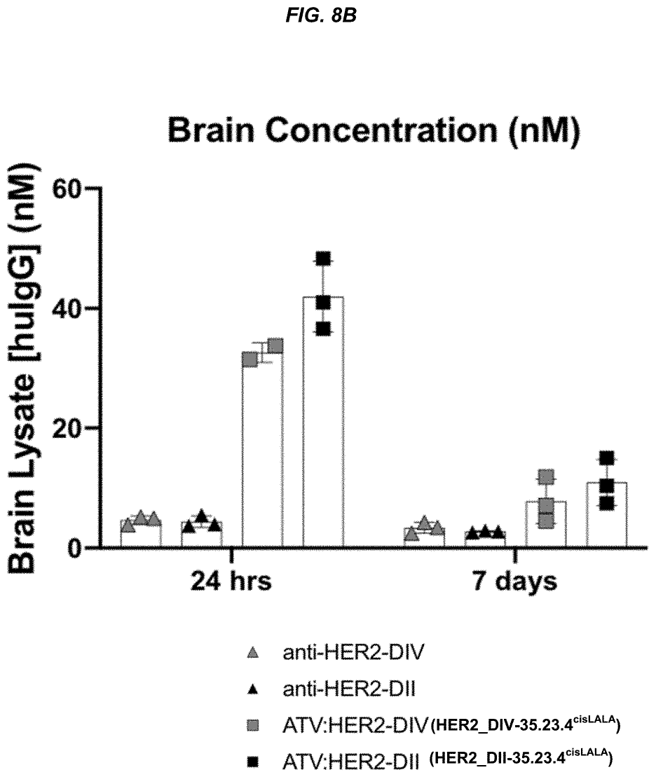

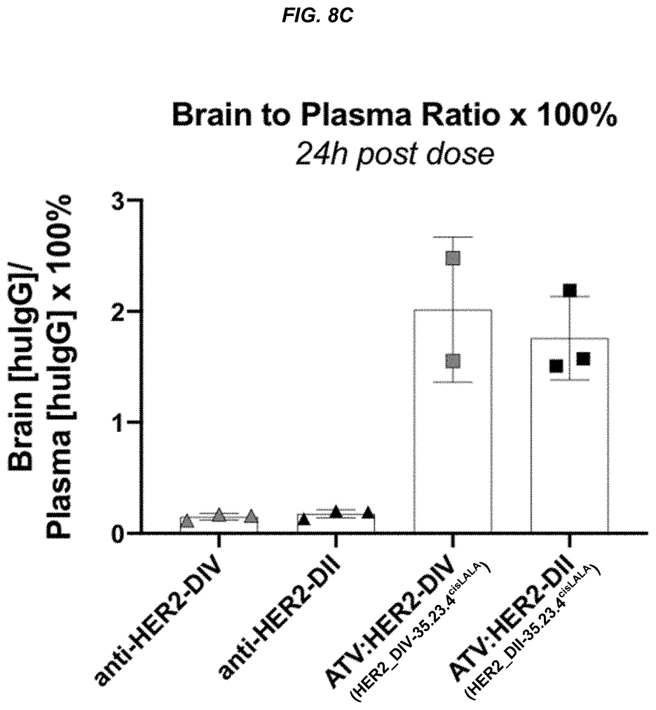

To evaluate the plasma exposure and brain uptake of ATV:HER2-DIV (HER2_DIV-35.23.4^{cisLALA}) and ATV:HER2-DII (HER2_DII-35.23.4^{cisLALA}) molecules, TfR^{mu/hu} KI mice were treated with anti-HER2-DIV, anti-HER2-DII, ATV:HER2-DIV, and ATV:HER2-DII at 50 mg/kg on Day 0. Plasma samples were collected on Days 1, 2, 4, and 7, while brains were harvested on Days 1 and 7. Antibody concentrations by hIgG measurements in mouse plasma and brain samples were quantified using a sandwich ELISA (FIGS. 8A and 8B). Briefly, a 384-well MaxiSorp plate was coated overnight with a polyclonal donkey anti-human IgG capture antibody, specific for the Fc fragment. The respective dosing solutions were used as a standard for antibody quantification. Brain samples were homogenized in 1% NP40 lysis buffer. Standards, diluted plasma and brain lysates were added to the blocked plates for 2 hours at RT, followed by a 1-hour incubation with the detection antibody, an HRP-conjugated polyclonal goat anti-human IgG specific for the Fc fragment. The hIgG concentrations were determined using the standard curve.

Target-mediated drug disposition is expected with TfR-binding molecules since TfR is ubiquitously expressed in peripheral tissues. Indeed, the ability of ATV:HER2-DIV and ATV:HER2-DII to bind TfR enables these molecules to be transported across the blood brain barrier. Following 24h post dose at 50 mg/kg, the brain concentration was 33-42 nM for HER2-ATVs and about 4.5 nM for anti-HER2 molecules (FIG. 8B), while the brain to plasma ratio was about 10 fold higher for HER2-ATVs anti-HER2 antibodies (FIG. 8C). Taken together, these data support that these polypeptide dimer-Fab fusion proteins could have the effects of anti-HER2 antibodies but with addition BBB-penetrant features.

### Example 8. Characterization of Additional Fc Polypeptide Dimer-Fab Fusion Proteins

Additional Fc polypeptide dimer-antibody variable region fusion proteins were constructed using CH3C.35.23.4 in the first Fc polypeptide. The binding affinities of these anti-HER2 constructs for human TfR apical domain and the extracellular domain of HER2 were tested and compared to Fc polypeptide dimer-antibody variable region fusion proteins wherein the first Fc polypeptide comprised CH3C.35.23.1.1.

The affinities of Fc polypeptide dimer-antibody variable region fusion proteins for human TfR apical domain and HER2 ECD were determined by surface plasmon resonance using a Biacore™ 8K. Fc polypeptide dimer-antibody variable region fusion proteins were captured using a Human Fab Capture Kit (GE, #28-9583-25) on Biacore™ Series S CM5 sensor chips (GE, #29149604) for measurement of human TfR apical domain binding, or using a Human Fc capture Kit (GE, #29234600) for measurement of human HER2-ECD binding (ACROBiosystems, HE2-H5225). Serial 3-fold dilutions of each antigen were injected at a flow rate of 30 μL/min. The binding of the antigens to captured Fc polypeptide dimer-antibody variable region fusion proteins was monitored for 30 to 300 seconds and then their dissociation was monitored for 30-4,200 seconds in HBS-EP+ running buffer. Binding responses were corrected by subtracting the RU from a blank flow cell. A 1:1 Languir model of simultaneous fitting of kₒₙ and k_{off} was used for kinetics analysis.

As shown in Table 7 below, the 35.23.4 anti-HER2 constructs and the 35.23.1.1 anti-HER2 constructs all bound TfR (“WT_IgG” indicates that neither Fc polypeptide contains LALA modification; “cisLALA” indicates that the LALA mutations are present the Fc polypeptide that contains the mutations that create a TfR-binding site, but not on the other Fc polypeptide). An upward drift in the curve for HER2_DII-35.23.1.1^{cisLALA} likely caused an artifactual increase in the K_{D} value for this anti-HER2 construct. Furthermore, no difference in TfR affinity was observed as a result of the Fab domain in this assay format (i.e., where the Fc polypeptide was captured via anti-human Fab).

TABLE 7
Binding affinities for apical hTfR.

| Molecule | K_{D} (nM) |
|---|---|
| HER2_DIV-35.23.4^{WT}^{_{—}}^{IgG} | 498 |
| HER2_DIV-35.23.4^{cisLALA} | 458 |
| HER2_DII-35.23.4^{cisLALA} | 445 |
| HER2_DIV-35.23.1.1^{cisLALA} | 660 |
| HER2_DII-35.23.1.1^{cisLALA} | 534 |
| Anti-BACE-35.23.4^{WT}^{_{—}}^{IgG} | 439 |

As shown in Table 8 below, Fc polypeptide dimer-antibody variable region fusion proteins comprising anti-HER2 subdomain IV and subdomain II antibody variable regions bound to HER2-ECD with affinity (and kinetics) similar to anti-HER2_DIV (used as the reference standard).

TABLE 8
Binding affinities for HER2 ECD.

| Molecule | K_{D} (nM) |
|---|---|
| HER2_DIV-35.23.4^{WT}^{_{—}}^{IgG} | 2.0 |
| HER2_DIV-35.23.4^{cisLALA} | 1.4 |
| HER2_DII-35.23.4^{cisLALA} | 1.8 |
| HER2_DIV-35.23.1.1^{cisLALA} | 1.8 |
| HER2_DII-35.23.1.1^{cisLALA} | 2.1 |
| Anti-HER2_DIV | 2.1 |

### Example 9. HER2-Targeting Fabs Fused to Modified Fc Polypeptides that Target TfR

We generated human IgG1 anti-HER2 antibodies that bind HER2 domains I, II, and IV, which we refer to as anti-HER2-DI, anti-HER2-DII, and anti-HER2-DIV, respectively. The heavy chains Fd regions derived from these mAbs (VH+CH1) were cloned into expression vectors comprising a sequence encoding an Fc polypeptide engineered to bind to the human TfR (CH3C.35.23.4). The Fc polypeptide-encoding sequence also contained a “knob” (T366W) mutation to prevent homodimerization and to promote heterodimerization with an Fc polypeptide comprising “hole” (T366S/L368A/Y407V) mutations. Additionally, the modified Fc polypeptide sequence contained mutations L234A and L235A, which attenuate FcγR binding. The Fd region was also cloned into corresponding “hole” vectors comprising a sequence encoding an Fc polypeptide with hole mutations, but lacking both the TfR binding mutations and the L234A and L235A mutations.

The corresponding aforementioned knob and hole vectors were co-transfected to ExpiCHO or Expi293 cells along with the corresponding light chain vector in the ratio knob:hole:light chain of 1:1:2. The expressed protein was purified by Protein A chromatography followed by preparative size-exclusion chromatography (SEC) to isolate purified proteins, which we refer to as ATV:-HER2-DI (HER2_DI-35.23.4^{cisLALA}), ATV:-HER2-DII (HER2_DII-35.23.4^{cisLALA}), and ATV:-HER2-DIV (HER2_DIV-35.23.4^{cisLALA}) We also made ATV:ctrl, which contains the same modified Fc polypeptide as HER2_DI/DII/DIV-35.23.4^{cisLALA} but has Fabs that bind to an irrelevant antigen (Abeta) that is not expressed on cells of interest in subsequent studies.

Specifically, HER2_D-35.23.4^{cisLALA} has a first heavy chain comprising SEQ TD NO:275, a second heavy chain comprising SEQ ID NO:290, and two light chains comprising SEQ ID NO:293. HER2_DII-35.23.4^{cisLALA} has a first heavy chain comprising SEQ ID NO:46, a second heavy chain comprising SEQ ID NO:55, and two light chains comprising SEQ ID NO:58. HER2_DIV-35.23.4^{cisLALA} has a first heavy chain comprising SEQ ID NO:18, a second heavy chain comprising SEQ ID NO:27, and two light chains comprising SEQ ID NO:57.

Further, anti-HER2-DI, anti-HER2-DII, anti-HER2-DIV, and anti-TfR mAb containing unmodified human IgG1 constant regions were generated analogously.

### Example 10. Measuring the Affinities of HER2- and TfR-Binding Molecules

Affinities of mAbs and TfR-binding Fc polypeptides were measured by SPR using a Biacore T200 or a Biacore 8K. Biacore™ Series S CM5 sensor chips were immobilized with monoclonal mouse anti-human IgG (Fc) antibody for HER2 affinity measurements or mouse anti-human Fab for TfR affinity measurements (human antibody or Fab capture kit from GE Healthcare). Serial 3-fold dilutions of analyte (recombinant HER2 extracellular domain or recombinant TfR apical domain) were injected at a flow rate of 30 μL/min. Each sample was analyzed using a 3-minute association and a 10-minute dissociation. After each injection, the chip was regenerated using 3 M MgCl₂. Binding response was corrected by subtracting the RU from a flow cell capturing an irrelevant IgG at similar density. A 1:1 Languir model of simultaneous fitting of kₒₙ and k_{off} was used for kinetics analysis.

TABLE 10
SPR data.

| | HER2 kₒₙ | HER2 k_{off} | HER2 K_{D} | huTfR Steady- |
|---|---|---|---|---|
| Molecule | (M⁻¹ s⁻¹) | (s⁻¹) | (M) | State Affinity (M) |
| Anti-HER2-DI | 1.54E+05 | 1.01E−03 | 6.5E−09 | NB |
| Anti-HER2-DII | 1.75E+05 | 2.28E−04 | 1.3E−09 | NB |
| Anti-HER2-DIV | 2.44E+05 | 1.90E−04 | 7.8E−10 | NB |
| ATV:HER2-DI | 1.39E+05 | 1.05E−03 | 7.5E−09 | 3.7E−07 |
| ATV:HER2-DII | 1.66E+05 | 7.75E−04 | 4.7E−09 | 4.5E−07 |
| ATV:HER2-DIV | 2.42E+05 | 1.67E−04 | 6.9E−10 | 5.0E−07 |

NB = no binding

### Example 11. Co-Targeting of TfR and HER2-DIV

Many tumor cells and tumor cell lines, such as BT474 and OE19, express both HER2 and TfR. While it is well established that antibodies targeting HER2-DIV are capable of inhibiting tumor cell growth and reducing tumor cell viability in some HER2⁺ cell lines, we sought to understand whether co-targeting HER2-DIV and TfR would lead to enhanced cell killing.

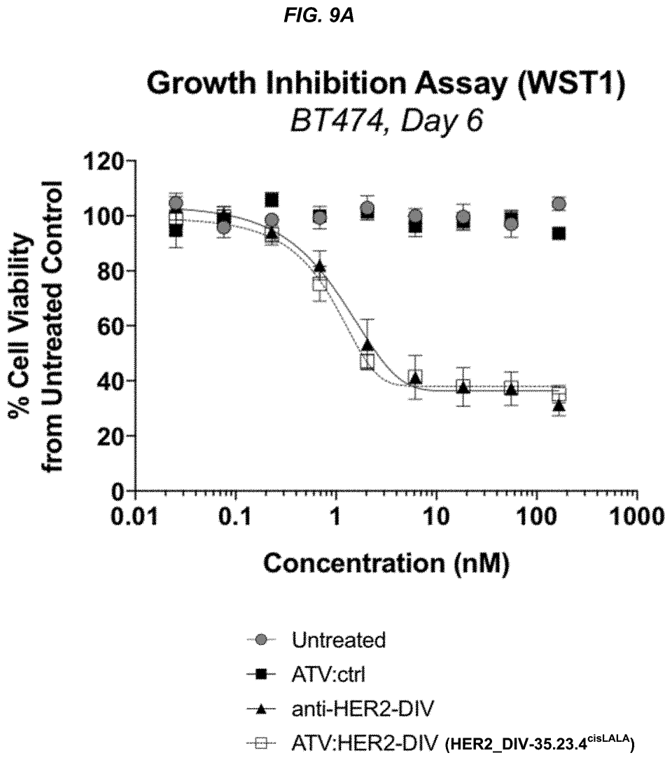

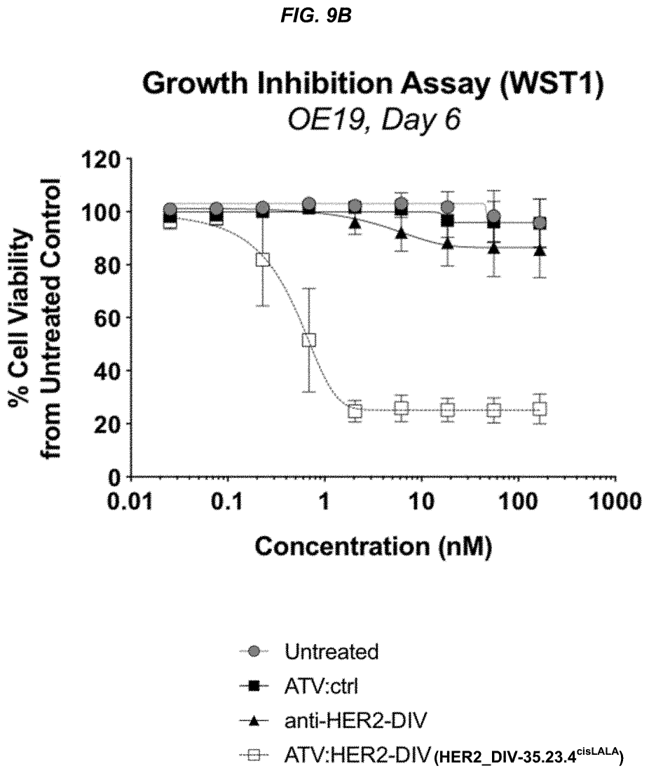

We first compared HER2_DIV-35.23.4^{cisLALA} to anti-HER2-DIV and ATV:ctrl in a growth inhibition assay with a HER2⁺ tumor cell line BT474, which is sensitive to anti-HER2 therapies. BT474 cells were plated overnight at 10,000 cells/well in a 96-well plate, treated with 60 μL of 1:3 serial dilution of molecules of interest beginning at 166 nM (25,000 ng/mL). Culture media (RPMI) and drugs were replenished on Day 3. On Day 6, cell growth was determined using 5 μL of WST-1 reagent (Sigma Aldrich) in 50 μL of growth media. The plate was incubated for 4 hours in the presence of WST-1 reagent, and absorbance was determined at 440 nm. The percent of growth inhibition/proliferation was calculated based on A440 nM and was normalized to the untreated control. Anti-HER2-DIV reduced BT474 cell viability relative to the control with an IC50 of 1.1 nM and a maximum inhibition of 64%. Conversely, ATV:ctrl, which does not bind to any cell antigen, did not have any effect on cell viability, while HER2_DIV-35.23.4^{cisLALA} (ATV:HER2-DIV) showed similar growth inhibition compared to anti-HER2-DIV (FIG. 9A).

Next, we compared HER2_DIV-35.23.4^{cisLALA} to anti-HER2-DIV and ATV:ctrl with another HER2⁺ cancer cell line OE19 that is resistant to anti-HER2 therapies. Unlike BT474, in which there was no difference between the effects of HER2_DIV-35.23.4^{cisLALA} and anti-HER2-DIV, OE19 cell line had a maximum inhibition of 75% upon HER2_DIV-35.23.4^{cisLALA} treatment while it was not responsive to anti-HER2-DIV (FIG. 9B). These results suggest that co-targeting of HER2-DIV and TfR results in enhanced cell growth inhibition in an anti-HER2-resistant cell line as compared to targeting HER2-DIV alone.

### Example 12. Co-Targeting of TfR and HER2-DII

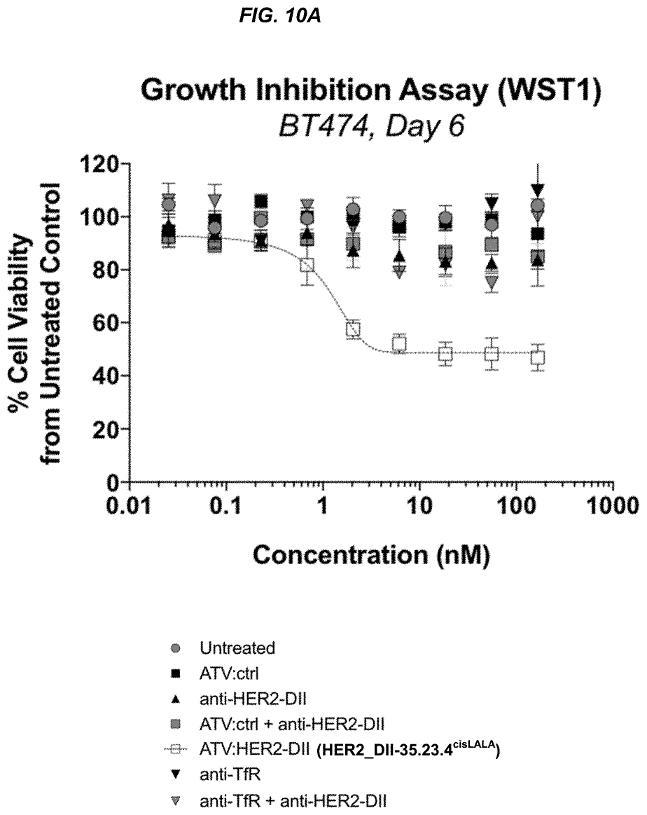

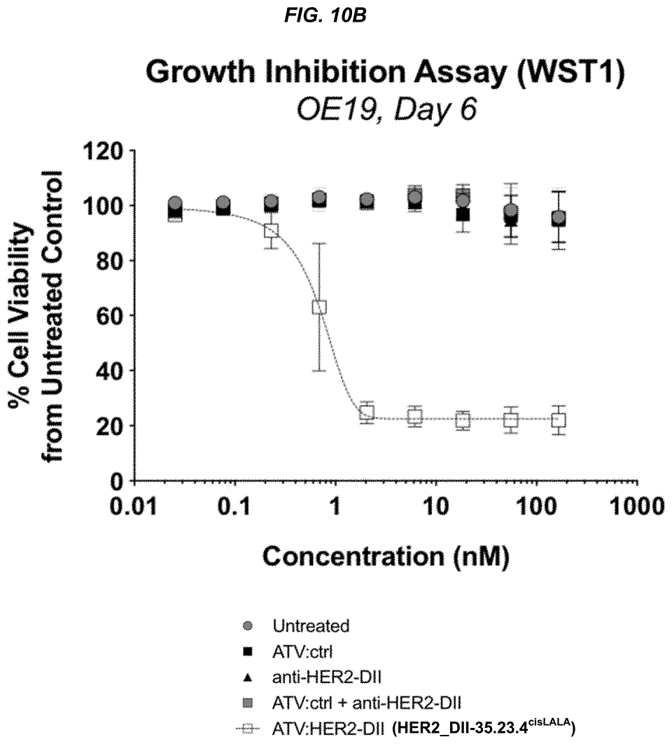

We next determined whether co-targeting HER2-DII and TfR using anti-HER2-DII Fabs fused to TfR-binding Fc polypeptides (ATV:HER2-DII; HER2_DII-35.23.4^{cisLALA}) could enhance cell growth inhibition against BT474 and OE19 tumor cells. Using the same growth inhibition assay described previously, we determined that, in contrast to anti-HER2-DIV treatment, anti-HER2-DII treatment has no impact on the viability of BT474 and OE19 cells (FIGS. 10A and 10B). Similarly, cells treated with ATV:ctrl or anti-TfR alone, or when combined with anti-HER2-DII, did not inhibit BT474 cell growth. In contrast, treatment of BT474 and OE19 cells with HER2_DII-35.23.4^{cisLALA} led to reduced cell viability with an IC50 of 1.17 nM and 0.725 nM, and max inhibition of 51.2% and 77.5%, respectively (FIGS. 10A and 10B). Since neither anti-TfR alone nor the combination of anti-TfR and anti-HER2-DII had any impact on BT474 cell viability (FIG. 10A), we conclude that binding to both TfR and HER2-DII with the same molecule (HER2_DII-35.23.4^{cisLALA}) was required to achieve cell killing in this context. These results provide support that the simultaneous binding, and likely crosslinking, of TfR and HER2-DII by a single molecule can potentiate the growth inhibition of HER2⁺ cancer cell lines.

### Example 13. Co-Targeting of TfR and HER2-DI

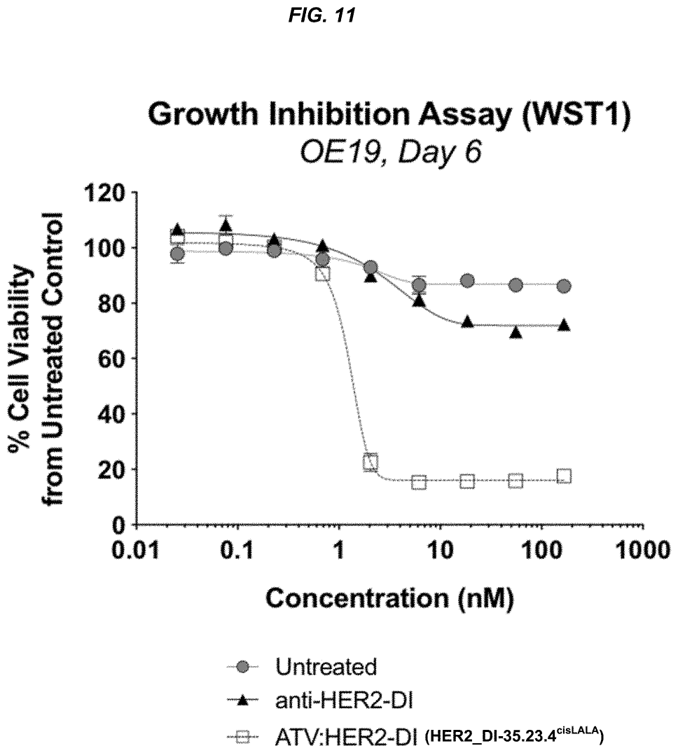

To further determine if co-targeting TfR and the HER2 protein would apply more broadly to other domains of HER2, we also targeted HER2-DI by using anti-HER2-DI and HER2_DI-35.23.4^{cisLALA} (ATV:HER2-DI) in a growth inhibition assay with OE19 cell line. Similar to HER2-DII, OE19 cells did not respond to anti-HER2-DI but had maximum inhibition of 84% upon HER2_DI-35.23.4^{cisLALA} treatment (FIG. 11). This data is consistent with our hypothesis that the simultaneous binding, and likely crosslinking, of TfR and HER2-DI by a single molecule can potentiate growth inhibition of HER2⁺ cancer cells. Taken together, tumor growth inhibition may be potentiated in a HER2 subdomain target upon simultaneous TfR engagement, even when the domain is not normally responsive when targeted alone.

### Example 14. Combination of ATV:HER2-DIV and ATV:HER2-DII

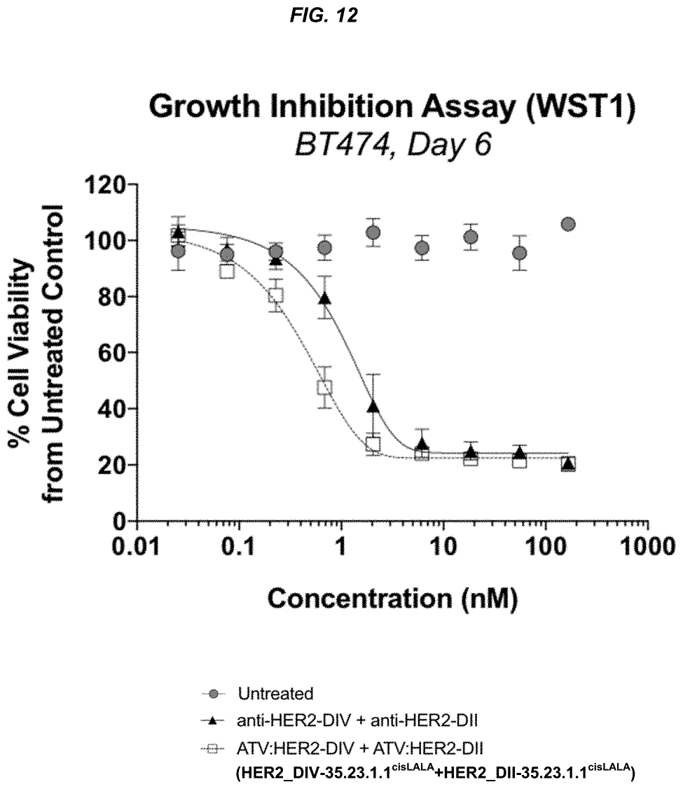

We next used the growth inhibition assay described previously to evaluate the effects of the combination of ATV:HER2-DIV (HER2_DIV-35.23.4^{cisLALA}) and ATV:HER2-DII (HER2_DII-35.23.4^{cisLALA}) versus the combination of anti-HER2-DIV and anti-HER2-DII in BT474 cells. Indeed, we observed approximately a 2.5-fold increase in growth inhibition potency upon treatment of the combination of ATV:HER2-DIV and ATV:HER2-DII compared to the combination of anti-HER2_DIV and anti-HER2_DII (FIG. 12).

We also tested whether the combination ATV:HER2-DIV (HER2_DIV-35.23.1.1^{cisLALA}) and ATV:HER2-DII (HER2_DII-35.23.1.1^{cisLALA}) could overcome tumor resistance in a neuregulin 1-induced resistance model. The Fc polypeptide engineered to bind to the human TfR used here is CH3C.35.23.1.1. Specifically, HER2_DIV-35.23.1.1^{cisLALA} has a first heavy chain comprising SEQ ID NO:2, a second heavy chain comprising SEQ ID NO:27, and two light chains comprising SEQ ID NO:57. HER2_DII-35.23.1.1^{cisLALA} has a first heavy chain comprising SEQ ID NO:30, a second heavy chain comprising SEQ ID NO:55, and two light chains comprising SEQ ID NO:58.

Neuregulin 1 (NRG-1) is a ligand for HER3 that is enriched in the brain microenvironment that activates HER3 signaling pathways and initiates HER2-HER3 dimerization. This in turn leads to activation of the PI3K/AKT pathway and renders tumor cells resistant to anti-HER2-DIV treatment. In the presence of NRG1 (50 ng/mL), BT474 breast cancer cells were indeed resistant to anti-HER2-DIV, which showed cell growth inhibition of only around 15% (FIG. 4). Cross-linking HER2-DIV and TfR with HER2_DIV-35.23.1.1^{cisLALA} (ATV:HER2-DIV) led to enhanced growth inhibition of up to around 40% (FIG. 4), but this was still attenuated relative to cells not treated with NRG-1 (inhibition up to about 70%, FIGS. 9A and 9B). Nonetheless, these results demonstrate that enhancement of tumor cell killing by cross-linking HER2 and TfR can be achieved using molecules targeting HER2-DIV, in addition to those targeting HER2-DII as demonstrated previously.

Co-treatment of NRG1-treated BT474 cells with a combination of molecules targeting both HER2-DIV and HER2-DII led to >80% cell killing. As expected, the effect was enhanced with HER2_DIV-35.23.1.1^{cisLALA}+HER2_DII-35.23.1.1^{cisLALA} relative to anti-HER2-DIV+anti-HER2-DII (FIG. 4), again presumably due to cross-linking of HER2 and TfR.

### Example 15. Cell Surface TfR Expression Following Anti-HER2/TfR Treatment

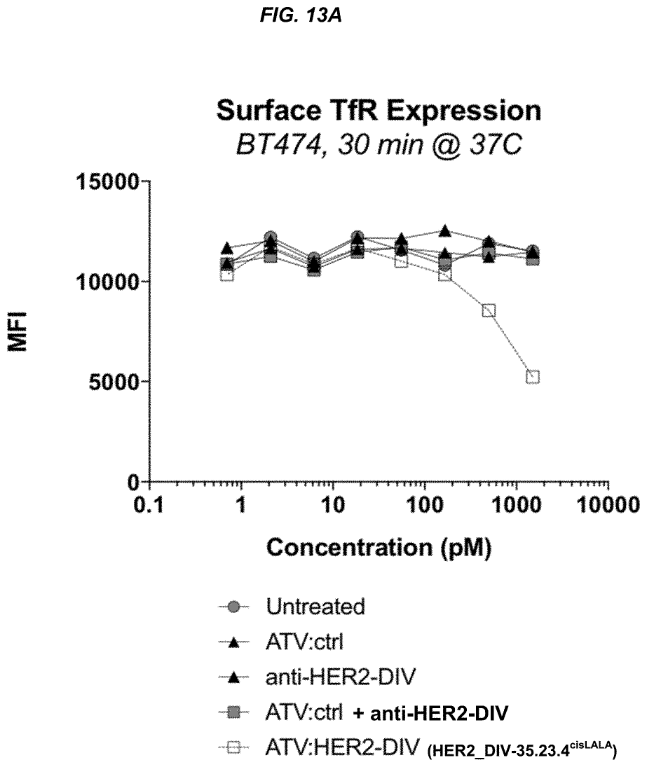

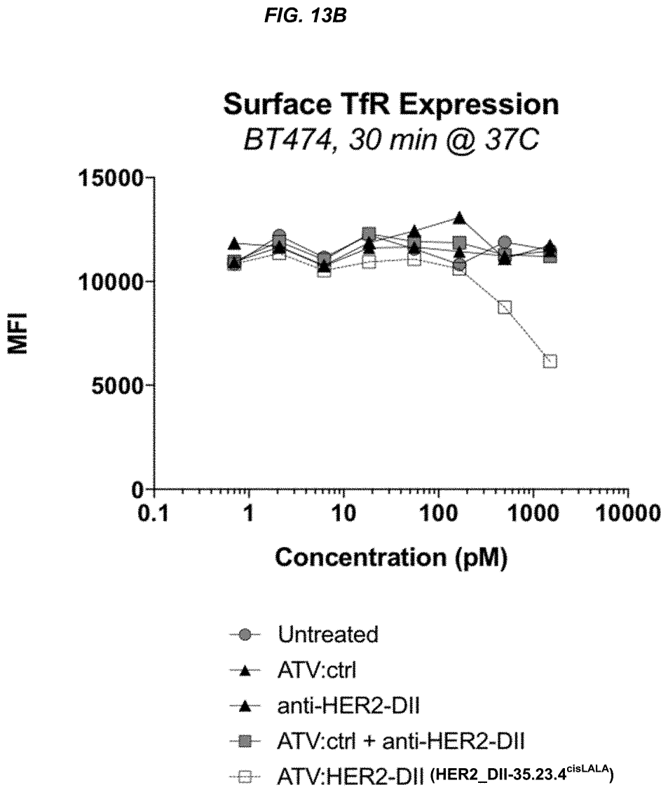

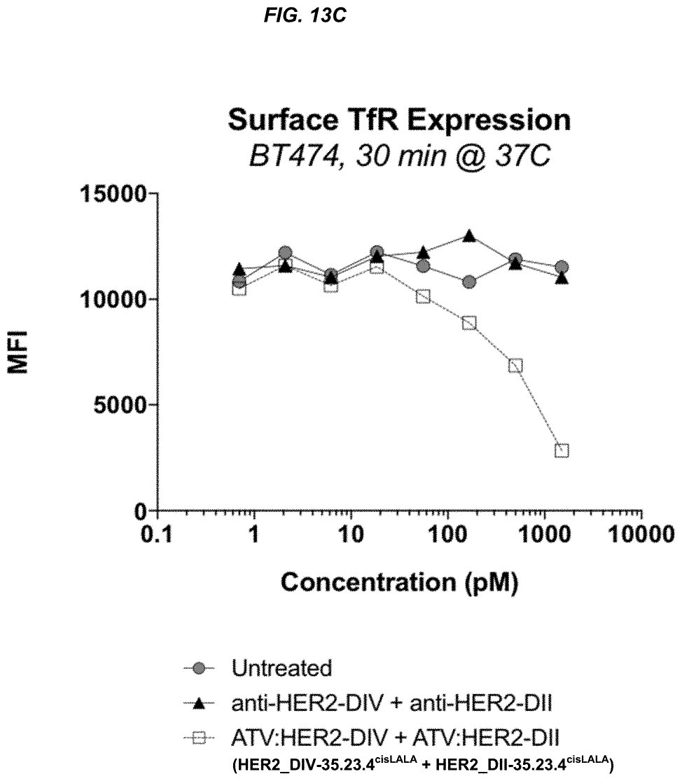

We tested whether treatment of ATV:HER2-DII (HER2_DII-35.23.4^{cisLALA}) and/or ATV:HER2-DIV (HER2_DIV-35.23.4^{cisLALA}) could lead to increased TfR internalization via the measurement of surface TfR protein expression by flow cytometry. BT474 cells were incubated for 30 minutes at 37° C. with test articles or controls, as indicated in FIGS. 13A-13C, including: PBS, ATV:ctrl, anti-HER2-DIV and/or anti-HER2-DII, the combination of ATV:ctrl and anti-HER2-DIV or anti-HER2-DII, and ATV:HER2-DIV and/or ATV:HER2-DII. Following incubation, cells were washed 2× with cold PBS, stained with anti-TfR (CD71) antibody conjugated with APC (Fisher Scientific) for 20 min on ice, and evaluated for the median fluorescence intensity (MFI) by flow cytometry using a BD Canto II. Results were analyzed by the FlowJo Software.

BT474 cells that were treated with greater than around 100 pM ATV:HER2-DIV and/or ATV:HER2-DII had significantly reduced surface TfR expression following 30 min incubation at 37° C. (FIGS. 13A-13C), whereas anti-HER2-DII or anti-HER2-DIV treatment had no impact on TfR expression. Also of note, ATV:ctrl, which binds TfR but not HER2, had no impact on TfR expression, suggesting that HER2-TfR cross-linking contributes to receptor depletion. This mechanism may contribute toward the enhanced cell killing observed for TfR-HER2 crosslinking molecules (see previous Examples).

### Example 16. Modified Fc Polypeptides that Bind to TfR

This example describes modifications to Fc polypeptides to confer TfR binding and transport across the BBB.

Unless otherwise indicated, the positions of amino acid residues in this section are numbered based on EU index numbering for a human IgG1 wild-type Fc region.

### Generation and Characterization of Fc Polypeptides Comprising Modifications at Positions 384, 386, 387, 388, 389, 390, 413, 416, and 421 (CH3C Clones)

Yeast libraries containing Fc regions having modifications introduced into positions including amino acid positions 384, 386, 387, 388, 389, 390, 413, 416, and 421 were generated as described below. Illustrative clones that bind to TfR are shown in Tables 5 and 6.

After an additional two rounds of sorting, single clones were sequenced and four unique sequences were identified. These sequences had a conserved Trp at position 388, and all had an aromatic residue (i.e., Trp, Tyr, or His) at position 421. There was a great deal of diversity at other positions.

The four clones selected from the library were expressed as Fc fusions to Fab fragments in CHO or 293 cells, and purified by Protein A and size-exclusion chromatography, and then screened for binding to human TfR in the presence or absence of holo-Tf by ELISA. The clones all bound to human TfR and the binding was not affected by the addition of excess (5 μM) holo-Tf Clones were also tested for binding to 293F cells, which endogenously express human TfR. The clones bound to 293F cells, although the overall binding was substantially weaker than the high-affinity positive control.

Next, it was tested whether clones could internalize in TfR-expressing cells using clone CH3C.3 as a test clone. Adherent HEK 293 cells were grown in 96-well plates to about 80% confluence, media was removed, and samples were added at 1 μM concentrations: clone CH3C.3, anti-TfR benchmark positive control antibody (Ab204), anti-BACE1 benchmark negative control antibody (Ab107), and human IgG isotype control (obtained from Jackson Immunoresearch). The cells were incubated at 37° C. and 8% CO₂ concentration for 30 minutes, then washed, permeabilized with 0.1% Triton™ X-100, and stained with anti-human-IgG-Alexa Fluor® 488 secondary antibody. After additional washing, the cells were imaged under a high content fluorescence microscope (i.e., an Opera Phenix™ system), and the number of puncta per cell was quantified. At 1 μM, clone CH3C.3 showed a similar propensity for internalization to the positive anti-TfR control, while the negative controls showed no internalization.

### Further Engineering of Clones

Additional libraries were generated to improve the affinity of the initial hits against human TfR using a soft randomization approach, wherein DNA oligos were generated to introduce soft mutagenesis based on each of the original four hits. Additional clones were identified that bound TfR and were selected. The selected clones fell into two general sequence groups. Group 1 clones (i.e., clones CH3C.18, CH3C.21, CH3C.25, and CH3C.34) had a semi-conserved Leu at position 384, a Leu or His at position 386, a conserved and a semi-conserved Val at positions 387 and 389, respectively, and a semi-conserved P-T-W motif at positions 413, 416, and 421, respectively. Group 2 clones had a conserved Tyr at position 384, the motif TXWSX at positions 386-390, and the conserved motif S/T-E-F at positions 413, 416, and 421, respectively. Clones CH3C.18 and CH3C.35 were used in additional studies as representative members of each sequence group.

### Epitope Mapping

To determine whether the engineered Fc regions bound to the apical domain of TfR, TfR apical domain was expressed on the surface of phage. To properly fold and display the apical domain, one of the loops had to be truncated and the sequence needed to be circularly permuted. Clones CH3C.18 and CH3C.35 were coated on ELISA plates and a phage ELISA protocol was followed. Briefly, after washing and blocking with 1% PBSA, dilutions of phage displaying were added and incubated at room temperature for 1 hour. The plates were subsequently washed and anti-M13-HRP was added, and after additional washing the plates were developed with TMB substrate and quenched with 2N H₂SO₄. Both clones CH3C.18 and CH3C.35 bound to the apical domain in this assay.

### Paratope Mapping

To understand which residues in the Fe domain were most important for TfR binding, a series of mutant clone CH3C.18 and clone CH3C.35 Fc regions was created in which each mutant had a single position in the TfR binding register mutated back to wild-type. The resulting variants were expressed recombinantly as Fab-Fc fusions and tested for binding to human or cyno TfR. For clone CH3C.35, positions 388 and 421 were important for binding; reversion of either of these to wild-type completely ablated binding to human TfR.

### Binding Characterization of Maturation Clones

Binding ELISAs were conducted with purified Fab-Fc fusion variants with human or cyno TfR coated on the plate, as described above. The variants from the clone CH3C.18 maturation library, clone CH3C.3.2-1, clone CH3C.3.2-5, and clone CH3C.3.2-19, bound human and cyno TfR with approximately equivalent EC₅₀ values, whereas the parent clones CH3C.18 and CH3C.35 had greater than 10-fold better binding to human versus cyno TfR.

Next, it was tested whether the modified Fc polypeptides internalized in human and monkey cells. Using the protocol described above, internalization in human HEK 293 cells and rhesus LLC-MIK2 cells was tested. The variants that similarly bound human and cyno TfR, clones CH3C.3.2-5 and CH3C.3.2-19, had significantly improved internalization in LLC-MIK2 cells as compared with clone CH3C.35.

### Additional Engineering of Clones

Additional engineering to further affinity mature clones CH3C.18 and CH3C.35 involved adding additional mutations to the positions that enhanced binding through direct interactions, second-shell interactions, or structure stabilization. This was achieved via generation and selection from an “NNK walk” or “NNK patch” library. The NNK walk library involved making one-by-one NNK mutations of residues that are near to the paratope. By looking at the structure of Fc bound to FcγRI (PDB ID: 4W40), 44 residues near the original modification positions were identified as candidates for interrogation. Specifically, the following residues were targeted for NNK mutagenesis: K248, R255, Q342, R344, E345, Q347, T359, K360, N361, Q362, S364, K370, E380, E382, S383, G385, Y391, K392, T393, D399, S400, D401, S403, K409, L410, T411, V412, K414, S415, Q418, Q419, G420, V422, F423, S424, S426, Q438, S440, S442, L443, S444, P4458, G446, and K447. The 44 single point NNK libraries were generated using Kunkel mutagenesis, and the products were pooled and introduced to yeast via electroporation, as described above for other yeast libraries.

The combination of these mini-libraries (each of which had one position mutated, resulting in 20 variants) generated a small library that was selected using yeast surface display for any positions that lead to higher affinity binding. Selections were performed as described above, using TfR apical domain proteins. After three rounds of sorting, clones from the enriched yeast library were sequenced, and several “hot-spot” positions were identified where certain point mutations significantly improved the binding to apical domain proteins. For clone CH3C.35, these mutations included E380 (mutated to Trp, Tyr, Leu, or Gln) and S415 (mutated to Glu). The sequences of the clone CH3C.35 single and combination mutants are set forth in SEQ ID NOs:177 and 185-195. For clone CH3C.18, these mutations included E380 (mutated to Trp, Tyr, or Leu) and K392 (mutated to Gln, Phe, or His). The sequences of the clone CH3C.18 single mutants are set forth in SEQ ID NOs:181-186.

### Additional Maturation Libraries to Improve Clone CH3C.35 Affinity

An additional library to identify combinations of mutations from the NNK walk library, while adding several additional positions on the periphery of these, was generated as described for previous yeast libraries. In this library, the YxTEWSS (SEQ ID NO:196) and TxxExxxxF (SEQ ID NO:197) motifs were kept constant, and six positions were completely randomized: E380, K392, K414, S415, S424, and S426. Positions E380 and S415 were included because they were “hot spots” in the NNK walk library. Positions K392, S424, and S426 were included because they make up part of the core that may position the binding region, while K414 was selected due to its adjacency to position 415.

This library was sorted, as previously described, with the cyno TfR apical domain only. The enriched pool was sequenced after five rounds, and the sequences of the modified regions of the identified unique clones are set forth in SEQ ID NOs:198-215.

The next libraries were designed to further explore acceptable diversity in the main binding paratope. Each of the original positions (384, 386, 387, 388, 389, 390, 413, 416, and 421) plus the two hot spots (380 and 415) were individually randomized with NNK codons to generate a series of single-position saturation mutagenesis libraries on yeast. In addition, each position was individually reverted to the wild-type residue, and these individual clones were displayed on yeast. It was noted that positions 380, 389, 390, and 415 were the only positions that retained substantial binding to TfR upon reversion to the wild-type residue (some residual but greatly diminished binding was observed for reversion of 413 to wild-type).

The single-position NNK libraries were sorted for three rounds against the human TfR apical domain to collect the top ˜5% of binders, and then at least 16 clones were sequenced from each library. The results indicate what amino acids at each position can be tolerated without significantly reducing binding to human TfR, in the context of clone CH3C.35. A summary is below:

### Position 380: Trp, Leu, or Glu;

### Position 384: Tyr or Phe;

Position 386: Thr only;
Position 387: Glu only;
Position 388: Trp only;
Position 389: Ser, Ala, or Val (although the wild type Asn residue seems to retain some binding, it did not appear following library sorting);

### Position 390: Ser or Asn;

### Position 413: Thr or Ser;

### Position 415: Glu or Ser;

Position 416: Glu only; and
Position 421: Phe only.

The above residues, when substituted into clone CH3C.35 as single changes or in combinations, represent paratope diversity that retains binding to TfR apical domain. Clones having mutations at these positions include those shown in Table 6, and the sequences of the CH3 domains of these clones are set forth in SEQ ID NOs:177-180, 192-195, 214, and 216-249.

### Example 17. Methods

### Generation of Phage-Display Libraries

A DNA template coding for the wild-type human Fc sequence was synthesized and incorporated into a phagemid vector. The phagemid vector contained an ompA or pelB leader sequence, the Fc insert fused to c-Myc and 6×His epitope tags, and an amber stop codon followed by M13 coat protein pIII.

Primers containing “NNK” tricodons at the desired positions for modifications were generated, where N is any DNA base (i.e., A, C, G, or T) and K is either G or T. Alternatively, primers for “soft” randomization were used, where a mix of bases corresponding to 70% wild-type base and 10% of each of the other three bases was used for each randomization position. Libraries were generated by performing PCR amplification of fragments of the Fc region corresponding to regions of randomization and then assembled using end primers containing SfiI restriction sites, then digested with SfiI and ligated into the phagemid vectors. Alternatively, the primers were used to conduct Kunkel mutagenesis. The ligated products or Kunkel products were transformed into electrocompetent E. coli cells of strain TG1 (obtained from Lucigen®). The E. coli cells were infected with M13K07 helper phage after recovery and grown overnight, after which library phage were precipitated with 5% PEG/NaCl, resuspended in 15% glycerol in PBS, and frozen until use. Typical library sizes ranged from about 10⁹ to about 10¹¹ transformants. Fc-dimers were displayed on phage via pairing between pIII-fused Fc and soluble Fc not attached to pIII (the latter being generated due to the amber stop codon before pIII).

### Generation of Yeast-Display Libraries

A DNA template coding for the wild-type human Fc sequence was synthesized and incorporated into a yeast display vector. For CH2 and CH3 libraries, the Fc polypeptides were displayed on the Aga2p cell wall protein. Both vectors contained prepro leader peptides with a Kex2 cleavage sequence, and a c-Myc epitope tag fused to the terminus of the Fc.

Yeast display libraries were assembled using methods similar to those described for the phage libraries, except that amplification of fragments was performed with primers containing homologous ends for the vector. Freshly prepared electrocompetent yeast (i.e., strain EBY100) were electroporated with linearized vector and assembled library inserts. Electroporation methods will be known to one of skill in the art. After recovery in selective SD-CAA media, the yeast were grown to confluence and split twice, then induced for protein expression by transferring to SG-CAA media. Typical library sizes ranged from about 10⁷ to about 10⁹ transformants. Fc-dimers were formed by pairing of adjacently displayed Fc monomers.

### General Methods for Phage Selection

Phage methods were adapted from Phage Display: A Laboratory Manual (Barbas, 2001). Additional protocol details can be obtained from this reference.

### Plate Sorting Methods

Human TfR target was coated on MaxiSorp® microtiter plates (typically 200 μL at 1-10 μg/mL in PBS) overnight at 4° C. All binding was done at room temperature unless otherwise specified. The phage libraries were added into each well and incubated overnight for binding. Microtiter wells were washed extensively with PBS containing 0.05% Tween® 20 (PBST) and bound phage were eluted by incubating the wells with acid (typically 50 mM HCl with 500 mM KCl, or 100 mM glycine, pH 2.7) for 30 minutes. Eluted phage were neutralized with 1 M Tris (pH 8) and amplified using TG1 cells and M13/KO7 helper phage and grown overnight at 37° C. in 2YT media containing 50 μg/mL carbenacillin and 50 μg/mL Kanamycin. The titers of phage eluted from a target-containing well were compared to titers of phage recovered from a non-target-containing well to assess enrichment. Selection stringency was increased by subsequently decreasing the incubation time during binding and increasing washing time and number of washes.

### Bead Sorting Methods

Antigen was biotinylated through free amines using NHS-PEG4-Biotin (obtained from Pierce™). For biotinylation reactions, a 3- to 5-fold molar excess of biotin reagent was used in PBS. Reactions were quenched with Tris followed by extensive dialysis in PBS. The biotinylated antigen was immobilized on streptavidin-coated magnetic beads, (i.e., M280-streptavidin beads obtained Thermo Fisher). The phage display libraries were incubated with the antigen-coated beads at room temperature for 1 hour. The unbound phage were then removed and beads were washed with PBST. The bound phage were eluted by incubating with 50 mM HCl containing 500 mM KCl (or 0.1 M glycine, pH 2.7) for 30 minutes, and then neutralized and propagated as described above for plate sorting.

After three to five rounds of panning, single clones were screened by either expressing Fc on phage or solubly in the E. coli periplasm. Such expression methods will be known to one of skill in the art. Individual phage supernatants or periplasmic extracts were exposed to blocked ELISA plates coated with antigen or a negative control and were subsequently detected using HRP-conjugated goat anti-Fc (obtained from Jackson Immunoresearch) for periplasmic extracts or anti-M13 (GE Healthcare) for phage, and then developed with TMB reagent (obtained from Thermo Fisher). Wells with OD₄₅₀ values greater than around 5-fold over background were considered positive clones and sequenced, after which some clones were expressed either as a soluble Fc fragment or fused to Fab fragments.

### General Methods for Yeast Selection

### Bead Sorting (Magnetic-Assisted Cell Sorting (MACS)) Methods

MACS and FACS selections were performed similarly to as described in Ackerman, et al. 2009 Biotechnol. Prog. 25(3), 774. Streptavidin magnetic beads (e.g., M-280 streptavidin beads from Thermo Fisher) were labeled with biotinylated antigen and incubated with yeast (typically 5-10× library diversity). Unbound yeast were removed, the beads were washed, and bound yeast were grown in selective media and induced for subsequent rounds of selection.

### Fluorescence-Activated Cell Sorting (FACS) Methods

Yeast were labeled with anti-c-Myc antibody to monitor expression and biotinylated antigen (concentration varied depending on the sorting round). In some experiments, the antigen was pre-mixed with streptavidin-Alexa Fluor® 647 in order to enhance the avidity of the interaction. In other experiments, the biotinylated antigen was detected after binding and washing with streptavidin-Alexa Fluor® 647. Singlet yeast with binding were sorted using a FACS Aria III cell sorter. The sorted yeast were grown in selective media then induced for subsequent selection rounds.

After an enriched yeast population was achieved, yeast were plated on SD-CAA agar plates and single colonies were grown and induced for expression, then labeled as described above to determine their propensity to bind to the target. Positive single clones were subsequently sequenced for binding antigen, after which some clones were expressed either as a soluble Fc fragment or as fused to Fab fragments.

### General Methods for Screening

### Screening by ELISA

Clones were selected from panning outputs and grown in individual wells of 96-well deep-well plates. The clones were either induced for periplasmic expression using autoinduction media (obtained from EMD Millipore) or infected with helper phage for phage-display of the individual Fc variants on phage. The cultures were grown overnight and spun to pellet E. coli. For phage ELISA, phage containing supernatant was used directly. For periplasmic expression, pellets were resuspended in 20% sucrose, followed by dilution at 4:1 with water, and shaken at 4° C. for 1 hour. Plates were spun to pellet the solids and supernatant was used in the ELISA.

ELISA plates were coated with antigen, typically at 0.5 mg/mL overnight, then blocked with 1% BSA before addition of phage or periplasmic extracts. After a 1-hour incubation and washing off unbound protein, HRP-conjugated secondary antibody was added (i.e., anti-Fc or anti-M13 for soluble Fc or phage-displayed Fc, respectively) and incubated for 30 minutes. The plates were washed again, and then developed with TMB reagent and quenched with 2N sulfuric acid. Absorbance at 450 nm was quantified using a plate reader (BioTek®) and binding curves were plotted using Prism software where applicable. Absorbance signal for tested clones was compared to negative control (phage or paraplasmic extract lacking Fc). In some assays, soluble transferrin or other competitor was added during the binding step, typically at significant molar excess (greater than 10-fold excess).

### Screening by Flow Cytometry

Fc variant polypeptides (expressed either on phage, in periplasmic extracts, or solubly as fusions to Fab fragments) were added to cells in 96-well V-bottom plates (about 100,000 cells per well in PBS+1% BSA (PBSA)), and incubated at 4° C. for 1 hour. The plates were subsequently spun and the media was removed, and then the cells were washed once with PBSA. The cells were resuspended in PBSA containing secondary antibody (typically goat anti-human-IgG-Alexa Fluor® 647 (obtained from Thermo Fisher)). After 30 minutes, the plates were spun and the media was removed, the cells were washed 1-2 times with PBSA, and then the plates were read on a flow cytometer (i.e., a FACSCanto™ II flow cytometer). Median fluorescence values were calculated for each condition using FlowJo software and binding curves were plotted with Prism software.

### Example 18. Construction of CH3C.18 Variants

This example describes the construction of a library of CH3C.18 variants.

Single clones were isolated, and grown overnight in SG-CAA media supplemented with 0.2% glucose overnight to induce surface expression of CH3C.18 variants. For each clone, two million cells were washed three times in PBS+0.5% BSA at pH 7.4. Cells were stained with biotinylated target, 250 nM human TfR, 250 nM cyno TfR, or 250 nM of an unrelated biotinylated protein for 1 hour at 4° C. with shaking, then washed twice with the same buffer. Cells were stained with nuetravidin-Alexafluor647 (AF647) for 30 minutes at 4° C., then washed twice again. Expression was measured using anti-c-myc antibody with anti-chicken-Alexfluor488 (AF488) secondary antibody. Cells were resuspended, and median fluorescence intensity (MFI) of AF647 and AF488 was measured on a BD FACS CantoII. MFI was calculated for the TfR-binding population for each population and plotted with human TfR, cyno TfR, or control binding.

Table 9 shows the library of CH3C.18 variants. Each row represents a variant that contains the indicated amino acid substitutions at each position and the amino acids at the rest of the positions are the same as those in CH3C.18. The positions shown in Table 9 are numbered according to the EU numbering scheme.

TABLE 9
CH3C.18 Variants

| Position | 384 | 386 | 387 | 389 | 390 | 391 | 413 | 416 | 421 |
|---|---|---|---|---|---|---|---|---|---|
| Wild-type Fc | N | Q | P | N | N | Y | D | R | N |
| CH3C.4 (CH3C.18.1) | V | T | P | A | L | Y | L | E | W |
| CH3C.2 (CH3C.18.2) | Y | T | V | S | H | Y | S | E | Y |
| CH3C.3 (CH3C.18.3) | Y | T | E | S | Q | Y | E | D | H |
| CH3C.1 (CH3C.18.4) | L | L | V | V | G | Y | A | T | W |
| CH3C.18 (CH3C.18.1.18) | L | H | V | A | V | Y | P | T | W |
| CH3C.3.1-3 (CH3C.18.3.1-3) | L | H | V | V | A | T | P | T | W |
| CH3C.3.1-9 (CH3C.18.3.1-9) | L | P | V | V | H | T | P | T | W |
| CH3C.3.2-1 (CH3C.18.3.2-1) | L | H | V | V | N | F | P | T | W |
| CH3C.3.2-5 (CH3C.18.3.2-5) | L | H | V | V | D | Q | P | T | W |
| CH3C.3.2-19 (CH3C.18.3.2-19) | L | H | V | V | N | Q | P | T | W |
| CH3C.3.4-1 (CH3C.18.3.4-1) | W | F | V | S | T | T | P | N | F |
| CH3C.3.4-19 (CH3C.18.3.4-19) | W | H | V | S | T | T | P | N | Y |
| CH3C.3.2-3 (CH3C.18.3.2-3) | L | H | V | V | E | Q | P | T | W |
| CH3C.3.2-14 (CH3C.18.3.2-14) | L | H | V | V | G | V | P | T | W |
| CH3C.3.2-24 (CH3C.18.3.2-24) | L | H | V | V | H | T | P | T | W |
| CH3C.3.4-26 (CH3C.18.3.4-26) | W | T | V | G | T | Y | P | N | Y |
| CH3C.3.2-17 (CH3C.18.3.2-17) | L | H | V | V | G | T | P | T | W |

It is understood that the examples and embodiments described herein are for illustrative purposes only and that various modifications or changes in light thereof will be suggested to persons skilled in the art and are to be included within the spirit and purview of this application and scope of the appended claims. The sequences of the sequence accession numbers cited herein are hereby incorporated by reference.

Unless otherwise defined, all technical and scientific terms used herein have the same meaning as commonly understood by one of ordinary skill in the art to which this disclosure belongs.

The disclosure illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure claimed.

The amino acid substitutions for each clone described in the Tables (e.g., Table 6) dictate the amino acid substitutions at the register positions of that clone over the amino acids found in the sequence set forth in the Sequence Listing, in case of discrepancy.

All publications, patent applications, patents, and other references mentioned herein are expressly incorporated by reference in their entirety, to the same extent as if each were incorporated by reference individually. In case of conflict, the present specification, including definitions, will control.

TABLE 5
CH3C Register Positions and Mutations

| Clone | | | | | | | | | | | | | | | | | | | |
|---|---|---|---|---|---|---|---|---|---|---|---|---|---|---|---|---|---|---|---|
| name | Group | 384 | 385 | 386 | 387 | 388 | 389 | 390 | 391 | . . . | 413 | 414 | 415 | 416 | 417 | 418 | 419 | 420 | 421 |
| Wild-type | n/a | N | G | Q | P | E | N | N | Y | . . . | D | K | S | R | W | Q | Q | G | N |
| 1 | | L | G | L | V | W | V | G | Y | . . . | A | K | S | T | W | Q | Q | G | W |
| 2 | | Y | G | T | V | W | S | H | Y | . . . | S | K | S | E | W | Q | Q | G | Y |
| 3 | | Y | G | T | E | W | S | Q | Y | . . . | E | K | S | D | W | Q | Q | G | H |
| 4 | | V | G | T | P | W | A | L | Y | . . . | L | K | S | E | W | Q | Q | G | W |
| 17 | 2 | Y | G | T | V | W | S | K | Y | . . . | S | K | S | E | W | Q | Q | G | F |
| 18 | 1 | L | G | H | V | W | A | V | Y | . . . | P | K | S | T | W | Q | Q | G | W |
| 21 | 1 | L | G | L | V | W | V | G | Y | . . . | P | K | S | T | W | Q | Q | G | W |
| 25 | 1 | M | G | H | V | W | V | G | Y | . . . | D | K | S | T | W | Q | Q | G | W |
| 34 | 1 | L | G | L | V | W | V | F | S | . . . | P | K | S | T | W | Q | Q | G | W |
| 35 | 2 | Y | G | T | E | W | S | S | Y | . . . | T | K | S | E | W | Q | Q | G | F |
| 44 | 2 | Y | G | T | E | W | S | N | Y | . . . | S | K | S | E | W | Q | Q | G | F |
| 51 | 1/2 | L | G | H | V | W | V | G | Y | . . . | S | K | S | E | W | Q | Q | G | W |
| 3.1-3 | 1 | L | G | H | V | W | V | A | T | . . . | P | K | S | T | W | Q | Q | G | W |
| 3.1-9 | 1 | L | G | P | V | W | V | H | T | . . . | P | K | S | T | W | Q | Q | G | W |
| 3.2-5 | 1 | L | G | H | V | W | V | D | Q | . . . | P | K | S | T | W | Q | Q | G | W |
| 3.2-19 | 1 | L | G | H | V | W | V | N | Q | . . . | P | K | S | T | W | Q | Q | G | W |
| 3.2-1 | 1 | L | G | H | V | W | V | N | F | . . . | P | K | S | T | W | Q | Q | G | W |
| 3.4-1 | | W | G | F | V | W | S | T | Y | | P | K | S | N | W | Q | Q | G | F |
| 3.4-19 | | W | G | H | V | W | S | T | Y | | P | K | S | N | W | Q | Q | G | Y |
| 3.2-3 | | L | G | H | V | W | V | E | Q | | P | K | S | T | W | Q | Q | G | W |
| 3.2-14 | | L | G | H | V | W | V | G | V | | P | K | S | T | W | Q | Q | G | W |
| 3.2-24 | | L | G | H | V | W | V | H | T | | P | K | S | T | W | Q | Q | G | W |
| 3.4-26 | | W | G | T | V | W | G | T | Y | | P | K | S | N | W | Q | Q | G | Y |
| 3.2-17 | | L | G | H | V | W | V | G | T | | P | K | S | T | W | Q | Q | G | W |

TABLE 6
Additional CH3C Register Positions and Mutations

| Clone name | 378 | 379 | 380 | 381 | 382 | 383 | 384 | 385 | 386 | 387 | 388 | 389 | 390 | 391 | 392 | 411 | 412 | 413 | 414 | 415 | 416 | 417 | 418 | 419 | 420 | 421 | 422 | 423 |
|---|---|---|---|---|---|---|---|---|---|---|---|---|---|---|---|---|---|---|---|---|---|---|---|---|---|---|---|---|
| Wild-type | A | V | E | W | E | S | N | G | Q | P | E | N | N | Y | K | T | V | D | K | S | R | W | Q | Q | G | N | V | F |
| 35.20.1 | . | . | . | . | . | . | F | . | T | E | W | S | S | . | . | . | . | T | . | E | E | . | . | . | . | F | . | . |
| 35.20.2 | . | . | . | . | . | . | Y | . | T | E | W | A | S | . | . | . | . | T | . | E | E | . | . | . | . | F | . | . |
| 35.20.3 | . | . | . | . | . | . | Y | . | T | E | W | V | S | . | . | . | . | T | . | E | E | . | . | . | . | F | . | . |
| 35.20.4 | . | . | . | . | . | . | Y | . | T | E | W | S | S | . | . | . | . | S | . | E | E | . | . | . | . | F | . | . |
| 35.20.5 | . | . | . | . | . | . | F | . | T | E | W | A | S | . | . | . | . | T | . | E | E | . | . | . | . | F | . | . |
| 35.20.6 | . | . | . | . | . | . | F | . | T | E | W | V | S | . | . | . | . | T | . | E | E | . | . | . | . | F | . | . |
| 35.21.a.1 | . | . | W | . | . | . | F | . | T | E | W | S | S | . | . | . | . | T | . | E | E | . | . | . | . | F | . | . |
| 35.21.a.2 | . | . | W | . | . | . | Y | . | T | E | W | A | S | . | . | . | . | T | . | E | E | . | . | . | . | F | . | . |
| 35.21.a.3 | . | . | W | . | . | . | Y | . | T | E | W | V | S | . | . | . | . | T | . | E | E | . | . | . | . | F | . | . |
| 35.21.a.4 | . | . | W | . | . | . | Y | . | T | E | W | S | S | . | . | . | . | S | . | E | E | . | . | . | . | F | . | . |
| 35.21.a.5 | . | . | W | . | . | . | F | . | T | E | W | A | S | . | . | . | . | T | . | E | E | . | . | . | . | F | . | . |
| 35.21.a.6 | . | . | W | . | . | . | F | . | T | E | W | V | S | . | . | . | . | T | . | E | E | . | . | . | . | F | . | . |
| 35.23.1 | . | . | . | . | . | . | F | . | T | E | W | S | . | . | . | . | . | T | . | E | E | . | . | . | . | F | . | . |
| 35.23.2 | . | . | . | . | . | . | Y | . | T | E | W | A | . | . | . | . | . | T | . | E | E | . | . | . | . | F | . | . |
| 35.23.3 | . | . | . | . | . | . | Y | . | T | E | W | V | . | . | . | . | . | T | . | E | E | . | . | . | . | F | . | . |
| 35.23.4 | . | . | . | . | . | . | Y | . | T | E | W | S | . | . | . | . | . | S | . | E | E | . | . | . | . | F | . | . |
| 35.23.5 | . | . | . | . | . | . | F | . | T | E | W | A | . | . | . | . | . | T | . | E | E | . | . | . | . | F | . | . |
| 35.23.6 | . | . | . | . | . | . | F | . | T | E | W | V | . | . | . | . | . | T | . | E | E | . | . | . | . | F | . | . |
| 35.24.1 | . | . | W | . | . | . | F | . | T | E | W | S | . | . | . | . | . | T | . | E | E | . | . | . | . | F | . | . |
| 35.24.2 | . | . | W | . | . | . | Y | . | T | E | W | A | . | . | . | . | . | T | . | E | E | . | . | . | . | F | . | . |
| 35.24.3 | . | . | W | . | . | . | Y | . | T | E | W | V | . | . | . | . | . | T | . | E | E | . | . | . | . | F | . | . |
| 35.24.4 | . | . | W | . | . | . | Y | . | T | E | W | S | . | . | . | . | . | S | . | E | E | . | . | . | . | F | . | . |
| 35.24.5 | . | . | W | . | . | . | F | . | T | E | W | A | . | . | . | . | . | T | . | E | E | . | . | . | . | F | . | . |
| 35.24.6 | . | . | W | . | . | . | F | . | T | E | W | V | . | . | . | . | . | T | . | E | E | . | . | . | . | F | . | . |
| 35.21.17.1 | . | . | L | . | . | . | F | . | T | E | W | S | S | . | . | . | . | T | . | E | E | . | . | . | . | F | . | . |
| 35.21.17.2 | . | . | L | . | . | . | Y | . | T | E | W | A | S | . | . | . | . | T | . | E | E | . | . | . | . | F | . | . |
| 35.21.17.3 | . | . | L | . | . | . | Y | . | T | E | W | V | S | . | . | . | . | T | . | E | E | . | . | . | . | F | . | . |
| 35.21.17.4 | . | . | L | . | . | . | Y | . | T | E | W | S | S | . | . | . | . | S | . | E | E | . | . | . | . | F | . | . |
| 35.21.17.5 | . | . | L | . | . | . | F | . | T | E | W | A | S | . | . | . | . | T | . | E | E | . | . | . | . | F | . | . |
| 35.21.17.6 | . | . | L | . | . | . | F | . | T | E | W | V | S | . | . | . | . | T | . | E | E | . | . | . | . | F | . | . |
| 35.20 | . | . | . | . | . | . | Y | . | T | E | W | S | S | . | . | . | . | T | . | E | E | . | . | . | . | F | . | . |
| 35.21 | . | . | W | . | . | . | Y | . | T | E | W | S | S | . | . | . | . | T | . | E | E | . | . | . | . | F | . | . |
| 35.22 | . | . | W | . | . | . | Y | . | T | E | W | S | . | . | . | . | . | T | . | . | E | . | . | . | . | F | . | . |
| 35.23 | . | . | . | . | . | . | Y | . | T | E | W | S | . | . | . | . | . | T | . | E | E | . | . | . | . | F | . | . |
| 35.24 | . | . | W | . | . | . | Y | . | T | E | W | S | . | . | . | . | . | T | . | E | E | . | . | . | . | F | . | . |
| 35.21.17 | . | . | L | . | . | . | Y | . | T | E | W | S | S | . | . | . | . | T | . | E | E | . | . | . | . | F | . | . |
| 35.N390 | . | . | . | . | . | . | Y | . | T | E | W | S | . | . | . | . | . | T | . | . | E | . | . | . | . | F | . | . |
| 35.20.1.1 | | | | | | | F | | T | E | W | S | S | | | | | S | | E | E | | | | | F | | |
| 35.23.2.1 | | | | | | | Y | | T | E | W | A | | | | | | S | | | E | | | | | F | | |
| 35.23.1.1 | | | | | | | F | | T | E | W | S | | | | | | S | | E | E | | | | | F | | |
| 35.S413 | | | | | | | Y | | T | E | W | S | S | | | | | S | | | E | | | | | F | | |
| 35.23.3.1 | | | | | | | Y | | T | E | W | V | | | | | | S | | E | E | | | | | F | | |
| 35.N390.1 | | | | | | | Y | | T | E | W | S | | | | | | S | | | E | | | | | F | | |
| 35.23.6.1 | | | | | | | F | | T | E | W | V | | | | | | S | | E | E | | | | | F | | |

INFORMAL SEQUENCE LISTING

| SEQ | | |
|---|---|---|
| ID | | |
| NO | Sequence | Description |
| 1 | EVQLVESGGGLVQPGGSLRLSCAASGFNIKDTYIHWVRQAPGKGLEWVARIY | Anti-HER2_DIV fused |
| | PTNGYTRYADSVKGRFTISADTSKNTAYLQMNSLRAEDTAVYYCSRWGGDG | to CH3C.35.23.1.1 with |
| | FYAMDYWGQGTLVTVSSASTKGPSVFPLAPSSKSTSGGTAALGCLVKDYFPE | knob mutation |
| | PVTVSWNSGALTSGVHTFPAVLQSSGLYSLSSVVTVPSSSLGTQTYICNVNHK | |
| | PSNTKVDKKVEPKSCDKTHTCPPCPAPELLGGPSVFLFPPKPKDTLMISRTPEV | |
| | TCVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLWCLVKGFYPSDIAVEWESFGTEWSNYKTTPPVLDSDGSFFLYSKLTVSKE | |
| | EWQQGFVFSCSVMHEALHNHYTQKSLSLSPGK | |
| 2 | EVQLVESGGGLVQPGGSLRLSCAASGFNIKDTYIHWVRQAPGKGLEWVARIY | Anti-HER2_DIV fused |
| | PTNGYTRYADSVKGRFTISADTSKNTAYLQMNSLRAEDTAVYYCSRWGGDG | to CH3C.35.23.1.1 with |
| | FYAMDYWGQGTLVTVSSASTKGPSVFPLAPSSKSTSGGTAALGCLVKDYFPE | knob and LALA |
| | PVTVSWNSGALTSGVHTFPAVLQSSGLYSLSSVVTVPSSSLGTQTYICNVNHK | mutations |
| | PSNTKVDKKVEPKSCDKTHTCPPCPAPEAAGGPSVFLFPPKPKDTLMISRTPEV | |
| | TCVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLWCLVKGFYPSDIAVEWESFGTEWSNYKTTPPVLDSDGSFFLYSKLTVSKE | |
| | EWQQGFVFSCSVMHEALHNHYTQKSLSLSPGK | |
| 3 | EVQLVESGGGLVQPGGSLRLSCAASGFNIKDTYIHWVRQAPGKGLEWVARIY | Anti-HER2_DIV fused |
| | PTNGYTRYADSVKGRFTISADTSKNTAYLQMNSLRAEDTAVYYCSRWGGDG | to CH3C.35.23.1.1 with |
| | FYAMDYWGQGTLVTVSSASTKGPSVFPLAPSSKSTSGGTAALGCLVKDYFPE | knob and M428L and |
| | PVTVSWNSGALTSGVHTFPAVLQSSGLYSLSSVVTVPSSSLGTQTYICNVNHK | N434S mutations |
| | PSNTKVDKKVEPKSCDKTHTCPPCPAPELLGGPSVFLFPPKPKDTLMISRTPEV | |
| | TCVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLWCLVKGFYPSDIAVEWESFGTEWSNYKTTPPVLDSDGSFFLYSKLTVSKE | |
| | EWQQGFVFSCSVLHEALHSHYTQKSLSLSPGK | |
| 4 | EVQLVESGGGLVQPGGSLRLSCAASGFNIKDTYIHWVRQAPGKGLEWVARIY | Anti-HER2_DIV fused |
| | PTNGYTRYADSVKGRFTISADTSKNTAYLQMNSLRAEDTAVYYCSRWGGDG | to CH3C.35.23.1.1 with |
| | FYAMDYWGQGTLVTVSSASTKGPSVFPLAPSSKSTSGGTAALGCLVKDYFPE | knob, LALA, and |
| | PVTVSWNSGALTSGVHTFPAVLQSSGLYSLSSVVTVPSSSLGTQTYICNVNHK | M428L and N434S |
| | PSNTKVDKKVEPKSCDKTHTCPPCPAPEAAGGPSVFLFPPKPKDTLMISRTPEV | mutations |
| | TCVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLWCLVKGFYPSDIAVEWESFGTEWSNYKTTPPVLDSDGSFFLYSKLTVSKE | |
| | EWQQGFVFSCSVLHEALHSHYTQKSLSLSPGK | |
| 5 | EVQLVESGGGLVQPGGSLRLSCAASGFNIKDTYIHWVRQAPGKGLEWVARIY | Anti-HER2_DIV fused |
| | PTNGYTRYADSVKGRFTISADTSKNTAYLQMNSLRAEDTAVYYCSRWGGDG | to CH3C.35.23.1.1 with |
| | FYAMDYWGQGTLVTVSSASTKGPSVFPLAPSSKSTSGGTAALGCLVKDYFPE | hole mutations |
| | PVTVSWNSGALTSGVHTFPAVLQSSGLYSLSSVVTVPSSSLGTQTYICNVNHK | |
| | PSNTKVDKKVEPKSCDKTHTCPPCPAPELLGGPSVFLFPPKPKDTLMISRTPEV | |
| | TCVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLSCAVKGFYPSDIAVEWESFGTEWSNYKTTPPVLDSDGSFFLVSKLTVSKE | |
| | EWQQGFVFSCSVMHEALHNHYTQKSLSLSPGK | |
| 6 | EVQLVESGGGLVQPGGSLRLSCAASGFNIKDTYIHWVRQAPGKGLEWVARIY | Anti-HER2_DIV fused |
| | PTNGYTRYADSVKGRFTISADTSKNTAYLQMNSLRAEDTAVYYCSRWGGDG | to CH3C.35.23.1.1 with |
| | FYAMDYWGQGTLVTVSSASTKGPSVFPLAPSSKSTSGGTAALGCLVKDYFPE | hole and LALA |
| | PVTVSWNSGALTSGVHTFPAVLQSSGLYSLSSVVTVPSSSLGTQTYICNVNHK | mutations |
| | PSNTKVDKKVEPKSCDKTHTCPPCPAPEAAGGPSVFLFPPKPKDTLMISRTPEV | |
| | TCVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLSCAVKGFYPSDIAVEWESFGTEWSNYKTTPPVLDSDGSFFLVSKLTVSKE | |
| | EWQQGFVFSCSVMHEALHNHYTQKSLSLSPGK | |
| 7 | EVQLVESGGGLVQPGGSLRLSCAASGFNIKDTYIHWVRQAPGKGLEWVARIY | Anti-HER2_DIV fused |
| | PTNGYTRYADSVKGRFTISADTSKNTAYLQMNSLRAEDTAVYYCSRWGGDG | to CH3C.35.23.1.1 with |
| | FYAMDYWGQGTLVTVSSASTKGPSVFPLAPSSKSTSGGTAALGCLVKDYFPE | hole and M428L and |
| | PVTVSWNSGALTSGVHTFPAVLQSSGLYSLSSVVTVPSSSLGTQTYICNVNHK | N434S mutations |
| | PSNTKVDKKVEPKSCDKTHTCPPCPAPELLGGPSVFLFPPKPKDTLMISRTPEV | |
| | TCVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLSCAVKGFYPSDIAVEWESFGTEWSNYKTTPPVLDSDGSFFLVSKLTVSKE | |
| | EWQQGFVFSCSVLHEALHSHYTQKSLSLSPGK | |
| 8 | EVQLVESGGGLVQPGGSLRLSCAASGFNIKDTYIHWVRQAPGKGLEWVARIY | Anti-HER2_DIV fused |
| | PTNGYTRYADSVKGRFTISADTSKNTAYLQMNSLRAEDTAVYYCSRWGGDG | to CH3C.35.23.1.1 with |
| | FYAMDYWGQGTLVTVSSASTKGPSVFPLAPSSKSTSGGTAALGCLVKDYFPE | hole, LALA, and |
| | PVTVSWNSGALTSGVHTFPAVLQSSGLYSLSSVVTVPSSSLGTQTYICNVNHK | M428L and N434S |
| | PSNTKVDKKVEPKSCDKTHTCPPCPAPEAAGGPSVFLFPPKPKDTLMISRTPEV | mutations |
| | TCVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLSCAVKGFYPSDIAVEWESFGTEWSNYKTTPPVLDSDGSFFLVSKLTVSKE | |
| | EWQQGFVFSCSVLHEALHSHYTQKSLSLSPGK | |
| 9 | EVQLVESGGGLVQPGGSLRLSCAASGFNIKDTYIHWVRQAPGKGLEWVARIY | Anti-HER2_DIV fused |
| | PTNGYTRYADSVKGRFTISADTSKNTAYLQMNSLRAEDTAVYYCSRWGGDG | to CH3C.35.23.3 with |
| | FYAMDYWGQGTLVTVSSASTKGPSVFPLAPSSKSTSGGTAALGCLVKDYFPE | knob mutation |
| | PVTVSWNSGALTSGVHTFPAVLQSSGLYSLSSVVTVPSSSLGTQTYICNVNHK | |
| | PSNTKVDKKVEPKSCDKTHTCPPCPAPELLGGPSVFLFPPKPKDTLMISRTPEV | |
| | TCVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLWCLVKGFYPSDIAVEWESYGTEWVNYKTTPPVLDSDGSFFLYSKLTVTK | |
| | EEWQQGFVFSCSVMHEALHNHYTQKSLSLSPGK | |
| 10 | EVQLVESGGGLVQPGGSLRLSCAASGFNIKDTYIHWVRQAPGKGLEWVARIY | Anti-HER2_DIV fused |
| | PTNGYTRYADSVKGRFTISADTSKNTAYLQMNSLRAEDTAVYYCSRWGGDG | to CH3C.35.23.3 with |
| | FYAMDYWGQGTLVTVSSASTKGPSVFPLAPSSKSTSGGTAALGCLVKDYFPE | knob and LALA |
| | PVTVSWNSGALTSGVHTFPAVLQSSGLYSLSSVVTVPSSSLGTQTYICNVNHK | mutations |
| | PSNTKVDKKVEPKSCDKTHTCPPCPAPEAAGGPSVFLFPPKPKDTLMISRTPEV | |
| | TCVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLWCLVKGFYPSDIAVEWESYGTEWVNYKTTPPVLDSDGSFFLYSKLTVTK | |
| | EEWQQGFVFSCSVMHEALHNHYTQKSLSLSPGK | |
| 11 | EVQLVESGGGLVQPGGSLRLSCAASGFNIKDTYIHWVRQAPGKGLEWVARIY | Anti-HER2_DIV fused |
| | PTNGYTRYADSVKGRFTISADTSKNTAYLQMNSLRAEDTAVYYCSRWGGDG | knob and M428L and |
| | FYAMDYWGQGTLVTVSSASTKGPSVFPLAPSSKSTSGGTAALGCLVKDYFPE | to CH3C.35.23.3 with |
| | PVTVSWNSGALTSGVHTFPAVLQSSGLYSLSSVVTVPSSSLGTQTYICNVNHK | N434S mutations |
| | PSNTKVDKKVEPKSCDKTHTCPPCPAPELLGGPSVFLFPPKPKDTLMISRTPEV | |
| | TCVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLWCLVKGFYPSDIAVEWESYGTEWVNYKTTPPVLDSDGSFFLYSKLTVTK | |
| | EEWQQGFVFSCSVLHEALHSHYTQKSLSLSPGK | |
| 12 | EVQLVESGGGLVQPGGSLRLSCAASGFNIKDTYIHWVRQAPGKGLEWVARIY | Anti-HER2_DIV fused |
| | PTNGYTRYADSVKGRFTISADTSKNTAYLQMNSLRAEDTAVYYCSRWGGDG | to CH3C.35.23.3 with |
| | FYAMDYWGQGTLVTVSSASTKGPSVFPLAPSSKSTSGGTAALGCLVKDYFPE | knob, LALA, and |
| | PVTVSWNSGALTSGVHTFPAVLQSSGLYSLSSVVTVPSSSLGTQTYICNVNHK | M428L and N434S |
| | PSNTKVDKKVEPKSCDKTHTCPPCPAPEAAGGPSVFLFPPKPKDTLMISRTPEV | mutations |
| | TCVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLWCLVKGFYPSDIAVEWESYGTEWVNYKTTPPVLDSDGSFFLYSKLTVTK | |
| | EEWQQGFVFSCSVLHEALHSHYTQKSLSLSPGK | |
| 13 | EVQLVESGGGLVQPGGSLRLSCAASGFNIKDTYIHWVRQAPGKGLEWVARIY | Anti-HER2_DIV fused |
| | PTNGYTRYADSVKGRFTISADTSKNTAYLQMNSLRAEDTAVYYCSRWGGDG | to CH3C.35.23.3 with |
| | FYAMDYWGQGTLVTVSSASTKGPSVFPLAPSSKSTSGGTAALGCLVKDYFPE | hole mutations |
| | PVTVSWNSGALTSGVHTFPAVLQSSGLYSLSSVVTVPSSSLGTQTYICNVNHK | |
| | PSNTKVDKKVEPKSCDKTHTCPPCPAPELLGGPSVFLFPPKPKDTLMISRTPEV | |
| | TCVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLSCAVKGFYPSDIAVEWESYGIEWVNYKTTPPVLDSDGSFFLVSKLTVTKE | |
| | EWQQGFVFSCSVMHEALHNHYTQKSLSLSPGK | |
| 14 | EVQLVESGGGLVQPGGSLRLSCAASGFNIKDTYIHWVRQAPGKGLEWVARIY | Anti-HER2_DIV fused |
| | PTNGYTRYADSVKGRFTISADTSKNTAYLQMNSLRAEDTAVYYCSRWGGDG | to CH3C.35.23.3 with |
| | FYAMDYWGQGTLVTVSSASTKGPSVFPLAPSSKSTSGGTAALGCLVKDYFPE | hole and LALA |
| | PVTVSWNSGALTSGVHTFPAVLQSSGLYSLSSVVTVPSSSLGTQTYICNVNHK | mutations |
| | PSNTKVDKKVEPKSCDKTHTCPPCPAPEAAGGPSVFLFPPKPKDTLMISRTPEV | |
| | TCVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLSCAVKGFYPSDIAVEWESYGTEWVNYKTTPPVLDSDGSFFLVSKLTVTKE | |
| | EWQQGFVFSCSVMHEALHNHYTQKSLSLSPGK | |
| 15 | EVQLVESGGGLVQPGGSLRLSCAASGFNIKDTYIHWVRQAPGKGLEWVARIY | Anti-HER2_DIV fused |
| | PTNGYTRYADSVKGRFTISADTSKNTAYLQMNSLRAEDTAVYYCSRWGGDG | to CH3C.35.23.3 with |
| | FYAMDYWGQGTLVTVSSASTKGPSVFPLAPSSKSTSGGTAALGCLVKDYFPE | hole and M428L and |
| | PVTVSWNSGALTSGVHTFPAVLQSSGLYSLSSVVTVPSSSLGTQTYICNVNHK | N434S mutations |
| | PSNTKVDKKVEPKSCDKTHTCPPCPAPELLGGPSVFLFPPKPKDTLMISRTPEV | |
| | TCVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLSCAVKGFYPSDIAVEWESYGTEWVNYKTTPPVLDSDGSFFLVSKLTVTKE | |
| | EWQQGFVFSCSVLHEALHSHYTQKSLSLSPGK | |
| 16 | EVQLVESGGGLVQPGGSLRLSCAASGFNIKDTYIHWVRQAPGKGLEWVARIY | Anti-HER2_DIV fused |
| | PTNGYTRYADSVKGRFTISADTSKNTAYLQMNSLRAEDTAVYYCSRWGGDG | to CH3C.35.23.3 with |
| | FYAMDYWGQGTLVTVSSASTKGPSVFPLAPSSKSTSGGTAALGCLVKDYFPE | hole, LALA, and |
| | PVTVSWNSGALTSGVHTFPAVLQSSGLYSLSSVVTVPSSSLGTQTYICNVNHK | M428L and N434S |
| | PSNTKVDKKVEPKSCDKTHTCPPCPAPEAAGGPSVFLFPPKPKDTLMISRTPEV | mutations |
| | TCVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLSCAVKGFYPSDIAVEWESYGTEWVNYKTTPPVLDSDGSFFLVSKLTVTKE | |
| | EWQQGFVFSCSVLHEALHSHYTQKSLSLSPGK | |
| 17 | EVQLVESGGGLVQPGGSLRLSCAASGFNIKDTYIHWVRQAPGKGLEWVARIY | Anti-HER2_DIV fused |
| | PTNGYTRYADSVKGRFTISADTSKNTAYLQMNSLRAEDTAVYYCSRWGGDG | to CH3C.35.23.4 with |
| | FYAMDYWGQGTLVTVSSASTKGPSVFPLAPSSKSTSGGTAALGCLVKDYFPE | knob mutation |
| | PVTVSWNSGALTSGVHTFPAVLQSSGLYSLSSVVTVPSSSLGTQTYICNVNHK | |
| | PSNTKVDKKVEPKSCDKTHTCPPCPAPELLGGPSVFLFPPKPKDTLMISRTPEV | |
| | TCVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLWCLVKGFYPSDIAVEWESYGTEWSNYKTTPPVLDSDGSFFLYSKLTVSK | |
| | EEWQQGFVFSCSVMHEALHNHYTQKSLSLSPGK | |
| 18 | EVQLVESGGGLVQPGGSLRLSCAASGFNIKDTYIHWVRQAPGKGLEWVARIY | Anti-HER2_DIV fused |
| | PTNGYTRYADSVKGRFTISADTSKNTAYLQMNSLRAEDTAVYYCSRWGGDG | to CH3C.35.23.4 with |
| | FYAMDYWGQGTLVTVSSASTKGPSVFPLAPSSKSTSGGTAALGCLVKDYFPE | knob and LALA |
| | PVTVSWNSGALTSGVHTFPAVLQSSGLYSLSSVVTVPSSSLGTQTYICNVNHK | mutations |
| | PSNTKVDKKVEPKSCDKTHTCPPCPAPEAAGGPSVFLFPPKPKDTLMISRTPEV | |
| | TCVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLWCLVKGFYPSDIAVEWESYGTEWSNYKTTPPVLDSDGSFFLYSKLTVSK | |
| | EEWQQGFVFSCSVMHEALHNHYTQKSLSLSPGK | |
| 19 | EVQLVESGGGLVQPGGSLRLSCAASGFNIKDTYIHWVRQAPGKGLEWVARIY | Anti-HER2_DIV fused |
| | PTNGYTRYADSVKGRFTISADTSKNTAYLQMNSLRAEDTAVYYCSRWGGDG | to CH3C.35.23.4 with |
| | FYAMDYWGQGTLVTVSSASTKGPSVFPLAPSSKSTSGGTAALGCLVKDYFPE | knob and M428L and |
| | PVTVSWNSGALTSGVHTFPAVLQSSGLYSLSSVVTVPSSSLGTQTYICNVNHK | N434S mutations |
| | PSNTKVDKKVEPKSCDKTHTCPPCPAPELLGGPSVFLFPPKPKDTLMISRTPEV | |
| | TCVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLWCLVKGFYPSDIAVEWESYGTEWSNYKTTPPVLDSDGSFFLYSKLTVSK | |
| | EEWQQGFVFSCSVLHEALHSHYTQKSLSLSPGK | |
| 20 | EVQLVESGGGLVQPGGSLRLSCAASGFNIKDTYIHWVRQAPGKGLEWVARIY | Anti-HER2_DIV fused |
| | PTNGYTRYADSVKGRFTISADTSKNTAYLQMNSLRAEDTAVYYCSRWGGDG | to CH3C.35.23.4 with |
| | FYAMDYWGQGTLVTVSSASTKGPSVFPLAPSSKSTSGGTAALGCLVKDYFPE | knob, LALA, and |
| | PVTVSWNSGALTSGVHTFPAVLQSSGLYSLSSVVTVPSSSLGTQTYICNVNHK | M428L and N434S |
| | PSNTKVDKKVEPKSCDKTHTCPPCPAPEAAGGPSVFLFPPKPKDTLMISRTPEV | mutations |
| | TCVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLWCLVKGFYPSDIAVEWESYGTEWSNYKTTPPVLDSDGSFFLYSKLTVSK | |
| | EEWQQGFVFSCSVLHEALHSHYTQKSLSLSPGK | |
| 21 | EVQLVESGGGLVQPGGSLRLSCAASGFNIKDTYIHWVRQAPGKGLEWVARIY | Anti-HER2_DIV fused |
| | PTNGYTRYADSVKGRFTISADTSKNTAYLQMNSLRAEDTAVYYCSRWGGDG | to CH3C.35.23.4 with |
| | FYAMDYWGQGTLVTVSSASTKGPSVFPLAPSSKSTSGGTAALGCLVKDYFPE | hole mutations |
| | PVTVSWNSGALTSGVHTFPAVLQSSGLYSLSSVVTVPSSSLGTQTYICNVNHK | |
| | PSNTKVDKKVEPKSCDKTHTCPPCPAPELLGGPSVFLFPPKPKDTLMISRTPEV | |
| | TCVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLSCAVKGFYPSDIAVEWESYGTEWSNYKTTPPVLDSDGSFFLVSKLTVSKE | |
| | EWQQGFVFSCSVMHEALHNHYTQKSLSLSPGK | |
| 22 | EVQLVESGGGLVQPGGSLRLSCAASGFNIKDTYIHWVRQAPGKGLEWVARIY | Anti-HER2_DIV fused |
| | PTNGYTRYADSVKGRFTISADTSKNTAYLQMNSLRAEDTAVYYCSRWGGDG | to CH3C.35.23.4 with |
| | FYAMDYWGQGTLVTVSSASTKGPSVFPLAPSSKSTSGGTAALGCLVKDYFPE | hole and LALA |
| | PVTVSWNSGALTSGVHTFPAVLQSSGLYSLSSVVTVPSSSLGTQTYICNVNHK | mutations |
| | PSNTKVDKKVEPKSCDKTHTCPPCPAPEAAGGPSVFLFPPKPKDTLMISRTPEV | |
| | TCVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLSCAVKGFYPSDIAVEWESYGTEWSNYKTTPPVLDSDGSFFLVSKLTVSKE | |
| | EWQQGFVFSCSVMHEALHNHYTQKSLSLSPGK | |
| 23 | EVQLVESGGGLVQPGGSLRLSCAASGFNIKDTYIHWVRQAPGKGLEWVARIY | Anti-HER2_DIV fused |
| | PTNGYTRYADSVKGRFTISADTSKNTAYLQMNSLRAEDTAVYYCSRWGGDG | to CH3C.35.23.4 with |
| | FYAMDYWGQGTLVTVSSASTKGPSVFPLAPSSKSTSGGTAALGCLVKDYFPE | hole and M428L and |
| | PVTVSWNSGALTSGVHTFPAVLQSSGLYSLSSVVTVPSSSLGTQTYICNVNHK | N434S mutations |
| | PSNTKVDKKVEPKSCDKTHTCPPCPAPELLGGPSVFLFPPKPKDTLMISRTPEV | |
| | TCVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLSCAVKGFYPSDIAVEWESYGTEWSNYKTTPPVLDSDGSFFLVSKLTVSKE | |
| | EWQQGFVFSCSVLHEALHSHYTQKSLSLSPGK | |
| 24 | EVQLVESGGGLVQPGGSLRLSCAASGFNIKDTYIHWVRQAPGKGLEWVARIY | Anti-HER2_DIV fused |
| | PTNGYTRYADSVKGRFTISADTSKNTAYLQMNSLRAEDTAVYYCSRWGGDG | to CH3C.35.23.4 with |
| | FYAMDYWGQGTLVTVSSASTKGPSVFPLAPSSKSTSGGTAALGCLVKDYFPE | hole, LALA, and |
| | PVTVSWNSGALTSGVHTFPAVLQSSGLYSLSSVVTVPSSSLGTQTYICNVNHK | M428L and N434S |
| | PSNTKVDKKVEPKSCDKTHTCPPCPAPEAAGGPSVFLFPPKPKDTLMISRTPEV | mutations |
| | TCVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLSCAVKGFYPSDIAVEWESYGTEWSNYKTTPPVLDSDGSFFLVSKLTVSKE | |
| | EWQQGFVFSCSVLHEALHSHYTQKSLSLSPGK | |
| 25 | EVQLVESGGGLVQPGGSLRLSCAASGFNIKDTYIHWVRQAPGKGLEWVARIY | Anti-HER2 DIV fused |
| | PTNGYTRYADSVKGRFTISADTSKNTAYLQMNSLRAEDTAVYYCSRWGGDG | to Fc with knob |
| | FYAMDYWGQGTLVTVSSASTKGPSVFPLAPSSKSTSGGTAALGCLVKDYFPE | mutation |
| | PVTVSWNSGALTSGVHTFPAVLQSSGLYSLSSVVTVPSSSLGTQTYICNVNHK | |
| | PSNTKVDKKVEPKSCDKTHTCPPCPAPELLGGPSVFLFPPKPKDTLMISRTPEV | |
| | TCVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLWCLVKGFYPSDIAVEWESNGQPENNYKTTPPVLDSDGSFFLYSKLTVDK | |
| | SRWQQGNVFSCSVMHEALHNHYTQKSLSLSPGK | |
| 26 | EVQLVESGGGLVQPGGSLRLSCAASGFNIKDTYIHWVRQAPGKGLEWVARIY | Anti-HER2_DIV fused |
| | PTNGYTRYADSVKGRFTISADTSKNTAYLQMNSLRAEDTAVYYCSRWGGDG | to Fc with knob and |
| | FYAMDYWGQGTLVTVSSASTKGPSVFPLAPSSKSTSGGTAALGCLVKDYFPE | M428L and N434S |
| | PVTVSWNSGALTSGVHTFPAVLQSSGLYSLSSVVTVPSSSLGTQTYICNVNHK | mutations |
| | PSNTKVDKKVEPKSCDKTHTCPPCPAPELLGGPSVFLFPPKPKDTLMISRTPEV | |
| | TCVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLWCLVKGFYPSDIAVEWESNGQPENNYKTTPPVLDSDGSFFLYSKLTVDK | |
| | SRWQQGNVFSCSVLHEALHSHYTQKSLSLSPGK | |
| 27 | EVQLVESGGGLVQPGGSLRLSCAASGFNIKDTYIHWVRQAPGKGLEWVARIY | Anti-HER2_DIV fused |
| | PTNGYTRYADSVKGRFTISADTSKNTAYLQMNSLRAEDTAVYYCSRWGGDG | to Fc with hole |
| | FYAMDYWGQGTLVTVSSASTKGPSVFPLAPSSKSTSGGTAALGCLVKDYFPE | mutations |
| | PVTVSWNSGALTSGVHTFPAVLQSSGLYSLSSVVTVPSSSLGTQTYICNVNHK | |
| | PSNTKVDKKVEPKSCDKTHTCPPCPAPELLGGPSVFLFPPKPKDTLMISRTPEV | |
| | TCVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLSCAVKGFYPSDIAVEWESNGQPENNYKTTPPVLDSDGSFFLVSKLTVDKS | |
| | RWQQGNVFSCSVMHEALHNHYTQKSLSLSPGK | |
| 28 | EVQLVESGGGLVQPGGSLRLSCAASGFNIKDTYIHWVRQAPGKGLEWVARIY | Anti-HER2_DIV fused |
| | PTNGYTRYADSVKGRFTISADTSKNTAYLQMNSLRAEDTAVYYCSRWGGDG | to Fc with hole and |
| | FYAMDYWGQGTLVTVSSASTKGPSVFPLAPSSKSTSGGTAALGCLVKDYFPE | M428L and N434S |
| | PVTVSWNSGALTSGVHTFPAVLQSSGLYSLSSVVTVPSSSLGTQTYICNVNHK | mutations |
| | PSNTKVDKKVEPKSCDKTHTCPPCPAPELLGGPSVFLFPPKPKDTLMISRTPEV | |
| | TCVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLSCAVKGFYPSDIAVEWESNGQPENNYKTTPPVLDSDGSFFLVSKLTVDKS | |
| | RWQQGNVFSCSVLHEALHSHYTQKSLSLSPGK | |
| 29 | EVQLVESGGGLVQPGGSLRLSCAASGFTFTDYTMDWVRQAPGKGLEWVAD | Anti-HER2_DII fused |
| | VNPNSGGSIYNQRFKGRFTLSVDRSKNTLYLQMNSLRAEDTAVYYCARNLGP | to CH3C.35.23.1.1 with |
| | SFYFDYWGQGTLVTVSSASTKGPSVFPLAPSSKSTSGGTAALGCLVKDYFPEP | knob mutation |
| | VTVSWNSGALTSGVHTFPAVLQSSGLYSLSSVVTVPSSSLGTQTYICNVNHKP | |
| | SNTKVDKKVEPKSCDKTHTCPPCPAPELLGGPSVFLFPPKPKDTLMISRTPEVT | |
| | CVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLWCLVKGFYPSDIAVEWESFGTEWSNYKTTPPVLDSDGSFFLYSKLTVSKE | |
| | EWQQGFVFSCSVMHEALHNHYTQKSLSLSPGK | |
| 30 | EVQLVESGGGLVQPGGSLRLSCAASGFTFTDYTMDWVRQAPGKGLEWVAD | Anti-HER2_DII fused |
| | VNPNSGGSIYNQRFKGRFTLSVDRSKNTLYLQMNSLRAEDTAVYYCARNLGP | to CH3C.35.23.1.1 with |
| | SFYFDYWGQGTLVTVSSASTKGPSVFPLAPSSKSTSGGTAALGCLVKDYFPEP | knob and LALA |
| | VTVSWNSGALTSGVHTFPAVLQSSGLYSLSSVVTVPSSSLGTQTYICNVNHKP | mutations |
| | SNTKVDKKVEPKSCDKTHTCPPCPAPEAAGGPSVFLFPPKPKDTLMISRTPEV | |
| | TCVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLWCLVKGFYPSDIAVEWESFGTEWSNYKTTPPVLDSDGSFFLYSKLTVSKE | |
| | EWQQGFVFSCSVMHEALHNHYTQKSLSLSPGK | |
| 31 | EVQLVESGGGLVQPGGSLRLSCAASGFTFTDYTMDWVRQAPGKGLEWVAD | Anti-HER2_DII fused |
| | VNPNSGGSIYNQRFKGRFTLSVDRSKNTLYLQMNSLRAEDTAVYYCARNLGP | to CH3C.35.23.1.1 with |
| | SFYFDYWGQGTLVTVSSASTKGPSVFPLAPSSKSTSGGTAALGCLVKDYFPEP | knob and M428L and |
| | VTVSWNSGALTSGVHTFPAVLQSSGLYSLSSVVTVPSSSLGTQTYICNVNHKP | N434S mutations |
| | SNTKVDKKVEPKSCDKTHTCPPCPAPELLGGPSVFLFPPKPKDTLMISRTPEVT | |
| | CVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLWCLVKGFYPSDIAVEWESFGTEWSNYKTTPPVLDSDGSFFLYSKLTVSKE | |
| | EWQQGFVFSCSVLHEALHSHYTQKSLSLSPGK | |
| 32 | EVQLVESGGGLVQPGGSLRLSCAASGFTFTDYTMDWVRQAPGKGLEWVAD | Anti-HER2_DII fused |
| | VNPNSGGSIYNQRFKGRFTLSVDRSKNTLYLQMNSLRAEDTAVYYCARNLGP | to CH3C.35.23.1.1 with |
| | SFYFDYWGQGTLVTVSSASTKGPSVFPLAPSSKSTSGGTAALGCLVKDYFPEP | knob, LALA, and |
| | VTVSWNSGALTSGVHTFPAVLQSSGLYSLSSVVTVPSSSLGTQTYICNVNHKP | M428L and N434S |
| | SNTKVDKKVEPKSCDKTHTCPPCPAPEAAGGPSVFLFPPKPKDTLMISRTPEV | mutations |
| | TCVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLWCLVKGFYPSDIAVEWESFGTEWSNYKTTPPVLDSDGSFFLYSKLTVSKE | |
| | EWQQGFVFSCSVLHEALHSHYTQKSLSLSPGK | |
| 33 | EVQLVESGGGLVQPGGSLRLSCAASGFTFTDYTMDWVRQAPGKGLEWVAD | Anti-HER2_DII fused |
| | VNPNSGGSIYNQRFKGRFTLSVDRSKNTLYLQMNSLRAEDTAVYYCARNLGP | to CH3C.35.23.1.1 with |
| | SFYFDYWGQGTLVTVSSASTKGPSVFPLAPSSKSTSGGTAALGCLVKDYFPEP | hole mutations |
| | VTVSWNSGALTSGVHTFPAVLQSSGLYSLSSVVTVPSSSLGTQTYICNVNHKP | |
| | SNTKVDKKVEPKSCDKTHTCPPCPAPELLGGPSVFLFPPKPKDTLMISRTPEVT | |
| | CVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLSCAVKGFYPSDIAVEWESFGTEWSNYKTTPPVLDSDGSFFLVSKLTVSKE | |
| | EWQQGFVFSCSVMHEALHNHYTQKSLSLSPGK | |
| 34 | EVQLVESGGGLVQPGGSLRLSCAASGFTFTDYTMDWVRQAPGKGLEWVAD | Anti-HER2_DII fused |
| | VNPNSGGSIYNQRFKGRFTLSVDRSKNTLYLQMNSLRAEDTAVYYCARNLGP | to CH3C.35.23.1.1 with |
| | SFYFDYWGQGTLVTVSSASTKGPSVFPLAPSSKSTSGGTAALGCLVKDYFPEP | hole and LALA |
| | VTVSWNSGALTSGVHTFPAVLQSSGLYSLSSVVTVPSSSLGTQTYICNVNHKP | mutations |
| | SNTKVDKKVEPKSCDKTHTCPPCPAPEAAGGPSVFLFPPKPKDTLMISRTPEV | |
| | TCVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLSCAVKGFYPSDIAVEWESFGTEWSNYKTTPPVLDSDGSFFLVSKLTVSKE | |
| | EWQQGFVFSCSVMHEALHNHYTQKSLSLSPGK | |
| 35 | EVQLVESGGGLVQPGGSLRLSCAASGFTFTDYTMDWVRQAPGKGLEWVAD | Anti-HER2_DII fused |
| | VNPNSGGSIYNQRFKGRFTLSVDRSKNTLYLQMNSLRAEDTAVYYCARNLGP | to CH3C.35.23.1.1 with |
| | SFYFDYWGQGTLVTVSSASTKGPSVFPLAPSSKSTSGGTAALGCLVKDYFPEP | hole and M428L and |
| | VTVSWNSGALTSGVHTFPAVLQSSGLYSLSSVVTVPSSSLGTQTYICNVNHKP | N434S mutations |
| | SNTKVDKKVEPKSCDKTHTCPPCPAPELLGGPSVFLFPPKPKDTLMISRTPEVT | |
| | CVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLSCAVKGFYPSDIAVEWESFGTEWSNYKTTPPVLDSDGSFFLVSKLTVSKE | |
| | EWQQGFVFSCSVLHEALHSHYTQKSLSLSPGK | |
| 36 | EVQLVESGGGLVQPGGSLRLSCAASGFTFTDYTMDWVRQAPGKGLEWVAD | Anti-HER2_DII fused |
| | VNPNSGGSIYNQRFKGRFTLSVDRSKNTLYLQMNSLRAEDTAVYYCARNLGP | to CH3C.35.23.1.1 with |
| | SFYFDYWGQGTLVTVSSASTKGPSVFPLAPSSKSTSGGTAALGCLVKDYFPEP | hole, LALA, and |
| | VTVSWNSGALTSGVHTFPAVLQSSGLYSLSSVVTVPSSSLGTQTYICNVNHKP | M428L and N434S |
| | SNTKVDKKVEPKSCDKTHTCPPCPAPEAAGGPSVFLFPPKPKDTLMISRTPEV | mutations |
| | TCVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLSCAVKGFYPSDIAVEWESFGTEWSNYKTTPPVLDSDGSFFLVSKLTVSKE | |
| | EWQQGFVFSCSVLHEALHSHYTQKSLSLSPGK | |
| 37 | EVQLVESGGGLVQPGGSLRLSCAASGFTFTDYTMDWVRQAPGKGLEWVAD | Anti-HER2_DII fused |
| | VNPNSGGSIYNQRFKGRFTLSVDRSKNTLYLQMNSLRAEDTAVYYCARNLGP | to CH3C.35.23.3 with |
| | SFYFDYWGQGTLVTVSSASTKGPSVFPLAPSSKSTSGGTAALGCLVKDYFPEP | knob mutation |
| | VTVSWNSGALTSGVHTFPAVLQSSGLYSLSSVVTVPSSSLGTQTYICNVNHKP | |
| | SNTKVDKKVEPKSCDKTHTCPPCPAPELLGGPSVFLFPPKPKDTLMISRTPEVT | |
| | CVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLWCLVKGFYPSDIAVEWESYGTEWVNYKTTPPVLDSDGSFFLYSKLTVTK | |
| | EEWQQGFVFSCSVMHEALHNHYTQKSLSLSPGK | |
| 38 | EVQLVESGGGLVQPGGSLRLSCAASGFTFTDYTMDWVRQAPGKGLEWVAD | Anti-HER2_DII fused |
| | VNPNSGGSIYNQRFKGRFTLSVDRSKNTLYLQMNSLRAEDTAVYYCARNLGP | to CH3C.35.23.3 with |
| | SFYFDYWGQGTLVTVSSASTKGPSVFPLAPSSKSTSGGTAALGCLVKDYFPEP | knob and LALA |
| | VTVSWNSGALTSGVHTFPAVLQSSGLYSLSSVVTVPSSSLGTQTYICNVNHKP | mutations |
| | SNTKVDKKVEPKSCDKTHTCPPCPAPEAAGGPSVFLFPPKPKDTLMISRTPEV | |
| | TCVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLWCLVKGFYPSDIAVEWESYGTEWVNYKTTPPVLDSDGSFFLYSKLTVTK | |
| | EEWQQGFVFSCSVMHEALHNHYTQKSLSLSPGK | |
| 39 | EVQLVESGGGLVQPGGSLRLSCAASGFTFTDYTMDWVRQAPGKGLEWVAD | Anti-HER2_DII fused |
| | VNPNSGGSIYNQRFKGRFTLSVDRSKNTLYLQMNSLRAEDTAVYYCARNLGP | to CH3C.35.23.3 with |
| | SFYFDYWGQGTLVTVSSASTKGPSVFPLAPSSKSTSGGTAALGCLVKDYFPEP | knob and M428L and |
| | VTVSWNSGALTSGVHTFPAVLQSSGLYSLSSVVTVPSSSLGTQTYICNVNHKP | N434S mutations |
| | SNTKVDKKVEPKSCDKTHTCPPCPAPELLGGPSVFLFPPKPKDTLMISRTPEVT | |
| | CVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLWCLVKGFYPSDIAVEWESYGTEWVNYKTTPPVLDSDGSFFLYSKLTVTK | |
| | EEWQQGFVFSCSVLHEALHSHYTQKSLSLSPGK | |
| 40 | EVQLVESGGGLVQPGGSLRLSCAASGFTFTDYTMDWVRQAPGKGLEWVAD | Anti-HER2_DII fused |
| | VNPNSGGSIYNQRFKGRFTLSVDRSKNTLYLQMNSLRAEDTAVYYCARNLGP | to CH3C.35.23.3 with |
| | SFYFDYWGQGTLVTVSSASTKGPSVFPLAPSSKSTSGGTAALGCLVKDYFPEP | knob, LALA, and |
| | VTVSWNSGALTSGVHTFPAVLQSSGLYSLSSVVTVPSSSLGTQTYICNVNHKP | M428L and N434S |
| | SNTKVDKKVEPKSCDKTHTCPPCPAPEAAGGPSVFLFPPKPKDTLMISRTPEV | mutations |
| | TCVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLWCLVKGFYPSDIAVEWESYGTEWVNYKTTPPVLDSDGSFFLYSKLTVTK | |
| | EEWQQGFVFSCSVLHEALHSHYTQKSLSLSPGK | |
| 41 | EVQLVESGGGLVQPGGSLRLSCAASGFTFTDYTMDWVRQAPGKGLEWVAD | Anti-HER2_DII fused |
| | VNPNSGGSIYNQRFKGRFTLSVDRSKNTLYLQMNSLRAEDTAVYYCARNLGP | to CH3C.35.23.3 with |
| | SFYFDYWGQGTLVTVSSASTKGPSVFPLAPSSKSTSGGTAALGCLVKDYFPEP | hole mutations |
| | VTVSWNSGALTSGVHTFPAVLQSSGLYSLSSVVTVPSSSLGTQTYICNVNHKP | |
| | SNTKVDKKVEPKSCDKTHTCPPCPAPELLGGPSVFLFPPKPKDTLMISRTPEVT | |
| | CVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLSCAVKGFYPSDIAVEWESYGTEWVNYKTTPPVLDSDGSFFLVSKLTVTKE | |
| | EWQQGFVFSCSVMHEALHNHYTQKSLSLSPGK | |
| 42 | EVQLVESGGGLVQPGGSLRLSCAASGFTFTDYTMDWVRQAPGKGLEWVAD | Anti-HER2_DII fused |
| | VNPNSGGSIYNQRFKGRFTLSVDRSKNTLYLQMNSLRAEDTAVYYCARNLGP | to CH3C.35.23.3 with |
| | SFYFDYWGQGTLVTVSSASTKGPSVFPLAPSSKSTSGGTAALGCLVKDYFPEP | hole and LALA |
| | VTVSWNSGALTSGVHTFPAVLQSSGLYSLSSVVTVPSSSLGTQTYICNVNHKP | mutations |
| | SNTKVDKKVEPKSCDKTHTCPPCPAPEAAGGPSVFLFPPKPKDTLMISRTPEV | |
| | TCVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLSCAVKGFYPSDIAVEWESYGTEWVNYKTTPPVLDSDGSFFLVSKLTVTKE | |
| | EWQQGFVFSCSVMHEALHNHYTQKSLSLSPGK | |
| 43 | EVQLVESGGGLVQPGGSLRLSCAASGFTFTDYTMDWVRQAPGKGLEWVAD | Anti-HER2_DII fused |
| | VNPNSGGSIYNQRFKGRFTLSVDRSKNTLYLQMNSLRAEDTAVYYCARNLGP | to CH3C.35.23.3 with |
| | SFYFDYWGQGTLVTVSSASTKGPSVFPLAPSSKSTSGGTAALGCLVKDYFPEP | hole and M428L and |
| | VTVSWNSGALTSGVHTFPAVLQSSGLYSLSSVVTVPSSSLGTQTYICNVNHKP | N434S mutations |
| | SNTKVDKKVEPKSCDKTHTCPPCPAPELLGGPSVFLFPPKPKDTLMISRTPEVT | |
| | CVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLSCAVKGFYPSDIAVEWESYGTEWVNYKTTPPVLDSDGSFFLVSKLTVTKE | |
| | EWQQGFVFSCSVLHEALHSHYTQKSLSLSPGK | |
| 44 | EVQLVESGGGLVQPGGSLRLSCAASGFTFTDYTMDWVRQAPGKGLEWVAD | Anti-HER2_DII fused |
| | VNPNSGGSIYNQRFKGRFTLSVDRSKNTLYLQMNSLRAEDTAVYYCARNLGP | to CH3C.35.23.3 with |
| | SFYFDYWGQGTLVTVSSASTKGPSVFPLAPSSKSTSGGTAALGCLVKDYFPEP | hole, LALA, and |
| | VTVSWNSGALTSGVHTFPAVLQSSGLYSLSSVVTVPSSSLGTQTYICNVNHKP | M428L and N434S |
| | SNTKVDKKVEPKSCDKTHTCPPCPAPEAAGGPSVFLFPPKPKDTLMISRTPEV | mutations |
| | TCVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLSCAVKGFYPSDIAVEWESYGTEWVNYKTTPPVLDSDGSFFLVSKLTVTKE | |
| | EWQQGFVFSCSVLHEALHSHYTQKSLSLSPGK | |
| 45 | EVQLVESGGGLVQPGGSLRLSCAASGFTFTDYTMDWVRQAPGKGLEWVAD | Anti-HER2_DII fused |
| | VNPNSGGSIYNQRFKGRFTLSVDRSKNTLYLQMNSLRAEDTAVYYCARNLGP | to CH3C.35.23.4 with |
| | SFYFDYWGQGTLVTVSSASTKGPSVFPLAPSSKSTSGGTAALGCLVKDYFPEP | knob mutation |
| | VTVSWNSGALTSGVHTFPAVLQSSGLYSLSSVVTVPSSSLGTQTYICNVNHKP | |
| | SNTKVDKKVEPKSCDKTHTCPPCPAPELLGGPSVFLFPPKPKDTLMISRTPEVT | |
| | CVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLWCLVKGFYPSDIAVEWESYGTEWSNYKTTPPVLDSDGSFFLYSKLTVSK | |
| | EEWQQGFVFSCSVMHEALHNHYTQKSLSLSPGK | |
| 46 | EVQLVESGGGLVQPGGSLRLSCAASGFTFTDYTMDWVRQAPGKGLEWVAD | Anti-HER2_DII fused |
| | VNPNSGGSIYNQRFKGRFTLSVDRSKNTLYLQMNSLRAEDTAVYYCARNLGP | to CH3C.35.23.4 with |
| | SFYFDYWGQGTLVTVSSASTKGPSVFPLAPSSKSTSGGTAALGCLVKDYFPEP | knob and LALA |
| | VTVSWNSGALTSGVHTFPAVLQSSGLYSLSSVVTVPSSSLGTQTYICNVNHKP | mutations |
| | SNTKVDKKVEPKSCDKTHTCPPCPAPEAAGGPSVFLFPPKPKDTLMISRTPEV | |
| | TCVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLWCLVKGFYPSDIAVEWESYGTEWSNYKTTPPVLDSDGSFFLYSKLTVSK | |
| | EEWQQGFVFSCSVMHEALHNHYTQKSLSLSPGK | |
| 47 | EVQLVESGGGLVQPGGSLRLSCAASGFTFTDYTMDWVRQAPGKGLEWVAD | Anti-HER2_DII fused |
| | VNPNSGGSIYNQRFKGRFTLSVDRSKNTLYLQMNSLRAEDTAVYYCARNLGP | to CH3C.35.23.4 with |
| | SFYFDYWGQGTLVTVSSASTKGPSVFPLAPSSKSTSGGTAALGCLVKDYFPEP | knob and M428L and |
| | VTVSWNSGALTSGVHTFPAVLQSSGLYSLSSVVTVPSSSLGTQTYICNVNHKP | N434S mutations |
| | SNTKVDKKVEPKSCDKTHTCPPCPAPELLGGPSVFLFPPKPKDTLMISRTPEVT | |
| | CVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLWCLVKGFYPSDIAVEWESYGTEWSNYKTTPPVLDSDGSFFLYSKLTVSK | |
| | EEWQQGFVFSCSVLHEALHSHYTQKSLSLSPGK | |
| 48 | EVQLVESGGGLVQPGGSLRLSCAASGFTFTDYTMDWVRQAPGKGLEWVAD | Anti-HER2_DII fused |
| | VNPNSGGSIYNQRFKGRFTLSVDRSKNTLYLQMNSLRAEDTAVYYCARNLGP | to CH3C.35.23.4 with |
| | SFYFDYWGQGTLVTVSSASTKGPSVFPLAPSSKSTSGGTAALGCLVKDYFPEP | knob, LALA, and |
| | VTVSWNSGALTSGVHTFPAVLQSSGLYSLSSVVTVPSSSLGTQTYICNVNHKP | M428L and N434S |
| | SNTKVDKKVEPKSCDKTHTCPPCPAPEAAGGPSVFLFPPKPKDTLMISRTPEV | mutations |
| | TCVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLWCLVKGFYPSDIAVEWESYGTEWSNYKTTPPVLDSDGSFFLYSKLTVSK | |
| | EEWQQGFVFSCSVLHEALHSHYTQKSLSLSPGK | |
| 49 | EVQLVESGGGLVQPGGSLRLSCAASGFTFTDYTMDWVRQAPGKGLEWVAD | Anti-HER2_DII fused |
| | VNPNSGGSIYNQRFKGRFTLSVDRSKNTLYLQMNSLRAEDTAVYYCARNLGP | to CH3C.35.23.4 with |
| | SFYFDYWGQGTLVTVSSASTKGPSVFPLAPSSKSTSGGTAALGCLVKDYFPEP | hole mutations |
| | VTVSWNSGALTSGVHTFPAVLQSSGLYSLSSVVTVPSSSLGTQTYICNVNHKP | |
| | SNTKVDKKVEPKSCDKTHTCPPCPAPELLGGPSVFLFPPKPKDTLMISRTPEVT | |
| | CVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLSCAVKGFYPSDIAVEWESYGTEWSNYKTTPPVLDSDGSFFLVSKLTVSKE | |
| | EWQQGFVFSCSVMHEALHNHYTQKSLSLSPGK | |
| 50 | EVQLVESGGGLVQPGGSLRLSCAASGFTFTDYTMDWVRQAPGKGLEWVAD | Anti-HER2_DII fused |
| | VNPNSGGSIYNQRFKGRFTLSVDRSKNTLYLQMNSLRAEDTAVYYCARNLGP | to CH3C.35.23.4 with |
| | SFYFDYWGQGTLVTVSSASTKGPSVFPLAPSSKSTSGGTAALGCLVKDYFPEP | hole and LALA |
| | VTVSWNSGALTSGVHTFPAVLQSSGLYSLSSVVTVPSSSLGTQTYICNVNHKP | mutations |
| | SNTKVDKKVEPKSCDKTHTCPPCPAPEAAGGPSVFLFPPKPKDTLMISRTPEV | |
| | TCVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLSCAVKGFYPSDIAVEWESYGTEWSNYKTTPPVLDSDGSFFLVSKLTVSKE | |
| | EWQQGFVFSCSVMHEALHNHYTQKSLSLSPGK | |
| 51 | EVQLVESGGGLVQPGGSLRLSCAASGFTFTDYTMDWVRQAPGKGLEWVAD | Anti-HER2_DII fused |
| | VNPNSGGSIYNQRFKGRFTLSVDRSKNTLYLQMNSLRAEDTAVYYCARNLGP | to CH3C.35.23.4 with |
| | SFYFDYWGQGTLVTVSSASTKGPSVFPLAPSSKSTSGGTAALGCLVKDYFPEP | hole and M428L and |
| | VTVSWNSGALTSGVHTFPAVLQSSGLYSLSSVVTVPSSSLGTQTYICNVNHKP | N434S mutations |
| | SNTKVDKKVEPKSCDKTHTCPPCPAPELLGGPSVFLFPPKPKDTLMISRTPEVT | |
| | CVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLSCAVKGFYPSDIAVEWESYGTEWSNYKTTPPVLDSDGSFFLVSKLTVSKE | |
| | EWQQGFVFSCSVLHEALHSHYTQKSLSLSPGK | |
| 52 | EVQLVESGGGLVQPGGSLRLSCAASGFTFTDYTMDWVRQAPGKGLEWVAD | Anti-HER2_DII fused |
| | VNPNSGGSIYNQRFKGRFTLSVDRSKNTLYLQMNSLRAEDTAVYYCARNLGP | to CH3C.35.23.4 with |
| | SFYFDYWGQGTLVTVSSASTKGPSVFPLAPSSKSTSGGTAALGCLVKDYFPEP | hole, LALA, and |
| | VTVSWNSGALTSGVHTFPAVLQSSGLYSLSSVVTVPSSSLGTQTYICNVNHKP | M428L and N434S |
| | SNTKVDKKVEPKSCDKTHTCPPCPAPEAAGGPSVFLFPPKPKDTLMISRTPEV | mutations |
| | TCVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLSCAVKGFYPSDIAVEWESYGTEWSNYKTTPPVLDSDGSFFLVSKLTVSKE | |
| | EWQQGFVFSCSVLHEALHSHYTQKSLSLSPGK | |
| 53 | EVQLVESGGGLVQPGGSLRLSCAASGFTFTDYTMDWVRQAPGKGLEWVAD | Anti-HER2_DII fused |
| | VNPNSGGSIYNQRFKGRFTLSVDRSKNTLYLQMNSLRAEDTAVYYCARNLGP | to Fc with knob |
| | SFYFDYWGQGTLVTVSSASTKGPSVFPLAPSSKSTSGGTAALGCLVKDYFPEP | mutation |
| | VTVSWNSGALTSGVHTFPAVLQSSGLYSLSSVVTVPSSSLGTQTYICNVNHKP | |
| | SNTKVDKKVEPKSCDKTHTCPPCPAPELLGGPSVFLFPPKPKDTLMISRTPEVT | |
| | CVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLWCLVKGFYPSDIAVEWESNGQPENNYKTTPPVLDSDGSFFLYSKLTVDK | |
| | SRWQQGNVFSCSVMHEALHNHYTQKSLSLSPGK | |
| 54 | EVQLVESGGGLVQPGGSLRLSCAASGFTFTDYTMDWVRQAPGKGLEWVAD | Anti-HER2_DII fused |
| | VNPNSGGSIYNQRFKGRFTLSVDRSKNTLYLQMNSLRAEDTAVYYCARNLGP | to Fc with knob and |
| | SFYFDYWGQGTLVTVSSASTKGPSVFPLAPSSKSTSGGTAALGCLVKDYFPEP | M428L and N434S |
| | VTVSWNSGALTSGVHTFPAVLQSSGLYSLSSVVTVPSSSLGTQTYICNVNHKP | mutations |
| | SNTKVDKKVEPKSCDKTHTCPPCPAPELLGGPSVFLFPPKPKDTLMISRTPEVT | |
| | CVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLWCLVKGFYPSDIAVEWESNGQPENNYKTTPPVLDSDGSFFLYSKLTVDK | |
| | SRWQQGNVFSCSVLHEALHSHYTQKSLSLSPGK | |
| 55 | EVQLVESGGGLVQPGGSLRLSCAASGFTFTDYTMDWVRQAPGKGLEWVAD | Anti-HER2_DII fused |
| | VNPNSGGSIYNQRFKGRFTLSVDRSKNTLYLQMNSLRAEDTAVYYCARNLGP | to Fc with hole |
| | SFYFDYWGQGTLVTVSSASTKGPSVFPLAPSSKSTSGGTAALGCLVKDYFPEP | mutations |
| | VTVSWNSGALTSGVHTFPAVLQSSGLYSLSSVVTVPSSSLGTQTYICNVNHKP | |
| | SNTKVDKKVEPKSCDKTHTCPPCPAPELLGGPSVFLFPPKPKDTLMISRTPEVT | |
| | CVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLSCAVKGFYPSDIAVEWESNGQPENNYKTTPPVLDSDGSFFLVSKLTVDKS | |
| | RWQQGNVFSCSVMHEALHNHYTQKSLSLSPGK | |
| 56 | EVQLVESGGGLVQPGGSLRLSCAASGFTFTDYTMDWVRQAPGKGLEWVAD | Anti-HER2_DII fused |
| | VNPNSGGSIYNQRFKGRFTLSVDRSKNTLYLQMNSLRAEDTAVYYCARNLGP | to Fc with hole and |
| | SFYFDYWGQGTLVTVSSASTKGPSVFPLAPSSKSTSGGTAALGCLVKDYFPEP | M428L and N434S |
| | VTVSWNSGALTSGVHTFPAVLQSSGLYSLSSVVTVPSSSLGTQTYICNVNHKP | mutations |
| | SNTKVDKKVEPKSCDKTHTCPPCPAPELLGGPSVFLFPPKPKDTLMISRTPEVT | |
| | CVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLSCAVKGFYPSDIAVEWESNGQPENNYKTTPPVLDSDGSFFLVSKLTVDKS | |
| | RWQQGNVFSCSVLHEALHSHYTQKSLSLSPGK | |
| 57 | DIQMTQSPSSLSASVGDRVTITCRASQDVNTAVAWYQQKPGKAPKLLIYSASF | Anti-HER2_DIV light |
| | LYSGVPSRFSGSRSGTDFTLTISSLQPEDFATYYCQQHYTTPPTFGQGTKVEIK | chain |
| | RTVAAPSVFIFPPSDEQLKSGTASVVCLLNNFYPREAKVQWKVDNALQSGNS | |
| | QESVTEQDSKDSTYSLSSTLTLSKADYEKHKVYACEVTHQGLSSPVTKSFNRG | |
| | EC | |
| 58 | DIQMTQSPSSLSASVGDRVTITCKASQDVSIGVAWYQQKPGKAPKLLIYSASY | Anti-HER2_DII light |
| | RYTGVPSRFSGSGSGTDFTLTISSLQPEDFATYYCQQYYIYPYTFGQGTKVEIK | chain |
| | RTVAAPSVFIFPPSDEQLKSGTASVVCLLNNFYPREAKVQWKVDNALQSGNS | |
| | QESVTEQDSKDSTYSLSSTLTLSKADYEKHKVYACEVTHQGLSSPVTKSFNRG | |
| | EC | |
| 59 | EVQLVESGGGLVQPGGSLRLSCAASGFNIKDTYIHWVRQAPGKGLEWVARIY | Anti-HER2_DIV VH |
| | PTNGYTRYADSVKGRFTISADTSKNTAYLQMNSLRAEDTAVYYCSRWGGDG | sequence |
| | FYAMDYWGQGTLVTVSS | |
| 60 | DIQMTQSPSSLSASVGDRVTITCRASQDVNTAVAWYQQKPGKAPKLLIYSASF | Anti-HER2_DIV VL |
| | LYSGVPSRFSGSRSGTDFTLTISSLQPEDFATYYCQQHYTTPPTFGQGTKVEIK | sequence |
| 61 | EVQLVESGGGLVQPGGSLRLSCAASGFTFTDYTMDWVRQAPGKGLEWVAD | Anti-HER2 DII VH |
| | VNPNSGGSIYNQRFKGRFTLSVDRSKNTLYLQMNSLRAEDTAVYYCARNLGP | sequence |
| | SFYFDYWGQGTLVTVSS | |
| 62 | DIQMTQSPSSLSASVGDRVTITCKASQDVSIGVAWYQQKPGKAPKLLIYSASY | Anti-HER2_DII VL |
| | RYTGVPSRFSGSGSGTDFTLTISSLQPEDFATYYCQQYYTYPYTFGQGTKVEIK | sequence |
| 63 | APEX₁X₂GGPSVFLFPPKPKDTLMISRTPEVTCVVVDVSHEDPEVKFNWYVDG | Consensus sequence for |
| | VEVHNAKTKPREEQYNSTYRVVSVLTVLHQDWLNGKEYKCKVSNKALPAPI | CH3C.35.23.1.1, |
| | EKTISKAKGQPREPQVYTLPPSRDELTKNQVSLWCLVKGFYPSDIAVEWESX₃ | CH3C.35.23.3, |
| | GTEWX₄NYKTTPPVLDSDGSFFLYSKLTVX₅KEEWQQGFVFSCSVX₆HEALHX₇ | CH3C.35.23.4, and |
| | HYTQKSLSLSPGK, wherein X₁ is L or A; X₂ is L or A; X₃ is F | CH3C.35.23 with knob |
| | or Y; X₄ is S or V; X₅ is S or T; X₆ is M or L; | and LALA mutations; |
| | and X₇ is N or S | M428L and N434S |
| | | mutations are part of |
| | | consensus sequence |
| 64 | APEX₁X₂GGPSVFLFPPKPKDTLMISRTPEVTCVVVDVSHEDPEVKFNWYVDG | Consensus sequence for |
| | VEVHNAKTKPREEQYNSTYRVVSVLTVLHQDWLNGKEYKCKVSNKALPAPI | CH3C.35.23.1.1, |
| | EKTISKAKGQPREPQVYTLPPSRDELTKNQVSLSCAVKGFYPSDIAVEWESX₃G | CH3C.35.23.3, |
| | TEWX₄NYKTTPPVLDSDGSFFLVSKLTVX₅KEEWQQGFVFSCSVX₆HEALHX₇H | CH3C.35.23.4,, and |
| | YTQKSLSLSPGK, wherein X₁ is L or A; X₂ is L or A; X₃ is F | CH3C.35.23 with hole |
| | or Y; X₄ is S or V; X₅ is S or T; X₆ is M or L; | and LALA mutations; |
| | and X₇ is N or S | M428L and N434S |
| | | mutations are part of |
| | | consensus sequence |
| 65 | APELLGGPSVFLFPPKPKDTLMISRTPEVTCVVVDVSHEDPEVKFNWYVDGVE | Fc sequence with knob |
| | VHNAKTKPREEQYNSTYRVVSVLTVLHQDWLNGKEYKCKVSNKALPAPIEK | mutation |
| | TISKAKGQPREPQVYTLPPSRDELTKNQVSLWCLVKGFYPSDIAVEWESNGQP | |
| | ENNYKTTPPVLDSDGSFFLYSKLTVDKSRWQQGNVFSCSVMHEALHNHYTQ | |
| | KSLSLSPGK | |
| 66 | APELLGGPSVFLFPPKPKDTLMISRTPEVTCVVVDVSHEDPEVKFNWYVDGVE | Fc sequence with knob |
| | VHNAKTKPREEQYNSTYRVVSVLTVLHQDWLNGKEYKCKVSNKALPAPIEK | and M428L and N434S |
| | TISKAKGQPREPQVYTLPPSRDELTKNQVSLWCLVKGFYPSDIAVEWESNGQP | mutations |
| | ENNYKTTPPVLDSDGSFFLYSKLTVDKSRWQQGNVFSCSVLHEALHSHYTQK | |
| | SLSLSPGK | |
| 67 | APELLGGPSVFLFPPKPKDTLMISRTPEVTCVVVDVSHEDPEVKFNWYVDGVE | Fc sequence with hole |
| | VHNAKTKPREEQYNSTYRVVSVLTVLHQDWLNGKEYKCKVSNKALPAPIEK | mutations |
| | TISKAKGQPREPQVYTLPPSRDELTKNQVSLSCAVKGFYPSDIAVEWESNGQP | |
| | ENNYKTTPPVLDSDGSFFLVSKLTVDKSRWQQGNVFSCSVMHEALHNHYTQ | |
| | KSLSLSPGK | |
| 68 | APELLGGPSVFLFPPKPKDTLMISRTPEVTCVVVDVSHEDPEVKFNWYVDGVE | Fc sequence with hole |
| | VHNAKTKPREEQYNSTYRVVSVLTVLHQDWLNGKEYKCKVSNKALPAPIEK | and M428L and N434S |
| | TISKAKGQPREPQVYTLPPSRDELTKNQVSLSCAVKGFYPSDIAVEWESNGQP | mutations |
| | ENNYKTTPPVLDSDGSFFLVSKLTVDKSRWQQGNVFSCSVLHEALHSHYTQK | |
| | SLSLSPGK | |
| 69 | GFNIKDTYIH | Anti-HER2_DIV CDR- |
| | | H1 |
| 70 | RIYPTNGYTRYADSVKG | Anti-HER2_DIV CDR- |
| | | H2 |
| 71 | SRWGGDGFYAMDY | Anti-HER2_DIV CDR- |
| | | H3 |
| 72 | RASQDVNTAVA | Anti-HER2_DIV CDR- |
| | | L1 |
| 73 | SASFLYS | Anti-HER2_DIV CDR- |
| | | L2 |
| 74 | QQHYTTPPT | Anti-HER2_DIV CDR- |
| | | L3 |
| 75 | GFTFTDYTMD | Anti-HER2_DII CDR- |
| | | H1 |
| 76 | DVNPNSGGSIYNQRFKG | Anti-HER2_DII CDR- |
| | | H2 |
| 77 | ARNLGPSFYFDY | Anti-HER2_DII CDR- |
| | | H3 |
| 78 | KASQDVSIGVA | Anti-HER2_DII CDR- |
| | | L1 |
| 79 | SASYRYT | Anti-HER2_DII CDR- |
| | | L2 |
| 80 | QQYYIYPYT | Anti-HER2_DII CDR- |
| | | L3 |
| 81 | EVQLVESGGGLVQPGGSLRLSCAASGFNIKDTYIHWVRQAPGKGLEWVARIY | Anti-HER2_DIV fused |
| | PTNGYTRYADSVKGRFTISADTSKNTAYLQMNSLRAEDTAVYYCSRWGGDG | to CH3C.35.23 with |
| | FYAMDYWGQGTLVTVSSASTKGPSVFPLAPSSKSTSGGTAALGCLVKDYFPE | knob mutation |
| | PVTVSWNSGALTSGVHTFPAVLQSSGLYSLSSVVTVPSSSLGTQTYICNVNHK | |
| | PSNTKVDKKVEPKSCDKTHTCPPCPAPELLGGPSVFLFPPKPKDTLMISRTPEV | |
| | TCVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLWCLVKGFYPSDIAVEWESYGTEWSNYKTTPPVLDSDGSFFLYSKLTVTK | |
| | EEWQQGFVFSCSVMHEALHNHYTQKSLSLSPGK | |
| 82 | EVQLVESGGGLVQPGGSLRLSCAASGFNIKDTYIHWVRQAPGKGLEWVARIY | Anti-HER2_DIV fused |
| | PTNGYTRYADSVKGRFTISADTSKNTAYLQMNSLRAEDTAVYYCSRWGGDG | to CH3C.35.23 with |
| | FYAMDYWGQGTLVTVSSASTKGPSVFPLAPSSKSTSGGTAALGCLVKDYFPE | knob and LALA |
| | PVTVSWNSGALTSGVHTFPAVLQSSGLYSLSSVVTVPSSSLGTQTYICNVNHK | mutations |
| | PSNTKVDKKVEPKSCDKTHTCPPCPAPEAAGGPSVFLFPPKPKDTLMISRTPEV | |
| | TCVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLWCLVKGFYPSDIAVEWESYGTEWSNYKTTPPVLDSDGSFFLYSKLTVTK | |
| | EEWQQGFVFSCSVMHEALHNHYTQKSLSLSPGK | |
| 83 | EVQLVESGGGLVQPGGSLRLSCAASGFNIKDTYIHWVRQAPGKGLEWVARIY | Anti-HER2_DIV fused |
| | PTNGYTRYADSVKGRFTISADTSKNTAYLQMNSLRAEDTAVYYCSRWGGDG | to CH3C.35.23 with |
| | FYAMDYWGQGTLVTVSSASTKGPSVFPLAPSSKSTSGGTAALGCLVKDYFPE | knob and M428L and |
| | PVTVSWNSGALTSGVHTFPAVLQSSGLYSLSSVVTVPSSSLGTQTYICNVNHK | N434S mutations |
| | PSNTKVDKKVEPKSCDKTHTCPPCPAPELLGGPSVFLFPPKPKDTLMISRTPEV | |
| | TCVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLWCLVKGFYPSDIAVEWESYGTEWSNYKTTPPVLDSDGSFFLYSKLTVTK | |
| | EEWQQGFVFSCSVLHEALHSHYTQKSLSLSPGK | |
| 84 | EVQLVESGGGLVQPGGSLRLSCAASGFNIKDTYIHWVRQAPGKGLEWVARIY | Anti-HER2_DIV fused |
| | PTNGYTRYADSVKGRFTISADTSKNTAYLQMNSLRAEDTAVYYCSRWGGDG | to CH3C.35.23 with |
| | FYAMDYWGQGTLVTVSSASTKGPSVFPLAPSSKSTSGGTAALGCLVKDYFPE | knob, LALA, and |
| | PVTVSWNSGALTSGVHTFPAVLQSSGLYSLSSVVTVPSSSLGTQTYICNVNHK | M428L and N434S |
| | PSNTKVDKKVEPKSCDKTHTCPPCPAPEAAGGPSVFLFPPKPKDTLMISRTPEV | mutations |
| | TCVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLWCLVKGFYPSDIAVEWESYGTEWSNYKTTPPVLDSDGSFFLYSKLTVTK | |
| | EEWQQGFVFSCSVLHEALHSHYTQKSLSLSPGK | |
| 85 | EVQLVESGGGLVQPGGSLRLSCAASGFNIKDTYIHWVRQAPGKGLEWVARIY | Anti-HER2_DIV fused |
| | PTNGYTRYADSVKGRFTISADTSKNTAYLQMNSLRAEDTAVYYCSRWGGDG | to CH3C.35.23 with |
| | FYAMDYWGQGTLVTVSSASTKGPSVFPLAPSSKSTSGGTAALGCLVKDYFPE | hole mutations |
| | PVTVSWNSGALTSGVHTFPAVLQSSGLYSLSSVVTVPSSSLGTQTYICNVNHK | |
| | PSNTKVDKKVEPKSCDKTHTCPPCPAPELLGGPSVFLFPPKPKDTLMISRTPEV | |
| | TCVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLSCAVKGFYPSDIAVEWESYGTEWSNYKTTPPVLDSDGSFFLVSKLTVTKE | |
| | EWQQGFVFSCSVMHEALHNHYTQKSLSLSPGK | |
| 86 | EVQLVESGGGLVQPGGSLRLSCAASGFNIKDTYIHWVRQAPGKGLEWVARIY | Anti-HER2_DIV fused |
| | PTNGYTRYADSVKGRFTISADTSKNTAYLQMNSLRAEDTAVYYCSRWGGDG | to CH3C.35.23 with |
| | FYAMDYWGQGTLVTVSSASTKGPSVFPLAPSSKSTSGGTAALGCLVKDYFPE | hole and LALA |
| | PVTVSWNSGALTSGVHTFPAVLQSSGLYSLSSVVTVPSSSLGTQTYICNVNHK | mutations |
| | PSNTKVDKKVEPKSCDKTHTCPPCPAPEAAGGPSVFLFPPKPKDTLMISRTPEV | |
| | TCVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLSCAVKGFYPSDIAVEWESYGTEWSNYKTTPPVLDSDGSFFLVSKLTVTKE | |
| | EWQQGFVFSCSVMHEALHNHYTQKSLSLSPGK | |
| 87 | EVQLVESGGGLVQPGGSLRLSCAASGFNIKDTYIHWVRQAPGKGLEWVARIY | Anti-HER2_DIV fused |
| | PTNGYTRYADSVKGRFTISADTSKNTAYLQMNSLRAEDTAVYYCSRWGGDG | to CH3C.35.23 with |
| | FYAMDYWGQGTLVTVSSASTKGPSVFPLAPSSKSTSGGTAALGCLVKDYFPE | hole and M428L and |
| | PVTVSWNSGALTSGVHTFPAVLQSSGLYSLSSVVTVPSSSLGTQTYICNVNHK | N434S mutations |
| | PSNTKVDKKVEPKSCDKTHTCPPCPAPELLGGPSVFLFPPKPKDTLMISRTPEV | |
| | TCVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLSCAVKGFYPSDIAVEWESYGTEWSNYKTTPPVLDSDGSFFLVSKLTVTKE | |
| | EWQQGFVFSCSVLHEALHSHYTQKSLSLSPGK | |
| 88 | EVQLVESGGGLVQPGGSLRLSCAASGFNIKDTYIHWVRQAPGKGLEWVARIY | Anti-HER2_DIV fused |
| | PTNGYTRYADSVKGRFTISADTSKNTAYLQMNSLRAEDTAVYYCSRWGGDG | to CH3C.35.23 with |
| | FYAMDYWGQGTLVTVSSASTKGPSVFPLAPSSKSTSGGTAALGCLVKDYFPE | hole, LALA, and |
| | PVTVSWNSGALTSGVHTFPAVLQSSGLYSLSSVVTVPSSSLGTQTYICNVNHK | M428L and N434S |
| | PSNTKVDKKVEPKSCDKTHTCPPCPAPEAAGGPSVFLFPPKPKDTLMISRTPEV | mutations |
| | TCVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLSCAVKGFYPSDIAVEWESYGTEWSNYKTTPPVLDSDGSFFLVSKLTVTKE | |
| | EWQQGFVFSCSVLHEALHSHYTQKSLSLSPGK | |
| 89 | EVQLVESGGGLVQPGGSLRLSCAASGFTFTDYTMDWVRQAPGKGLEWVAD | Anti-HER2_DII fused |
| | VNPNSGGSIYNQRFKGRFTLSVDRSKNTLYLQMNSLRAEDTAVYYCARNLGP | CH3C.35.23 to with |
| | SFYFDYWGQGTLVTVSSASTKGPSVFPLAPSSKSTSGGTAALGCLVKDYFPEP | knob mutation |
| | VTVSWNSGALTSGVHTFPAVLQSSGLYSLSSVVTVPSSSLGTQTYICNVNHKP | |
| | SNTKVDKKVEPKSCDKTHTCPPCPAPELLGGPSVFLFPPKPKDTLMISRTPEVT | |
| | CVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLWCLVKGFYPSDIAVEWESYGTEWSNYKTTPPVLDSDGSFFLYSKLTVTK | |
| | EEWQQGFVFSCSVMHEALHNHYTQKSLSLSPGK | |
| 90 | EVQLVESGGGLVQPGGSLRLSCAASGFTFTDYTMDWVRQAPGKGLEWVAD | Anti-HER2_DII fused |
| | VNPNSGGSIYNQRFKGRFTLSVDRSKNTLYLQMNSLRAEDTAVYYCARNLGP | to CH3C.35.23 with |
| | SFYFDYWGQGTLVTVSSASTKGPSVFPLAPSSKSTSGGTAALGCLVKDYFPEP | knob and LALA |
| | VTVSWNSGALTSGVHTFPAVLQSSGLYSLSSVVTVPSSSLGTQTYICNVNHKP | mutations |
| | SNTKVDKKVEPKSCDKTHTCPPCPAPEAAGGPSVFLFPPKPKDTLMISRTPEV | |
| | TCVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLWCLVKGFYPSDIAVEWESYGTEWSNYKTTPPVLDSDGSFFLYSKLTVTK | |
| | EEWQQGFVFSCSVMHEALHNHYTQKSLSLSPGK | |
| 91 | EVQLVESGGGLVQPGGSLRLSCAASGFTFTDYTMDWVRQAPGKGLEWVAD | Anti-HER2_DII fused |
| | VNPNSGGSIYNQRFKGRFTLSVDRSKNTLYLQMNSLRAEDTAVYYCARNLGP | to CH3C.35.23 with |
| | SFYFDYWGQGTLVTVSSASTKGPSVFPLAPSSKSTSGGTAALGCLVKDYFPEP | knob and M428L and |
| | VTVSWNSGALTSGVHTFPAVLQSSGLYSLSSVVTVPSSSLGTQTYICNVNHKP | N434S mutations |
| | SNTKVDKKVEPKSCDKTHTCPPCPAPELLGGPSVFLFPPKPKDTLMISRTPEVT | |
| | CVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLWCLVKGFYPSDIAVEWESYGTEWSNYKTTPPVLDSDGSFFLYSKLTVTK | |
| | EEWQQGFVFSCSVLHEALHSHYTQKSLSLSPGK | |
| 92 | EVQLVESGGGLVQPGGSLRLSCAASGFTFTDYTMDWVRQAPGKGLEWVAD | Anti-HER2_DII fused |
| | VNPNSGGSIYNQRFKGRFTLSVDRSKNTLYLQMNSLRAEDTAVYYCARNLGP | to CH3C.35.23 with |
| | SFYFDYWGQGTLVTVSSASTKGPSVFPLAPSSKSTSGGTAALGCLVKDYFPEP | knob, LALA, and |
| | VTVSWNSGALTSGVHTFPAVLQSSGLYSLSSVVTVPSSSLGTQTYICNVNHKP | M428L and N434S |
| | SNTKVDKKVEPKSCDKTHTCPPCPAPEAAGGPSVFLFPPKPKDTLMISRTPEV | mutations |
| | TCVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLWCLVKGFYPSDIAVEWESYGTEWSNYKTTPPVLDSDGSFFLYSKLTVTK | |
| | EEWQQGFVFSCSVLHEALHSHYTQKSLSLSPGK | |
| 93 | EVQLVESGGGLVQPGGSLRLSCAASGFTFTDYTMDWVRQAPGKGLEWVAD | Anti-HER2_DII fused |
| | VNPNSGGSIYNQRFKGRFTLSVDRSKNTLYLQMNSLRAEDTAVYYCARNLGP | to CH3C.35.23 with |
| | SFYFDYWGQGTLVTVSSASTKGPSVFPLAPSSKSTSGGTAALGCLVKDYFPEP | hole mutations |
| | VTVSWNSGALTSGVHTFPAVLQSSGLYSLSSVVTVPSSSLGTQTYICNVNHKP | |
| | SNTKVDKKVEPKSCDKTHTCPPCPAPELLGGPSVFLFPPKPKDTLMISRTPEVT | |
| | CVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLSCAVKGFYPSDIAVEWESYGTEWSNYKTTPPVLDSDGSFFLVSKLTVTKE | |
| | EWQQGFVFSCSVMHEALHNHYTQKSLSLSPGK | |
| 94 | EVQLVESGGGLVQPGGSLRLSCAASGFTFTDYTMDWVRQAPGKGLEWVAD | Anti-HER2_DII fused |
| | VNPNSGGSIYNQRFKGRFTLSVDRSKNTLYLQMNSLRAEDTAVYYCARNLGP | to CH3C.35.23 with |
| | SFYFDYWGQGTLVTVSSASTKGPSVFPLAPSSKSTSGGTAALGCLVKDYFPEP | hole and LALA |
| | VTVSWNSGALTSGVHTFPAVLQSSGLYSLSSVVTVPSSSLGTQTYICNVNHKP | mutations |
| | SNTKVDKKVEPKSCDKTHTCPPCPAPEAAGGPSVFLFPPKPKDTLMISRTPEV | |
| | TCVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLSCAVKGFYPSDIAVEWESYGTEWSNYKTTPPVLDSDGSFFLVSKLTVTKE | |
| | EWQQGFVFSCSVMHEALHNHYTQKSLSLSPGK | |
| 95 | EVQLVESGGGLVQPGGSLRLSCAASGFTFTDYTMDWVRQAPGKGLEWVAD | Anti-HER2_DII fused |
| | VNPNSGGSIYNQRFKGRFTLSVDRSKNTLYLQMNSLRAEDTAVYYCARNLGP | to CH3C.35.23 with |
| | SFYFDYWGQGTLVTVSSASTKGPSVFPLAPSSKSTSGGTAALGCLVKDYFPEP | hole and M428L and |
| | VTVSWNSGALTSGVHTFPAVLQSSGLYSLSSVVTVPSSSLGTQTYICNVNHKP | N434S mutations |
| | SNTKVDKKVEPKSCDKTHTCPPCPAPELLGGPSVFLFPPKPKDTLMISRTPEVT | |
| | CVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLSCAVKGFYPSDIAVEWESYGTEWSNYKTTPPVLDSDGSFFLVSKLTVTKE | |
| | EWQQGFVFSCSVLHEALHSHYTQKSLSLSPGK | |
| 96 | EVQLVESGGGLVQPGGSLRLSCAASGFTFTDYTMDWVRQAPGKGLEWVAD | Anti-HER2_DII fused |
| | VNPNSGGSIYNQRFKGRFTLSVDRSKNTLYLQMNSLRAEDTAVYYCARNLGP | to CH3C.35.23 with |
| | SFYFDYWGQGTLVTVSSASTKGPSVFPLAPSSKSTSGGTAALGCLVKDYFPEP | hole, LALA, and |
| | VTVSWNSGALTSGVHTFPAVLQSSGLYSLSSVVTVPSSSLGTQTYICNVNHKP | M428L and N434S |
| | SNTKVDKKVEPKSCDKTHTCPPCPAPEAAGGPSVFLFPPKPKDTLMISRTPEV | mutations |
| | TCVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLSCAVKGFYPSDIAVEWESYGTEWSNYKTTPPVLDSDGSFFLVSKLTVTKE | |
| | EWQQGFVFSCSVLHEALHSHYTQKSLSLSPGK | |
| 97 | EVQLVESGGGLVQPGGSLRLSCAASGFNIKDTYIHWVRQAPGKGLEWVARIY | Anti-HER2_DIV HC |
| | PTNGYTRYADSVKGRFTISADTSKNTAYLQMNSLRAEDTAVYYCSRWGGDG | with wild-type human |
| | FYAMDYWGQGTLVTVSSASTKGPSVFPLAPSSKSTSGGTAALGCLVKDYFPE | Fc |
| | PVTVSWNSGALTSGVHTFPAVLQSSGLYSLSSVVTVPSSSLGTQTYICNVNHK | |
| | PSNTKVDKKVEPKSCDKTHTCPPCPAPELLGGPSVFLFPPKPKDTLMISRTPEV | |
| | TCVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLTCLVKGFYPSDIAVEWESNGQPENNYKTTPPVLDSDGSFFLYSKLTVDKS | |
| | RWQQGNVFSCSVMHEALHNHYTQKSLSLSPGK | |
| 98 | EVQLVESGGGLVQPGGSLRLSCAASGFTFTDYTMDWVRQAPGKGLEWVAD | Anti-HER2_DII_HC |
| | VNPNSGGSIYNQRFKGRFTLSVDRSKNTLYLQMNSLRAEDTAVYYCARNLGP | with wild-type human |
| | SFYFDYWGQGTLVTVSSASTKGPSVFPLAPSSKSTSGGTAALGCLVKDYFPEP | Fc |
| | VTVSWNSGALTSGVHTFPAVLQSSGLYSLSSVVTVPSSSLGTQTYICNVNHKP | |
| | SNTKVDKKVEPKSCDKTHTCPPCPAPELLGGPSVFLFPPKPKDTLMISRTPEVT | |
| | CVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLTCLVKGFYPSDIAVEWESNGQPENNYKTTPPVLDSDGSFFLYSKLTVDKS | |
| | RWQQGNVFSCSVMHEALHNHYTQKSLSLSPGK | |
| 99 | APELLGGPSVFLFPPKPKDTLMISRTPEVTCVVVDVSHEDPEVKFNWYVDGVE | Wild-type human Fc |
| | VHNAKTKPREEQYNSTYRVVSVLTVLHQDWLNGKEYKCKVSNKALPAPIEK | sequence |
| | TISKAKGQPREPQVYTLPPSRDELTKNQVSLTCLVKGFYPSDIAVEWESNGQP | |
| | ENNYKTTPPVLDSDGSFFLYSKLTVDKSRWQQGNVFSCSVMHEALHNHYTQ | |
| | KSLSLSPGK | |
| 100 | APELLGGPSVFLFPPKPKDTLMISRTPEVTCVVVDVSHEDPEVKFNWYVDGVE | CH2 domain sequence |
| | VHNAKTKPREEQYNSTYRVVSVLTVLHQDWLNGKEYKCKVSNKALPAPIEK | |
| | TISKAK | |
| 101 | GQPREPQVYTLPPSRDELTKNQVSLTCLVKGFYPSDIAVEWESNGQPENNYKT | CH3 domain sequence |
| | TPPVLDSDGSFFLYSKLTVDKSRWQQGNVFSCSVMHEALHNHYTQKSLSLSP | |
| | GK | |
| 102 | MMDQARSAFSNLFGGEPLSYTRFSLARQVDGDNSHVEMKLAVDEEENADNN | Human transferrin |
| | TKANVTKPKRCSGSICYGTIAVIVFFLIGFMIGYLGYCKGVEPKTECERLAGTE | receptor protein 1 |
| | SPVREEPGEDFPAARRLYWDDLKRKLSEKLDSTDFTGTIKLLNENSYVPREAG | (TFR1) |
| | SQKDENLALYVENQFREFKLSKVWRDQHFVKIQVKDSAQNSVIIVDKNGRLV | |
| | YLVENPGGYVAYSKAATVTGKLVHANFGTKKDFEDLYTPVNGSIVIVRAGKI | |
| | TFAEKVANAESLNAIGVLIYMDQTKFPIVNAELSFFGHAHLGTGDPYTPGFPSF | |
| | NHTQFPPSRSSGLPNIPVQTISRAAAEKLFGNMEGDCPSDWKTDSTCRMVTSE | |
| | SKNVKLTVSNVLKEIKILNIFGVIKGFVEPDHYVVVGAQRDAWGPGAAKSGV | |
| | GTALLLKLAQMFSDMVLKDGFQPSRSIIFASWSAGDFGSVGATEWLEGYLSS | |
| | LHLKAFTYINLDKAVLGTSNFKVSASPLLYTLIEKTMQNVKHPVTGQFLYQDS | |
| | NVVASKVEKLTLDNAAFPFLAYSGIPAVSFCFCEDTDYPYLGTTMDTYKELIER | |
| | IPELNKVARAAAEVAGQFVIKLTHDVELNLDYERYNSQLLSFVRDLNQYRAD | |
| | IKEMGLSLQWLYSARGDFFRATSRLTTDFGNAEKTDRFVMKKLNDRVMRVE | |
| | YHFLSPYVSPKESPFRHVFWGSGSHTLPALLENLKLRKQNNGAFNETLFRNQL | |
| | ALATWTIQGAANALSGDVWDIDNEF | |
| 103 | NSVIIVDKNGRLVYLVENPGGYVAYSKAATVTGKLVHANFGTKKDFEDLYTP | Human TfR apical |
| | VNGSIVIVRAGKITFAEKVANAESLNAIGVLIYMDQTKFPIVNAELSFFGHAHL | domain |
| | GTGDPYTPGFPSFNHTQFPPSRSSGLPNIPVQTISRAAAEKLFGNMEGDCPSDW | |
| | KTDSTCRMVTSESKNVKLTVS | |
| 104 | EPKSCDKTHTCPPCP | Human IgG1 hinge |
| | | amino acid sequence |
| 105 | APELLGGPSVFLFPPKPKDTLMISRTPEVTCVVVDVSHEDPEVKFNWYVDGVE | Clone CH3C.35.23.3 |
| | VHNAKTKPREEQYNSTYRVVSVLTVLHQDWLNGKEYKCKVSNKALPAPIEK | with knob mutation |
| | TISKAKGQPREPQVYTLPPSRDELTKNQVSLWCLVKGFYPSDIAVEWESYGTE | |
| | WVNYKTTPPVLDSDGSFFLYSKLTVTKEEWQQGFVFSCSVMHEALHNHYTQ | |
| | KSLSLSPGK | |
| 106 | APEAAGGPSVFLFPPKPKDTLMISRTPEVTCVVVDVSHEDPEVKFNWYVDGV | Clone CH3C.35.23.3 |
| | EVHNAKTKPREEQYNSTYRVVSVLTVLHQDWLNGKEYKCKVSNKALPAPIE | with knob and LALA |
| | KTISKAKGQPREPQVYTLPPSRDELTKNQVSLWCLVKGFYPSDIAVEWESYGT | mutations |
| | EWVNYKTTPPVLDSDGSFFLYSKLTVTKEEWQQGFVFSCSVMHEALHNHYT | |
| | QKSLSLSPGK | |
| 107 | APEAAGGPSVFLFPPKPKDTLMISRTPEVTCVVVDVSHEDPEVKFNWYVDGV | Clone CH3C.35.23.3 |
| | EVHNAKTKPREEQYNSTYRVVSVLTVLHQDWLNGKEYKCKVSNKALGAPIE | with knob and |
| | KTISKAKGQPREPQVYTLPPSRDELTKNQVSLWCLVKGFYPSDIAVEWESYGT | LALAPG mutations |
| | EWVNYKTTPPVLDSDGSFFLYSKLTVTKEEWQQGFVFSCSVMHEALHNHYT | |
| | QKSLSLSPGK | |
| 108 | APELLGGPSVFLFPPKPKDTLYITREPEVTCVVVDVSHEDPEVKFNWYVDGVE | Clone CH3C.35.23.3 |
| | VHNAKTKPREEQYNSTYRVVSVLTVLHQDWLNGKEYKCKVSNKALPAPIEK | with knob and YTE |
| | TISKAKGQPREPQVYTLPPSRDELTKNQVSLWCLVKGFYPSDIAVEWESYGTE | mutations |
| | WVNYKTTPPVLDSDGSFFLYSKLTVTKEEWQQGFVFSCSVMHEALHNHYTQ | |
| | KSLSLSPGK | |
| 109 | APELLGGPSVFLFPPKPKDTLMISRTPEVTCVVVDVSHEDPEVKFNWYVDGVE | Clone CH3C.35.23.3 |
| | VHNAKTKPREEQYNSTYRVVSVLTVLHQDWLNGKEYKCKVSNKALPAPIEK | with knob and M198L |
| | TISKAKGQPREPQVYTLPPSRDELTKNQVSLWCLVKGFYPSDIAVEWESYGTE | and N204S mutations |
| | WVNYKTTPPVLDSDGSFFLYSKLTVTKEEWQQGFVFSCSVLHEALHSHYTQK | |
| | SLSLSPGK | |
| 110 | APEAAGGPSVFLFPPKPKDTLYITREPEVTCVVVDVSHEDPEVKFNWYVDGV | Clone CH3C.35.23.3 |
| | EVHNAKTKPREEQYNSTYRVVSVLTVLHQDWLNGKEYKCKVSNKALPAPIE | with knob, LALA, and |
| | KTISKAKGQPREPQVYTLPPSRDELTKNQVSLWCLVKGFYPSDIAVEWESYGT | YTE mutations |
| | EWVNYKTTPPVLDSDGSFFLYSKLTVTKEEWQQGFVFSCSVMHEALHNHYT | |
| | QKSLSLSPGK | |
| 111 | APEAAGGPSVFLFPPKPKDTLYITREPEVTCVVVDVSHEDPEVKFNWYVDGV | Clone CH3C.35.23.3 |
| | EVHNAKTKPREEQYNSTYRVVSVLTVLHQDWLNGKEYKCKVSNKALGAPIE | with knob, LALAPG, |
| | KTISKAKGQPREPQVYTLPPSRDELTKNQVSLWCLVKGFYPSDIAVEWESYGT | and YTE mutations |
| | EWVNYKTTPPVLDSDGSFFLYSKLTVTKEEWQQGFVFSCSVMHEALHNHYT | |
| | QKSLSLSPGK | |
| 112 | APEAAGGPSVFLFPPKPKDTLMISRTPEVTCVVVDVSHEDPEVKFNWYVDGV | Clone CH3C.35.23.3 |
| | EVHNAKTKPREEQYNSTYRVVSVLTVLHQDWLNGKEYKCKVSNKALPAPIE | with knob, LALA, and |
| | KTISKAKGQPREPQVYTLPPSRDELTKNQVSLWCLVKGFYPSDIAVEWESYGT | M198L and N204S |
| | EWVNYKTTPPVLDSDGSFFLYSKLTVTKEEWQQGFVFSCSVLHEALHSHYTQ | mutations |
| | KSLSLSPGK | |
| 113 | APEAAGGPSVFLFPPKPKDTLMISRTPEVTCVVVDVSHEDPEVKFNWYVDGV | Clone CH3C.35.23.3 |
| | EVHNAKTKPREEQYNSTYRVVSVLTVLHQDWLNGKEYKCKVSNKALGAPIE | with knob, LALAPG, |
| | KTISKAKGQPREPQVYTLPPSRDELTKNQVSLWCLVKGFYPSDIAVEWESYGT | and M198L and N204S |
| | EWVNYKTTPPVLDSDGSFFLYSKLTVTKEEWQQGFVFSCSVLHEALHSHYTQ | mutations |
| | KSLSLSPGK | |
| 114 | APELLGGPSVFLFPPKPKDTLMISRTPEVTCVVVDVSHEDPEVKFNWYVDGVE | Clone CH3C.3523.3 |
| | VHNAKTKPREEQYNSTYRVVSVLTVLHQDWLNGKEYKCKVSNKALPAPIEK | with hole mutations |
| | TISKAKGQPREPQVYTLPPSRDELTKNQVSLSCAVKGFYPSDIAVEWESYGTE | |
| | WVNYKTTPPVLDSDGSFFLVSKLTVTKEEWQQGFVFSCSVMHEALHNHYTQ | |
| | KSLSLSPGK | |
| 115 | APEAAGGPSVFLFPPKPKDTLMISRTPEVTCVVVDVSHEDPEVKFNWYVDGV | Clone CH3C.35.23.3 |
| | EVHNAKTKPREEQYNSTYRVVSVLTVLHQDWLNGKEYKCKVSNKALPAPIE | with hole and LALA |
| | KTISKAKGQPREPQVYTLPPSRDELTKNQVSLSCAVKGFYPSDIAVEWESYGT | mutations |
| | EWVNYKTTPPVLDSDGSFFLVSKLTVTKEEWQQGFVFSCSVMHEALHNHYT | |
| | QKSLSLSPGK | |
| 116 | APEAAGGPSVFLFPPKPKDTLMISRTPEVTCVVVDVSHEDPEVKFNWYVDGV | Clone CH3C.35.23.3 |
| | EVHNAKTKPREEQYNSTYRVVSVLTVLHQDWLNGKEYKCKVSNKALGAPIE | with hole and LALAPG |
| | KTISKAKGQPREPQVYTLPPSRDELTKNQVSLSCAVKGFYPSDIAVEWESYGT | mutations |
| | EWVNYKTTPPVLDSDGSFFLVSKLTVTKEEWQQGFVFSCSVMHEALHNHYT | |
| | QKSLSLSPGK | |
| 117 | APELLGGPSVFLFPPKPKDTLYITREPEVTCVVVDVSHEDPEVKFNWYVDGVE | Clone CH3C.35.23.3 |
| | VHNAKTKPREEQYNSTYRVVSVLTVLHQDWLNGKEYKCKVSNKALPAPIEK | with hole and YTE |
| | TISKAKGQPREPQVYTLPPSRDELTKNQVSLSCAVKGFYPSDIAVEWESYGTE | mutations |
| | WVNYKTTPPVLDSDGSFFLVSKLTVTKEEWQQGFVFSCSVMHEALHNHYTQ | |
| | KSLSLSPGK | |
| 118 | APELLGGPSVFLFPPKPKDTLMISRTPEVTCVVVDVSHEDPEVKFNWYVDGVE | Clone CH3C.35.23.3 |
| | VHNAKTKPREEQYNSTYRVVSVLTVLHQDWLNGKEYKCKVSNKALPAPIEK | with hole and M198L |
| | TISKAKGQPREPQVYTLPPSRDELTKNQVSLSCAVKGFYPSDIAVEWESYGTE | and N204S mutations |
| | WVNYKTTPPVLDSDGSFFLVSKLTVTKEEWQQGFVFSCSVLHEALHSHYTQK | |
| | SLSLSPGK | |
| 119 | APEAAGGPSVFLFPPKPKDTLYITREPEVTCVVVDVSHEDPEVKFNWYVDGV | Clone CH3C.35.23.3 |
| | EVHNAKTKPREEQYNSTYRVVSVLTVLHQDWLNGKEYKCKVSNKALPAPIE | with hole, LALA, and |
| | KTISKAKGQPREPQVYTLPPSRDELTKNQVSLSCAVKGFYPSDIAVEWESYGT | YTE mutations |
| | EWVNYKTTPPVLDSDGSFFLVSKLTVTKEEWQQGFVFSCSVMHEALHNHYT | |
| | QKSLSLSPGK | |
| 120 | APEAAGGPSVFLFPPKPKDTLYITREPEVTCVVVDVSHEDPEVKFNWYVDGV | Clone CH3C.35.23.3 |
| | EVHNAKTKPREEQYNSTYRVVSVLTVLHQDWLNGKEYKCKVSNKALGAPIE | with hole, LALAPG, |
| | KTISKAKGQPREPQVYTLPPSRDELTKNQVSLSCAVKGFYPSDIAVEWESYGT | and YTE mutations |
| | EWVNYKTTPPVLDSDGSFFLVSKLTVTKEEWQQGFVFSCSVMHEALHNHYT | |
| | QKSLSLSPGK | |
| 121 | APEAAGGPSVFLFPPKPKDTLMISRTPEVTCVVVDVSHEDPEVKFNWYVDGV | Clone CH3C.35.23.3 |
| | EVHNAKTKPREEQYNSTYRVVSVLTVLHQDWLNGKEYKCKVSNKALPAPIE | with hole, LALA, and |
| | KTISKAKGQPREPQVYTLPPSRDELTKNQVSLSCAVKGFYPSDIAVEWESYGT | M198L and N204S |
| | EWVNYKTTPPVLDSDGSFFLVSKLTVTKEEWQQGFVFSCSVLHEALHSHYTQ | mutations |
| | KSLSLSPGK | |
| 122 | APEAAGGPSVFLFPPKPKDTLMISRTPEVTCVVVDVSHEDPEVKFNWYVDGV | Clone CH3C.35.23.3 |
| | EVHNAKTKPREEQYNSTYRVVSVLTVLHQDWLNGKEYKCKVSNKALGAPIE | with hole, LALAPG, |
| | KTISKAKGQPREPQVYTLPPSRDELTKNQVSLSCAVKGFYPSDIAVEWESYGT | and M198L and N204S |
| | EWVNYKTTPPVLDSDGSFFLVSKLTVTKEEWQQGFVFSCSVLHEALHSHYTQ | mutations |
| | KSLSLSPGK | |
| 123 | APELLGGPSVFLFPPKPKDTLMISRTPEVTCVVVDVSHEDPEVKFNWYVDGVE | Clone CH3C.35.23.4 |
| | VHNAKTKPREEQYNSTYRVVSVLTVLHQDWLNGKEYKCKVSNKALPAPIEK | with knob mutation |
| | TISKAKGQPREPQVYTLPPSRDELTKNQVSLWCLVKGFYPSDIAVEWESYGTE | |
| | WSNYKTTPPVLDSDGSFFLYSKLTVSKEEWQQGFVFSCSVMHEALHNHYTQ | |
| | KSLSLSPGK | |
| 124 | APEAAGGPSVFLFPPKPKDTLMISRTPEVTCVVVDVSHEDPEVKFNWYVDGV | Clone CH3C.35.23.4 |
| | EVHNAKTKPREEQYNSTYRVVSVLTVLHQDWLNGKEYKCKVSNKALPAPIE | with knob and LALA |
| | KTISKAKGQPREPQVYTLPPSRDELTKNQVSLWCLVKGFYPSDIAVEWESYGT | mutations |
| | EWSNYKTTPPVLDSDGSFFLYSKLTVSKEEWQQGFVFSCSVMHEALHNHYTQ | |
| | KSLSLSPGK | |
| 125 | APEAAGGPSVFLFPPKPKDTLMISRTPEVTCVVVDVSHEDPEVKFNWYVDGV | Clone CH3C.35.23.4 |
| | EVHNAKTKPREEQYNSTYRVVSVLTVLHQDWLNGKEYKCKVSNKALGAPIE | with knob and |
| | KTISKAKGQPREPQVYTLPPSRDELTKNQVSLWCLVKGFYPSDIAVEWESYGT | LALAPG mutations |
| | EWSNYKTTPPVLDSDGSFFLYSKLTVSKEEWQQGFVFSCSVMHEALHNHYTQ | |
| | KSLSLSPGK | |
| 126 | APELLGGPSVFLFPPKPKDTLYITREPEVTCVVVDVSHEDPEVKFNWYVDGVE | Clone CH3C.35.23.4 |
| | VHNAKTKPREEQYNSTYRVVSVLTVLHQDWLNGKEYKCKVSNKALPAPIEK | with knob and YTE |
| | TISKAKGQPREPQVYTLPPSRDELTKNQVSLWCLVKGFYPSDIAVEWESYGTE | mutations |
| | WSNYKTTPPVLDSDGSFFLYSKLTVSKEEWQQGFVFSCSVMHEALHNHYTQ | |
| | KSLSLSPGK | |
| 127 | APELLGGPSVFLFPPKPKDTLMISRTPEVTCVVVDVSHEDPEVKFNWYVDGVE | Clone CH3C.35.23.4 |
| | VHNAKTKPREEQYNSTYRVVSVLTVLHQDWLNGKEYKCKVSNKALPAPIEK | with knob and M198L |
| | TISKAKGQPREPQVYTLPPSRDELTKNQVSLWCLVKGFYPSDIAVEWESYGTE | and N204S mutations |
| | WSNYKTTPPVLDSDGSFFLYSKLTVSKEEWQQGFVFSCSVLHEALHSHYTQK | |
| | SLSLSPGK | |
| 128 | APEAAGGPSVFLFPPKPKDTLYITREPEVTCVVVDVSHEDPEVKFNWYVDGV | Clone CH3C.35.23.4 |
| | EVHNAKTKPREEQYNSTYRVVSVLTVLHQDWLNGKEYKCKVSNKALPAPIE | with knob, LALA, and |
| | KTISKAKGQPREPQVYTLPPSRDELTKNQVSLWCLVKGFYPSDIAVEWESYGT | YTE mutations |
| | EWSNYKTTPPVLDSDGSFFLYSKLTVSKEEWQQGFVFSCSVMHEALHNHYTQ | |
| | KSLSLSPGK | |
| 129 | APEAAGGPSVFLFPPKPKDTLYITREPEVTCVVVDVSHEDPEVKFNWYVDGV | Clone CH3C.35.23.4 |
| | EVHNAKTKPREEQYNSTYRVVSVLTVLHQDWLNGKEYKCKVSNKALGAPIE | with knob, LALAPG, |
| | KTISKAKGQPREPQVYTLPPSRDELTKNQVSLWCLVKGFYPSDIAVEWESYGT | and YTE mutations |
| | EWSNYKTTPPVLDSDGSFFLYSKLTVSKEEWQQGFVFSCSVMHEALHNHYTQ | |
| | KSLSLSPGK | |
| 130 | APEAAGGPSVFLFPPKPKDTLMISRTPEVTCVVVDVSHEDPEVKFNWYVDGV | Clone CH3C.35.23.4 |
| | EVHNAKTKPREEQYNSTYRVVSVLTVLHQDWLNGKEYKCKVSNKALPAPIE | with knob, LALA, and |
| | KTISKAKGQPREPQVYTLPPSRDELTKNQVSLWCLVKGFYPSDIAVEWESYGT | M198L and N204S |
| | EWSNYKTTPPVLDSDGSFFLYSKLTVSKEEWQQGFVFSCSVLHEALHSHYTQ | mutations |
| | KSLSLSPGK | |
| 131 | APEAAGGPSVFLFPPKPKDTLMISRTPEVTCVVVDVSHEDPEVKFNWYVDGV | Clone CH3C.35.23.4 |
| | EVHNAKTKPREEQYNSTYRVVSVLTVLHQDWLNGKEYKCKVSNKALGAPIE | with knob, LALAPG, |
| | KTISKAKGQPREPQVYTLPPSRDELTKNQVSLWCLVKGFYPSDIAVEWESYGT | and M198L and N204S |
| | EWSNYKTTPPVLDSDGSFFLYSKLTVSKEEWQQGFVFSCSVLHEALHSHYTQ | mutations |
| | KSLSLSPGK | |
| 132 | APELLGGPSVFLFPPKPKDTLMISRTPEVTCVVVDVSHEDPEVKFNWYVDGVE | Clone CH3C.35.23.4 |
| | VHNAKTKPREEQYNSTYRVVSVLTVLHQDWLNGKEYKCKVSNKALPAPIEK | with hole mutations |
| | TISKAKGQPREPQVYTLPPSRDELTKNQVSLSCAVKGFYPSDIAVEWESYGTE | |
| | WSNYKTTPPVLDSDGSFFLVSKLTVSKEEWQQGFVFSCSVMHEALHNHYTQ | |
| | KSLSLSPGK | |
| 133 | APEAAGGPSVFLFPPKPKDTLMISRTPEVTCVVVDVSHEDPEVKFNWYVDGV | Clone CH3C.35.23.4 |
| | EVHNAKTKPREEQYNSTYRVVSVLTVLHQDWLNGKEYKCKVSNKALPAPIE | with hole and LALA |
| | KTISKAKGQPREPQVYTLPPSRDELTKNQVSLSCAVKGFYPSDIAVEWESYGT | mutations |
| | EWSNYKTTPPVLDSDGSFFLVSKLTVSKEEWQQGFVFSCSVMHEALHNHYTQ | |
| | KSLSLSPGK | |
| 134 | APEAAGGPSVFLFPPKPKDTLMISRTPEVTCVVVDVSHEDPEVKFNWYVDGV | Clone CH3C.35.23.4 |
| | EVHNAKTKPREEQYNSTYRVVSVLTVLHQDWLNGKEYKCKVSNKALGAPIE | with hole and LALAPG |
| | KTISKAKGQPREPQVYTLPPSRDELTKNQVSLSCAVKGFYPSDIAVEWESYGT | mutations |
| | EWSNYKTTPPVLDSDGSFFLVSKLTVSKEEWQQGFVFSCSVMHEALHNHYTQ | |
| | KSLSLSPGK | |
| 135 | APELLGGPSVFLFPPKPKDTLYITREPEVTCVVVDVSHEDPEVKFNWYVDGVE | Clone CH3C.35.23.4 |
| | VHNAKTKPREEQYNSTYRVVSVLTVLHQDWLNGKEYKCKVSNKALPAPIEK | with hole and YTE |
| | TISKAKGQPREPQVYTLPPSRDELTKNQVSLSCAVKGFYPSDIAVEWESYGTE | mutations |
| | WSNYKTTPPVLDSDGSFFLVSKLTVSKEEWQQGFVFSCSVMHEALHNHYTQ | |
| | KSLSLSPGK | |
| 136 | APELLGGPSVFLFPPKPKDTLMISRTPEVTCVVVDVSHEDPEVKFNWYVDGVE | Clone CH3C.35.23.4 |
| | VHNAKTKPREEQYNSTYRVVSVLTVLHQDWLNGKEYKCKVSNKALPAPIEK | with hole and M198L |
| | TISKAKGQPREPQVYTLPPSRDELTKNQVSLSCAVKGFYPSDIAVEWESYGTE | and N204S mutations |
| | WSNYKTTPPVLDSDGSFFLVSKLTVSKEEWQQGFVFSCSVLHEALHSHYTQK | |
| | SLSLSPGK | |
| 137 | APEAAGGPSVFLFPPKPKDTLYITREPEVTCVVVDVSHEDPEVKFNWYVDGV | Clone CH3C.35.23.4 |
| | EVHNAKTKPREEQYNSTYRVVSVLTVLHQDWLNGKEYKCKVSNKALPAPIE | with hole, LALA, and |
| | KTISKAKGQPREPQVYTLPPSRDELTKNQVSLSCAVKGFYPSDIAVEWESYGT | YTE mutations |
| | EWSNYKTTPPVLDSDGSFFLVSKLTVSKEEWQQGFVFSCSVMHEALHNHYTQ | |
| | KSLSLSPGK | |
| 138 | APEAAGGPSVFLFPPKPKDTLYITREPEVTCVVVDVSHEDPEVKFNWYVDGV | Clone CH3C.35.23.4 |
| | EVHNAKTKPREEQYNSTYRVVSVLTVLHQDWLNGKEYKCKVSNKALGAPIE | with hole, LALAPG, |
| | KTISKAKGQPREPQVYTLPPSRDELTKNQVSLSCAVKGFYPSDIAVEWESYGT | and YTE mutations |
| | EWSNYKTTPPVLDSDGSFFLVSKLTVSKEEWQQGFVFSCSVMHEALHNHYTQ | |
| | KSLSLSPGK | |
| 139 | APEAAGGPSVFLFPPKPKDTLMISRTPEVTCVVVDVSHEDPEVKFNWYVDGV | Clone CH3C.35.23.4 |
| | EVHNAKTKPREEQYNSTYRVVSVLTVLHQDWLNGKEYKCKVSNKALPAPIE | with hole, LALA, and |
| | KTISKAKGQPREPQVYTLPPSRDELTKNQVSLSCAVKGFYPSDIAVEWESYGT | M198L and N204S |
| | EWSNYKTTPPVLDSDGSFFLVSKLTVSKEEWQQGFVFSCSVLHEALHSHYTQ | mutations |
| | KSLSLSPGK | |
| 140 | APEAAGGPSVFLFPPKPKDTLMISRTPEVTCVVVDVSHEDPEVKFNWYVDGV | Clone CH3C.35.23.4 |
| | EVHNAKTKPREEQYNSTYRVVSVLTVLHQDWLNGKEYKCKVSNKALGAPIE | with hole, LALAPG, |
| | KTISKAKGQPREPQVYTLPPSRDELTKNQVSLSCAVKGFYPSDIAVEWESYGT | and M198L and N204S |
| | EWSNYKTTPPVLDSDGSFFLVSKLTVSKEEWQQGFVFSCSVLHEALHSHYTQ | mutations |
| | KSLSLSPGK | |
| 141 | APELLGGPSVFLFPPKPKDTLMISRTPEVTCVVVDVSHEDPEVKFNWYVDGVE | Clone CH3C.35.23 |
| | VHNAKTKPREEQYNSTYRVVSVLTVLHQDWLNGKEYKCKVSNKALPAPIEK | with knob mutation |
| | TISKAKGQPREPQVYTLPPSRDELTKNQVSLWCLVKGFYPSDIAVEWESYGTE | |
| | WSNYKTTPPVLDSDGSFFLYSKLTVTKEEWQQGFVFSCSVMHEALHNHYTQ | |
| | KSLSLSPGK | |
| 142 | APEAAGGPSVFLFPPKPKDTLMISRTPEVTCVVVDVSHEDPEVKFNWYVDGV | Clone CH3C.35.23 |
| | EVHNAKTKPREEQYNSTYRVVSVLTVLHQDWLNGKEYKCKVSNKALPAPIE | with knob and LALA |
| | KTISKAKGQPREPQVYTLPPSRDELTKNQVSLWCLVKGFYPSDIAVEWESYGT | mutations |
| | EWSNYKTTPPVLDSDGSFFLYSKLTVTKEEWQQGFVFSCSVMHEALHNHYTQ | |
| | KSLSLSPGK | |
| 143 | APEAAGGPSVFLFPPKPKDTLMISRTPEVTCVVVDVSHEDPEVKFNWYVDGV | Clone CH3C.35.23 |
| | EVHNAKTKPREEQYNSTYRVVSVLTVLHQDWLNGKEYKCKVSNKALGAPIE | with knob and |
| | KTISKAKGQPREPQVYTLPPSRDELTKNQVSLWCLVKGFYPSDIAVEWESYGT | LALAPG mutations |
| | EWSNYKTTPPVLDSDGSFFLYSKLTVTKEEWQQGFVFSCSVMHEALHNHYTQ | |
| | KSLSLSPGK | |
| 144 | APELLGGPSVFLFPPKPKDTLYITREPEVTCVVVDVSHEDPEVKFNWYVDGVE | Clone CH3C.35.23 |
| | VHNAKTKPREEQYNSTYRVVSVLTVLHQDWLNGKEYKCKVSNKALPAPIEK | with knob and YTE |
| | TISKAKGQPREPQVYTLPPSRDELTKNQVSLWCLVKGFYPSDIAVEWESYGTE | mutations |
| | WSNYKTTPPVLDSDGSFFLYSKLTVTKEEWQQGFVFSCSVMHEALHNHYTQ | |
| | KSLSLSPGK | |
| 145 | APELLGGPSVFLFPPKPKDTLMISRTPEVTCVVVDVSHEDPEVKFNWYVDGVE | Clone CH3C.35.23 |
| | VHNAKTKPREEQYNSTYRVVSVLTVLHQDWLNGKEYKCKVSNKALPAPIEK | with knob and M198L |
| | TISKAKGQPREPQVYTLPPSRDELTKNQVSLWCLVKGFYPSDIAVEWESYGTE | and N204S mutations |
| | WSNYKTTPPVLDSDGSFFLYSKLTVTKEEWQQGFVFSCSVLHEALHSHYTQK | |
| | SLSLSPGK | |
| 146 | APEAAGGPSVFLFPPKPKDTLYITREPEVTCVVVDVSHEDPEVKFNWYVDGV | Clone CH3C.35.23 |
| | EVHNAKTKPREEQYNSTYRVVSVLTVLHQDWLNGKEYKCKVSNKALPAPIE | with knob, LALA, and |
| | KTISKAKGQPREPQVYTLPPSRDELTKNQVSLWCLVKGFYPSDIAVEWESYGT | YTE mutations |
| | EWSNYKTTPPVLDSDGSFFLYSKLTVTKEEWQQGFVFSCSVMHEALHNHYTQ | |
| | KSLSLSPGK | |
| 147 | APEAAGGPSVFLFPPKPKDTLYITREPEVTCVVVDVSHEDPEVKFNWYVDGV | Clone CH3C.35.23 |
| | EVHNAKTKPREEQYNSTYRVVSVLTVLHQDWLNGKEYKCKVSNKALGAPIE | with knob, LALAPG, |
| | KTISKAKGQPREPQVYTLPPSRDELTKNQVSLWCLVKGFYPSDIAVEWESYGT | and YTE mutations |
| | EWSNYKTTPPVLDSDGSFFLYSKLTVTKEEWQQGFVFSCSVMHEALHNHYTQ | |
| | KSLSLSPGK | |
| 148 | APEAAGGPSVFLFPPKPKDTLMISRTPEVTCVVVDVSHEDPEVKFNWYVDGV | Clone CH3C.35.23 |
| | EVHNAKTKPREEQYNSTYRVVSVLTVLHQDWLNGKEYKCKVSNKALPAPIE | with knob, LALA, and |
| | KTISKAKGQPREPQVYTLPPSRDELTKNQVSLWCLVKGFYPSDIAVEWESYGT | M198L and N204S |
| | EWSNYKTTPPVLDSDGSFFLYSKLTVTKEEWQQGFVFSCSVLHEALHSHYTQ | mutations |
| | KSLSLSPGK | |
| 149 | APEAAGGPSVFLFPPKPKDTLMISRTPEVTCVVVDVSHEDPEVKFNWYVDGV | Clone CH3C.35.23 |
| | EVHNAKTKPREEQYNSTYRVVSVLTVLHQDWLNGKEYKCKVSNKALGAPIE | with knob, LALAPG, |
| | KTISKAKGQPREPQVYTLPPSRDELTKNQVSLWCLVKGFYPSDIAVEWESYGT | and M198L and N204S |
| | EWSNYKTTPPVLDSDGSFFLYSKLTVTKEEWQQGFVFSCSVLHEALHSHYTQ | mutations |
| | KSLSLSPGK | |
| 150 | APELLGGPSVFLFPPKPKDTLMISRTPEVTCVVVDVSHEDPEVKFNWYVDGVE | Clone CH3C.35.23 |
| | VHNAKTKPREEQYNSTYRVVSVLTVLHQDWLNGKEYKCKVSNKALPAPIEK | with hole mutations |
| | TISKAKGQPREPQVYTLPPSRDELTKNQVSLSCAVKGFYPSDIAVEWESYGTE | |
| | WSNYKTTPPVLDSDGSFFLVSKLTVTKEEWQQGFVFSCSVMHEALHNHYTQ | |
| | KSLSLSPGK | |
| 151 | APEAAGGPSVFLFPPKPKDTLMISRTPEVTCVVVDVSHEDPEVKFNWYVDGV | Clone CH3C.35.23 |
| | EVHNAKTKPREEQYNSTYRVVSVLTVLHQDWLNGKEYKCKVSNKALPAPIE | with hole and LALA |
| | KTISKAKGQPREPQVYTLPPSRDELTKNQVSLSCAVKGFYPSDIAVEWESYGT | mutations |
| | EWSNYKTTPPVLDSDGSFFLVSKLTVTKEEWQQGFVFSCSVMHEALHNHYTQ | |
| | KSLSLSPGK | |
| 152 | APEAAGGPSVFLFPPKPKDTLMISRTPEVTCVVVDVSHEDPEVKFNWYVDGV | Clone CH3C.35.23 |
| | EVHNAKTKPREEQYNSTYRVVSVLTVLHQDWLNGKEYKCKVSNKALGAPIE | with hole and LALAPG |
| | KTISKAKGQPREPQVYTLPPSRDELTKNQVSLSCAVKGFYPSDIAVEWESYGT | mutations |
| | EWSNYKTTPPVLDSDGSFFLVSKLTVTKEEWQQGFVFSCSVMHEALHNHYTQ | |
| | KSLSLSPGK | |
| 153 | APELLGGPSVFLFPPKPKDTLYITREPEVTCVVVDVSHEDPEVKFNWYVDGVE | Clone CH3C.35.23 |
| | VHNAKTKPREEQYNSTYRVVSVLTVLHQDWLNGKEYKCKVSNKALPAPIEK | with hole and YTE |
| | TISKAKGQPREPQVYTLPPSRDELTKNQVSLSCAVKGFYPSDIAVEWESYGTE | mutations |
| | WSNYKTTPPVLDSDGSFFLVSKLTVTKEEWQQGFVFSCSVMHEALHNHYTQ | |
| | KSLSLSPGK | |
| 154 | APELLGGPSVFLFPPKPKDTLMISRTPEVTCVVVDVSHEDPEVKFNWYVDGVE | Clone CH3C.35.23 |
| | VHNAKTKPREEQYNSTYRVVSVLTVLHQDWLNGKEYKCKVSNKALPAPIEK | with hole and M198L |
| | TISKAKGQPREPQVYTLPPSRDELTKNQVSLSCAVKGFYPSDIAVEWESYGTE | and N204S mutations |
| | WSNYKTTPPVLDSDGSFFLVSKLTVTKEEWQQGFVFSCSVLHEALHSHYTQK | |
| | SLSLSPGK | |
| 155 | APEAAGGPSVFLFPPKPKDTLYITREPEVTCVVVDVSHEDPEVKFNWYVDGV | Clone CH3C.35.23 |
| | EVHNAKTKPREEQYNSTYRVVSVLTVLHQDWLNGKEYKCKVSNKALPAPIE | with hole, LALA, and |
| | KTISKAKGQPREPQVYTLPPSRDELTKNQVSLSCAVKGFYPSDIAVEWESYGT | YTE mutations |
| | EWSNYKTTPPVLDSDGSFFLVSKLTVTKEEWQQGFVFSCSVMHEALHNHYTQ | |
| | KSLSLSPGK | |
| 156 | APEAAGGPSVFLFPPKPKDTLYITREPEVTCVVVDVSHEDPEVKFNWYVDGV | Clone CH3C.35.23 |
| | EVHNAKTKPREEQYNSTYRVVSVLTVLHQDWLNGKEYKCKVSNKALGAPIE | with hole, LALAPG, |
| | KTISKAKGQPREPQVYTLPPSRDELTKNQVSLSCAVKGFYPSDIAVEWESYGT | and YTE mutations |
| | EWSNYKTTPPVLDSDGSFFLVSKLTVTKEEWQQGFVFSCSVMHEALHNHYTQ | |
| | KSLSLSPGK | |
| 157 | APEAAGGPSVFLFPPKPKDTLMISRTPEVTCVVVDVSHEDPEVKFNWYVDGV | Clone CH3C.35.23 |
| | EVHNAKTKPREEQYNSTYRVVSVLTVLHQDWLNGKEYKCKVSNKALPAPIE | with hole, LALA, and |
| | KTISKAKGQPREPQVYTLPPSRDELTKNQVSLSCAVKGFYPSDIAVEWESYGT | M198L and N204S |
| | EWSNYKTTPPVLDSDGSFFLVSKLTVTKEEWQQGFVFSCSVLHEALHSHYTQ | mutations |
| | KSLSLSPGK | |
| 158 | APEAAGGPSVFLFPPKPKDTLMISRTPEVTCVVVDVSHEDPEVKFNWYVDGV | Clone CH3C.35.23 |
| | EVHNAKTKPREEQYNSTYRVVSVLTVLHQDWLNGKEYKCKVSNKALGAPIE | with ole, h LALAP G, |
| | KTISKAKGQPREPQVYTLPPSRDELTKNQVSLSCAVKGFYPSDIAVEWESYGT | and M198L and N204S |
| | EWSNYKTTPPVLDSDGSFFLVSKLTVTKEEWQQGFVFSCSVLHEALHSHYTQ | mutations |
| | KSLSLSPGK | |
| 159 | APELLGGPSVFLFPPKPKDTLMISRTPEVTCVVVDVSHEDPEVKFNWYVDGVE | Clone CH3C.35.23.1.1 |
| | VHNAKTKPREEQYNSTYRVVSVLTVLHQDWLNGKEYKCKVSNKALPAPIEK | with knob mutation |
| | TISKAKGQPREPQVYTLPPSRDELTKNQVSLWCLVKGFYPSDIAVEWESFGTE | |
| | WSNYKTTPPVLDSDGSFFLYSKLTVSKEEWQQGFVFSCSVMHEALHNHYTQ | |
| | KSLSLSPGK | |
| 160 | APEAAGGPSVFLFPPKPKDTLMISRTPEVTCVVVDVSHEDPEVKFNWYVDGV | Clone CH3C.35.23.1.1 |
| | EVHNAKTKPREEQYNSTYRVVSVLTVLHQDWLNGKEYKCKVSNKALPAPIE | with knob and LALA |
| | KTISKAKGQPREPQVYTLPPSRDELTKNQVSLWCLVKGFYPSDIAVEWESFGT | mutations |
| | EWSNYKTTPPVLDSDGSFFLYSKLTVSKEEWQQGFVFSCSVMHEALHNHYTQ | |
| | KSLSLSPGK | |
| 161 | APEAAGGPSVFLFPPKPKDTLMISRTPEVTCVVVDVSHEDPEVKFNWYVDGV | Clone CH3C.35.23.1.1 |
| | EVHNAKTKPREEQYNSTYRVVSVLTVLHQDWLNGKEYKCKVSNKALGAPIE | with knob and |
| | KTISKAKGQPREPQVYTLPPSRDELTKNQVSLWCLVKGFYPSDIAVEWESFGT | LALAPG mutations |
| | EWSNYKTTPPVLDSDGSFFLYSKLTVSKEEWQQGFVFSCSVMHEALHNHYTQ | |
| | KSLSLSPGK | |
| 162 | APELLGGPSVFLFPPKPKDTLYITREPEVTCVVVDVSHEDPEVKFNWYVDGVE | Clone CH3C.35.23.1.1 |
| | VHNAKTKPREEQYNSTYRVVSVLTVLHQDWLNGKEYKCKVSNKALPAPIEK | with knob and YTE |
| | TISKAKGQPREPQVYTLPPSRDELTKNQVSLWCLVKGFYPSDIAVEWESFGTE | mutations |
| | WSNYKTTPPVLDSDGSFFLYSKLTVSKEEWQQGFVFSCSVMHEALHNHYTQ | |
| | KSLSLSPGK | |
| 163 | APELLGGPSVFLFPPKPKDTLMISRTPEVTCVVVDVSHEDPEVKFNWYVDGVE | Clone CH3C.35.23.1.1 |
| | VHNAKTKPREEQYNSTYRVVSVLTVLHQDWLNGKEYKCKVSNKALPAPIEK | with knob and M198L |
| | TISKAKGQPREPQVYTLPPSRDELTKNQVSLWCLVKGFYPSDIAVEWESFGTE | and N204S mutations |
| | WSNYKTTPPVLDSDGSFFLYSKLTVSKEEWQQGFVFSCSVLHEALHSHYTQK | |
| | SLSLSPGK | |
| 164 | APEAAGGPSVFLFPPKPKDTLYITREPEVTCVVVDVSHEDPEVKFNWYVDGV | Clone CH3C.35.23.1.1 |
| | EVHNAKTKPREEQYNSTYRVVSVLTVLHQDWLNGKEYKCKVSNKALPAPIE | with knob, LALA, and |
| | KTISKAKGQPREPQVYTLPPSRDELTKNQVSLWCLVKGFYPSDIAVEWESFGT | YTE mutations |
| | EWSNYKTTPPVLDSDGSFFLYSKLTVSKEEWQQGFVFSCSVMHEALHNHYTQ | |
| | KSLSLSPGK | |
| 165 | APEAAGGPSVFLFPPKPKDTLYITREPEVTCVVVDVSHEDPEVKFNWYVDGV | Clone CH3C.35.23.1.1 |
| | EVHNAKTKPREEQYNSTYRVVSVLTVLHQDWLNGKEYKCKVSNKALGAPIE | with knob, LALAPG, |
| | KTISKAKGQPREPQVYTLPPSRDELTKNQVSLWCLVKGFYPSDIAVEWESFGT | and YTE mutations |
| | EWSNYKTTPPVLDSDGSFFLYSKLTVSKEEWQQGFVFSCSVMHEALHNHYTQ | |
| | KSLSLSPGK | |
| 166 | APEAAGGPSVFLFPPKPKDTLMISRTPEVTCVVVDVSHEDPEVKFNWYVDGV | Clone CH3C.35.23.1.1 |
| | EVHNAKTKPREEQYNSTYRVVSVLTVLHQDWLNGKEYKCKVSNKALPAPIE | with knob, LALA, and |
| | KTISKAKGQPREPQVYTLPPSRDELTKNQVSLWCLVKGFYPSDIAVEWESFGT | M198L and N204S |
| | EWSNYKTTPPVLDSDGSFFLYSKLTVSKEEWQQGFVFSCSVLHEALHSHYTQ | mutations |
| | KSLSLSPGK | |
| 167 | APEAAGGPSVFLFPPKPKDTLMISRTPEVTCVVVDVSHEDPEVKFNWYVDGV | Clone CH3C.35.23.1.1 |
| | EVHNAKTKPREEQYNSTYRVVSVLTVLHQDWLNGKEYKCKVSNKALGAPIE | with knob, LALAPG, |
| | KTISKAKGQPREPQVYTLPPSRDELTKNQVSLWCLVKGFYPSDIAVEWESFGT | and M198L and N204S |
| | EWSNYKTTPPVLDSDGSFFLYSKLTVSKEEWQQGFVFSCSVLHEALHSHYTQ | mutations |
| | KSLSLSPGK | |
| 168 | APELLGGPSVFLFPPKPKDTLMISRTPEVTCVVVDVSHEDPEVKFNWYVDGVE | Clone CH3C.35.23.1.1 |
| | VHNAKTKPREEQYNSTYRVVSVLTVLHQDWLNGKEYKCKVSNKALPAPIEK | with hole mutations |
| | TISKAKGQPREPQVYTLPPSRDELTKNQVSLSCAVKGFYPSDIAVEWESFGTE | |
| | WSNYKTTPPVLDSDGSFFLVSKLTVSKEEWQQGFVFSCSVMHEALHNHYTQ | |
| | KSLSLSPGK | |
| 169 | APEAAGGPSVFLFPPKPKDTLMISRTPEVTCVVVDVSHEDPEVKFNWYVDGV | Clone CH3C.35.23.1.1 |
| | EVHNAKTKPREEQYNSTYRVVSVLTVLHQDWLNGKEYKCKVSNKALPAPIE | with hole and LALA |
| | KTISKAKGQPREPQVYTLPPSRDELTKNQVSLSCAVKGFYPSDIAVEWESFGT | mutations |
| | EWSNYKTTPPVLDSDGSFFLVSKLTVSKEEWQQGFVFSCSVMHEALHNHYTQ | |
| | KSLSLSPGK | |
| 170 | APEAAGGPSVFLFPPKPKDTLMISRTPEVTCVVVDVSHEDPEVKFNWYVDGV | Clone CH3C.35.23.1.1 |
| | EVHNAKTKPREEQYNSTYRVVSVLTVLHQDWLNGKEYKCKVSNKALGAPIE | with hole and LALAPG |
| | KTISKAKGQPREPQVYTLPPSRDELTKNQVSLSCAVKGFYPSDIAVEWESFGT | mutations |
| | EWSNYKTTPPVLDSDGSFFLVSKLTVSKEEWQQGFVFSCSVMHEALHNHYTQ | |
| | KSLSLSPGK | |
| 171 | APELLGGPSVFLFPPKPKDTLYITREPEVTCVVVDVSHEDPEVKFNWYVDGVE | Clone CH3C.35.23.1.1 |
| | VHNAKTKPREEQYNSTYRVVSVLTVLHQDWLNGKEYKCKVSNKALPAPIEK | with hole and YTE |
| | TISKAKGQPREPQVYTLPPSRDELTKNQVSLSCAVKGFYPSDIAVEWESFGTE | mutations |
| | WSNYKTTPPVLDSDGSFFLVSKLTVSKEEWQQGFVFSCSVMHEALHNHYTQ | |
| | KSLSLSPGK | |
| 172 | APELLGGPSVFLFPPKPKDTLMISRTPEVTCVVVDVSHEDPEVKFNWYVDGVE | Clone CH3C.35.23.1.1 |
| | VHNAKTKPREEQYNSTYRVVSVLTVLHQDWLNGKEYKCKVSNKALPAPIEK | with hole and M198L |
| | TISKAKGQPREPQVYTLPPSRDELTKNQVSLSCAVKGFYPSDIAVEWESFGTE | and N204S mutations |
| | WSNYKTTPPVLDSDGSFFLVSKLTVSKEEWQQGFVFSCSVLHEALHSHYTQK | |
| | SLSLSPGK | |
| 173 | APEAAGGPSVFLFPPKPKDTLYITREPEVTCVVVDVSHEDPEVKFNWYVDGV | Clone CH3C.35.23.1.1 |
| | EVHNAKTKPREEQYNSTYRVVSVLTVLHQDWLNGKEYKCKVSNKALPAPIE | with hole, LALA, and |
| | KTISKAKGQPREPQVYTLPPSRDELTKNQVSLSCAVKGFYPSDIAVEWESFGT | YTE mutations |
| | EWSNYKTTPPVLDSDGSFFLVSKLTVSKEEWQQGFVFSCSVMHEALHNHYTQ | |
| | KSLSLSPGK | |
| 174 | APEAAGGPSVFLFPPKPKDTLYITREPEVTCVVVDVSHEDPEVKFNWYVDGV | Clone CH3C.35.23.1.1 |
| | EVHNAKTKPREEQYNSTYRVVSVLTVLHQDWLNGKEYKCKVSNKALGAPIE | with hole, LALAPG, |
| | KTISKAKGQPREPQVYTLPPSRDELTKNQVSLSCAVKGFYPSDIAVEWESFGT | and YTE mutations |
| | EWSNYKTTPPVLDSDGSFFLVSKLTVSKEEWQQGFVFSCSVMHEALHNHYTQ | |
| | KSLSLSPGK | |
| 175 | APEAAGGPSVFLFPPKPKDTLMISRTPEVTCVVVDVSHEDPEVKFNWYVDGV | Clone CH3C.35.23.1.1 |
| | EVHNAKTKPREEQYNSTYRVVSVLTVLHQDWLNGKEYKCKVSNKALPAPIE | with hole, LALA, and |
| | KTISKAKGQPREPQVYTLPPSRDELTKNQVSLSCAVKGFYPSDIAVEWESFGT | M198L and N204S |
| | EWSNYKTTPPVLDSDGSFFLVSKLTVSKEEWQQGFVFSCSVLHEALHSHYTQ | mutations |
| | KSLSLSPGK | |
| 176 | APEAAGGPSVFLFPPKPKDTLMISRTPEVTCVVVDVSHEDPEVKFNWYVDGV | Clone CH3C.35.23.1.1 |
| | EVHNAKTKPREEQYNSTYRVVSVLTVLHQDWLNGKEYKCKVSNKALGAPIE | with hole, LALAPG, |
| | KTISKAKGQPREPQVYTLPPSRDELTKNQVSLSCAVKGFYPSDIAVEWESFGT | and M198L and N204S |
| | EWSNYKTTPPVLDSDGSFFLVSKLTVSKEEWQQGFVFSCSVLHEALHSHYTQ | mutations |
| | KSLSLSPGK | |
| 177 | APELLGGPSVFLFPPKPKDTLMISRTPEVTCVVVDVSHEDPEVKFNWYVDGVE | Clone CH3C.35.23 |
| | VHNAKTKPREEQYNSTYRVVSVLTVLHQDWLNGKEYKCKVSNKALPAPIEK | |
| | TISKAKGQPREPQVYTLPPSRDELTKNQVSLTCLVKGFYPSDIAVEWESYGTE | |
| | WSNYKTTPPVLDSDGSFFLYSKLTVTKEEWQQGFVFSCSVMHEALHNHYTQ | |
| | KSLSLSPGK | |
| 178 | APELLGGPSVFLFPPKPKDTLMISRTPEVTCVVVDVSHEDPEVKFNWYVDGVE | Clone CH3C.35.23.3 |
| | VHNAKTKPREEQYNSTYRVVSVLTVLHQDWLNGKEYKCKVSNKALPAPIEK | |
| | TISKAKGQPREPQVYTLPPSRDELTKNQVSLTCLVKGFYPSDIAVEWESYGTE | |
| | WVNYKTTPPVLDSDGSFFLYSKLTVTKEEWQQGFVFSCSVMHEALHNHYTQ | |
| | KSLSLSPGK | |
| 179 | APELLGGPSVFLFPPKPKDTLMISRTPEVTCVVVDVSHEDPEVKFNWYVDGVE | Clone CH3C.35.23.4 |
| | VHNAKTKPREEQYNSTYRVVSVLTVLHQDWLNGKEYKCKVSNKALPAPIEK | |
| | TISKAKGQPREPQVYTLPPSRDELTKNQVSLTCLVKGFYPSDIAVEWESYGTE | |
| | WSNYKTTPPVLDSDGSFFLYSKLTVSKEEWQQGFVFSCSVMHEALHNHYTQ | |
| | KSLSLSPGK | |
| 180 | APELLGGPSVFLFPPKPKDTLMISRTPEVTCVVVDVSHEDPEVKFNWYVDGVE | Clone CH3C.35.23.1.1 |
| | VHNAKTKPREEQYNSTYRVVSVLTVLHQDWLNGKEYKCKVSNKALPAPIEK | |
| | TISKAKGQPREPQVYTLPPSRDELTKNQVSLTCLVKGFYPSDIAVEWESFGTE | |
| | WSNYKTTPPVLDSDGSFFLYSKLTVSKEEWQQGFVFSCSVMHEALHNHYTQ | |
| | KSLSLSPGK | |
| 181 | APELLGGPSVFLFPPKPKDTLMISRTPEVTCVVVDVSHEDPEVKFNWYVDGVE | Clone CH3C.18 variant |
| | VHNAKTKPREEQYNSTYRVVSVLTVLHQDWLNGKEYKCKVSNKALPAPIEK | 1 |
| | TISKAKGQPREPQVYTLPPSRDELTKNQVSLTCLVKGFYPSDIAVLWESLGHV | |
| | WAVYKTTPPVLDSDGSFFLYSKLTVPKSTWQQGWVFSCSVMHEALHNHYTQ | |
| | KSLSLSPGK | |
| 182 | APELLGGPSVFLFPPKPKDTLMISRTPEVTCVVVDVSHEDPEVKFNWYVDGVE | Clone CH3C.18 variant |
| | VHNAKTKPREEQYNSTYRVVSVLTVLHQDWLNGKEYKCKVSNKALPAPIEK | 2 |
| | TISKAKGQPREPQVYTLPPSRDELTKNQVSLTCLVKGFYPSDIAVYWESLGHV | |
| | WAVYKTTPPVLDSDGSFFLYSKLTVPKSTWQQGWVFSCSVMHEALHNHYTQ | |
| | KSLSLSPGK | |
| 183 | APELLGGPSVFLFPPKPKDTLMISRTPEVTCVVVDVSHEDPEVKFNWYVDGVE | Clone CH3C.18 variant |
| | VHNAKTKPREEQYNSTYRVVSVLTVLHQDWLNGKEYKCKVSNKALPAPIEK | 3 |
| | TISKAKGQPREPQVYTLPPSRDELTKNQVSLTCLVKGFYPSDIAVEWESLGHV | |
| | WAVYFTTPPVLDSDGSFFLYSKLTVPKSTWQQGWVFSCSVMHEALHNHYTQ | |
| | KSLSLSPGK | |
| 184 | APELLGGPSVFLFPPKPKDTLMISRTPEVTCVVVDVSHEDPEVKFNWYVDGVE | Clone CH3C.18 variant |
| | VHNAKTKPREEQYNSTYRVVSVLTVLHQDWLNGKEYKCKVSNKALPAPIEK | 4 |
| | TISKAKGQPREPQVYTLPPSRDELTKNQVSLTCLVKGFYPSDIAVEWESLGHV | |
| | WAVYHTTPPVLDSDGSFFLYSKLTVPKSTWQQGWVFSCSVMHEALHNHYTQ | |
| | KSLSLSPGK | |
| 185 | APELLGGPSVFLFPPKPKDTLMISRTPEVTCVVVDVSHEDPEVKFNWYVDGVE | Clone CH3C.35.13 |
| | VHNAKTKPREEQYNSTYRVVSVLTVLHQDWLNGKEYKCKVSNKALPAPIEK | |
| | TISKAKGQPREPQVYTLPPSRDELTKNQVSLTCLVKGFYPSDIAVWWESLGHV | |
| | WAVYKTTPPVLDSDGSFFLYSKLTVPKSTWQQGWVFSCSVMHEALHNHYTQ | |
| | KSLSLSPGK | |
| 186 | APELLGGPSVFLFPPKPKDTLMISRTPEVTCVVVDVSHEDPEVKFNWYVDGVE | Clone CH3C.35.14 |
| | VHNAKTKPREEQYNSTYRVVSVLTVLHQDWLNGKEYKCKVSNKALPAPIEK | |
| | TISKAKGQPREPQVYTLPPSRDELTKNQVSLTCLVKGFYPSDIAVEWESLGHV | |
| | WAVYQTTPPVLDSDGSFFLYSKLTVPKSTWQQGWVFSCSVMHEALHNHYTQ | |
| | KSLSLSPGK | |
| 187 | APELLGGPSVFLFPPKPKDTLMISRTPEVTCVVVDVSHEDPEVKFNWYVDGVE | Clone CH3C.35.15 |
| | VHNAKTKPREEQYNSTYRVVSVLTVLHQDWLNGKEYKCKVSNKALPAPIEK | |
| | TISKAKGQPREPQVYTLPPSRDELTKNQVSLTCLVKGFYPSDIAVWWESLGHV | |
| | WAVYQTTPPVLDSDGSFFLYSKLTVPKSTWQQGWVFSCSVMHEALHNHYTQ | |
| | KSLSLSPGK | |
| 188 | APELLGGPSVFLFPPKPKDTLMISRTPEVTCVVVDVSHEDPEVKFNWYVDGVE | Clone CH3C.35.16 |
| | VHNAKTKPREEQYNSTYRVVSVLTVLHQDWLNGKEYKCKVSNKALPAPIEK | |
| | TISKAKGQPREPQVYTLPPSRDELTKNQVSLTCLVKGFYPSDIAVWWESLGHV | |
| | WVNQKTTPPVLDSDGSFFLYSKLTVPKSTWQQGWVFSCSVMHEALHNHYTQ | |
| | KSLSLSPGK | |
| 189 | APELLGGPSVFLFPPKPKDTLMISRTPEVTCVVVDVSHEDPEVKFNWYVDGVE | Clone CH3C.35.17 |
| | VHNAKTKPREEQYNSTYRVVSVLTVLHQDWLNGKEYKCKVSNKALPAPIEK | |
| | TISKAKGQPREPQVYTLPPSRDELTKNQVSLTCLVKGFYPSDIAVEWESLGHV | |
| | WVNQQTTPPVLDSDGSFFLYSKLTVPKSTWQQGWVFSCSVMHEALHNHYTQ | |
| | KSLSLSPGK | |
| 190 | APELLGGPSVFLFPPKPKDTLMISRTPEVTCVVVDVSHEDPEVKFNWYVDGVE | Clone CH3C.35.18 |
| | VHNAKTKPREEQYNSTYRVVSVLTVLHQDWLNGKEYKCKVSNKALPAPIEK | |
| | TISKAKGQPREPQVYTLPPSRDELTKNQVSLTCLVKGFYPSDIAVWWESLGHV | |
| | WVNQQTTPPVLDSDGSFFLYSKLTVPKSTWQQGWVFSCSVMHEALHNHYTQ | |
| | KSLSLSPGK | |
| 191 | APELLGGPSVFLFPPKPKDTLMISRTPEVTCVVVDVSHEDPEVKFNWYVDGVE | Clone CH3 C.35.19 |
| | VHNAKTKPREEQYNSTYRVVSVLTVLHQDWLNGKEYKCKVSNKALPAPIEK | |
| | TISKAKGQPREPQVYTLPPSRDELTKNQVSLTCLVKGFYPSDIAVWWESYGTE | |
| | WSSYKTTPPVLDSDGSFFLYSKLTVTKSEWQQGFVFSCSVMHEALHNHYTQK | |
| | SLSLSPGK | |
| 192 | APELLGGPSVFLFPPKPKDTLMISRTPEVTCVVVDVSHEDPEVKFNWYVDGVE | Clone CH3C.35.20 |
| | VHNAKTKPREEQYNSTYRVVSVLTVLHQDWLNGKEYKCKVSNKALPAPIEK | |
| | TISKAKGQPREPQVYTLPPSRDELTKNQVSLTCLVKGFYPSDIAVEWESYGTE | |
| | WSSYKTTPPVLDSDGSFFLYSKLTVTKEEWQQGFVFSCSVMHEALHNHYTQK | |
| | SLSLSPGK | |
| 193 | APELLGGPSVFLFPPKPKDTLMISRTPEVTCVVVDVSHEDPEVKFNWYVDGVE | Clone CH3C.35.21 |
| | VHNAKTKPREEQYNSTYRVVSVLTVLHQDWLNGKEYKCKVSNKALPAPIEK | |
| | TISKAKGQPREPQVYTLPPSRDELTKNQVSLTCLVKGFYPSDIAVWWESYGTE | |
| | WSSYKTTPPVLDSDGSFFLYSKLTVTKEEWQQGFVFSCSVMHEALHNHYTQK | |
| | SLSLSPGK | |
| 194 | APELLGGPSVFLFPPKPKDTLMISRTPEVTCVVVDVSHEDPEVKFNWYVDGVE | Clone CH3C.35.22 |
| | VHNAKTKPREEQYNSTYRVVSVLTVLHQDWLNGKEYKCKVSNKALPAPIEK | |
| | TISKAKGQPREPQVYTLPPSRDELTKNQVSLTCLVKGFYPSDIAVWWESYGTE | |
| | WSNYKTTPPVLDSDGSFFLYSKLTVTKSEWQQGFVFSCSVMHEALHNHYTQ | |
| | KSLSLSPGK | |
| 195 | APELLGGPSVFLFPPKPKDTLMISRTPEVTCVVVDVSHEDPEVKFNWYVDGVE | Clone CH3C.35.24 |
| | VHNAKTKPREEQYNSTYRVVSVLTVLHQDWLNGKEYKCKVSNKALPAPIEK | |
| | TISKAKGQPREPQVYTLPPSRDELTKNQVSLTCLVKGFYPSDIAVWWESYGTE | |
| | WSNYKTTPPVLDSDGSFFLYSKLTVTKEEWQQGFVFSCSVMHEALHNHYTQ | |
| | KSLSLSPGK | |
| 196 | YxTEWSS | Consensus motif for |
| | | CH3C.35 |
| 197 | TxxExxxxF | Consensus motif for |
| | | CH3C.35 |
| 198 | APELLGGPSVFLFPPKPKDTLMISRTPEVTCVVVDVSHEDPEVKFNWYVDGVE | Clone CH3C.35.21.1 |
| | VHNAKTKPREEQYNSTYRVVSVLTVLHQDWLNGKEYKCKVSNKALPAPIEK | |
| | TISKAKGQPREPQVYTLPPSRDELTKNQVSLTCLVKGFYPSDIAVLWESYGTE | |
| | WSSYKTTPPVLDSDGSFFLYSKLTVTKSEWQQGFVFSCSVMHEALHNHYTQK | |
| | SLSLSPGK | |
| 199 | APELLGGPSVFLFPPKPKDTLMISRTPEVTCVVVDVSHEDPEVKFNWYVDGVE | Clone CH3C.35.21.2 |
| | VHNAKTKPREEQYNSTYRVVSVLTVLHQDWLNGKEYKCKVSNKALPAPIEK | |
| | TISKAKGQPREPQVYTLPPSRDELTKNQVSLTCLVKGFYPSDIAVLWESYGTE | |
| | WSSYRTTPPVLDSDGSFFLYSKLTVTKSEWQQGFVFSCSVMHEALHNHYTQK | |
| | SLSLSPGK | |
| 200 | APELLGGPSVFLFPPKPKDTLMISRTPEVTCVVVDVSHEDPEVKFNWYVDGVE | Clone CH3C.35.21.3 |
| | VHNAKTKPREEQYNSTYRVVSVLTVLHQDWLNGKEYKCKVSNKALPAPIEK | |
| | TISKAKGQPREPQVYTLPPSRDELTKNQVSLTCLVKGFYPSDIAVLWESYGTE | |
| | WSSYRTTPPVLDSDGSFFLYSKLTVTREEWQQGFVFSCSVMHEALHNHYTQK | |
| | SLSLSPGK | |
| 201 | APELLGGPSVFLFPPKPKDTLMISRTPEVTCVVVDVSHEDPEVKFNWYVDGVE | Clone CH3C.35.21.4 |
| | VHNAKTKPREEQYNSTYRVVSVLTVLHQDWLNGKEYKCKVSNKALPAPIEK | |
| | TISKAKGQPREPQVYTLPPSRDELTKNQVSLTCLVKGFYPSDIAVLWESYGTE | |
| | WSSYRTTPPVLDSDGSFFLYSKLTVTGEEWQQGFVFSCSVMHEALHNHYTQK | |
| | SLSLSPGK | |
| 202 | APELLGGPSVFLFPPKPKDTLMISRTPEVTCVVVDVSHEDPEVKFNWYVDGVE | Clone CH3C.35.21.5 |
| | VHNAKTKPREEQYNSTYRVVSVLTVLHQDWLNGKEYKCKVSNKALPAPIEK | |
| | TISKAKGQPREPQVYTLPPSRDELTKNQVSLTCLVKGFYPSDIAVLWESYGTE | |
| | WSSYRTTPPVLDSDGSFFLYSKLTVTREEWQQGFVFSCWVMHEALHNHYTQ | |
| | KSLSLSPGK | |
| 203 | APELLGGPSVFLFPPKPKDTLMISRTPEVTCVVVDVSHEDPEVKFNWYVDGVE | Clone CH3C.35.21.6 |
| | VHNAKTKPREEQYNSTYRVVSVLTVLHQDWLNGKEYKCKVSNKALPAPIEK | |
| | TISKAKGQPREPQVYTLPPSRDELTKNQVSLTCLVKGFYPSDIAVLWESYGTE | |
| | WSSYRTTPPVLDSDGSFFLYSKLTVTKEEWQQGFVFSCWVMHEALHNHYTQ | |
| | KSLSLSPGK | |
| 204 | APELLGGPSVFLFPPKPKDTLMISRTPEVTCVVVDVSHEDPEVKFNWYVDGVE | Clone CH3C.35.21.7 |
| | VHNAKTKPREEQYNSTYRVVSVLTVLHQDWLNGKEYKCKVSNKALPAPIEK | |
| | TISKAKGQPREPQVYTLPPSRDELTKNQVSLTCLVKGFYPSDIAVLWESYGTE | |
| | WSSYRTTPPVLDSDGSFFLYSKLTVTREEWQQGFVFTCWVMHEALHNHYTQ | |
| | KSLSLSPGK | |
| 205 | APELLGGPSVFLFPPKPKDTLMISRTPEVTCVVVDVSHEDPEVKFNWYVDGVE | Clone CH3C.35.21.8 |
| | VHNAKTKPREEQYNSTYRVVSVLTVLHQDWLNGKEYKCKVSNKALPAPIEK | |
| | TISKAKGQPREPQVYTLPPSRDELTKNQVSLTCLVKGFYPSDIAVLWESYGTE | |
| | WSSYRTTPPVLDSDGSFFLYSKLTVTREEWQQGFVFTCGVMHEALHNHYTQ | |
| | KSLSLSPGK | |
| 206 | APELLGGPSVFLFPPKPKDTLMISRTPEVTCVVVDVSHEDPEVKFNWYVDGVE | Clone CH3C.35.21.9 |
| | VHNAKTKPREEQYNSTYRVVSVLTVLHQDWLNGKEYKCKVSNKALPAPIEK | |
| | TISKAKGQPREPQVYTLPPSRDELTKNQVSLTCLVKGFYPSDIAVLWESYGTE | |
| | WSSYRTTPPVLDSDGSFFLYSKLTVTREEWQQGFVFECWVMHEALHNHYTQ | |
| | KSLSLSPGK | |
| 207 | APELLGGPSVFLFPPKPKDTLMISRTPEVTCVVVDVSHEDPEVKFNWYVDGVE | Clone CH3C.35.21.10 |
| | VHNAKTKPREEQYNSTYRVVSVLTVLHQDWLNGKEYKCKVSNKALPAPIEK | |
| | TISKAKGQPREPQVYTLPPSRDELTKNQVSLTCLVKGFYPSDIAVLWESYGTE | |
| | WSSYRTTPPVLDSDGSFFLYSKLTVTREEWQQGFVFKCWVMHEALHNHYTQ | |
| | KSLSLSPGK | |
| 208 | APELLGGPSVFLFPPKPKDTLMISRTPEVTCVVVDVSHEDPEVKFNWYVDGVE | Clone CH3C.35.21.11 |
| | VHNAKTKPREEQYNSTYRVVSVLTVLHQDWLNGKEYKCKVSNKALPAPIEK | |
| | TISKAKGQPREPQVYTLPPSRDELTKNQVSLTCLVKGFYPSDIAVLWESYGTE | |
| | WSSYRTTPPVLDSDGSFFLYSKLTVTPEEWQQGFVFKCWVMHEALHNHYTQ | |
| | KSLSLSPGK | |
| 209 | APELLGGPSVFLFPPKPKDTLMISRTPEVTCVVVDVSHEDPEVKFNWYVDGVE | Clone CH3C.35.21.12 |
| | VHNAKTKPREEQYNSTYRVVSVLTVLHQDWLNGKEYKCKVSNKALPAPIEK | |
| | TISKAKGQPREPQVYTLPPSRDELTKNQVSLTCLVKGFYPSDIAVWWESYGTE | |
| | WSSYRTTPPVLDSDGSFFLYSKLTVTREEWQQGFVFSCSVMHEALHNHYTQK | |
| | SLSLSPGK | |
| 210 | APELLGGPSVFLFPPKPKDTLMISRTPEVTCVVVDVSHEDPEVKFNWYVDGVE | Clone CH3C.35.21.13 |
| | VHNAKTKPREEQYNSTYRVVSVLTVLHQDWLNGKEYKCKVSNKALPAPIEK | |
| | TISKAKGQPREPQVYTLPPSRDELTKNQVSLTCLVKGFYPSDIAVWWESYGTE | |
| | WSSYRTTPPVLDSDGSFFLYSKLTVTGEEWQQGFVFSCSVMHEALHNHYTQK | |
| | SLSLSPGK | |
| 211 | APELLGGPSVFLFPPKPKDTLMISRTPEVTCVVVDVSHEDPEVKFNWYVDGVE | Clone CH3C.35.21.14 |
| | VHNAKTKPREEQYNSTYRVVSVLTVLHQDWLNGKEYKCKVSNKALPAPIEK | |
| | TISKAKGQPREPQVYTLPPSRDELTKNQVSLTCLVKGFYPSDIAVWWESYGTE | |
| | WSSYRTTPPVLDSDGSFFLYSKLTVTREEWQQGFVFTCWVMHEALHNHYTQ | |
| | KSLSLSPGK | |
| 212 | APELLGGPSVFLFPPKPKDTLMISRTPEVTCVVVDVSHEDPEVKFNWYVDGVE | Clone CH3C.35.21.15 |
| | VHNAKTKPREEQYNSTYRVVSVLTVLHQDWLNGKEYKCKVSNKALPAPIEK | |
| | TISKAKGQPREPQVYTLPPSRDELTKNQVSLTCLVKGFYPSDIAVWWESYGTE | |
| | WSSYRTTPPVLDSDGSFFLYSKLTVTGEEWQQGFVFTCWVMHEALHNHYTQ | |
| | KSLSLSPGK | |
| 213 | APELLGGPSVFLFPPKPKDTLMISRTPEVTCVVVDVSHEDPEVKFNWYVDGVE | Clone CH3C.35.21.16 |
| | VHNAKTKPREEQYNSTYRVVSVLTVLHQDWLNGKEYKCKVSNKALPAPIEK | |
| | TISKAKGQPREPQVYTLPPSRDELTKNQVSLTCLVKGFYPSDIAVWWESYGTE | |
| | WSSYRTTPPVLDSDGSFFLYSKLTVTREEWQQGFVFTCGVMHEALHNHYTQ | |
| | KSLSLSPGK | |
| 214 | APELLGGPSVFLFPPKPKDTLMISRTPEVTCVVVDVSHEDPEVKFNWYVDGVE | Clone CH3C.35.21.17 |
| | VHNAKTKPREEQYNSTYRVVSVLTVLHQDWLNGKEYKCKVSNKALPAPIEK | |
| | TISKAKGQPREPQVYTLPPSRDELTKNQVSLTCLVKGFYPSDIAVLWESYGTE | |
| | WSSYKTTPPVLDSDGSFFLYSKLTVTKEEWQQGFVFSCSVMHEALHNHYTQK | |
| | SLSLSPGK | |
| 215 | APELLGGPSVFLFPPKPKDTLMISRTPEVTCVVVDVSHEDPEVKFNWYVDGVE | Clone CH3C.35.21.18 |
| | VHNAKTKPREEQYNSTYRVVSVLTVLHQDWLNGKEYKCKVSNKALPAPIEK | |
| | TISKAKGQPREPQVYTLPPSRDELTKNQVSLTCLVKGFYPSDIAVLWESYGTE | |
| | WSSYRTTPPVLDSDGSFFLYSKLTVTKEEWQQGFVFSCSVMHEALHNHYTQK | |
| | SLSLSPGK | |
| 216 | APELLGGPSVFLFPPKPKDTLMISRTPEVTCVVVDVSHEDPEVKFNWYVDGVE | Clone CH3C.35.20.1 |
| | VHNAKTKPREEQYNSTYRVVSVLTVLHQDWLNGKEYKCKVSNKALPAPIEK | |
| | TISKAKGQPREPQVYTLPPSRDELTKNQVSLTCLVKGFYPSDIAVEWESFGTE | |
| | WSSYKTTPPVLDSDGSFFLYSKLTVTKEEWQQGFVFSCSVMHEALHNHYTQK | |
| | SLSLSPGK | |
| 217 | APELLGGPSVFLFPPKPKDTLMISRTPEVTCVVVDVSHEDPEVKFNWYVDGVE | Clone CH3C.35.20.2 |
| | VHNAKTKPREEQYNSTYRVVSVLTVLHQDWLNGKEYKCKVSNKALPAPIEK | |
| | TISKAKGQPREPQVYTLPPSRDELTKNQVSLTCLVKGFYPSDIAVEWESYGTE | |
| | WASYKTTPPVLDSDGSFFLYSKLTVTKEEWQQGFVFSCSVMHEALHNHYTQ | |
| | KSLSLSPGK | |
| 218 | APELLGGPSVFLFPPKPKDTLMISRTPEVTCVVVDVSHEDPEVKFNWYVDGVE | Clone CH3C.35.20.3 |
| | VHNAKTKPREEQYNSTYRVVSVLTVLHQDWLNGKEYKCKVSNKALPAPIEK | |
| | TISKAKGQPREPQVYTLPPSRDELTKNQVSLTCLVKGFYPSDIAVEWESYGTE | |
| | WVSYKTTPPVLDSDGSFFLYSKLTVTKEEWQQGFVFSCSVMHEALHNHYTQ | |
| | KSLSLSPGK | |
| 219 | APELLGGPSVFLFPPKPKDTLMISRTPEVTCVVVDVSHEDPEVKFNWYVDGVE | Clone CH3C.35.20.4 |
| | VHNAKTKPREEQYNSTYRVVSVLTVLHQDWLNGKEYKCKVSNKALPAPIEK | |
| | TISKAKGQPREPQVYTLPPSRDELTKNQVSLTCLVKGFYPSDIAVEWESYGTE | |
| | WSSYKTTPPVLDSDGSFFLYSKLTVSKEEWQQGFVFSCSVMHEALHNHYTQK | |
| | SLSLSPGK | |
| 220 | APELLGGPSVFLFPPKPKDTLMISRTPEVTCVVVDVSHEDPEVKFNWYVDGVE | Clone CH3C.35.20.5 |
| | VHNAKTKPREEQYNSTYRVVSVLTVLHQDWLNGKEYKCKVSNKALPAPIEK | |
| | TISKAKGQPREPQVYTLPPSRDELTKNQVSLTCLVKGFYPSDIAVEWESFGTE | |
| | WASYKTTPPVLDSDGSFFLYSKLTVTKEEWQQGFVFSCSVMHEALHNHYTQ | |
| | KSLSLSPGK | |
| 221 | APELLGGPSVFLFPPKPKDTLMISRTPEVTCVVVDVSHEDPEVKFNWYVDGVE | Clone CH3C.35.20.6 |
| | VHNAKTKPREEQYNSTYRVVSVLTVLHQDWLNGKEYKCKVSNKALPAPIEK | |
| | TISKAKGQPREPQVYTLPPSRDELTKNQVSLTCLVKGFYPSDIAVEWESFGTE | |
| | WVSYKTTPPVLDSDGSFFLYSKLTVTKEEWQQGFVFSCSVMHEALHNHYTQ | |
| | KSLSLSPGK | |
| 222 | APELLGGPSVFLFPPKPKDTLMISRTPEVTCVVVDVSHEDPEVKFNWYVDGVE | Clone CH3C.35.21.a.1 |
| | VHNAKTKPREEQYNSTYRVVSVLTVLHQDWLNGKEYKCKVSNKALPAPIEK | |
| | TISKAKGQPREPQVYTLPPSRDELTKNQVSLTCLVKGFYPSDIAVWWESFGTE | |
| | WSSYKTTPPVLDSDGSFFLYSKLTVTKEEWQQGFVFSCSVMHEALHNHYTQK | |
| | SLSLSPGK | |
| 223 | APELLGGPSVFLFPPKPKDTLMISRTPEVTCVVVDVSHEDPEVKFNWYVDGVE | Clone CH3C.35.21.a.2 |
| | VHNAKTKPREEQYNSTYRVVSVLTVLHQDWLNGKEYKCKVSNKALPAPIEK | |
| | TISKAKGQPREPQVYTLPPSRDELTKNQVSLTCLVKGFYPSDIAVWWESYGTE | |
| | WASYKTTPPVLDSDGSFFLYSKLTVTKEEWQQGFVFSCSVMHEALHNHYTQ | |
| | KSLSLSPGK | |
| 224 | APELLGGPSVFLFPPKPKDTLMISRTPEVTCVVVDVSHEDPEVKFNWYVDGVE | Clone CH3C.35.21.a.3 |
| | VHNAKTKPREEQYNSTYRVVSVLTVLHQDWLNGKEYKCKVSNKALPAPIEK | |
| | TISKAKGQPREPQVYTLPPSRDELTKNQVSLTCLVKGFYPSDIAVWWESYGTE | |
| | WVSYKTTPPVLDSDGSFFLYSKLTVTKEEWQQGFVFSCSVMHEALHNHYTQ | |
| | KSLSLSPGK | |
| 225 | APELLGGPSVFLFPPKPKDTLMISRTPEVTCVVVDVSHEDPEVKFNWYVDGVE | Clone CH3C.35.21.a.4 |
| | VHNAKTKPREEQYNSTYRVVSVLTVLHQDWLNGKEYKCKVSNKALPAPIEK | |
| | TISKAKGQPREPQVYTLPPSRDELTKNQVSLTCLVKGFYPSDIAVWWESYGTE | |
| | WSSYKTTPPVLDSDGSFFLYSKLTVSKEEWQQGFVFSCSVMHEALHNHYTQK | |
| | SLSLSPGK | |
| 226 | APELLGGPSVFLFPPKPKDTLMISRTPEVTCVVVDVSHEDPEVKFNWYVDGVE | Clone CH3C.35.21.a.5 |
| | VHNAKTKPREEQYNSTYRVVSVLTVLHQDWLNGKEYKCKVSNKALPAPIEK | |
| | TISKAKGQPREPQVYTLPPSRDELTKNQVSLTCLVKGFYPSDIAVWWESFGTE | |
| | WASYKTTPPVLDSDGSFFLYSKLTVTKEEWQQGFVFSCSVMHEALHNHYTQ | |
| | KSLSLSPGK | |
| 227 | APELLGGPSVFLFPPKPKDTLMISRTPEVTCVVVDVSHEDPEVKFNWYVDGVE | Clone CH3C.35.21.a.6 |
| | VHNAKTKPREEQYNSTYRVVSVLTVLHQDWLNGKEYKCKVSNKALPAPIEK | |
| | TISKAKGQPREPQVYTLPPSRDELTKNQVSLTCLVKGFYPSDIAVWWESFGTE | |
| | WVSYKTTPPVLDSDGSFFLYSKLTVTKEEWQQGFVFSCSVMHEALHNHYTQ | |
| | KSLSLSPGK | |
| 228 | APELLGGPSVFLFPPKPKDTLMISRTPEVTCVVVDVSHEDPEVKFNWYVDGVE | Clone CH3C.35.23.1 |
| | VHNAKTKPREEQYNSTYRVVSVLTVLHQDWLNGKEYKCKVSNKALPAPIEK | |
| | TISKAKGQPREPQVYTLPPSRDELTKNQVSLTCLVKGFYPSDIAVEWESFGTE | |
| | WSNYKTTPPVLDSDGSFFLYSKLTVTKEEWQQGFVFSCSVMHEALHNHYTQ | |
| | KSLSLSPGK | |
| 229 | APELLGGPSVFLFPPKPKDTLMISRTPEVTCVVVDVSHEDPEVKFNWYVDGVE | Clone CH3C.35.23.2 |
| | VHNAKTKPREEQYNSTYRVVSVLTVLHQDWLNGKEYKCKVSNKALPAPIEK | |
| | TISKAKGQPREPQVYTLPPSRDELTKNQVSLTCLVKGFYPSDIAVEWESYGTE | |
| | WANYKTTPPVLDSDGSFFLYSKLTVTKEEWQQGFVFSCSVMHEALHNHYTQ | |
| | KSLSLSPGK | |
| 230 | APELLGGPSVFLFPPKPKDTLMISRTPEVTCVVVDVSHEDPEVKFNWYVDGVE | Clone CH3C.35.23.5 |
| | VHNAKTKPREEQYNSTYRVVSVLTVLHQDWLNGKEYKCKVSNKALPAPIEK | |
| | TISKAKGQPREPQVYTLPPSRDELTKNQVSLTCLVKGFYPSDIAVEWESFGTE | |
| | WANYKTTPPVLDSDGSFFLYSKLTVTKEEWQQGFVFSCSVMHEALHNHYTQ | |
| | KSLSLSPGK | |
| 231 | APELLGGPSVFLFPPKPKDTLMISRTPEVTCVVVDVSHEDPEVKFNWYVDGVE | Clone CH3C.35.23.6 |
| | VHNAKTKPREEQYNSTYRVVSVLTVLHQDWLNGKEYKCKVSNKALPAPIEK | |
| | TISKAKGQPREPQVYTLPPSRDELTKNQVSLTCLVKGFYPSDIAVEWESFGTE | |
| | WVNYKTTPPVLDSDGSFFLYSKLTVTKEEWQQGFVFSCSVMHEALHNHYTQ | |
| | KSLSLSPGK | |
| 232 | APELLGGPSVFLFPPKPKDTLMISRTPEVTCVVVDVSHEDPEVKFNWYVDGVE | Clone CH3C.35.24.1 |
| | VHNAKTKPREEQYNSTYRVVSVLTVLHQDWLNGKEYKCKVSNKALPAPIEK | |
| | TISKAKGQPREPQVYTLPPSRDELTKNQVSLTCLVKGFYPSDIAVWWESFGTE | |
| | WSNYKTTPPVLDSDGSFFLYSKLTVTKEEWQQGFVFSCSVMHEALHNHYTQ | |
| | KSLSLSPGK | |
| 233 | APELLGGPSVFLFPPKPKDTLMISRTPEVTCVVVDVSHEDPEVKFNWYVDGVE | Clone CH3C.35.24.2 |
| | VHNAKTKPREEQYNSTYRVVSVLTVLHQDWLNGKEYKCKVSNKALPAPIEK | |
| | TISKAKGQPREPQVYTLPPSRDELTKNQVSLTCLVKGFYPSDIAVWWESYGTE | |
| | WANYKTTPPVLDSDGSFFLYSKLTVTKEEWQQGFVFSCSVMHEALHNHYTQ | |
| | KSLSLSPGK | |
| 234 | APELLGGPSVFLFPPKPKDTLMISRTPEVTCVVVDVSHEDPEVKFNWYVDGVE | Clone CH3C.35.24.3 |
| | VHNAKTKPREEQYNSTYRVVSVLTVLHQDWLNGKEYKCKVSNKALPAPIEK | |
| | TISKAKGQPREPQVYTLPPSRDELTKNQVSLTCLVKGFYPSDIAVWWESYGTE | |
| | WVNYKTTPPVLDSDGSFFLYSKLTVTKEEWQQGFVFSCSVMHEALHNHYTQ | |
| | KSLSLSPGK | |
| 235 | APELLGGPSVFLFPPKPKDTLMISRTPEVTCVVVDVSHEDPEVKFNWYVDGVE | Clone CH3C.35.24.4 |
| | VHNAKTKPREEQYNSTYRVVSVLTVLHQDWLNGKEYKCKVSNKALPAPIEK | |
| | TISKAKGQPREPQVYTLPPSRDELTKNQVSLTCLVKGFYPSDIAVWWESYGTE | |
| | WSNYKTTPPVLDSDGSFFLYSKLTVSKEEWQQGFVFSCSVMHEALHNHYTQ | |
| | KSLSLSPGK | |
| 236 | APELLGGPSVFLFPPKPKDTLMISRTPEVTCVVVDVSHEDPEVKFNWYVDGVE | Clone CH3C.35.24.5 |
| | VHNAKTKPREEQYNSTYRVVSVLTVLHQDWLNGKEYKCKVSNKALPAPIEK | |
| | TISKAKGQPREPQVYTLPPSRDELTKNQVSLTCLVKGFYPSDIAVWWESFGTE | |
| | WANYKTTPPVLDSDGSFFLYSKLTVTKEEWQQGFVFSCSVMHEALHNHYTQ | |
| | KSLSLSPGK | |
| 237 | APELLGGPSVFLFPPKPKDTLMISRTPEVTCVVVDVSHEDPEVKFNWYVDGVE | Clone CH3C.35.24.6 |
| | VHNAKTKPREEQYNSTYRVVSVLTVLHQDWLNGKEYKCKVSNKALPAPIEK | |
| | TISKAKGQPREPQVYTLPPSRDELTKNQVSLTCLVKGFYPSDIAVWWESFGTE | |
| | WVNYKTTPPVLDSDGSFFLYSKLTVTKEEWQQGFVFSCSVMHEALHNHYTQ | |
| | KSLSLSPGK | |
| 238 | APELLGGPSVFLFPPKPKDTLMISRTPEVTCVVVDVSHEDPEVKFNWYVDGVE | Clone |
| | VHNAKTKPREEQYNSTYRVVSVLTVLHQDWLNGKEYKCKVSNKALPAPIEK | CH3C.35.21.17.1 |
| | TISKAKGQPREPQVYTLPPSRDELTKNQVSLTCLVKGFYPSDIAVLWESFGTE | |
| | WSSYKTTPPVLDSDGSFFLYSKLTVTKEEWQQGFVFSCSVMHEALHNHYTQK | |
| | SLSLSPGK | |
| 239 | APELLGGPSVFLFPPKPKDTLMISRTPEVTCVVVDVSHEDPEVKFNWYVDGVE | Clone |
| | VHNAKTKPREEQYNSTYRVVSVLTVLHQDWLNGKEYKCKVSNKALPAPIEK | CH3C.35.21.17.2 |
| | TISKAKGQPREPQVYTLPPSRDELTKNQVSLTCLVKGFYPSDIAVLWESYGTE | |
| | WASYKTTPPVLDSDGSFFLYSKLTVTKEEWQQGFVFSCSVMHEALHNHYTQ | |
| | KSLSLSPGK | |
| 240 | APELLGGPSVFLFPPKPKDTLMISRTPEVTCVVVDVSHEDPEVKFNWYVDGVE | Clone |
| | VHNAKTKPREEQYNSTYRVVSVLTVLHQDWLNGKEYKCKVSNKALPAPIEK | CH3C.35.21.17.3 |
| | TISKAKGQPREPQVYTLPPSRDELTKNQVSLTCLVKGFYPSDIAVLWESYGTE | |
| | WVSYKTTPPVLDSDGSFFLYSKLTVTKEEWQQGFVFSCSVMHEALHNHYTQ | |
| | KSLSLSPGK | |
| 241 | APELLGGPSVFLFPPKPKDTLMISRTPEVTCVVVDVSHEDPEVKFNWYVDGVE | Clone |
| | VHNAKTKPREEQYNSTYRVVSVLTVLHQDWLNGKEYKCKVSNKALPAPIEK | CH3C.35.21.17.4 |
| | TISKAKGQPREPQVYTLPPSRDELTKNQVSLTCLVKGFYPSDIAVLWESYGTE | |
| | WSSYKTTPPVLDSDGSFFLYSKLTVSKEEWQQGFVFSCSVMHEALHNHYTQK | |
| | SLSLSPGK | |
| 242 | APELLGGPSVFLFPPKPKDTLMISRTPEVTCVVVDVSHEDPEVKFNWYVDGVE | Clone |
| | VHNAKTKPREEQYNSTYRVVSVLTVLHQDWLNGKEYKCKVSNKALPAPIEK | CH3C.35.21.17.5 |
| | TISKAKGQPREPQVYTLPPSRDELTKNQVSLTCLVKGFYPSDIAVLWESFGTE | |
| | WASYKTTPPVLDSDGSFFLYSKLTVTKEEWQQGFVFSCSVMHEALHNHYTQ | |
| | KSLSLSPGK | |
| 243 | APELLGGPSVFLFPPKPKDTLMISRTPEVTCVVVDVSHEDPEVKFNWYVDGVE | Clone |
| | VHNAKTKPREEQYNSTYRVVSVLTVLHQDWLNGKEYKCKVSNKALPAPIEK | CH3C.35.21.17.6 |
| | TISKAKGQPREPQVYTLPPSRDELTKNQVSLTCLVKGFYPSDIAVLWESFGTE | |
| | WVSYKTTPPVLDSDGSFFLYSKLTVTKEEWQQGFVFSCSVMHEALHNHYTQ | |
| | KSLSLSPGK | |
| 244 | APELLGGPSVFLFPPKPKDTLMISRTPEVTCVVVDVSHEDPEVKFNWYVDGVE | Clone CH3C.35.N390 |
| | VHNAKTKPREEQYNSTYRVVSVLTVLHQDWLNGKEYKCKVSNKALPAPIEK | |
| | TISKAKGQPREPQVYTLPPSRDELTKNQVSLTCLVKGFYPSDIAVEWESYGTE | |
| | WSNYKTTPPVLDSDGSFFLYSKLTVTKSEWQQGFVFSCSVMHEALHNHYTQ | |
| | KSLSLSPGK | |
| 245 | APELLGGPSVFLFPPKPKDTLMISRTPEVTCVVVDVSHEDPEVKFNWYVDGVE | Clone CH3C.35.20.1.1 |
| | VHNAKTKPREEQYNSTYRVVSVLTVLHQDWLNGKEYKCKVSNKALPAPIEK | |
| | TISKAKGQPREPQVYTLPPSRDELTKNQVSLTCLVKGFYPSDIAVEWESFGTE | |
| | WSSYKTTPPVLDSDGSFFLYSKLTVSKEEWQQGFVFSCSVMHEALHNHYTQK | |
| | SLSLSPGK | |
| 246 | APELLGGPSVFLFPPKPKDTLMISRTPEVTCVVVDVSHEDPEVKFNWYVDGVE | Clone CH3C.35.23.2.1 |
| | VHNAKTKPREEQYNSTYRVVSVLTVLHQDWLNGKEYKCKVSNKALPAPIEK | |
| | TISKAKGQPREPQVYTLPPSRDELTKNQVSLTCLVKGFYPSDIAVEWESYGTE | |
| | WANYKTTPPVLDSDGSFFLYSKLTVSKSEWQQGFVFSCSVMHEALHNHYTQ | |
| | KSLSLSPGK | |
| 247 | APELLGGPSVFLFPPKPKDTLMISRTPEVTCVVVDVSHEDPEVKFNWYVDGVE | Clone CH3C.35.S413 |
| | VHNAKTKPREEQYNSTYRVVSVLTVLHQDWLNGKEYKCKVSNKALPAPIEK | |
| | TISKAKGQPREPQVYTLPPSRDELTKNQVSLTCLVKGFYPSDIAVEWESYGTE | |
| | WSSYKTTPPVLDSDGSFFLYSKLTVSKSEWQQGFVFSCSVMHEALHNHYTQK | |
| | SLSLSPGK | |
| 248 | APELLGGPSVFLFPPKPKDTLMISRTPEVTCVVVDVSHEDPEVKFNWYVDGVE | Clone CH3C.35.23.3.1 |
| | VHNAKTKPREEQYNSTYRVVSVLTVLHQDWLNGKEYKCKVSNKALPAPIEK | |
| | TISKAKGQPREPQVYTLPPSRDELTKNQVSLTCLVKGFYPSDIAVEWESYGTE | |
| | WVNYKTTPPVLDSDGSFFLYSKLTVSKEEWQQGFVFSCSVMHEALHNHYTQ | |
| | KSLSLSPGK | |
| 249 | APELLGGPSVFLFPPKPKDTLMISRTPEVTCVVVDVSHEDPEVKFNWYVDGVE | Clone CH3C.35.23.6.1 |
| | VHNAKTKPREEQYNSTYRVVSVLTVLHQDWLNGKEYKCKVSNKALPAPIEK | |
| | TISKAKGQPREPQVYTLPPSRDELTKNQVSLTCLVKGFYPSDIAVEWESFGTE | |
| | WVNYKTTPPVLDSDGSFFLYSKLTVSKEEWQQGFVFSCSVMHEALHNHYTQ | |
| | KSLSLSPGK | |
| 250 | GYSFTGYWMN | Anti-HER2_DI CDR- |
| | | H1 |
| 251 | MIHPSDSEIRANQKFRD | Anti-HER2_DI CDR- |
| | | H2 |
| 252 | ARGTYDGGFEY | Anti-HER2_DI CDR- |
| | | H3 |
| 253 | RASQSVSGSRFTYMH | Anti-HER2_DI CDR- |
| | | L1 |
| 254 | YASILES | Anti-HER2_DI CDR- |
| | | L2 |
| 255 | QHSWEIPP | Anti-HER2_DI CDR- |
| | | L3 |
| 256 | QVQLQQPGAELVRPGASVKLSCKASGYSFTGYWMNWLKQRPGQGLEWIGMI | Anti-HER2_DI VH |
| | HPSDSEIRANQKFRDKATLTVDKSSTTAYMQLSSPTSEDSAVYYCARGTYDG | sequence |
| | GFEYWGQGTTLTVSS | |
| 257 | DIVLTQSPASLVVSLGQRATISCRASQSVSGSRFTYMHWYQQKPGQPPKLLIK | Anti-HER2_DI VL |
| | YASILESGVPARFSGGGSGTDFTLNIHPVEEDDTATYYCQHSWEIPPWTFGGG | sequence |
| | TKLEIK | |
| 258 | QVQLQQPGAELVRPGASVKLSCKASGYSFTGYWMNWLKQRPGQGLEWIGMI | Anti-HER2_DI fused to |
| | HPSDSEIRANQKFRDKATLTVDKSSTTAYMQLSSPTSEDSAVYYCARGTYDG | CH3C.35.23.1.1 with |
| | GFEYWGQGTTLTVSSASTKGPSVFPLAPSSKSTSGGTAALGCLVKDYFPEPVT | knob mutation |
| | VSWNSGALTSGVHTFPAVLQSSGLYSLSSVVTVPSSSLGTQTYICNVNHKPSN | |
| | TKVDKKVEPKSCDKTHTCPPCPAPELLGGPSVFLFPPKPKDTLMISRTPEVTCV | |
| | VVDVSHEDPEVKFNWYVDGVEVHNAKTKPREEQYNSTYRVVSVLTVLHQD | |
| | WLNGKEYKCKVSNKALPAPIEKTISKAKGQPREPQVYTLPPSRDELTKNQVSL | |
| | WCLVKGFYPSDIAVEWESFGTEWSNYKTTPPVLDSDGSFFLYSKLTVSKEEW | |
| | QQGFVFSCSVMHEALHNHYTQKSLSLSPGK | |
| 259 | QVQLQQPGAELVRPGASVKLSCKASGYSFTGYWMNWLKQRPGQGLEWIGMI | Anti-HER2_DI fused to |
| | HPSDSEIRANQKFRDKATLTVDKSSTTAYMQLSSPTSEDSAVYYCARGTYDG | CH3C.35.23.1.1 with |
| | GFEYWGQGTTLTVSSASTKGPSVFPLAPSSKSTSGGTAALGCLVKDYFPEPVT | knob and LALA |
| | VSWNSGALTSGVHTFPAVLQSSGLYSLSSVVTVPSSSLGTQTYICNVNHKPSN | mutations |
| | TKVDKKVEPKSCDKTHTCPPCPAPEAAGGPSVFLFPPKPKDTLMISRTPEVTC | |
| | VVVDVSHEDPEVKFNWYVDGVEVHNAKTKPREEQYNSTYRVVSVLTVLHQ | |
| | DWLNGKEYKCKVSNKALPAPIEKTISKAKGQPREPQVYTLPPSRDELTKNQVS | |
| | LWCLVKGFYPSDIAVEWESFGTEWSNYKTTPPVLDSDGSFFLYSKLTVSKEE | |
| | WQQGFVFSCSVMHEALHNHYTQKSLSLSPGK | |
| 260 | QVQLQQPGAELVRPGASVKLSCKASGYSFTGYWMNWLKQRPGQGLEWIGMI | Anti-HER2_DI fused to |
| | HPSDSEIRANQKFRDKATLTVDKSSTTAYMQLSSPTSEDSAVYYCARGTYDG | CH3C.35.23.1.1 with |
| | GFEYWGQGTTLTVSSASTKGPSVFPLAPSSKSTSGGTAALGCLVKDYFPEPVT | knob and M428L and |
| | VSWNSGALTSGVHTFPAVLQSSGLYSLSSVVTVPSSSLGTQTYICNVNHKPSN | N434S mutations |
| | TKVDKKVEPKSCDKTHTCPPCPAPELLGGPSVFLFPPKPKDTLMISRTPEVTCV | |
| | VVDVSHEDPEVKFNWYVDGVEVHNAKTKPREEQYNSTYRVVSVLTVLHQD | |
| | WLNGKEYKCKVSNKALPAPIEKTISKAKGQPREPQVYTLPPSRDELTKNQVSL | |
| | WCLVKGFYPSDIAVEWESFGTEWSNYKTTPPVLDSDGSFFLYSKLTVSKEEW | |
| | QQGFVFSCSVLHEALHSHYTQKSLSLSPGK | |
| 261 | QVQLQQPGAELVRPGASVKLSCKASGYSFTGYWMNWLKQRPGQGLEWIGMI | Anti-HER2_DI fused to |
| | HPSDSEIRANQKFRDKATLTVDKSSTTAYMQLSSPTSEDSAVYYCARGTYDG | CH3C.35.23.1.1 with |
| | GFEYWGQGTTLTVSSASTKGPSVFPLAPSSKSTSGGTAALGCLVKDYFPEPVT | knob, LALA, and |
| | VSWNSGALTSGVHTFPAVLQSSGLYSLSSVVTVPSSSLGTQTYICNVNHKPSN | M428L and N434S |
| | TKVDKKVEPKSCDKTHTCPPCPAPEAAGGPSVFLFPPKPKDTLMISRTPEVTC | mutations |
| | VVVDVSHEDPEVKFNWYVDGVEVHNAKTKPREEQYNSTYRVVSVLTVLHQ | |
| | DWLNGKEYKCKVSNKALPAPIEKTISKAKGQPREPQVYTLPPSRDELTKNQVS | |
| | LWCLVKGFYPSDIAVEWESFGTEWSNYKTTPPVLDSDGSFFLYSKLTVSKEE | |
| | WQQGFVFSCSVLHEALHSHYTQKSLSLSPGK | |
| 262 | QVQLQQPGAELVRPGASVKLSCKASGYSFTGYWMNWLKQRPGQGLEWIGMI | Anti-HER2_DI fused to |
| | HPSDSEIRANQKFRDKATLTVDKSSTTAYMQLSSPTSEDSAVYYCARGTYDG | CH3C.35.23.1.1 with |
| | GFEYWGQGTTLTVSSASTKGPSVFPLAPSSKSTSGGTAALGCLVKDYFPEPVT | hole mutations |
| | VSWNSGALTSGVHTFPAVLQSSGLYSLSSVVTVPSSSLGTQTYICNVNHKPSN | |
| | TKVDKKVEPKSCDKTHTCPPCPAPELLGGPSVFLFPPKPKDTLMISRTPEVTCV | |
| | VVDVSHEDPEVKFNWYVDGVEVHNAKTKPREEQYNSTYRVVSVLTVLHQD | |
| | WLNGKEYKCKVSNKALPAPIEKTISKAKGQPREPQVYTLPPSRDELTKNQVSL | |
| | SCAVKGFYPSDIAVEWESFGTEWSNYKTTPPVLDSDGSFFLVSKLTVSKEEWQ | |
| | QGFVFSCSVMHEALHNHYTQKSLSLSPGK | |
| 263 | QVQLQQPGAELVRPGASVKLSCKASGYSFTGYWMNWLKQRPGQGLEWIGMI | Anti-HER2_DI fused to |
| | HPSDSEIRANQKFRDKATLTVDKSSTTAYMQLSSPTSEDSAVYYCARGTYDG | CH3C.35.23.1.1 with |
| | GFEYWGQGTTLTVSSASTKGPSVFPLAPSSKSTSGGTAALGCLVKDYFPEPVT | hole and LALA |
| | VSWNSGALTSGVHTFPAVLQSSGLYSLSSVVTVPSSSLGTQTYICNVNHKPSN | mutations |
| | TKVDKKVEPKSCDKTHTCPPCPAPEAAGGPSVFLFPPKPKDTLMISRTPEVTC | |
| | VVVDVSHEDPEVKFNWYVDGVEVHNAKTKPREEQYNSTYRVVSVLTVLHQ | |
| | DWLNGKEYKCKVSNKALPAPIEKTISKAKGQPREPQVYTLPPSRDELTKNQVS | |
| | LSCAVKGFYPSDIAVEWESFGTEWSNYKTTPPVLDSDGSFFLVSKLTVSKEEW | |
| | QQGFVFSCSVMHEALHNHYTQKSLSLSPGK | |
| 264 | QVQLQQPGAELVRPGASVKLSCKASGYSFTGYWMNWLKQRPGQGLEWIGMI | Anti-HER2_DI fused to |
| | HPSDSEIRANQKFRDKATLTVDKSSTTAYMQLSSPTSEDSAVYYCARGTYDG | CH3C.35.23.1.1 with |
| | GFEYWGQGTTLTVSSASTKGPSVFPLAPSSKSTSGGTAALGCLVKDYFPEPVT | hole and M428L and |
| | VSWNSGALTSGVHTFPAVLQSSGLYSLSSVVTVPSSSLGTQTYICNVNHKPSN | N434S mutations |
| | TKVDKKVEPKSCDKTHTCPPCPAPELLGGPSVFLFPPKPKDTLMISRTPEVTCV | |
| | VVDVSHEDPEVKFNWYVDGVEVHNAKTKPREEQYNSTYRVVSVLTVLHQD | |
| | WLNGKEYKCKVSNKALPAPIEKTISKAKGQPREPQVYTLPPSRDELTKNQVSL | |
| | SCAVKGFYPSDIAVEWESFGTEWSNYKTTPPVLDSDGSFFLVSKLTVSKEEWQ | |
| | QGFVFSCSVLHEALHSHYTQKSLSLSPGK | |
| 265 | QVQLQQPGAELVRPGASVKLSCKASGYSFTGYWMNWLKQRPGQGLEWIGMI | Anti-HER2_DI fused to |
| | HPSDSEIRANQKFRDKATLTVDKSSTTAYMQLSSPTSEDSAVYYCARGTYDG | CH3C.35.23.1.1 with |
| | GFEYWGQGTTLTVSSASTKGPSVFPLAPSSKSTSGGTAALGCLVKDYFPEPVT | hole, LALA, and |
| | VSWNSGALTSGVHTFPAVLQSSGLYSLSSVVTVPSSSLGTQTYICNVNHKPSN | M428L and N434S |
| | TKVDKKVEPKSCDKTHTCPPCPAPEAAGGPSVFLFPPKPKDTLMISRTPEVTC | mutations |
| | VVVDVSHEDPEVKFNWYVDGVEVHNAKTKPREEQYNSTYRVVSVLTVLHQ | |
| | DWLNGKEYKCKVSNKALPAPIEKTISKAKGQPREPQVYTLPPSRDELTKNQVS | |
| | LSCAVKGFYPSDIAVEWESFGTEWSNYKTTPPVLDSDGSFFLVSKLTVSKEEW | |
| | QQGFVFSCSVLHEALHSHYTQKSLSLSPGK | |
| 266 | QVQLQQPGAELVRPGASVKLSCKASGYSFTGYWMNWLKQRPGQGLEWIGMI | Anti-HER2_DI fused to |
| | HPSDSEIRANQKFRDKATLTVDKSSTTAYMQLSSPTSEDSAVYYCARGTYDG | CH3C.35.23.3 with |
| | GFEYWGQGTTLTVSSASTKGPSVFPLAPSSKSTSGGTAALGCLVKDYFPEPVT | knob mutation |
| | VSWNSGALTSGVHTFPAVLQSSGLYSLSSVVTVPSSSLGTQTYICNVNHKPSN | |
| | TKVDKKVEPKSCDKTHTCPPCPAPELLGGPSVFLFPPKPKDTLMISRTPEVTCV | |
| | VVDVSHEDPEVKFNWYVDGVEVHNAKTKPREEQYNSTYRVVSVLTVLHQD | |
| | WLNGKEYKCKVSNKALPAPIEKTISKAKGQPREPQVYTLPPSRDELTKNQVSL | |
| | WCLVKGFYPSDIAVEWESYGTEWVNYKTTPPVLDSDGSFFLYSKLTVTKEEW | |
| | QQGFVFSCSVMHEALHNHYTQKSLSLSPGK | |
| 267 | QVQLQQPGAELVRPGASVKLSCKASGYSFTGYWMNWLKQRPGQGLEWIGMI | Anti-HER2_DI fused to |
| | HPSDSEIRANQKFRDKATLTVDKSSTTAYMQLSSPTSEDSAVYYCARGTYDG | CH3C.35.23.3 with |
| | GFEYWGQGTTLTVSSASTKGPSVFPLAPSSKSTSGGTAALGCLVKDYFPEPVT | knob and LALA |
| | VSWNSGALTSGVHTFPAVLQSSGLYSLSSVVTVPSSSLGTQTYICNVNHKPSN | mutations |
| | TKVDKKVEPKSCDKTHTCPPCPAPEAAGGPSVFLFPPKPKDTLMISRTPEVTC | |
| | VVVDVSHEDPEVKFNWYVDGVEVHNAKTKPREEQYNSTYRVVSVLTVLHQ | |
| | DWLNGKEYKCKVSNKALPAPIEKTISKAKGQPREPQVYTLPPSRDELTKNQVS | |
| | LWCLVKGFYPSDIAVEWESYGTEWVNYKTTPPVLDSDGSFFLYSKLTVTKEE | |
| | WQQGFVFSCSVMHEALHNHYTQKSLSLSPGK | |
| 268 | QVQLQQPGAELVRPGASVKLSCKASGYSFTGYWMNWLKQRPGQGLEWIGMI | Anti-HER2_DI fused to |
| | HPSDSEIRANQKFRDKATLTVDKSSTTAYMQLSSPTSEDSAVYYCARGTYDG | CH3C.35.23.3 with |
| | GFEYWGQGTTLTVSSASTKGPSVFPLAPSSKSTSGGTAALGCLVKDYFPEPVT | knob and M428L and |
| | VSWNSGALTSGVHTFPAVLQSSGLYSLSSVVTVPSSSLGTQTYICNVNHKPSN | N434S mutations |
| | TKVDKKVEPKSCDKTHTCPPCPAPELLGGPSVFLFPPKPKDTLMISRTPEVTCV | |
| | VVDVSHEDPEVKFNWYVDGVEVHNAKTKPREEQYNSTYRVVSVLTVLHQD | |
| | WLNGKEYKCKVSNKALPAPIEKTISKAKGQPREPQVYTLPPSRDELTKNQVSL | |
| | WCLVKGFYPSDIAVEWESYGTEWVNYKTTPPVLDSDGSFFLYSKLTVTKEEW | |
| | QQGFVFSCSVLHEALHSHYTQKSLSLSPGK | |
| 269 | QVQLQQPGAELVRPGASVKLSCKASGYSFTGYWMNWLKQRPGQGLEWIGMI | Anti-HER2_DI fused to |
| | HPSDSEIRANQKFRDKATLTVDKSSTTAYMQLSSPTSEDSAVYYCARGTYDG | CH3C.35.23.3 with |
| | GFEYWGQGTTLTVSSASTKGPSVFPLAPSSKSTSGGTAALGCLVKDYFPEPVT | knob, LALA, and |
| | VSWNSGALTSGVHTFPAVLQSSGLYSLSSVVTVPSSSLGTQTYICNVNHKPSN | M428L and N434S |
| | TKVDKKVEPKSCDKTHTCPPCPAPEAAGGPSVFLFPPKPKDTLMISRTPEVTC | mutations |
| | VVVDVSHEDPEVKFNWYVDGVEVHNAKTKPREEQYNSTYRVVSVLTVLHQ | |
| | DWLNGKEYKCKVSNKALPAPIEKTISKAKGQPREPQVYTLPPSRDELTKNQVS | |
| | LWCLVKGFYPSDIAVEWESYGTEWVNYKTTPPVLDSDGSFFLYSKLTVTKEE | |
| | WQQGFVFSCSVLHEALHSHYTQKSLSLSPGK | |
| 270 | QVQLQQPGAELVRPGASVKLSCKASGYSFTGYWMNWLKQRPGQGLEWIGMI | Anti-HER2_DI fused to |
| | HPSDSEIRANQKFRDKATLTVDKSSTTAYMQLSSPTSEDSAVYYCARGTYDG | CH3C.35.23.3 with |
| | GFEYWGQGTTLTVSSASTKGPSVFPLAPSSKSTSGGTAALGCLVKDYFPEPVT | hole mutations |
| | VSWNSGALTSGVHTFPAVLQSSGLYSLSSVVTVPSSSLGTQTYICNVNHKPSN | |
| | TKVDKKVEPKSCDKTHTCPPCPAPELLGGPSVFLFPPKPKDTLMISRTPEVTCV | |
| | VVDVSHEDPEVKFNWYVDGVEVHNAKTKPREEQYNSTYRVVSVLTVLHQD | |
| | WLNGKEYKCKVSNKALPAPIEKTISKAKGQPREPQVYTLPPSRDELTKNQVSL | |
| | SCAVKGFYPSDIAVEWESYGTEWVNYKTTPPVLDSDGSFFLVSKLTVTKEEW | |
| | QQGFVFSCSVMHEALHNHYTQKSLSLSPGK | |
| 271 | QVQLQQPGAELVRPGASVKLSCKASGYSFTGYWMNWLKQRPGQGLEWIGMI | Anti-HER2_DI fused to |
| | HPSDSEIRANQKFRDKATLTVDKSSTTAYMQLSSPTSEDSAVYYCARGTYDG | CH3C.35.23.3 with |
| | GFEYWGQGTTLTVSSASTKGPSVFPLAPSSKSTSGGTAALGCLVKDYFPEPVT | hole and LALA |
| | VSWNSGALTSGVHTFPAVLQSSGLYSLSSVVTVPSSSLGTQTYICNVNHKPSN | mutations |
| | TKVDKKVEPKSCDKTHTCPPCPAPEAAGGPSVFLFPPKPKDTLMISRTPEVTC | |
| | VVVDVSHEDPEVKFNWYVDGVEVHNAKTKPREEQYNSTYRVVSVLTVLHQ | |
| | DWLNGKEYKCKVSNKALPAPIEKTISKAKGQPREPQVYTLPPSRDELTKNQVS | |
| | LSCAVKGFYPSDIAVEWESYGTEWVNYKTTPPVLDSDGSFFLVSKLTVTKEE | |
| | WQQGFVFSCSVMHEALHNHYTQKSLSLSPGK | |
| 272 | QVQLQQPGAELVRPGASVKLSCKASGYSFTGYWMNWLKQRPGQGLEWIGMI | Anti-HER2_DI fused to |
| | HPSDSEIRANQKFRDKATLTVDKSSTTAYMQLSSPTSEDSAVYYCARGTYDG | CH3C.35.23.3 with |
| | GFEYWGQGTTLTVSSASTKGPSVFPLAPSSKSTSGGTAALGCLVKDYFPEPVT | hole and M428L and |
| | VSWNSGALTSGVHTFPAVLQSSGLYSLSSVVTVPSSSLGTQTYICNVNHKPSN | N434S mutations |
| | TKVDKKVEPKSCDKTHTCPPCPAPELLGGPSVFLFPPKPKDTLMISRTPEVTCV | |
| | VVDVSHEDPEVKFNWYVDGVEVHNAKTKPREEQYNSTYRVVSVLTVLHQD | |
| | WLNGKEYKCKVSNKALPAPIEKTISKAKGQPREPQVYTLPPSRDELTKNQVSL | |
| | SCAVKGFYPSDIAVEWESYGTEWVNYKTTPPVLDSDGSFFLVSKLTVTKEEW | |
| | QQGFVFSCSVLHEALHSHYTQKSLSLSPGK | |
| 273 | QVQLQQPGAELVRPGASVKLSCKASGYSFTGYWMNWLKQRPGQGLEWIGMI | Anti-HER2_DI fused to |
| | HPSDSEIRANQKFRDKATLTVDKSSTTAYMQLSSPTSEDSAVYYCARGTYDG | CH3C.35.23.3 with |
| | GFEYWGQGTTLTVSSASTKGPSVFPLAPSSKSTSGGTAALGCLVKDYFPEPVT | hole, LALA, and |
| | VSWNSGALTSGVHTFPAVLQSSGLYSLSSVVTVPSSSLGTQTYICNVNHKPSN | M428L and N434S |
| | TKVDKKVEPKSCDKTHTCPPCPAPEAAGGPSVFLFPPKPKDTLMISRTPEVTC | mutations |
| | VVVDVSHEDPEVKFNWYVDGVEVHNAKTKPREEQYNSTYRVVSVLTVLHQ | |
| | DWLNGKEYKCKVSNKALPAPIEKTISKAKGQPREPQVYTLPPSRDELTKNQVS | |
| | LSCAVKGFYPSDIAVEWESYGTEWVNYKTTPPVLDSDGSFFLVSKLTVTKEE | |
| | WQQGFVFSCSVLHEALHSHYTQKSLSLSPGK | |
| 274 | QVQLQQPGAELVRPGASVKLSCKASGYSFTGYWMNWLKQRPGQGLEWIGMI | Anti-HER2_DI fused to |
| | HPSDSEIRANQKFRDKATLTVDKSSTTAYMQLSSPTSEDSAVYYCARGTYDG | CH3C.35.23.4 with |
| | GFEYWGQGTTLTVSSASTKGPSVFPLAPSSKSTSGGTAALGCLVKDYFPEPVT | knob mutation |
| | VSWNSGALTSGVHTFPAVLQSSGLYSLSSVVTVPSSSLGTQTYICNVNHKPSN | |
| | TKVDKKVEPKSCDKTHTCPPCPAPELLGGPSVFLFPPKPKDTLMISRTPEVTCV | |
| | VVDVSHEDPEVKFNWYVDGVEVHNAKTKPREEQYNSTYRVVSVLTVLHQD | |
| | WLNGKEYKCKVSNKALPAPIEKTISKAKGQPREPQVYTLPPSRDELTKNQVSL | |
| | WCLVKGFYPSDIAVEWESYGTEWSNYKTTPPVLDSDGSFFLYSKLTVSKEEW | |
| | QQGFVFSCSVMHEALHNHYTQKSLSLSPGK | |
| 275 | QVQLQQPGAELVRPGASVKLSCKASGYSFTGYWMNWLKQRPGQGLEWIGMI | Anti-HER2_DI fused to |
| | HPSDSEIRANQKFRDKATLTVDKSSTTAYMQLSSPTSEDSAVYYCARGTYDG | CH3C.35.23.4 with |
| | GFEYWGQGTTLTVSSASTKGPSVFPLAPSSKSTSGGTAALGCLVKDYFPEPVT | knob and LALA |
| | VSWNSGALTSGVHTFPAVLQSSGLYSLSSVVTVPSSSLGTQTYICNVNHKPSN | mutations |
| | TKVDKKVEPKSCDKTHTCPPCPAPEAAGGPSVFLFPPKPKDTLMISRTPEVTC | |
| | VVVDVSHEDPEVKFNWYVDGVEVHNAKTKPREEQYNSTYRVVSVLTVLHQ | |
| | DWLNGKEYKCKVSNKALPAPIEKTISKAKGQPREPQVYTLPPSRDELTKNQVS | |
| | LWCLVKGFYPSDIAVEWESYGTEWSNYKTTPPVLDSDGSFFLYSKLTVSKEE | |
| | WQQGFVFSCSVMHEALHNHYTQKSLSLSPGK | |
| 276 | QVQLQQPGAELVRPGASVKLSCKASGYSFTGYWMNWLKQRPGQGLEWIGMI | Anti-HER2_DI fused to |
| | HPSDSEIRANQKFRDKATLTVDKSSTTAYMQLSSPTSEDSAVYYCARGTYDG | CH3C.35.23.4 with |
| | GFEYWGQGTTLTVSSASTKGPSVFPLAPSSKSTSGGTAALGCLVKDYFPEPVT | knob and M428L and |
| | VSWNSGALTSGVHTFPAVLQSSGLYSLSSVVTVPSSSLGTQTYICNVNHKPSN | N434S mutations |
| | TKVDKKVEPKSCDKTHTCPPCPAPELLGGPSVFLFPPKPKDTLMISRTPEVTCV | |
| | VVDVSHEDPEVKFNWYVDGVEVHNAKTKPREEQYNSTYRVVSVLTVLHQD | |
| | WLNGKEYKCKVSNKALPAPIEKTISKAKGQPREPQVYTLPPSRDELTKNQVSL | |
| | WCLVKGFYPSDIAVEWESYGTEWSNYKTTPPVLDSDGSFFLYSKLTVSKEEW | |
| | QQGFVFSCSVLHEALHSHYTQKSLSLSPGK | |
| 277 | QVQLQQPGAELVRPGASVKLSCKASGYSFTGYWMNWLKQRPGQGLEWIGMI | Anti-HER2_DI fused to |
| | HPSDSEIRANQKFRDKATLTVDKSSTTAYMQLSSPTSEDSAVYYCARGTYDG | CH3C.35.23.4 with |
| | GFEYWGQGTTLTVSSASTKGPSVFPLAPSSKSTSGGTAALGCLVKDYFPEPVT | knob, LALA, and |
| | VSWNSGALTSGVHTFPAVLQSSGLYSLSSVVTVPSSSLGTQTYICNVNHKPSN | M428L and N434S |
| | TKVDKKVEPKSCDKTHTCPPCPAPEAAGGPSVFLFPPKPKDTLMISRTPEVTC | mutations |
| | VVVDVSHEDPEVKFNWYVDGVEVHNAKTKPREEQYNSTYRVVSVLTVLHQ | |
| | DWLNGKEYKCKVSNKALPAPIEKTISKAKGQPREPQVYTLPPSRDELTKNQVS | |
| | LWCLVKGFYPSDIAVEWESYGTEWSNYKTTPPVLDSDGSFFLYSKLTVSKEE | |
| | WQQGFVFSCSVLHEALHSHYTQKSLSLSPGK | |
| 278 | QVQLQQPGAELVRPGASVKLSCKASGYSFTGYWMNWLKQRPGQGLEWIGMI | Anti-HER2_DI fused to |
| | HPSDSEIRANQKFRDKATLTVDKSSTTAYMQLSSPTSEDSAVYYCARGTYDG | CH3C.35.23.4 with |
| | GFEYWGQGTTLTVSSASTKGPSVFPLAPSSKSTSGGTAALGCLVKDYFPEPVT | hole mutations |
| | VSWNSGALTSGVHTFPAVLQSSGLYSLSSVVTVPSSSLGTQTYICNVNHKPSN | |
| | TKVDKKVEPKSCDKTHTCPPCPAPELLGGPSVFLFPPKPKDTLMISRTPEVTCV | |
| | VVDVSHEDPEVKFNWYVDGVEVHNAKTKPREEQYNSTYRVVSVLTVLHQD | |
| | WLNGKEYKCKVSNKALPAPIEKTISKAKGQPREPQVYTLPPSRDELTKNQVSL | |
| | SCAVKGFYPSDIAVEWESYGTEWSNYKTTPPVLDSDGSFFLVSKLTVSKEEW | |
| | QQGFVFSCSVMHEALHNHYTQKSLSLSPGK | |
| 279 | QVQLQQPGAELVRPGASVKLSCKASGYSFTGYWMNWLKQRPGQGLEWIGMI | Anti-HER2_DI fused to |
| | HPSDSEIRANQKFRDKATLTVDKSSTTAYMQLSSPTSEDSAVYYCARGTYDG | CH3C.35.23.4 with |
| | GFEYWGQGTTLTVSSASTKGPSVFPLAPSSKSTSGGTAALGCLVKDYFPEPVT | hole and LALA |
| | VSWNSGALTSGVHTFPAVLQSSGLYSLSSVVTVPSSSLGTQTYICNVNHKPSN | mutations |
| | TKVDKKVEPKSCDKTHTCPPCPAPEAAGGPSVFLFPPKPKDTLMISRTPEVTC | |
| | VVVDVSHEDPEVKFNWYVDGVEVHNAKTKPREEQYNSTYRVVSVLTVLHQ | |
| | DWLNGKEYKCKVSNKALPAPIEKTISKAKGQPREPQVYTLPPSRDELTKNQVS | |
| | LSCAVKGFYPSDIAVEWESYGTEWSNYKTTPPVLDSDGSFFLVSKLTVSKEE | |
| | WQQGFVFSCSVMHEALHNHYTQKSLSLSPGK | |
| 280 | QVQLQQPGAELVRPGASVKLSCKASGYSFTGYWMNWLKQRPGQGLEWIGMI | Anti-HER2_DI fused to |
| | HPSDSEIRANQKFRDKATLTVDKSSTTAYMQLSSPTSEDSAVYYCARGTYDG | CH3C.35.23.4 with |
| | GFEYWGQGTTLTVSSASTKGPSVFPLAPSSKSTSGGTAALGCLVKDYFPEPVT | hole and M428L and |
| | VSWNSGALTSGVHTFPAVLQSSGLYSLSSVVTVPSSSLGTQTYICNVNHKPSN | N434S mutations |
| | TKVDKKVEPKSCDKTHTCPPCPAPELLGGPSVFLFPPKPKDTLMISRTPEVTCV | |
| | VVDVSHEDPEVKFNWYVDGVEVHNAKTKPREEQYNSTYRVVSVLTVLHQD | |
| | WLNGKEYKCKVSNKALPAPIEKTISKAKGQPREPQVYTLPPSRDELTKNQVSL | |
| | SCAVKGFYPSDIAVEWESYGTEWSNYKTTPPVLDSDGSFFLVSKLTVSKEEW | |
| | QQGFVFSCSVLHEALHSHYTQKSLSLSPGK | |
| 281 | QVQLQQPGAELVRPGASVKLSCKASGYSFTGYWMNWLKQRPGQGLEWIGMI | Anti-HER2_DI fused to |
| | HPSDSEIRANQKFRDKATLTVDKSSTTAYMQLSSPTSEDSAVYYCARGTYDG | CH3C.35.23.4 with |
| | GFEYWGQGTTLTVSSASTKGPSVFPLAPSSKSTSGGTAALGCLVKDYFPEPVT | hole, LALA, and |
| | VSWNSGALTSGVHTFPAVLQSSGLYSLSSVVTVPSSSLGTQTYICNVNHKPSN | M428L and N434S |
| | TKVDKKVEPKSCDKTHTCPPCPAPEAAGGPSVFLFPPKPKDTLMISRTPEVTC | mutations |
| | VVVDVSHEDPEVKFNWYVDGVEVHNAKTKPREEQYNSTYRVVSVLTVLHQ | |
| | DWLNGKEYKCKVSNKALPAPIEKTISKAKGQPREPQVYTLPPSRDELTKNQVS | |
| | LSCAVKGFYPSDIAVEWESYGTEWSNYKTTPPVLDSDGSFFLVSKLTVSKEE | |
| | WQQGFVFSCSVLHEALHSHYTQKSLSLSPGK | |
| 282 | QVQLQQPGAELVRPGASVKLSCKASGYSFTGYWMNWLKQRPGQGLEWIGMI | Anti-HER2_DI fused to |
| | HPSDSEIRANQKFRDKATLTVDKSSTTAYMQLSSPTSEDSAVYYCARGTYDG | CH3C.35.23 with knob |
| | GFEYWGQGTTLTVSSASTKGPSVFPLAPSSKSTSGGTAALGCLVKDYFPEPVT | mutation |
| | VSWNSGALTSGVHTFPAVLQSSGLYSLSSVVTVPSSSLGTQTYICNVNHKPSN | |
| | TKVDKKVEPKSCDKTHTCPPCPAPELLGGPSVFLFPPKPKDTLMISRTPEVTCV | |
| | VVDVSHEDPEVKFNWYVDGVEVHNAKTKPREEQYNSTYRVVSVLTVLHQD | |
| | WLNGKEYKCKVSNKALPAPIEKTISKAKGQPREPQVYTLPPSRDELTKNQVSL | |
| | WCLVKGFYPSDIAVEWESYGTEWSNYKTTPPVLDSDGSFFLYSKLTVTKEEW | |
| | QQGFVFSCSVMHEALHNHYTQKSLSLSPGK | |
| 283 | QVQLQQPGAELVRPGASVKLSCKASGYSFTGYWMNWLKQRPGQGLEWIGMI | Anti-HER2_DI fused to |
| | HPSDSEIRANQKFRDKATLTVDKSSTTAYMQLSSPTSEDSAVYYCARGTYDG | CH3C.35.23 with knob |
| | GFEYWGQGTTLTVSSASTKGPSVFPLAPSSKSTSGGTAALGCLVKDYFPEPVT | and LALA mutations |
| | VSWNSGALTSGVHTFPAVLQSSGLYSLSSVVTVPSSSLGTQTYICNVNHKPSN | |
| | TKVDKKVEPKSCDKTHTCPPCPAPEAAGGPSVFLFPPKPKDTLMISRTPEVTC | |
| | VVVDVSHEDPEVKFNWYVDGVEVHNAKTKPREEQYNSTYRVVSVLTVLHQ | |
| | DWLNGKEYKCKVSNKALPAPIEKTISKAKGQPREPQVYTLPPSRDELTKNQVS | |
| | LWCLVKGFYPSDIAVEWESYGTEWSNYKTTPPVLDSDGSFFLYSKLTVTKEE | |
| | WQQGFVFSCSVMHEALHNHYTQKSLSLSPGK | |
| 284 | QVQLQQPGAELVRPGASVKLSCKASGYSFTGYWMNWLKQRPGQGLEWIGMI | Anti-HER2_DI fused to |
| | HPSDSEIRANQKFRDKATLTVDKSSTTAYMQLSSPTSEDSAVYYCARGTYDG | CH3C.35.23 with knob |
| | GFEYWGQGTTLTVSSASTKGPSVFPLAPSSKSTSGGTAALGCLVKDYFPEPVT | and M428L and N434S |
| | VSWNSGALTSGVHTFPAVLQSSGLYSLSSVVTVPSSSLGTQTYICNVNHKPSN | mutations |
| | TKVDKKVEPKSCDKTHTCPPCPAPELLGGPSVFLFPPKPKDTLMISRTPEVTCV | |
| | VVDVSHEDPEVKFNWYVDGVEVHNAKTKPREEQYNSTYRVVSVLTVLHQD | |
| | WLNGKEYKCKVSNKALPAPIEKTISKAKGQPREPQVYTLPPSRDELTKNQVSL | |
| | WCLVKGFYPSDIAVEWESYGTEWSNYKTTPPVLDSDGSFFLYSKLTVTKEEW | |
| | QQGFVFSCSVLHEALHSHYTQKSLSLSPGK | |
| 285 | QVQLQQPGAELVRPGASVKLSCKASGYSFTGYWMNWLKQRPGQGLEWIGMI | Anti-HER2_DI fused to |
| | HPSDSEIRANQKFRDKATLTVDKSSTTAYMQLSSPTSEDSAVYYCARGTYDG | CH3C.35.23 with knob, |
| | GFEYWGQGTTLTVSSASTKGPSVFPLAPSSKSTSGGTAALGCLVKDYFPEPVT | LALA, and M428L and |
| | VSWNSGALTSGVHTFPAVLQSSGLYSLSSVVTVPSSSLGTQTYICNVNHKPSN | N434S mutations |
| | TKVDKKVEPKSCDKTHTCPPCPAPEAAGGPSVFLFPPKPKDTLMISRTPEVTC | |
| | VVVDVSHEDPEVKFNWYVDGVEVHNAKTKPREEQYNSTYRVVSVLTVLHQ | |
| | DWLNGKEYKCKVSNKALPAPIEKTISKAKGQPREPQVYTLPPSRDELTKNQVS | |
| | LWCLVKGFYPSDIAVEWESYGTEWSNYKTTPPVLDSDGSFFLYSKLTVTKEE | |
| | WQQGFVFSCSVLHEALHSHYTQKSLSLSPGK | |
| 286 | QVQLQQPGAELVRPGASVKLSCKASGYSFTGYWMNWLKQRPGQGLEWIGMI | Anti-HER2_DI fused to |
| | HPSDSEIRANQKFRDKATLTVDKSSTTAYMQLSSPTSEDSAVYYCARGTYDG | CH3C.35.23 with hole |
| | GFEYWGQGTTLTVSSASTKGPSVFPLAPSSKSTSGGTAALGCLVKDYFPEPVT | mutations |
| | VSWNSGALTSGVHTFPAVLQSSGLYSLSSVVTVPSSSLGTQTYICNVNHKPSN | |
| | TKVDKKVEPKSCDKTHTCPPCPAPELLGGPSVFLFPPKPKDTLMISRTPEVTCV | |
| | VVDVSHEDPEVKFNWYVDGVEVHNAKTKPREEQYNSTYRVVSVLTVLHQD | |
| | WLNGKEYKCKVSNKALPAPIEKTISKAKGQPREPQVYTLPPSRDELTKNQVSL | |
| | SCAVKGFYPSDIAVEWESYGTEWSNYKTTPPVLDSDGSFFLVSKLTVTKEEW | |
| | QQGFVFSCSVMHEALHNHYTQKSLSLSPGK | |
| 287 | QVQLQQPGAELVRPGASVKLSCKASGYSFTGYWMNWLKQRPGQGLEWIGMI | Anti-HER2_DI fused to |
| | HPSDSEIRANQKFRDKATLTVDKSSTTAYMQLSSPTSEDSAVYYCARGTYDG | CH3C.35.23 with hole |
| | GFEYWGQGTTLTVSSASTKGPSVFPLAPSSKSTSGGTAALGCLVKDYFPEPVT | and LALA mutations |
| | VSWNSGALTSGVHTFPAVLQSSGLYSLSSVVTVPSSSLGTQTYICNVNHKPSN | |
| | TKVDKKVEPKSCDKTHTCPPCPAPEAAGGPSVFLFPPKPKDTLMISRTPEVTC | |
| | VVVDVSHEDPEVKFNWYVDGVEVHNAKTKPREEQYNSTYRVVSVLTVLHQ | |
| | DWLNGKEYKCKVSNKALPAPIEKTISKAKGQPREPQVYTLPPSRDELTKNQVS | |
| | LSCAVKGFYPSDIAVEWESYGTEWSNYKTTPPVLDSDGSFFLVSKLTVTKEE | |
| | WQQGFVFSCSVMHEALHNHYTQKSLSLSPGK | |
| 288 | QVQLQQPGAELVRPGASVKLSCKASGYSFTGYWMNWLKQRPGQGLEWIGMI | Anti-HER2_DI fused to |
| | HPSDSEIRANQKFRDKATLTVDKSSTTAYMQLSSPTSEDSAVYYCARGTYDG | CH3C.35.23 with hole |
| | GFEYWGQGTTLTVSSASTKGPSVFPLAPSSKSTSGGTAALGCLVKDYFPEPVT | and M428L and N434S |
| | VSWNSGALTSGVHTFPAVLQSSGLYSLSSVVTVPSSSLGTQTYICNVNHKPSN | mutations |
| | TKVDKKVEPKSCDKTHTCPPCPAPELLGGPSVFLFPPKPKDTLMISRTPEVTCV | |
| | VVDVSHEDPEVKFNWYVDGVEVHNAKTKPREEQYNSTYRVVSVLTVLHQD | |
| | WLNGKEYKCKVSNKALPAPIEKTISKAKGQPREPQVYTLPPSRDELTKNQVSL | |
| | SCAVKGFYPSDIAVEWESYGTEWSNYKTTPPVLDSDGSFFLVSKLTVTKEEW | |
| | QQGFVFSCSVLHEALHSHYTQKSLSLSPGK | |
| 289 | QVQLQQPGAELVRPGASVKLSCKASGYSFTGYWMNWLKQRPGQGLEWIGMI | Anti-HER2_DI fused to |
| | HPSDSEIRANQKFRDKATLTVDKSSTTAYMQLSSPTSEDSAVYYCARGTYDG | CH3C.35.23 with hole, |
| | GFEYWGQGTTLTVSSASTKGPSVFPLAPSSKSTSGGTAALGCLVKDYFPEPVT | LALA, and M428L and |
| | VSWNSGALTSGVHTFPAVLQSSGLYSLSSVVTVPSSSLGTQTYICNVNHKPSN | N434S mutations |
| | TKVDKKVEPKSCDKTHTCPPCPAPEAAGGPSVFLFPPKPKDTLMISRTPEVTC | |
| | VVVDVSHEDPEVKFNWYVDGVEVHNAKTKPREEQYNSTYRVVSVLTVLHQ | |
| | DWLNGKEYKCKVSNKALPAPIEKTISKAKGQPREPQVYTLPPSRDELTKNQVS | |
| | LSCAVKGFYPSDIAVEWESYGTEWSNYKTTPPVLDSDGSFFLVSKLTVTKEE | |
| | WQQGFVFSCSVLHEALHSHYTQKSLSLSPGK | |
| 290 | QVQLQQPGAELVRPGASVKLSCKASGYSFTGYWMNWLKQRPGQGLEWIGMI | Anti-HER2_DI fused to |
| | HPSDSEIRANQKFRDKATLTVDKSSTTAYMQLSSPTSEDSAVYYCARGTYDG | Fc with hole mutations |
| | GFEYWGQGTTLTVSSASTKGPSVFPLAPSSKSTSGGTAALGCLVKDYFPEPVT | |
| | VSWNSGALTSGVHTFPAVLQSSGLYSLSSVVTVPSSSLGTQTYICNVNHKPSN | |
| | TKVDKKVEPKSCDKTHTCPPCPAPELLGGPSVFLFPPKPKDTLMISRTPEVTCV | |
| | VVDVSHEDPEVKFNWYVDGVEVHNAKTKPREEQYNSTYRVVSVLTVLHQD | |
| | WLNGKEYKCKVSNKALPAPIEKTISKAKGQPREPQVYTLPPSRDELTKNQVSL | |
| | SCAVKGFYPSDIAVEWESNGQPENNYKTTPPVLDSDGSFFLVSKLTVDKSRW | |
| | QQGNVFSCSVMHEALHNHYTQKSLSLSPGK | |
| 291 | QVQLQQPGAELVRPGASVKLSCKASGYSFTGYWMNWLKQRPGQGLEWIGMI | Anti-HER2_DI fused to |
| | HPSDSEIRANQKFRDKATLTVDKSSTTAYMQLSSPTSEDSAVYYCARGTYDG | Fc with hole and |
| | GFEYWGQGTTLTVSSASTKGPSVFPLAPSSKSTSGGTAALGCLVKDYFPEPVT | M428L and N434S |
| | VSWNSGALTSGVHTFPAVLQSSGLYSLSSVVTVPSSSLGTQTYICNVNHKPSN | mutations |
| | TKVDKKVEPKSCDKTHTCPPCPAPELLGGPSVFLFPPKPKDTLMISRTPEVTCV | |
| | VVDVSHEDPEVKFNWYVDGVEVHNAKTKPREEQYNSTYRVVSVLTVLHQD | |
| | WLNGKEYKCKVSNKALPAPIEKTISKAKGQPREPQVYTLPPSRDELTKNQVSL | |
| | SCAVKGFYPSDIAVEWESNGQPENNYKTTPPVLDSDGSFFLVSKLTVDKSRW | |
| | QQGNVFSCSVLHEALHSHYTQKSLSLSPGK | |
| 292 | QVQLQQPGAELVRPGASVKLSCKASGYSFTGYWMNWLKQRPGQGLEWIGMI | Anti-HER2_DI fused to |
| | HPSDSEIRANQKFRDKATLTVDKSSTTAYMQLSSPTSEDSAVYYCARGTYDG | Fc with hole, LALA, |
| | GFEYWGQGTTLTVSSASTKGPSVFPLAPSSKSTSGGTAALGCLVKDYFPEPVT | and M428L and N434S |
| | VSWNSGALTSGVHTFPAVLQSSGLYSLSSVVTVPSSSLGTQTYICNVNHKPSN | mutations |
| | TKVDKKVEPKSCDKTHTCPPCPAPEAAGGPSVFLFPPKPKDTLMISRTPEVTC | |
| | VVVDVSHEDPEVKFNWYVDGVEVHNAKTKPREEQYNSTYRVVSVLTVLHQ | |
| | DWLNGKEYKCKVSNKALPAPIEKTISKAKGQPREPQVYTLPPSRDELTKNQVS | |
| | LSCAVKGFYPSDIAVEWESNGQPENNYKTTPPVLDSDGSFFLVSKLTVDKSR | |
| | WQQGNVFSCSVLHEALHSHYTQKSLSLSPGK | |
| 293 | DIVLTQSPASLVVSLGQRATISCRASQSVSGSRFTYMHWYQQKPGQPPKLLIK | Anti-HER2_DI light |
| | YASILESGVPARFSGGGSGTDFTLNIHPVEEDDTATYYCQHSWEIPPWTFGGG | chain |
| | TKLEIKRTVAAPSVFIFPPSDEQLKSGTASVVCLLNNFYPREAKVQWKVDNAL | |
| | QSGNSQESVTEQDSKDSTYSLSSTLTLSKADYEKHKVYACEVTHQGLSSPVTK | |
| | SFNRGEC | |
| 294 | QVQLQQPGAELVRPGASVKLSCKASGYSFTGYWMNWLKQRPGQGLEWIGMI | Anti-HER2_DI fused to |
| | HPSDSEIRANQKFRDKATLTVDKSSTTAYMQLSSPTSEDSAVYYCARGTYDG | Fc with knob mutation |
| | GFEYWGQGTTLTVSSASTKGPSVFPLAPSSKSTSGGTAALGCLVKDYFPEPVT | |
| | VSWNSGALTSGVHTFPAVLQSSGLYSLSSVVTVPSSSLGTQTYICNVNHKPSN | |
| | TKVDKKVEPKSCDKTHTCPPCPAPELLGGPSVFLFPPKPKDTLMISRTPEVTCV | |
| | VVDVSHEDPEVKFNWYVDGVEVHNAKTKPREEQYNSTYRVVSVLTVLHQD | |
| | WLNGKEYKCKVSNKALPAPIEKTISKAKGQPREPQVYTLPPSRDELTKNQVSL | |
| | WCLVKGFYPSDIAVEWESNGQPENNYKTTPPVLDSDGSFFLYSKLTVDKSRW | |
| | QQGNVFSCSVMHEALHNHYTQKSLSLSPGK | |
| 295 | QVQLQQPGAELVRPGASVKLSCKASGYSFTGYWMNWLKQRPGQGLEWIGMI | Anti-HER2_DI fused to |
| | HPSDSEIRANQKFRDKATLTVDKSSTTAYMQLSSPTSEDSAVYYCARGTYDG | Fc with knob and |
| | GFEYWGQGTTLTVSSASTKGPSVFPLAPSSKSTSGGTAALGCLVKDYFPEPVT | M428L and N434S |
| | VSWNSGALTSGVHTFPAVLQSSGLYSLSSVVTVPSSSLGTQTYICNVNHKPSN | mutations |
| | TKVDKKVEPKSCDKTHTCPPCPAPELLGGPSVFLFPPKPKDTLMISRTPEVTCV | |
| | VVDVSHEDPEVKFNWYVDGVEVHNAKTKPREEQYNSTYRVVSVLTVLHQD | |
| | WLNGKEYKCKVSNKALPAPIEKTISKAKGQPREPQVYTLPPSRDELTKNQVSL | |
| | WCLVKGFYPSDIAVEWESNGQPENNYKTTPPVLDSDGSFFLYSKLTVDKSRW | |
| | QQGNVFSCSVLHEALHSHYTQKSLSLSPGK | |
| 296 | EVQLVESGGGLVQPGGSLRLSCAASGFNIKDTYIHWVRQAPGKGLEWVARIY | Anti-HER2_DIV fused |
| | PTNGYTRYADSVKGRFTISADTSKNTAYLQMNSLRAEDTAVYYCSRWGGDG | to Fc with knob and |
| | FYAMDYWGQGTLVTVSSASTKGPSVFPLAPSSKSTSGGTAALGCLVKDYFPE | LALA mutations |
| | PVTVSWNSGALTSGVHTFPAVLQSSGLYSLSSVVTVPSSSLGTQTYICNVNHK | |
| | PSNTKVDKKVEPKSCDKTHTCPPCPAPEAAGGPSVFLFPPKPKDTLMISRTPEV | |
| | TCVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLWCLVKGFYPSDIAVEWESNGQPENNYKTTPPVLDSDGSFFLYSKLTVDK | |
| | SRWQQGNVFSCSVMHEALHNHYTQKSLSLSPGK | |
| 297 | EVQLVESGGGLVQPGGSLRLSCAASGFTFTDYTMDWVRQAPGKGLEWVAD | Anti-HER2_DII fused |
| | VNPNSGGSIYNQRFKGRFTLSVDRSKNTLYLQMNSLRAEDTAVYYCARNLGP | to Fc with knob and |
| | SFYFDYWGQGTLVTVSSASTKGPSVFPLAPSSKSTSGGTAALGCLVKDYFPEP | LALA mutations |
| | VTVSWNSGALTSGVHTFPAVLQSSGLYSLSSVVTVPSSSLGTQTYICNVNHKP | |
| | SNTKVDKKVEPKSCDKTHTCPPCPAPEAAGGPSVFLFPPKPKDTLMISRTPEV | |
| | TCVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLWCLVKGFYPSDIAVEWESNGQPENNYKTTPPVLDSDGSFFLYSKLTVDK | |
| | SRWQQGNVFSCSVMHEALHNHYTQKSLSLSPGK | |
| 298 | QVQLQQPGAELVRPGASVKLSCKASGYSFTGYWMNWLKQRPGQGLEWIGMI | Anti-HER2_DI fused to |
| | HPSDSEIRANQKFRDKATLTVDKSSTTAYMQLSSPTSEDSAVYYCARGTYDG | Fc with knob and |
| | GFEYWGQGTTLTVSSASTKGPSVFPLAPSSKSTSGGTAALGCLVKDYFPEPVT | LALA mutations |
| | VSWNSGALTSGVHTFPAVLQSSGLYSLSSVVTVPSSSLGTQTYICNVNHKPSN | |
| | TKVDKKVEPKSCDKTHTCPPCPAPEAAGGPSVFLFPPKPKDTLMISRTPEVTC | |
| | VVVDVSHEDPEVKFNWYVDGVEVHNAKTKPREEQYNSTYRVVSVLTVLHQ | |
| | DWLNGKEYKCKVSNKALPAPIEKTISKAKGQPREPQVYTLPPSRDELTKNQVS | |
| | LWCLVKGFYPSDIAVEWESNGQPENNYKTTPPVLDSDGSFFLYSKLTVDKSR | |
| | WQQGNVFSCSVMHEALHNHYTQKSLSLSPGK | |
| 299 | EVQLVESGGGLVQPGGSLRLSCAASGFNIKDTYIHWVRQAPGKGLEWVARIY | Anti-HER2_DIV fused |
| | PTNGYTRYADSVKGRFTISADTSKNTAYLQMNSLRAEDTAVYYCSRWGGDG | to Fc with knob, |
| | FYAMDYWGQGTLVTVSSASTKGPSVFPLAPSSKSTSGGTAALGCLVKDYFPE | LAAL, and M428L and |
| | PVTVSWNSGALTSGVHTFPAVLQSSGLYSLSSVVTVPSSSLGTQTYICNVNHK | N434S mutations |
| | PSNTKVDKKVEPKSCDKTHTCPPCPAPEAAGGPSVFLFPPKPKDTLMISRTPEV | |
| | TCVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLWCLVKGFYPSDIAVEWESNGQPENNYKTTPPVLDSDGSFFLYSKLTVDK | |
| | SRWQQGNVFSCSVLHEALHSHYTQKSLSLSPGK | |
| 300 | EVQLVESGGGLVQPGGSLRLSCAASGFTFTDYTMDWVRQAPGKGLEWVAD | Anti-HER2_DII fused |
| | VNPNSGGSIYNQRFKGRFTLSVDRSKNTLYLQMNSLRAEDTAVYYCARNLGP | to Fc with knob, |
| | SFYFDYWGQGTLVTVSSASTKGPSVFPLAPSSKSTSGGTAALGCLVKDYFPEP | LALA, and M428L and |
| | VTVSWNSGALTSGVHTFPAVLQSSGLYSLSSVVTVPSSSLGTQTYICNVNHKP | N434S mutations |
| | SNTKVDKKVEPKSCDKTHTCPPCPAPEAAGGPSVFLFPPKPKDTLMISRTPEV | |
| | TCVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLWCLVKGFYPSDIAVEWESNGQPENNYKTTPPVLDSDGSFFLYSKLTVDK | |
| | SRWQQGNVFSCSVLHEALHSHYTQKSLSLSPGK | |
| 301 | QVQLQQPGAELVRPGASVKLSCKASGYSFTGYWMNWLKQRPGQGLEWIGMI | Anti-HER2_DI fused to |
| | HPSDSEIRANQKFRDKATLTVDKSSTTAYMQLSSPTSEDSAVYYCARGTYDG | Fc with knob, LALA, |
| | GFEYWGQGTTLTVSSASTKGPSVFPLAPSSKSTSGGTAALGCLVKDYFPEPVT | M428L and N434S and |
| | VSWNSGALTSGVHTFPAVLQSSGLYSLSSVVTVPSSSLGTQTYICNVNHKPSN | mutations |
| | TKVDKKVEPKSCDKTHTCPPCPAPEAAGGPSVFLFPPKPKDTLMISRTPEVTC | |
| | VVVDVSHEDPEVKFNWYVDGVEVHNAKTKPREEQYNSTYRVVSVLTVLHQ | |
| | DWLNGKEYKCKVSNKALPAPIEKTISKAKGQPREPQVYTLPPSRDELTKNQVS | |
| | LWCLVKGFYPSDIAVEWESNGQPENNYKTTPPVLDSDGSFFLYSKLTVDKSR | |
| | WQQGNVFSCSVLHEALHSHYTQKSLSLSPGK | |
| 302 | EVQLVESGGGLVQPGGSLRLSCAASGFNIKDTYIHWVRQAPGKGLEWVARIY | Anti-HER2_DIV fused |
| | PTNGYTRYADSVKGRFTISADTSKNTAYLQMNSLRAEDTAVYYCSRWGGDG | to Fc with hole and |
| | FYAMDYWGQGTLVTVSSASTKGPSVFPLAPSSKSTSGGTAALGCLVKDYFPE | LALA mutations |
| | PVTVSWNSGALTSGVHTFPAVLQSSGLYSLSSVVTVPSSSLGTQTYICNVNHK | |
| | PSNTKVDKKVEPKSCDKTHTCPPCPAPEAAGGPSVFLFPPKPKDTLMISRTPEV | |
| | TCVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLSCAVKGFYPSDIAVEWESNGQPENNYKTTPPVLDSDGSFFLVSKLTVDKS | |
| | RWQQGNVFSCSVMHEALHNHYTQKSLSLSPGK | |
| 303 | EVQLVESGGGLVQPGGSLRLSCAASGFTFTDYTMDWVRQAPGKGLEWVAD | Anti-HER2_DII fused |
| | VNPNSGGSIYNQRFKGRFTLSVDRSKNTLYLQMNSLRAEDTAVYYCARNLGP | to Fc with hole and |
| | SFYFDYWGQGTLVTVSSASTKGPSVFPLAPSSKSTSGGTAALGCLVKDYFPEP | LALA mutations |
| | VTVSWNSGALTSGVHTFPAVLQSSGLYSLSSVVTVPSSSLGTQTYICNVNHKP | |
| | SNTKVDKKVEPKSCDKTHTCPPCPAPEAAGGPSVFLFPPKPKDTLMISRTPEV | |
| | TCVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLSCAVKGFYPSDIAVEWESNGQPENNYKTTPPVLDSDGSFFLVSKLTVDKS | |
| | RWQQGNVFSCSVMHEALHNHYTQKSLSLSPGK | |
| 304 | QVQLQQPGAELVRPGASVKLSCKASGYSFTGYWMNWLKQRPGQGLEWIGMI | Anti-HER2_DI fused to |
| | HPSDSEIRANQKFRDKATLTVDKSSTTAYMQLSSPTSEDSAVYYCARGTYDG | Fc with hole and LALA |
| | GFEYWGQGTTLTVSSASTKGPSVFPLAPSSKSTSGGTAALGCLVKDYFPEPVT | mutations |
| | VSWNSGALTSGVHTFPAVLQSSGLYSLSSVVTVPSSSLGTQTYICNVNHKPSN | |
| | TKVDKKVEPKSCDKTHTCPPCPAPEAAGGPSVFLFPPKPKDTLMISRTPEVTC | |
| | VVVDVSHEDPEVKFNWYVDGVEVHNAKTKPREEQYNSTYRVVSVLTVLHQ | |
| | DWLNGKEYKCKVSNKALPAPIEKTISKAKGQPREPQVYTLPPSRDELTKNQVS | |
| | LSCAVKGFYPSDIAVEWESNGQPENNYKTTPPVLDSDGSFFLVSKLTVDKSR | |
| | WQQGNVFSCSVMHEALHNHYTQKSLSLSPGK | |
| 305 | EVQLVESGGGLVQPGGSLRLSCAASGFNIKDTYIHWVRQAPGKGLEWVARIY | Anti-HER2_DIV fused |
| | PTNGYTRYADSVKGRFTISADTSKNTAYLQMNSLRAEDTAVYYCSRWGGDG | to Fc with hole, LALA, |
| | FYAMDYWGQGTLVTVSSASTKGPSVFPLAPSSKSTSGGTAALGCLVKDYFPE | and M428L and N434S |
| | PVTVSWNSGALTSGVHTFPAVLQSSGLYSLSSVVTVPSSSLGTQTYICNVNHK | mutations |
| | PSNTKVDKKVEPKSCDKTHTCPPCPAPEAAGGPSVFLFPPKPKDTLMISRTPEV | |
| | TCVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLSCAVKGFYPSDIAVEWESNGQPENNYKTTPPVLDSDGSFFLVSKLTVDKS | |
| | RWQQGNVFSCSVLHEALHSHYTQKSLSLSPGK | |
| 306 | EVQLVESGGGLVQPGGSLRLSCAASGFTFTDYTMDWVRQAPGKGLEWVAD | Anti-HER2_DII fused |
| | VNPNSGGSIYNQRFKGRFTLSVDRSKNTLYLQMNSLRAEDTAVYYCARNLGP | to Fc with hole, LALA, |
| | SFYFDYWGQGTLVTVSSASTKGPSVFPLAPSSKSTSGGTAALGCLVKDYFPEP | and M428L and N434S |
| | VTVSWNSGALTSGVHTFPAVLQSSGLYSLSSVVTVPSSSLGTQTYICNVNHKP | mutations |
| | SNTKVDKKVEPKSCDKTHTCPPCPAPEAAGGPSVFLFPPKPKDTLMISRTPEV | |
| | TCVVVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLSCAVKGFYPSDIAVEWESNGQPENNYKTTPPVLDSDGSFFLVSKLTVDKS | |
| | RWQQGNVFSCSVLHEALHSHYTQKSLSLSPGK | |
| 307 | QVQLQQPGAELVRPGASVKLSCKASGYSFTGYWMNWLKQRPGQGLEWIGMI | Anti-HER2_DI HC |
| | HPSDSEIRANQKFRDKATLTVDKSSTTAYMQLSSPTSEDSAVYYCARGTYDG | with wild-type human |
| | GFEYWGQGTTLTVSSASTKGPSVFPLAPSSKSTSGGTAALGCLVKDYFPEPVT | Fc |
| | VSWNSGALTSGVHTFPAVLQSSGLYSLSSVVTVPSSSLGTQTYICNVNHKPSN | |
| | TKVDKKVEPKSCDKTHTCPPCPAPELLGGPSVFLFPPKPKDTLMISRTPEVTCV | |
| | VVDVSHEDPEVKFNWYVDGVEVHNAKTKPREEQYNSTYRVVSVLTVLHQD | |
| | WLNGKEYKCKVSNKALPAPIEKTISKAKGQPREPQVYTLPPSRDELTKNQVSL | |
| | TCLVKGFYPSDIAVEWESNGQPENNYKTTPPVLDSDGSFFLYSKLTVDKSRW | |
| | QQGNVFSCSVMHEALHNHYTQKSLSLSPGK | |
| 308 | EVQLVESGGGLVQPGGSLRLSCAASGFNIKDTYIHWVRQAPGKGLEWVARIY | Fd of Anti-HER2_DIV |
| | PTNGYTRYADSVKGRFTISADTSKNTAYLQMNSLRAEDTAVYYCSRWGGDG | HC |
| | FYAMDYWGQGTLVTVSSASTKGPSVFPLAPSSKSTSGGTAALGCLVKDYFPE | |
| | PVTVSWNSGALTSGVHTFPAVLQSSGLYSLSSVVTVPSSSLGTQTYICNVNHK | |
| | PSNTKVDKKVEPKSCDKTHT | |
| 309 | EVQLVESGGGLVQPGGSLRLSCAASGFTFTDYTMDWVRQAPGKGLEWVAD | Fd of Anti-HER2_DII |
| | VNPNSGGSIYNQRFKGRFTLSVDRSKNTLYLQMNSLRAEDTAVYYCARNLGP | HC |
| | SFYFDYWGQGTLVTVSSASTKGPSVFPLAPSSKSTSGGTAALGCLVKDYFPEP | |
| | VTVSWNSGALTSGVHTFPAVLQSSGLYSLSSVVTVPSSSLGTQTYICNVNHKP | |
| | SNTKVDKKVEPKSCDKTHT | |
| 310 | QVQLQQPGAELVRPGASVKLSCKASGYSFTGYWMNWLKQRPGQGLEWIGMI | Fd of Anti-HER2_DI |
| | HPSDSEIRANQKFRDKATLTVDKSSTTAYMQLSSPTSEDSAVYYCARGTYDG | HC |
| | GFEYWGQGTTLTVSSASTKGPSVFPLAPSSKSTSGGTAALGCLVKDYFPEPVT | |
| | VSWNSGALTSGVHTFPAVLQSSGLYSLSSVVTVPSSSLGTQTYICNVNHKPSN | |
| | TKVDKKVEPKSCDKTHT | |
| 311 | QVQLVESGGGVVQPGRSLRLSCAASGFAFSSYGMHWVRQAPGKGLEWVAVI | Control fused to |
| | WFDGTKKYYTDSVKGRFTISRDNSKNTLYLQMNTLRAEDTAVYYCARDRGI | CH3C.35.23.4 with |
| | GARRGPYYMDVWGKGTTVTVSSASTKGPSVFPLAPSSKSTSGGTAALGCLVK | knob and LALA |
| | DYFPEPVTVSWNSGALTSGVHTFPAVLQSSGLYSLSSVVTVPSSSLGTQTYICN | mutations (ATV:ctrl |
| | VNHKPSNTKVDKKVEPKSCDKTHTCPPCPAPEAAGGPSVFLFPPKPKDTLMIS | HC1) |
| | RTPEVTCVVVDVSHEDPEVKFNWYVDGVEVHNAKTKPREEQYNSTYRVVSV | |
| | LTVLHQDWLNGKEYKCKVSNKALPAPIEKTISKAKGQPREPQVYTLPPSRDEL | |
| | TKNQVSLWCLVKGFYPSDIAVEWESYGTEWSNYKTTPPVLDSDGSFFLYSKL | |
| | TVSKEEWQQGFVFSCSVMHEALHNHYTQKSLSLSPGK | |
| 312 | QVQLVESGGGVVQPGRSLRLSCAASGFAFSSYGMHWVRQAPGKGLEWVAVI | Control fused to Fc |
| | WFDGTKKYYTDSVKGRFTISRDNSKNTLYLQMNTLRAEDTAVYYCARDRGI | with hole mutations |
| | GARRGPYYMDVWGKGTTVTVSSASTKGPSVFPLAPSSKSTSGGTAALGCLVK | (ATV:ctrl HC2) |
| | DYFPEPVTVSWNSGALTSGVHTFPAVLQSSGLYSLSSVVTVPSSSLGTQTYICN | |
| | VNHKPSNTKVDKKVEPKSCDKTHTCPPCPAPELLGGPSVFLFPPKPKDTLMIS | |
| | RTPEVTCVVVDVSHEDPEVKFNWYVDGVEVHNAKTKPREEQYNSTYRVVSV | |
| | LTVLHQDWLNGKEYKCKVSNKALPAPIEKTISKAKGQPREPQVYTLPPSRDEL | |
| | TKNQVSLSCAVKGFYPSDIAVEWESNGQPENNYKTTPPVLDSDGSFFLVSKLT | |
| | VDKSRWQQGNVFSCSVMHEALHNHYTQKSLSLSPGK | |
| 313 | DIQMTQSPSSLSASVGDRVTITCRASQSISSYLNWYQQKPGKAPKLLIYAASSL | Control light chain |
| | QSGVPSRFSGSGSGTDFTLTISSLQPEDFATYYCQQSYSTPLTFGGGTKVEIKR | (ATV:ctrl LC) |
| | TVAAPSVFIFPPSDEQLKSGTASVVCLLNNFYPREAKVQWKVDNALQSGNSQ | |
| | ESVTEQDSKDSTYSLSSTLTLSKADYEKHKVYACEVTHQGLSSPVTKSFNRGE | |
| | C | |

1. An Fc polypeptide dimer-antibody variable region fusion protein comprising:
(a) an antibody variable region that is capable of binding human epidermal growth factor receptor 2 (HER2), or an antigen-binding fragment thereof, and
(b) a modified Fc polypeptide dimer comprising a first Fc polypeptide that contains modifications that create a TfR-binding site.

2. The Fc polypeptide dimer-antibody variable region fusion protein of claim 1, wherein the antibody variable region forms part of a Fab domain.

3. The Fc polypeptide dimer-antibody variable region fusion protein of claim 1, wherein the antibody variable region binds to subdomain IV, II, or I of human HER2.

4. The Fc polypeptide dimer-antibody variable region fusion protein of claim 3, wherein the antibody variable region binds to subdomain IV of human HER2 and comprises one or more complementarity determining regions (CDRs) selected from the group consisting of:
(a) a heavy chain CDR1 having at least 90% sequence identity to the amino acid sequence of SEQ ID NO:69 or having up to two amino acid substitutions relative to the amino acid sequence of SEQ ID NO:69;
(b) a heavy chain CDR2 having at least 90% sequence identity to the amino acid sequence of SEQ ID NO:70 or having up to two amino acid substitutions relative to the amino acid sequence of SEQ ID NO:70;
(c) a heavy chain CDR3 having at least 90% sequence identity to the amino acid sequence of SEQ ID NO:71 or having up to two amino acid substitutions relative to the amino acid sequence of SEQ ID NO:71;
(d) a light chain CDR1 having at least 90% sequence identity to the amino acid sequence of SEQ ID NO:72 or having up to two amino acid substitutions relative to the amino acid sequence of SEQ ID NO:72;
(e) a light chain CDR2 having up to two amino acid substitutions relative to the amino acid sequence of SEQ ID NO:73; and
(f) a light chain CDR3 having up to two amino acid substitutions relative to the amino acid sequence of SEQ ID NO:74.

5. (canceled)

6. (canceled)

7. The Fc polypeptide dimer-antibody variable region fusion protein of claim 4, wherein the antibody variable region comprises two antibody heavy chain variable regions comprising the amino acid sequence of SEQ ID NO:59 and two light chain variable regions comprising the amino acid sequence of SEQ ID NO:60.

8. The Fc polypeptide dimer-antibody variable region fusion protein of claim 3, wherein the antibody variable region binds to subdomain II of human HER2 and comprises one or more CDRs selected from the group consisting of:
(a) a heavy chain CDR1 having at least 90% sequence identity to the amino acid sequence of SEQ ID NO:75 or having up to two amino acid substitutions relative to the amino acid sequence of SEQ ID NO:75;
(b) a heavy chain CDR2 having at least 90% sequence identity to the amino acid sequence of SEQ ID NO:76 or having up to two amino acid substitutions relative to the amino acid sequence of SEQ ID NO:76;
(c) a heavy chain CDR3 having at least 90% sequence identity to the amino acid sequence of SEQ ID NO:77 or having up to two amino acid substitutions relative to the amino acid sequence of SEQ ID NO:77;
(d) a light chain CDR1 having at least 90% sequence identity to the amino acid sequence of SEQ ID NO:78 or having up to two amino acid substitutions relative to the amino acid sequence of SEQ ID NO:78;
(e) a light chain CDR2 having up to two amino acid substitutions relative to the amino acid sequence of SEQ ID NO:79; and
(f) a light chain CDR3 having up to two amino acid substitutions relative to the amino acid sequence of SEQ ID NO:80.

9. (canceled)

10. (canceled)

11. The Fc polypeptide dimer-antibody variable region fusion protein of claim 8, wherein the antibody variable region comprises two antibody heavy chain variable regions comprising the amino acid sequence of SEQ ID NO:61 and two light chain variable regions comprising the amino acid sequence of SEQ ID NO:62.

12-15. (canceled)

16. The Fc polypeptide dimer-antibody variable region fusion protein of claim 1, wherein the TfR-binding site is within the CH3 domain, wherein the modified CH3 domain is derived from a human IgG1, IgG2, IgG3, or IgG4 CH3 domain, and wherein the Fc polypeptide dimer-antibody variable region fusion protein binds to the apical domain of TfR.

17. (canceled)

18. The Fc polypeptide dimer-antibody variable region fusion protein of claim 16, wherein the modified CH3 domain comprises one, two, three, four, five, six, seven, eight, nine, ten, or eleven substitutions in a set of amino acid positions comprising 380, 384, 386, 387, 388, 389, 390, 413, 415, 416, and 421, according to EU numbering.

19. The Fc polypeptide dimer-antibody variable region fusion protein of claim 16, wherein the modified CH3 domain comprises Glu, Leu, Ser, Val, Trp, Tyr, or Gln at position 380; Leu, Tyr, Phe, Trp, Met, Pro, or Val at position 384; Leu, Thr, His, Pro, Asn, Val, or Phe at position 386; Val, Pro, Ile, or an acidic amino acid at position 387; Trp at position 388; an aliphatic amino acid, Gly, Ser, Thr, or Asn at position 389; Gly, His, Gln, Leu, Lys, Val, Phe, Ser, Ala, Asp, Glu, Asn, Arg, or Thr at position 390; an acidic amino acid, Ala, Ser, Leu, Thr, Pro, Ile, or His at position 413; Glu, Ser, Asp, Gly, Thr, Pro, Gln, or Arg at position 415; Thr, Arg, Asn, or an acidic amino acid at position 416; and/or an aromatic amino acid, His, or Lys at position 421, according to EU numbering.

20. (canceled)

21. The Fc polypeptide dimer-antibody variable region fusion protein of claim 1, wherein the first Fc polypeptide includes amino acid modifications that reduce FcγR binding when bound to TfR.

22. The Fc polypeptide dimer-antibody variable region fusion protein of claim 21, wherein the amino acid modifications comprise Ala at position 234 and at position 235, according to EU numbering.

23. (canceled)

24. (canceled)

25. The Fc polypeptide dimer-antibody variable region fusion protein of claim 1, wherein the first Fc polypeptide further comprises a knob mutation T366W and a second Fc polypeptide that is present in the Fc polypeptide dimer comprises hole mutations T366S, L368A, and Y407V, according to EU numbering.

26. The Fc polypeptide dimer-antibody variable region fusion protein of claim 25, wherein the first Fc polypeptide comprises the amino acid sequence of SEQ ID NO: 124.

27. The Fc polypeptide dimer-antibody variable region fusion protein of claim 25, wherein the second Fc polypeptide comprises an amino acid sequence selected from SEQ ID NOS:67 and 68.

28. The Fc polypeptide dimer-antibody variable region fusion protein of claim 25 wherein,
the second Fc polypeptide does not contain a TfR-binding site.

29-31. (canceled)

32. The Fc polypeptide dimer-antibody variable region fusion protein of claim 28, wherein the Fc polypeptide dimer-antibody variable region fusion protein comprises a first heavy chain comprising a amino acid sequence selected from SEQ ID NOS:1, 9, 17, and 81.

33. The Fc polypeptide dimer-antibody variable region fusion protein of claim 28, wherein the first Fc polypeptide further comprises amino acid modifications L234A and L235A, according to EU numbering.

34. The Fc polypeptide dimer-antibody variable region fusion protein of claim 33, wherein the Fc polypeptide dimer-antibody variable region fusion protein comprises a first heavy chain comprising an amino acid sequence selected from SEQ ID NOS:2, 10, 18, and 82.

35. The Fc polypeptide dimer-antibody variable region fusion protein of claim 28, wherein the Fc polypeptide dimer-antibody variable region fusion protein comprises a second heavy chain comprising the amino acid sequence of SEQ ID NO:27 and two light chains comprising the amino acid sequence of SEQ ID NO:57.

36. (canceled)

37. The Fc polypeptide dimer-antibody variable region fusion protein of claim 28, wherein the Fc polypeptide dimer-antibody variable region fusion protein comprises a first heavy chain comprising an amino acid sequence selected from SEQ ID NOS:29, 37, 45, and 89.

38. The Fc polypeptide dimer-antibody variable region fusion protein of claim 28, wherein the first Fc polypeptide further comprises amino acid modifications L234A and L235A and comprises an amino acid sequence selected from SEQ ID NOS:30, 38, 46, and 90.

39. (canceled)

40. The Fc polypeptide dimer-antibody variable region fusion protein of claim 28, wherein the Fc polypeptide dimer-antibody variable region fusion protein comprises a second heavy chain comprising the amino acid sequence of SEQ ID NO:55 and two light chains comprising the amino acid sequence of SEQ ID NO:58.

41-103. (canceled)

104. The Fc polypeptide dimer-antibody variable region fusion protein of claim 28, wherein the modified Fc polypeptide dimer does not substantially deplete reticulocytes or an amount of reticulocytes depleted after administering the Fc polypeptide dimer-antibody variable region fusion protein is less than an amount of reticulocytes depleted after administering a control, and wherein the control is a corresponding TfR-binding polypeptide dimer-antibody variable region fusion protein with full effector function and/or contains no mutations that reduce FcγR binding.

105. (canceled)

106. (canceled)

107. An antibody heavy chain comprising:
(a) an anti-human HER2 antibody heavy chain variable region, or a fragment thereof, and
(b) a modified Fc polypeptide that contains modifications that create a TfR-binding site.

108. The antibody heavy chain of claim 107, wherein the modified Fc polypeptide includes one or more amino acid modifications that reduce FcγR binding when bound to TfR.

109. The antibody heavy chain of claim 107, wherein the antibody heavy chain variable region comprises one or more CDRs selected from the group consisting of:
(a) a heavy chain CDR1 having at least 90% sequence identity to the amino acid sequence of SEQ ID NO:69 or having up to two amino acid substitutions relative to the amino acid sequence of SEQ ID NO:69;
(b) a heavy chain CDR2 having at least 90% sequence identity to the amino acid sequence of SEQ ID NO:70 or having up to two amino acid substitutions relative to the amino acid sequence of SEQ ID NO:70; and
(c) a heavy chain CDR3 having at least 90% sequence identity to the amino acid sequence of SEQ ID NO:71 or having up to two amino acid substitutions relative to the amino acid sequence of SEQ ID NO:71.

110. (canceled)

111. The antibody heavy chain of claim 107, wherein the antibody heavy chain variable region comprises the amino acid sequence of SEQ ID NO:59.

112. The antibody heavy chain of claim 107, wherein the antibody variable region comprises one or more CDRs selected from the group consisting of:
(a) a heavy chain CDR1 having at least 90% sequence identity to the amino acid sequence of SEQ ID NO:75 or having up to two amino acid substitutions relative to the amino acid sequence of SEQ ID NO:75;
(b) a heavy chain CDR2 having at least 90% sequence identity to the amino acid sequence of SEQ ID NO:76 or having up to two amino acid substitutions relative to the amino acid sequence of SEQ ID NO:76; and
(c) a heavy chain CDR3 having at least 90% sequence identity to the amino acid sequence of SEQ ID NO:77 or having up to two amino acid substitutions relative to the amino acid sequence of SEQ ID NO:77.

113. (canceled)

114. The antibody heavy chain of claim 107, wherein the antibody heavy chain variable region comprises the amino acid sequence of SEQ ID NO:61.

115-117. (canceled)

118. The antibody heavy chain of claim 107, wherein the TfR-binding site comprises a modified CH3 domain and the modified CH3 domain is derived from a human IgG1, IgG2, IgG3, or IgG4 CH3 domain.

119. (canceled)

120. The antibody heavy chain of claim 118, wherein the modified CH3 domain comprises one, two, three, four, five, six, seven, eight, nine, ten, or eleven substitutions in a set of amino acid positions comprising 380, 384, 386, 387, 388, 389, 390, 413, 415, 416, and 421, according to EU numbering.

121. The antibody heavy chain of claim 118, wherein the modified CH3 domain comprises Glu, Leu, Ser, Val, Trp, Tyr, or Gln at position 380; Leu, Tyr, Phe, Trp, Met, Pro, or Val at position 384; Leu, Thr, His, Pro, Asn, Val, or Phe at position 386; Val, Pro, Ile, or an acidic amino acid at position 387; Trp at position 388; an aliphatic amino acid, Gly, Ser, Thr, or Asn at position 389; Gly, His, Gln, Leu, Lys, Val, Phe, Ser, Ala, Asp, Glu, Asn, Arg, or Thr at position 390; an acidic amino acid, Ala, Ser, Leu, Thr, Pro, Ile, or His at position 413; Glu, Ser, Asp, Gly, Thr, Pro, Gln, or Arg at position 415; Thr, Arg, Asn, or an acidic amino acid at position 416; and/or an aromatic amino acid, His, or Lys at position 421, according to EU numbering.

122. The antibody heavy chain of claim 108, wherein the amino acid modifications that reduce FcγR binding when bound to TfR comprise Ala at position 234 and at position 235, according to EU numbering.

123. (canceled)

124. (canceled)

125. The antibody heavy chain of claim 122, wherein the modified Fc polypeptide further comprises a knob mutation T366W, according to EU numbering.

126. The antibody heavy chain of claim 125, wherein the modified Fc polypeptide comprises the amino acid sequence of SEQ ID NO: 124.

127-130. (canceled)

131. The antibody heavy chain of claim 125, wherein the antibody heavy chain comprises an amino acid sequence selected from SEQ ID NOS:1, 9, 17, and 81 and SEQ ID NOS:29, 37, 45, and 89.

132-134. (canceled)

135. The antibody heavy chain of claim 122, wherein the antibody heavy chain comprises an amino acid sequence selected from SEQ ID NOS:2, 10, 18, and 82 and SEQ ID NOS:30, 38, 46, and 90.

136-148. (canceled)

149. A pharmaceutical composition comprising the Fc polypeptide dimer-antibody variable region fusion protein of claim 1 and a pharmaceutically acceptable carrier.

150. A method of transcytosis of an antibody variable region that is capable of binding human HER2, or an antigen-binding fragment thereof, across an endothelium, the method comprising contacting the endothelium with a composition comprising an Fc polypeptide dimer-antibody variable region fusion protein of claim 1.

151. The method of claim 150, wherein the endothelium is the BBB.

152. A method for treating a cancer in a subject, the method comprising administering to the subject a therapeutically effective amount of a composition comprising an Fc polypeptide dimer-antibody variable region fusion protein of claim 1.

153-164. (canceled)
